(19) United States
(12) Patent Application Publication
(10) Pub. No.: US 2024/0148905 A1
(43) Pub. Date: May 9, 2024

(54) METHODS FOR TREATING SENSORINEURAL HEARING LOSS USING OTOFERLIN DUAL VECTOR SYSTEMS

(71) Applicant: Decibel Therapeutics, Inc., Boston, MA (US)

(72) Inventors: Adam PALERMO, Somerville, MA (US); Ning PAN, Newton, MA (US); Arun SENAPATI, Roslindale, MA (US); Jonathon WHITTON, Cambridge, MA (US); Xichun ZHANG, Belmont, MA (US)

(21) Appl. No.: 18/277,858

(22) PCT Filed: Feb. 18, 2022
(86) PCT No.: PCT/US2022/017058

§ 371 Date: Aug. 18, 2023

Related U.S. Application Data
(60) Provisional application No. 63/151,589, filed on Feb. 19, 2021.

(51) Int. Cl. A61K 48/00; A61P 27/16; C07K 14/705; C12N 15/86

(52) U.S. Cl. CPC A61K 48/0058; A61P 27/16; C07K 14/705; C12N 15/86; C12N 2750/14143

(57) ABSTRACT

The disclosure features compositions and methods for treating subjects 25 years of age or older having biallelic mutations in otoferlin (OTOF) by way of OTOF gene therapy. The disclosure provides a variety of compositions that include a first nucleic acid vector that contains a polynucleotide encoding an N-terminal portion of an OTOF protein and a second nucleic acid vector that contains a polynucleotide encoding a C-terminal portion of an OTOF protein. These vectors can be used to treat hearing loss or auditory neuropathy in a subject having biallelic OTOF mutations.

### SEQUENCE LISTING

The instant application contains a Sequence Listing which has been submitted electronically in ASCII format and is hereby incorporated by reference in its entirety. The ASCII copy, created on Feb. 18, 2022, is named 51471-008WO2_Sequence_Listing_2_17_22_ST25 and is 366,491 bytes in size.

### Field of the Invention

Described herein are compositions and methods for the treatment of sensorineural hearing loss and auditory neuropathy, particularly forms of the disease that are associated with mutations in otoferlin (OTOF) in a human subject 25 years of age or older, by way of OTOF gene therapy. The disclosure provides dual vector systems that include a first nucleic acid vector that contains a polynucleotide encoding an N-terminal portion of an OTOF protein and a second nucleic acid vector that contains a polynucleotide encoding a C-terminal portion of an OTOF protein. These vectors can be used to increase the expression of or provide wild-type OTOF to a subject, such as a human subject suffering from sensorineural hearing loss.

### BACKGROUND

Sensorineural hearing loss is a type of hearing loss caused by defects in the cells of the inner ear or the neural pathways that project from the inner ear to the brain. Although sensorineural hearing loss is often acquired, and can be caused by noise, infections, head trauma, ototoxic drugs, or aging, there are also congenital forms of sensorineural hearing loss associated with autosomal recessive mutations. One such form of autosomal recessive sensorineural hearing loss is associated with mutation of the otoferlin (OTOF) gene, which is implicated in prelingual nonsyndromic hearing loss. In recent years, efforts to treat hearing loss have increasingly focused on gene therapy as a possible solution; however, OTOF is too large to allow for treatment using standard gene therapy approaches. There is a need for new therapeutics to treat OTOF-related sensorineural hearing loss.

### SUMMARY OF THE INVENTION

The present invention provides compositions and methods for treating a human subject 25 years of age or older having biallelic otoferlin (OTOF) mutations, which are known to cause hearing loss and auditory neuropathy. The compositions described herein can be used to deliver wild-type (WT) OTOF to the subject by way of gene therapy, and can, therefore, be used to treat hearing loss and auditory neuropathy in the subject. Gene therapy for treating biallelic OTOF mutations is thought to be needed during the first year of life to restore hearing; however, the present inventors have determined that gene therapy can restore hearing that is lost due to biallelic OTOF mutations even if treatment is begun much later in life. The compositions described herein can also be used to treat a subject having biallelic OTOF mutations that is identified as having detectable otoacoustic emissions, detectable cochlear microphonics, and/or detectable summating potential.

In a first aspect, the invention provides a method of treating a human subject 25 years of age or older having biallelic otoferlin (OTOF) mutations by administering to the subject a therapeutically effective amount of a dual vector system including: a first nucleic acid vector containing a promoter operably linked to a first coding polynucleotide that encodes an N-terminal portion of an OTOF protein; and a second nucleic acid vector containing a second coding polynucleotide that encodes a C-terminal portion of an OTOF protein and a polyadenylation (poly(A)) sequence positioned 3′ of the second coding polynucleotide; in which neither the first nor the second nucleic acid vector encodes a full-length OTOF protein.

In another aspect, the invention provides a method of treating a human subject having biallelic otoferlin (OTOF) mutations and identified as having detectable otoacoustic emissions, detectable cochlear microphonics, and/or detectable summating potential by administering to the subject a therapeutically effective amount of a dual vector system including: a first nucleic acid vector containing a promoter operably linked to a first coding polynucleotide that encodes an N-terminal portion of an OTOF protein; and a second nucleic acid vector containing a second coding polynucleotide that encodes a C-terminal portion of an OTOF protein and a polyadenylation (poly(A)) sequence positioned 3′ of the second coding polynucleotide; in which neither the first nor the second nucleic acid vector encodes a full-length OTOF protein.

In some embodiments of any of the foregoing aspects, the first coding polynucleotide and the second coding polynucleotide do not overlap.

In some embodiments of any of the foregoing aspects, the first nucleic acid vector includes a splice donor signal sequence positioned 3′ of the first coding polynucleotide and the second nucleic acid vector includes a splice acceptor signal sequence positioned 5′ of the second coding polynucleotide. In some embodiments, the first nucleic acid vector includes a first recombinogenic region positioned 3′ of the splice donor signal sequence and the second nucleic acid vector includes a second recombinogenic region positioned 5′ of the splice acceptor signal sequence. In some embodiments, the first and second recombinogenic regions are the same. In some embodiments, the first and/or second recombinogenic region is an AP gene fragment or an F1 phage AK gene. In some embodiments, the F1 phage AK gene includes or has the sequence of SEQ ID NO: 19. In some embodiments, the AP gene fragment includes or has the sequence of any one of SEQ ID NOs: 62-67. In some embodiments, the AP gene fragment includes or has the sequence of SEQ ID NO: 65. In some embodiments, the splice donor sequence includes or has the sequence of SEQ ID NO: 20 or SEQ ID NO: 68. In some embodiments, splice acceptor sequence includes or has the sequence of SEQ ID NO: 21 or SEQ ID NO: 69. In some embodiments, the first nucleic acid vector further includes a degradation signal sequence positioned 3′ of the recombinogenic region, and the second nucleic acid vector further includes a degradation signal sequence positioned between the recombinogenic region and the splice acceptor signal sequence. In some embodiments, the degradation signal sequence includes or has the sequence of SEQ ID NO: 22.

In some embodiments of any of the foregoing aspects, the first and second coding polynucleotides are divided at an OTOF exon boundary. In some embodiments, the OTOF exon boundary is not within a portion of the first coding polynucleotide or the second coding polynucleotide that encodes a C2 domain.

In some embodiments of any of the foregoing aspects, the first coding polynucleotide partially overlaps with the second coding polynucleotide. In some embodiments, the first coding polynucleotide overlaps with the second coding polynucleotide by at least 1 kilobase (kb). In some embodiments, the region of overlap between the first and second coding polynucleotides is centered at an OTOF exon boundary. In some embodiments, the first coding polynucleotide encodes an N-terminal portion of the OTOF protein and includes an OTOF N-terminus to 500 bp 3′ of the exon boundary at the center of the overlap region; and the second coding polynucleotide encodes a C-terminal portion of the OTOF protein and includes 500 bp 5′ of the exon boundary at the center of the overlap region to an OTOF C-terminus. In some embodiments, the OTOF exon boundary at the center of the overlap region is not within a portion of the first coding polynucleotide or second coding polynucleotide that encodes a C2 domain.

In some embodiments of any of the foregoing aspects, the OTOF exon boundary is selected such that the first coding polynucleotide encodes an entire C2C domain and the second coding polynucleotide encodes an entire C2D domain. In some embodiments, the OTOF exon boundary is an exon 19/20 boundary, an exon 20/21 boundary, or an exon 21/22 boundary.

In some embodiments of any of the foregoing aspects, the OTOF exon boundary is selected such that the first coding polynucleotide encodes an entire C2D domain and the second coding polynucleotide encodes an entire C2E domain. In some embodiments, the OTOF exon boundary is an exon 26/27 boundary or an exon 28/29 boundary.

In some embodiments of any of the foregoing aspects, the OTOF exon boundary is within a portion of the first coding polynucleotide and the second coding polynucleotide that encodes a C2D domain. In some embodiments, the OTOF exon boundary is an exon 24/25 boundary or an exon 25/26 boundary.

In some embodiments of any of the foregoing aspects, each of the first and second coding polynucleotides encode about half of the OTOF protein sequence.

In some embodiments of any of the foregoing aspects, the first nucleic acid vector and the second nucleic acid vector do not include OTOF untranslated regions (UTRs).

In some embodiments of any of the foregoing aspects, the first nucleic acid vector includes an OTOF 5′ UTR.

In some embodiments of any of the foregoing aspects, the second nucleic acid vector includes an OTOF 3′ UTR.

In some embodiments of any of the foregoing aspects, the first and second coding polynucleotides that encode the OTOF protein do not include introns.

In some embodiments of any of the foregoing aspects, the first and second coding polynucleotides that encode the OTOF protein do not contain introns.

In some embodiments of any of the foregoing aspects, the OTOF protein is a mammalian OTOF protein.

In some embodiments of any of the foregoing aspects, the OTOF protein is a murine OTOF protein. In some embodiments of any of the foregoing aspects, the murine OTOF protein has at least 85% sequence identity (e.g., 85%, 86%, 87%, 88%, 89%, 90%, 91%, 92%, 93%, 94%, 95%, 96%, 97%, 98%, 99%, or 100% sequence identity) to the sequence of SEQ ID NO: 6, SEQ ID NO: 7, SEQ ID NO: 8, or SEQ ID NO: 9. In some embodiments of any of the foregoing aspects, the OTOF protein comprises or consists of the sequence of SEQ ID NO: 6.

In some embodiments of any of the foregoing aspects, the OTOF protein is a human OTOF protein. In some embodiments of any of the foregoing aspects, the human OTOF protein has at least 85% sequence identity (e.g., 85%, 86%, 87%, 88%, 89%, 90%, 91%, 92%, 93%, 94%, 95%, 96%, 97%, 98%, 99%, or 100% sequence identity) to the sequence of SEQ ID NO: 1, SEQ ID NO: 2, SEQ ID NO:3, SEQ ID NO: 4, or SEQ ID NO: 5. In some embodiments of any of the foregoing aspects, the OTOF protein comprises or consists of the sequence of SEQ ID NO: 1. In some embodiments of any of the foregoing aspects, the OTOF protein comprises or consists of the sequence of SEQ ID NO: 2. In some embodiments of any of the foregoing aspects, the OTOF protein comprises or consists of the sequence of SEQ ID NO: 3. In some embodiments of any of the foregoing aspects, the OTOF protein comprises or consists of the sequence of SEQ ID NO: 4. In some embodiments of any of the foregoing aspects, the OTOF protein comprises or consists of the sequence of SEQ ID NO: 5. In some embodiments of any of the foregoing aspects, the human OTOF protein comprises the sequence of SEQ ID NO: 1, SEQ ID NO: 2, SEQ ID NO:3, SEQ ID NO: 4, or SEQ ID NO: 5 or a variant thereof having one or more e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the OTOF protein variant are conservative amino acid substitutions.

In some embodiments of any of the foregoing aspects, the OTOF protein is encoded by any one of SEQ ID NOs: 10-14. In some embodiments, the OTOF protein is encoded by SEQ ID NO: 10. In some embodiments, the OTOF protein is encoded by SEQ ID NO: 14.

In some embodiments of any of the foregoing aspects, the OTOF protein is encoded by any one of SEQ ID NOs: 15-18.

In some embodiments of any of the foregoing aspects, the first coding polynucleotide encodes amino acids 1-802 of SEQ ID NO: 1 or SEQ ID NO: 5 and the second coding polynucleotide encodes amino acids 803-1997 of SEQ ID NO: 1 or SEQ ID NO: 5. In some embodiments, the first coding polynucleotide encodes amino acids 1-802 of SEQ ID NO: 1 and the second coding polynucleotide encodes amino acids 803-1997 of SEQ ID NO: 1. In some embodiments, the first coding polynucleotide encodes amino acids 1-802 of SEQ ID NO: 5 and the second coding polynucleotide encodes amino acids 803-1997 of SEQ ID NO: 5.

In some embodiments of any of the foregoing aspects, the N-terminal portion of the OTOF protein consists of the sequence of SEQ ID NO: 73 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the N-terminal portion of the OTOF protein variant are conservative amino acid substitutions. In some embodiments, the N-terminal portion of the OTOF protein consists of the sequence of SEQ ID NO: 73. In some embodiments, the N-terminal portion of the OTOF protein is encoded by the sequence of SEQ ID NO: 71.

In some embodiments of any of the foregoing aspects, the C-terminal portion of the OTOF protein consists of the sequence of SEQ ID NO: 74 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the C-terminal portion of the OTOF protein variant are conservative amino acid substitutions. In some embodiments, the C-terminal portion of the OTOF protein consists of the sequence of SEQ ID NO: 74. In some embodiments, the C-terminal portion of the OTOF protein is encoded by the sequence of SEQ ID NO: 72.

In some embodiments of any of the foregoing aspects, the first nucleic acid vector includes a Kozak sequence positioned 3′ of the promoter and 5′ of the first coding polynucleotide that encodes the N-terminal portion of the OTOF protein.

In some embodiments of any of the foregoing aspects, the promoter is a ubiquitous promoter. In some embodiments, the ubiquitous promoter is a CAG promoter, a cytomegalovirus (CMV) promoter, a chicken β-actin promoter, a truncated CMV-chicken β-actin promoter (smCBA), a CB7 promoter, a hybrid CMV enhancer/human β-actin promoter, a human β-actin promoter, an elongation factor-1α (EF1α) promoter, or a phosphoglycerate kinase (PGK) promoter. In some embodiments, the ubiquitous promoter is a CAG promoter. In some embodiments, the ubiquitous promoter is a smCBA promoter. In some embodiments, the smCBA promoter has the sequence of SEQ ID NO: 70.

In some embodiments of any of the foregoing aspects, the promoter is a cochlear hair cell-specific promoter. In some embodiments, the cochlear hair cell-specific promoter is a myosin 15 (Myo15) promoter, a myosin 7A (Myo7A) promoter, a myosin 6 (Myo6) promoter, a POU class 4 homeobox 3 (POU4F3) promoter, an atonal BHLH transcription factor 1 (ATOH1) promoter, a LIM homeobox 3 (LHX3) promoter, an α9 acetylcholine receptor (α9AChR) promoter, or an α10 acetylcholine receptor (α10AChR) promoter. In some embodiments, the cochlear hair cell-specific promoter is a Myo15 promoter.

In some embodiments of any of the foregoing aspects, the promoter is an inner hair cell-specific promoter. In some embodiments, the inner hair cell-specific promoter is a fibroblast growth factor 8 (FGF8) promoter, a vesicular glutamate transporter 3 (VGLUT3) promoter, an OTOF promoter, or a calcium binding protein 2 (CABP2) promoter. In some embodiments, the inner hair cell-specific promoter is a CABP2 promoter.

In some embodiments of any of the foregoing aspects, the promoter is a short promoter (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter). In some embodiments, the short promoter is a CAG promoter. In some embodiments, the short promoter is a CMV promoter. In some embodiments, the short promoter is a smCBA promoter. In some embodiments, the short promoter is a Myo15 promoter that is 1 kb or shorter (e.g., a Myo15 promoter having a sequence with at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 38, 39, or 49-60).

In some embodiments of any of the foregoing aspects, the promoter is a long promoter (e.g., a promoter that is longer than 1 kb, e.g., 1.1 kb, 1.25 kb, 1.5 kb, 1.75 kb, 2 kb, 2.5 kb, 3 kb or longer). In some embodiments, the long promoter is a Myo15 promoter that is longer than 1 kb (e.g., a Myo15 promoter comprising or consisting of a sequence with at least 85% sequence identity (e.g., 85%, 86%, 87%, 88%, 89%, 90%, 91%, 92%, 93%, 94%, 95%, 96%, 97%, 98%, 99%, or more, sequence identity) to the sequence of SEQ ID NO: 36).

In some embodiments of any of the foregoing aspects, the first and second nucleic acid vectors are a pair of nucleic acid vectors listed in Table 4.

In some embodiments of any of the foregoing aspects, the first nucleic acid vector contains a polynucleotide sequence comprising the sequence of nucleotides 2272 to 6041 of SEQ ID NO: 75. In some embodiments of any of the foregoing aspects, the first nucleic acid vector contains a polynucleotide sequence comprising or consisting of the sequence of nucleotides 2049 to 6264 of SEQ ID NO: 75.

In some embodiments of any of the foregoing aspects, the first nucleic acid vector contains a polynucleotide sequence comprising the sequence of nucleotides 182 to 3949 of SEQ ID NO: 77. In some embodiments of any of the foregoing aspects, the first nucleic acid vector contains a polynucleotide sequence comprising or consisting of the sequence of nucleotides 19 to 4115 of SEQ ID NO: 77.

In some embodiments of any of the foregoing aspects, the first nucleic acid vector contains a polynucleotide sequence comprising the sequence of nucleotides 2267 to 6014 of SEQ ID NO: 79. In some embodiments of any of the foregoing aspects, the first nucleic acid vector contains a polynucleotide sequence comprising or consisting of the sequence of nucleotides 2049 to 6237 of SEQ ID NO: 79.

In some embodiments of any of the foregoing aspects, the first nucleic acid vector contains a polynucleotide sequence comprising the sequence of nucleotides 177 to 3924 of SEQ ID NO: 80. In some embodiments of any of the foregoing aspects, the first nucleic acid vector contains a polynucleotide sequence comprising or consisting of the sequence of nucleotides 19 to 4090 of SEQ ID NO: 80.

In some embodiments of any of the foregoing aspects, the second nucleic acid vector contains a polynucleotide sequence comprising the sequence of nucleotides 2267 to 6476 of SEQ ID NO: 76. In some embodiments of any of the foregoing aspects, the second nucleic acid vector contains a polynucleotide sequence comprising or consisting of the sequence of nucleotides 2049 to 6693 of SEQ ID NO: 76.

In some embodiments of any of the foregoing aspects, the second nucleic acid vector contains a polynucleotide sequence comprising the sequence of nucleotides 187 to 4396 of SEQ ID NO: 78. In some embodiments of any of the foregoing aspects, the second nucleic acid vector contains a polynucleotide sequence comprising or consisting of the sequence of nucleotides 19 to 4589 of SEQ ID NO: 78.

In some embodiments of any of the foregoing aspects, the first nucleic acid vector contains a polynucleotide sequence comprising the sequence of nucleotides 235 to 4004 of SEQ ID NO: 81. In some embodiments of any of the foregoing aspects, the first nucleic acid vector contains a polynucleotide sequence comprising or consisting of the sequence of nucleotides 12 to 4227 of SEQ ID NO: 81.

In some embodiments of any of the foregoing aspects, the first nucleic acid vector contains a polynucleotide sequence comprising the sequence of nucleotides 230 to 3977 of SEQ ID NO: 83. In some embodiments of any of the foregoing aspects, the first nucleic acid vector contains a polynucleotide sequence comprising or consisting of the sequence of nucleotides 12 to 4200 of SEQ ID NO: 83.

In some embodiments of any of the foregoing aspects, the second nucleic acid vector contains a polynucleotide sequence comprising the sequence of nucleotides 229 to 4438 of SEQ ID NO: 72. In some embodiments of any of the foregoing aspects, the second nucleic acid vector contains a polynucleotide sequence comprising or consisting of the sequence of nucleotides 12 to 4655 of SEQ ID NO: 82.

In some embodiments of any of the foregoing aspects, the first and second nucleic acid vectors comprise an inverted terminal repeat (ITR) at each end of the nucleic acid sequence. In some embodiments, the first vector includes a first inverted terminal repeat (ITR) sequence 5′ of the promoter and a second ITR sequence 3′ of the recombinogenic region, and the second vector includes a first ITR sequence 5′ of the recombinogenic region and a second ITR sequence 3′ of the poly(A) sequence. In some embodiments, the ITRs in the first vector and second vector are AAV2 ITRs. In some embodiments, the ITRs in the first vector and second vector have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.

In some embodiments of any of the foregoing aspects, the poly(A) sequence is a bovine growth hormone (bGH) poly(A) signal sequence.

In some embodiments of any of the foregoing aspects, the second nucleic acid vector includes a Woodchuck Hepatitis Virus Posttranscriptional Regulatory Element (WPRE). In some embodiments, the WPRE comprises or consists of the sequence of SEQ ID NO: 23 or SEQ ID NO: 61.

In some embodiments of any of the foregoing aspects, the nucleic acid vectors are overlapping dual vectors.

In some embodiments of any of the foregoing aspects, the nucleic acid vectors are trans-splicing dual vectors.

In some embodiments of any of the foregoing aspects, the nucleic acid vectors are dual hybrid vectors.

In some embodiments of any of the foregoing aspects, the nucleic acid vectors are adeno-associated virus (AAV) vectors. In some embodiments, the AAV vectors have an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S capsid. In some embodiments, the AAV vectors have an AAV1 capsid. In some embodiments, the AAV vectors have an AAV9 capsid. In some embodiments, the AAV vectors have an AAV6 capsid. In some embodiments, the AAV vectors have an Anc80 capsid. In some embodiments, the AAV vectors have an Anc80L65 capsid. In some embodiments, the AAV vectors have a DJ/9 capsid. In some embodiments, the AAV vectors have a 7m8 capsid. In some embodiments, the AAV vectors have an AAV2 capsid. In some embodiments, the AAV vectors have an AAV2quad(Y-F) capsid. In some embodiments, the AAV vectors have a PHP.B capsid. In some embodiments, the AAV vectors have an AAV8 capsid.

In some embodiments of any of the foregoing aspects, the first and second nucleic acid vectors have the same capsid (e.g., both the first and second nucleic acid vectors are AAV vectors having an AAV1 capsid or an AAV9 capsid). In some embodiments of any of the foregoing aspects, the first and second nucleic acid vectors have different capsids (e.g., the first nucleic acid vector is an AAV having an AAV1 capsid, and the second nucleic acid vector is an AAV having an AAV9 capsid).

In some embodiments of any of the foregoing aspects, the subject is 30 years of age or older.

In some embodiments of any of the foregoing aspects, the subject is 35 years of age or older.

In some embodiments of any of the foregoing aspects, the subject is 40 years of age or older.

In some embodiments of any of the foregoing aspects, the subject is 45 years of age or older.

In some embodiments of any of the foregoing aspects, the subject is no older than 50 years old.

In some embodiments of any of the foregoing aspects, the subject has been identified as having biallelic OTOF mutations.

In some embodiments of any of the foregoing aspects, the method further includes the step of identifying the subject as having biallelic OTOF mutations prior to administering the dual vector system.

In some embodiments of any of the foregoing aspects, the subject is identified as having detectable otoacoustic emissions.

In some embodiments of any of the foregoing aspects, the method further includes the step of identifying the subject as having detectable otoacoustic emissions prior to administering the dual vector system.

In some embodiments of any of the foregoing aspects, the subject is identified as having detectable cochlear microphonics.

In some embodiments of any of the foregoing aspects, the method further includes the step of identifying the subject as having detectable cochlear microphonics prior to administering the dual vector system.

In some embodiments of any of the foregoing aspects, the subject is identified as having a detectable summating potential.

In some embodiments of any of the foregoing aspects, the method further includes the step of identifying the subject as having detectable summating potential prior to administering the dual vector system.

In some embodiments of any of the foregoing aspects, the method further includes the step of evaluating the hearing of the subject prior to administering the dual vector system.

In some embodiments of any of the foregoing aspects, the subject has or is identified as having Deafness, Autosomal Recessive 9 (DFNB9).

In some embodiments of any of the foregoing aspects, the method further includes the step of evaluating the hearing of the subject prior to administering the dual vector system.

In some embodiments of any of the foregoing aspects, the dual vector system is administered locally to the middle or inner ear. In some embodiments, the dual vector system is administered by injection through the round window membrane, injection into a semicircular canal, canalostomy, insertion of a catheter through the round window membrane, transtympanic injection, or intratympanic injection.

In some embodiments of any of the foregoing aspects, the method further includes the step of evaluating the hearing of the subject after administering the dual vector system.

In some embodiments of any of the foregoing aspects, the method increases OTOF expression in a cochlear hair cell. In some embodiments of any of the foregoing aspects, the cochlear hair cell is an inner hair cell.

In some embodiments of any of the foregoing aspects, the dual vector system increases OTOF expression in a cell (e.g., a cochlear hair cell), improves hearing (e.g., as assessed by standard tests, such as audiometry, auditory brainstem response (ABR), electrocochleography (ECOG), and otoacoustic emissions), prevents or reduces hearing loss, delays the development of hearing loss, slows the progression of hearing loss, improves speech discrimination, or improves hair cell function.

In some embodiments of any of the foregoing aspects, the dual vector system is administered in an amount sufficient to increase OTOF expression in a cochlear hair cell, prevent or reduce hearing loss, delay the development of hearing loss, slow the progression of hearing loss, improve hearing (e.g., as assessed by standard tests, such as audiometry, ABR, ECOG, and otoacoustic emissions), improve speech discrimination, or improve hair cell function.

In some embodiments of any of the foregoing aspects, the first vector and the second vector are administered concurrently.

In some embodiments of any of the foregoing aspects, the first vector and the second vector are administered sequentially.

In some embodiments of any of the foregoing aspects, the first vector and the second vector are administered at a concentration of 1×10⁷ vector genomes (VG)/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

In some embodiments of any of the foregoing aspects, the first vector and the second vector are administered in amounts that together are sufficient to transduce at least 20% of the subject's inner hair cells with both the first vector and the second vector (e.g., at least 20%, 30%, 40%, 50%, 60%, 70%, 80%, 90%, 95% or more of the subject's inner hair cells are transduced with both vectors).

In some embodiments of any of the foregoing aspects, the dual vectors are administered in a composition including a pharmaceutically acceptable excipient.

In some embodiments of any of the foregoing aspects, the Myo15 promoter comprises or consists of a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 25 or a functional portion or derivative thereof including the sequence of SEQ ID NO: 31 and/or SEQ ID NO: 32, optionally containing a linker including one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first region comprises or consists of the sequence of SEQ ID NO: 24. In some embodiments, the second region comprises or consists of the sequence of SEQ ID NO: 25.

In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 36. In some embodiments of any of the foregoing aspects, the Myo15 promoter comprises or consists of the sequence of SEQ ID NO: 36. In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 38. In some embodiments of any of the foregoing aspects, the Myo15 promoter comprises or consists of the sequence of SEQ ID NO: 38. In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 39. In some embodiments of any of the foregoing aspects, the Myo15 promoter comprises or consists of the sequence of SEQ ID NO: 39. In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 53. In some embodiments of any of the foregoing aspects, the Myo15 promoter comprises or consists of the sequence of SEQ ID NO: 53. In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 54. In some embodiments of any of the foregoing aspects, the Myo15 promoter comprises or consists of the sequence of SEQ ID NO: 54. In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59. In some embodiments of any of the foregoing aspects, the Myo15 promoter comprises or consists of the sequence of SEQ ID NO: 59. In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 60. In some embodiments of any of the foregoing aspects, the Myo15 promoter comprises or consists of the sequence of SEQ ID NO: 60.

In some embodiments of any of the foregoing aspects, the Myo15 promoter comprises or consists of a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 25 or a functional portion or derivative thereof including the sequence of SEQ ID NO: 31 and/or SEQ ID NO: 32, operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, optionally containing a linker including one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first region comprises or consists of the sequence of SEQ ID NO: 25. In some embodiments, the second region comprises or consists of the sequence of SEQ ID NO: 24.

In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 37. In some embodiments of any of the foregoing aspects, the Myo15 promoter comprises or consists of the sequence of SEQ ID NO: 37. In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 58. In some embodiments of any of the foregoing aspects, the Myo15 promoter comprises or consists of the sequence of SEQ ID NO: 58.

In some embodiments of any of the foregoing aspects, the Myo15 promoter comprises or consists of a region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 24 or a functional portion or derivative thereof including the sequence of SEQ ID NO: 26 and/or SEQ ID NO: 27. In some embodiments, the region comprises or consists of the sequence of SEQ ID NO: 24.

In some embodiments of any of the foregoing aspects, the Myo15 promoter comprises or consists of a region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 25 or a functional portion or derivative thereof including the sequence of SEQ ID NO: 31 and/or SEQ ID NO: 32. In some embodiments, the region comprises or consists of the sequence of SEQ ID NO: 25.

In some embodiments of any of the foregoing aspects, the functional portion of SEQ ID NO: 24 contains the sequence of SEQ ID NO: 26. In some embodiments of any of the foregoing aspects, the functional portion of SEQ ID NO: 24 contains the sequence of SEQ ID NO: 27. In some embodiments of any of the foregoing aspects, the functional portion of SEQ ID NO: 24 contains the sequence of SEQ ID NO: 26 and the sequence of SEQ ID NO: 27. In some embodiments of any of the foregoing aspects, the functional portion of SEQ ID NO: 24 contains the sequence of SEQ ID NO: 28. In some embodiments of any of the foregoing aspects, the functional portion of SEQ ID NO: 24 contains the sequence of SEQ ID NO: 29. In some embodiments of any of the foregoing aspects, the functional portion of SEQ ID NO: 24 contains the sequence of SEQ ID NO: 30. In some embodiments of any of the foregoing aspects, the functional portion of SEQ ID NO: 24 contains the sequence of SEQ ID NO: 50.

In some embodiments of any of the foregoing aspects, the functional portion of SEQ ID NO: 25 contains the sequence of SEQ ID NO: 31. In some embodiments of any of the foregoing aspects, the functional portion of SEQ ID NO: 25 contains the sequence of SEQ ID NO: 32. In some embodiments of any of the foregoing aspects, the functional portion of SEQ ID NO: 25 contains the sequence of SEQ ID NO: 51. In some embodiments of any of the foregoing aspects, the functional portion of SEQ ID NO: 25 contains the sequence of SEQ ID NO: 51. In some embodiments of any of the foregoing aspects, the functional portion of SEQ ID NO: 25 contains the sequence of SEQ ID NO: 31 and the sequence of SEQ ID NO: 32. In some embodiments of any of the foregoing aspects, the functional portion of SEQ ID NO: 25 contains the sequence of SEQ ID NO: 33. In some embodiments of any of the foregoing aspects, the functional portion of SEQ ID NO: 25 contains the sequence of SEQ ID NO: 34. In some embodiments of any of the foregoing aspects, the functional portion of SEQ ID NO: 25 contains the sequence of SEQ ID NO: 35. In some embodiments of any of the foregoing aspects, the functional portion of SEQ ID NO: 25 contains the sequence of SEQ ID NO: 55.

In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence of any one of SEQ ID NOs: 50-58. In some embodiments, the Myo15 promoter comprises or consists of the sequence of SEQ ID NO: 50. In some embodiments, the Myo15 promoter comprises or consists of the sequence of SEQ ID NO: 51. In some embodiments, the Myo15 promoter comprises or consists of the sequence of SEQ ID NO: 52. In some embodiments, the Myo15 promoter comprises or consists of the sequence of SEQ ID NO: 53. In some embodiments, the Myo15 promoter comprises or consists of the sequence of SEQ ID NO: 54. In some embodiments, the Myo15 promoter comprises or consists of the sequence of SEQ ID NO: 55. In some embodiments, the Myo15 promoter comprises or consists of the sequence of SEQ ID NO: 56. In some embodiments, the Myo15 promoter comprises or consists of the sequence of SEQ ID NO: 57. In some embodiments, the Myo15 promoter comprises or consists of the sequence of SEQ ID NO: 58.

In some embodiments of any of the foregoing aspects, the Myo15 promoter comprises or consists of a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 40 or a functional portion or derivative thereof including the sequence of SEQ ID NO: 42, joined (e.g., operably linked) to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 41 or a functional portion or derivative thereof including the sequence of SEQ ID NO: 43 and/or SEQ ID NO: 44, optionally containing a linker including one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region comprises or consists of the sequence of SEQ ID NO: 40. In some embodiments, the second region comprises or consists of the sequence of SEQ ID NO: 41.

In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48. In some embodiments of any of the foregoing aspects, the Myo15 promoter comprises or consists of the sequence of SEQ ID NO: 48.

In some embodiments of any of the foregoing aspects, the Myo15 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 49. In some embodiments of any of the foregoing aspects, the Myo15 promoter comprises or consists of the sequence of SEQ ID NO: 49.

In some embodiments of any of the foregoing aspects, the Myo15 promoter comprises or consists of a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 41 or a functional portion or derivative thereof including the sequence of SEQ ID NO: 43 and/or SEQ ID NO: 44, joined (e.g., operably linked) to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 40 or a functional portion or derivative thereof including the sequence of SEQ ID NO: 42, optionally containing a linker including one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region comprises or consists of the sequence of SEQ ID NO: 41. In some embodiments, the second region comprises or consists of the sequence of SEQ ID NO: 40.

In some embodiments of any of the foregoing aspects, the Myo15 promoter comprises or consists of a region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 40 or a functional portion or derivative thereof including the sequence of SEQ ID NO: 42. In some embodiments, the region comprises or consists of the sequence of SEQ ID NO: 40.

In some embodiments of any of the foregoing aspects, the Myo15 promoter comprises or consists of a region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 41 or a functional portion or derivative thereof including the sequence of SEQ ID NO: 43 and/or SEQ ID NO: 44. In some embodiments, the region comprises or consists of the sequence of SEQ ID NO: 41.

In some embodiments of any of the foregoing aspects, the functional portion of SEQ ID NO: 40 contains the sequence of SEQ ID NO: 42.

In some embodiments of any of the foregoing aspects, the functional portion of SEQ ID NO: 41 contains the sequence of SEQ ID NO: 43. In some embodiments of any of the foregoing aspects, the functional portion of SEQ ID NO: 41 contains the sequence of SEQ ID NO: 44. In some embodiments of any of the foregoing aspects, the functional portion of SEQ ID NO: 41 contains the sequence of SEQ ID NO: 43 and the sequence of SEQ ID NO: 44. In some embodiments of any of the foregoing aspects, the functional portion of SEQ ID NO: 41 contains the sequence of SEQ ID NO: 45. In some embodiments of any of the foregoing aspects, the functional portion of SEQ ID NO: 41 contains the sequence of SEQ ID NO: 46. In some embodiments of any of the foregoing aspects, the functional portion of SEQ ID NO: 41 contains the sequence of SEQ ID NO: 47.

In some embodiments of any of the foregoing aspects, the Myo15 promoter induces transgene expression when operably linked to a transgene and introduced into a hair cell.

### Definitions

As used herein, the term “about” refers to a value that is within 10% above or below the value being described.

As used herein, “administration” refers to providing or giving a subject a therapeutic agent (e.g., a composition containing a first nucleic acid vector containing a polynucleotide that encodes an N-terminal portion of an otoferlin protein and a second nucleic acid vector containing a polynucleotide that encodes a C-terminal portion of an otoferlin protein), by any effective route. Exemplary routes of administration are described herein below.

As used herein, the term “biallelic OTOF mutations” refers to a condition in which a mutation is present in both alleles (copies) of an OTOF gene. A subject having biallelic OTOF mutations may have two OTOF alleles that carry the same mutation or may have a different mutation on each allele.

As used herein, the phrase “administering to the inner ear” refers to providing or giving a therapeutic agent described herein to a subject by any route that allows for transduction of inner ear cells. Exemplary routes of administration to the inner ear include administration into the perilymph or endolymph, such as to or through the oval window, round window, or semicircular canal (e.g., horizontal canal), or by transtympanic or intratympanic injection, e.g., administration to a hair cell.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in table 1 below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “degradation signal sequence” refers to a sequence (e.g., a nucleotide sequence that can be translated into an amino acid sequence) that mediates the degradation of a polypeptide in which it is contained. Degradation signal sequences can be included in the nucleic acid vectors of the invention to reduce or prevent the expression of portions of otoferlin proteins that have not undergone recombination and/or splicing. An exemplary degradation signal sequence for use in the invention is GCCTGCAAGAACTGGTTCAGCAGCCTGAGCCACTTCGTGATCCACCTG (SEQ ID NO: 22).

As used herein, the terms “effective amount,” “therapeutically effective amount,” and a “sufficient amount” of a composition, vector construct, or viral vector described herein refer to a quantity sufficient to, when administered to the subject in need thereof, including a mammal, for example a human, effect beneficial or desired results, including clinical results, and, as such, an “effective amount” or synonym thereto depends upon the context in which it is being applied. For example, in the context of treating sensorineural hearing loss, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. Note that when a combination of active ingredients is administered, the effective amount of the combination may or may not include amounts of each ingredient that would have been effective if administered individually. As defined herein, a therapeutically effective amount of a composition, vector construct, viral vector or cell of the present disclosure may be readily determined by one of ordinary skill by routine methods known in the art. Dosage regime may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human cochlear hair cell). As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human cochlear hair cell). Exogenous materials include those that are provided from an external source to an organism or to cultured matter extracted there from.

As used herein, the term “hair cell-specific expression” refers to production of an RNA transcript or polypeptide primarily within hair cells (e.g., cochlear hair cells) as compared to other cell types of the inner ear (e.g., spiral ganglion neurons, glia, or other inner ear cell types). Hair cell-specific expression of a transgene can be confirmed by comparing transgene expression (e.g., RNA or protein expression) between various cell types of the inner ear (e.g., hair cells vs. non-hair cells) using any standard technique (e.g., quantitative RT PCR, immunohistochemistry, Western Blot analysis, or measurement of the fluorescence of a reporter (e.g., GFP) operably linked to a promoter). A hair cell-specific promoter induces expression (e.g., RNA or protein expression) of a transgene to which it is operably linked that is at least 50% greater (e.g., 50%, 75%, 100%, 125%, 150%, 175%, 200% greater or more) in hair cells (e.g., cochlear hair cells) compared to at least 3 (e.g., 3, 4, 5, 6, 7, 8, 9, 10, or more) of the following inner ear cell types: Border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, spiral ganglion neurons, Schwann cells.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., OTOF expression or auditory brainstem response) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “intron” refers to a region within the coding region of a gene, the nucleotide sequence of which is not translated into the amino acid sequence of the corresponding protein. The term intron also refers to the corresponding region of the RNA transcribed from a gene. Introns are transcribed into pre-mRNA, but are removed during processing, and are not included in the mature mRNA.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule that can be joined to a second molecule, wherein the molecules are so arranged that the first molecule affects the function of the second molecule. The term “operably linked” includes the juxtaposition of two or more components (e.g., a promoter and another sequence element) such that both components function normally and allow for the possibility that at least one of the components can mediate a function that is exerted upon at least one of the other components. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. In additional embodiments, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the terms “otoferlin” and “OTOF” refer to the gene associated with nonsyndromic recessive deafness DNFB9. The terms “otoferlin” and “OTOF” also refer to variants of wild-type OTOF protein and nucleic acids encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type OTOF protein (e.g., any one of SEQ ID NOs: 1-5) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the nucleic acid sequence of a wild-type OTOF gene, provided that the OTOF analog encoded retains the therapeutic function of wild-type OTOF. As used herein, OTOF may refer to the protein localized to inner hair cells or to the gene encoding this protein, depending upon the context, as will be appreciated by one of skill in the art.

As used herein, the terms “otoferlin isoform 5” and “OTOF isoform 5” refer to an isoform of the gene associated with nonsyndromic recessive deafness DFNB9. The human isoform of the gene is associated with reference sequence NM 001287489, and the transcript includes exons 1-45 and 47 of human otoferlin, but lacks exon 46 of the OTOF gene. The human OTOF isoform 5 protein is also known as Otoferlin isoform e. The terms “otoferlin isoform 5” and “OTOF isoform 5” also refer to variants of the wild-type OTOF isoform 5 protein and polynucleotides encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type OTOF isoform 5 protein (e.g., SEQ ID NO: 1) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the polynucleotide sequence of a wild-type OTOF isoform 5 gene, provided that the OTOF isoform 5 analog encoded retains the therapeutic function of wild-type OTOF isoform 5. OTOF isoform 5 protein variants can have one or more (e.g., 1, 2, 3, 4, 5, 6, 7, 8, 9, 10, 11, 12, 13, 14, 15, 16, 17, 18, 19, 20, or more) conservative amino acid substitutions relative to a wild-type OTOF isoform 5 (e.g., SEQ ID NO: 1), provided that the that the OTOF isoform 5 variant retains the therapeutic function of wild-type OTOF isoform 5 and has no more than 10% amino acid substitutions in an N-terminal portion of the amino acid sequence and no more than 10% amino acid substitutions in a C-terminal portion of the amino acid sequence. As used herein, OTOF isoform 5 may refer to the protein localized to inner hair cells or to the gene encoding this protein, depending upon the context, as will be appreciated by one of skill in the art. OTOF isoform 5 may refer to human OTOF isoform 5 or to a homolog from another mammalian species. Murine otoferlin contains one additional exon relative to human otoferlin (48 exons in murine otoferlin), and the exons of murine otoferlin that correspond to those that encode human OTOF isoform 5 are 1-5, 7-46, and 48. The exon numbering convention used herein is based on the exons currently understood to be present in the consensus transcripts of human OTOF.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules containing nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the terms “complementarity” or “complementary” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

As used herein, the term “promoter” refers to a recognition site on DNA that is bound by an RNA polymerase. The polymerase drives transcription of the transgene. Exemplary promoters suitable for use with the compositions and methods described herein include ubiquitous promoters (e.g., the CAG promoter, cytomegalovirus (CMV) promoter, and a truncated form of the chimeric CMV-chicken β-actin promoter (CBA), in which the hybrid chicken β-actin/rabbit β-globin intron is greatly shortened to produce a smaller version of the promoter called smCBA), cochlear hair cell-specific promoters (e.g., the Myosin 15 (Myo15) promoter, the Myosin 7A (Myo7A) promoter, the Myosin 6 (Myo6) promoter, the POU Class 4 Homeobox 3 (POU4F3) promoter), and inner hair cell-specific promoters (e.g., the Fibroblast growth factor 8 (FGF8) promoter, the vesicular glutamate transporter 3 (VGLUT3) promoter, and the OTOF promoter).

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “derivative” as used herein refers to a nucleic acid, peptide, or protein or a variant or analog thereof comprising one or more mutations and/or chemical modifications as compared to a corresponding full-length wild-type nucleic acid, peptide, or protein. Non-limiting examples of chemical modifications involving nucleic acids include, for example, modifications to the base moiety, sugar moiety, phosphate moiety, phosphate-sugar backbone, or a combination thereof.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “recombinogenic region” refers to a region of homology that mediates recombination between two different sequences.

As used herein, the term “regulatory sequence” includes promoters, enhancers, and other expression control elements (e.g., polyadenylation signals) that control the transcription or translation of the polynucleotides that encode OTOF. Such regulatory sequences are described, for example, in Goeddel, Gene Expression Technology: Methods in Enzymology 185 (Academic Press, San Diego, C A, 1990); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection and the like.

As used herein, the terms “subject” and “patient” refer to an animal (e.g., a mammal, such as a human), veterinary animals (e.g., cats, dogs, cows, horses, sheep, pigs, etc.) and experimental animal models of diseases (e.g., mice, rats). A subject to be treated according to the methods described herein may be one who has been diagnosed with hearing loss (e.g., hearing loss associated with a mutation in OTOF), or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” of a state, disorder or condition can include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO94/11026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of OTOF as described herein include vectors that contain regulatory sequences, such as promoter and enhancer regions, which direct gene transcription. Other useful vectors for expression of OTOF contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIG. 1 is a graph showing ABR threshold recovery in homozygous OTOF-Q828X mutant mice treated at 32 or 52 weeks of age. Animals were dosed with vehicle (n=5/age group) or dual hybrid AAV-Myo15-hOTOF vectors (n=10/age group) through the round window under isoflurane anesthesia. ABR recovery was observed in 10/10 of the 32-week-old animals and 9/10 of the 52-week-old animals treated with OTOF dual vectors 4 weeks after treatment.

FIGS. 2A-2B are a series of graphs showing the number of inner hair cells and outer hair cells over time in homozygous (Otof-Q828X hom) and heterozygous (Otof-Q828X het) Otof-Q828X mice. Numbers of IHCs (FIG. 2A) and OHCs (FIG. 2B) were counted in 50 mouse ears in animals from 5 to 42 weeks of age. Counts are shown in cochlear regions corresponding to 5.6 kHz, 8 kHz, 11.3 kHz, 16 kHz, 22.6 kHz, 32 kHz, and 45.2 kHz. There was a statistically significant loss in IHC count with increasing age for all tested frequencies in Otof-Q828X hom mice. A similar trend in IHC count was observed in het mice for fewer frequencies (Kendall's rank correlation). IHC counts in Otof-828X hom and het animals were stable up to 16 weeks. Beginning after 16 weeks, Otof-Q828X hom animals showed a decrease in IHC counts starting at 22.6-45.2 kHz and after 24 weeks at lower frequencies (8-16 kHz). Loss of IHC counts in Otof-Q828X het mice started after 24 weeks for 16 and 32 kHz. After 32 weeks, there were over 75% of IHCs retained for most tested frequencies (<45.2 kHz) (FIG. 2A). The outer hair cell numbers in the Otof-Q828X hom and het mice remained constant over the studied time course of 6 months over all frequencies except for 8 kHz for which het mice showed an age-related decrease in counts (Kendall's rank correlation). OHC counts in 5.6 kHz and 45.2 kHz were associated with greater variability, showing differences in counts between het and hom that were interspersed over the ages tested. The majority of OHCs remained after 32 weeks (FIG. 2B).

FIG. 3 is a graph showing ABR threshold recovery in homozygous OTOF-Q828X mutant mice. ABR thresholds measured at 22.6 kHz were plotted vs. percent inner hair cells (IHCs) expressing otoferlin across multiple studies of adult homozygous OTOF-Q828X mutant mice treated with OTOF dual vector systems. Measurements were performed 4 weeks after treatment.

### DETAILED DESCRIPTION

Described herein are compositions and methods for the treatment of sensorineural hearing loss or auditory neuropathy due to biallelic otoferlin (OTOF) mutations in a human subject that is at least 25 years old (e.g., 25-50, 25-45, 25-40, 25-35, 25-30, 30-50, 30-45, 30-40, 30-35, 35-50, 35-45, 35-40, 40-50, 40-45, or 45-50 years old, e.g., 25, 26, 27, 28, 29, 30, 31, 32, 33, 34, 35, 36, 37, 38, 39, 40, 41, 42, 43, 44, 45, 46, 47, 48, 49, or 50 years old) by administering to the subject a first nucleic acid vector containing a promoter and a polynucleotide encoding an N-terminal portion of an otoferlin (OTOF) protein (e.g., a wild-type (WT) OTOF protein) and a second nucleic acid vector containing a polynucleotide encoding a C-terminal portion of an OTOF protein and a polyadenylation (poly(A)) sequence. When introduced into a mammalian cell, such as a cochlear hair cell, the polynucleotides encoded by the two nucleic acid vectors can combine to form a polynucleotide that encodes the full-length OTOF protein. The compositions and methods described herein can, therefore, be used to induce or increase expression of WT OTOF in cochlear hair cells of a subject who has an OTOF deficiency (e.g., a homozygous or compound heterozygous mutation in OTOF). The compositions and methods described herein can also be used to treat a subject having biallelic OTOF mutations that is identified as having detectable otoacoustic emissions, detectable cochlear microphonics, and/or detectable summating potential.

### Otoferlin

OTOF is a 230 kDa membrane protein that contains at least six C2 domains implicated in calcium, phospholipid, and protein binding. It is encoded by a gene that contains 48 exons, and the full-length protein is made up of 1,997 amino acids. OTOF is located at ribbon synapses in inner hair cells, where it is believed to function as a calcium sensor in synaptic vesicle fusion, triggering the fusion of neurotransmitter-containing vesicles with the plasma membrane. It has also been implicated in vesicle replenishment and clathrin-mediated endocytosis, and has been shown to interact with Myosin VI, Rab8b, SNARE proteins, calcium channel Cav1.3, Ergic2, and AP-2. The mechanism by which OTOF mediates exocytosis and the physiological significance of its interactions with its binding partners remain to be determined.

### Otoferlin-Associated Hearing Loss

OTOF was first identified by a study investigating the genetics of a non-syndromic form of deafness, autosomal recessive deafness-9 (DFNB9). Mutations in OTOF have since been found to cause sensorineural hearing loss in patients throughout the world, with many patients carrying OTOF mutations having auditory neuropathy, a disorder in which the inner ear detects sound, but is unable to properly transmit sound from the ear to the brain. These patients have an abnormal auditory brainstem response (ABR) and impaired speech discrimination with initially normal otoacoustic emissions. Patients carrying homozygous or compound heterozygous mutations in OTOF often develop hearing loss in early childhood, and the severity of hearing impairment has been found to vary with the location and type of mutation in OTOF. At least 220 mutations in OTOF have been identified, including mutations that cause truncations and mutations that do not cause truncations.

The present invention is based, in part, on the discovery that administration of a first nucleic acid vector containing a polynucleotide encoding an N-terminal portion of an OTOF protein and a second nucleic acid vector containing a polynucleotide encoding a C-terminal portion of an OTOF protein to adult (32-week-old) and middle-aged (52-week-old) otoferlin-deficient mice was effective in rescuing hearing loss. These data indicate that delivery of otoferlin to adult and middle-aged otoferlin-deficient mice is able to restore hearing despite the loss of hair cells that occurs as otoferlin-deficient mice age, and suggest that otoferlin gene therapy could also be used to treat similarly aged human subjects (32-week-old and 52-week-old mice correspond approximately to 30-50 year old human subjects). Humans also experience age-dependent hair cell loss, which is expected to limit the efficacy of gene therapy approaches in older adults. However, the inventors also discovered that hearing was restored in otoferlin-deficient mice when about 20% of inner hair cells expressed otoferlin, indicating that hearing can be rescued even if a relatively small percentage of inner hair cells are transduced. Taken together, these data indicate that adult human subjects (e.g., human subjects aged 25 or older, such as 25-50, 25-45, 25-40, 25-35, 25-30, 30-50, 30-45, 30-40, 30-35, 35-50, 35-45, 35-40, 40-50, 40-45, or 45-50 years old, e.g., 25, 26, 27, 28, 29, 30, 31, 32, 33, 34, 35, 36, 37, 38, 39, 40, 41, 42, 43, 44, 45, 46, 47, 48, 49, or 50 years old) with biallelic OTOF mutations can be treated using dual vector systems encoding OTOF.

The compositions and methods described herein can be used to treat sensorineural hearing loss or auditory neuropathy caused by biallelic OTOF mutations by administering a first nucleic acid vector containing a polynucleotide encoding an N-terminal portion of an OTOF protein and a second nucleic acid vector containing a polynucleotide encoding a C-terminal portion of an OTOF protein. The full-length OTOF coding sequence is too large to include in the type of vector that is commonly used for gene therapy (e.g., an adeno-associated virus (AAV) vector, which is thought to have a packaging limit of 5 kb). The compositions and methods described herein overcome this problem by dividing the OTOF coding sequence between two different nucleic acid vectors that can recombine in a cell to reconstitute the full-length OTOF sequence. These compositions and methods can be used to treat subjects having one or more mutations in the OTOF gene, e.g., an OTOF mutation that reduces OTOF expression, reduces OTOF function, or is associated with hearing loss. When the first and second nucleic acid vectors are administered in a composition, the polynucleotides encoding the N-terminal and C-terminal portions of OTOF can combine within a cell (e.g., a human cell, e.g., a cochlear hair cell) to form a single nucleic acid molecule that contains the full-length OTOF coding sequence (e.g., through homologous recombination and/or splicing).

The nucleic acid vectors used in the compositions and methods described herein include nucleic acid sequences that encode wild-type OTOF, or a variant thereof, such as a nucleic acid sequences that, when combined, encode a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of wild-type human or mouse OTOF. The polynucleotides used in the nucleic acid vectors described herein encode an N-terminal portion and a C-terminal portion of an OTOF amino acid sequence in Table 2 below (e.g., two portions that, when combined, encode a full-length OTOF amino acid sequence listed in Table 2, e.g., any one of SEQ ID NOs: 1-5).

According to the methods described herein, a subject can be administered a composition containing a first nucleic acid vector and a second nucleic acid vector that contain an N-terminal and C-terminal portion, respectively, of a polynucleotide sequence encoding the amino acid sequence of any one of SEQ ID NOs: 1-5, or a polynucleotide sequence encoding an amino acid sequence having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of any one of SEQ ID NOs: 1-5, or a polynucleotide sequence encoding an amino acid sequence that contains one or more conservative amino acid substitutions relative to any one of SEQ ID NOs: 1-5 (e.g., 1, 2, 3, 4, 5, 6, 7, 8, 9, or 10 or more conservative amino acid substitutions), provided that the OTOF analog encoded retains the therapeutic function of wild-type OTOF (e.g., the ability to regulate exocytosis at ribbon synapses or rescue or improve ABR response in an animal model of hearing loss related to Otoferlin gene deficiency (e.g., OTOF mutation)). No more than 10% of the amino acids in the N-terminal portion of the OTOF protein and no more than 10% of the amino acids in the C-terminal portion of the OTOF protein may be replaced with conservative amino acid substitutions. The OTOF protein may be encoded by a polynucleotide having the sequence of any one of SEQ ID NOs: 10-14. The OTOF protein may also be encoded by a polynucleotide having single nucleotide variants (SNVs) that have been found to be non-pathogenic in human subjects. The OTOF protein may be a human OTOF protein or may be a homolog of the human OTOF protein from another mammalian species (e.g., mouse, rat, cow, horse, goat, sheep, donkey, cat, dog, rabbit, guinea pig, or other mammal). In some embodiments, the OTOF protein encoded has the sequence of SEQ ID NO: 1 (OTOF isoform 1). In some embodiments, the OTOF protein encoded has the sequence of SEQ ID NO: 5 (OTOF isoform 5).

TABLE 2
OTOF Sequences

| SEQ ID | | |
|---|---|---|
| NO. | Sequence Name | Sequence |
| 1 | OTOF-201 protein | MALLIHLKTVSELRGRGDRIAKVTFRGQSFYSRVLENCEDVADFDE |
| | (NP_919224.1), | TFRWPVASSIDRNEMLEIQVFNYSKVFSNKLIGTFRMVLQKVVEES |
| | human otoferlin | HVEVTDTLIDDNNAIIKTSLCVEVRYQATDGTVGSWDDGDFLGDES |
| | isoform a, also | LQEEEKDSQETDGLLPGSRPSSRPPGEKSFRRAGRSVFSAMKLGK |
| | referred to as | NRSHKEEPQRPDEPAVLEMEDLDHLAIRLGDGLDPDSVSLASVTAL |
| | human OTOF | TTNVSNKRSKPDIKMEPSAGRPMDYQVSITVIEARQLVGLNMDPVV |
| | isoform 1, 1997 aa | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLCNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTSFIWFLNPLKSARYFLWHTYRWLLLKLL |
| | | LLLLLLLLLALFLYSVPGYLVKKILGA |
| 2 | OTOF-202 protein | MIKTEKSYPERRLRGVLEELSCGCCRFLSLADKDQGHSSRTRLDRE |
| | (NP_004793.2), | RLKSCMRELENMGQQARMLRAQVKRHTVRDKLRLCQNFLQKLRF |
| | human otoferlin | LADEPQHSIPDIFIWMMSNNKRVAYARVPSKDLLFSIVEEETGKDCA |
| | isoform b, 1230 aa | KVKTLFLKLPGKRGFGSAGWTVQAKVELYLWLGLSKQRKEFLCGL |
| | | PCGFQEVKAAQGLGLHAFPPVSLVYTKKQAFQLRAHMYQARSLFA |
| | | ADSSGLSDPFARVFFINQSQCTEVLNETLCPTWDQMLVFDNLELYG |
| | | EAHELRDDPPIIVIEIYDQDSMGKADFMGRTFAKPLVKMADEAYCPP |
| | | RFPPQLEYYQIYRGNATAGDLLAAFELLQIGPAGKADLPPINGPVDV |
| | | DRGPIMPVPMGIRPVLSKYRVEVLFWGLRDLKRVNLAQVDRPRVDI |
| | | ECAGKGVQSSLIHNYKKNPNFNTLVKWFEVDLPENELLHPPLNIRV |
| | | VDCRAFGRYTLVGSHAVSSLRRFIYRPPDRSAPSWNTTGEVVVTM |
| | | EPEVPIKKLETMVKLDATSEAVVKVDVAEEEKEKKKKKKGTAEEPE |
| | | EEEPDESMLDWWSKYFASIDTMKEQLRQQEPSGIDLEEKEEVDNT |
| | | EGLKGSMKGKEKARAAKEEKKKKTQSSGSGQGSEAPEKKKPKIDE |
| | | LKVYPKELESEFDNFEDWLHTFNLLRGKTGDDEDGSTEEERIVGRF |
| | | KGSLCVYKVPLPEDVSREAGYDSTYGMFQGIPSNDPINVLVRVYVV |
| | | RATDLHPADINGKADPYIAIRLGKTDIRDKENYISKQLNPVFGKSFDI |
| | | EASFPMESMLTVAVYDWDLVGTDDLIGETKIDLENRFYSKHRATCG |
| | | IAQTYSTHGYNIWRDPMKPSQILTRLCKDGKVDGPHFGPPGRVKVA |
| | | NRVFTGPSEIEDENGQRKPTDEHVALLALRHWEDIPRAGCRLVPEH |
| | | VETRPLLNPDKPGIEQGRLELWVDMFPMDMPAPGTPLDISPRKPKK |
| | | YELRVIIWNTDEVVLEDDDFFTGEKSSDIFVRGWLKGQQEDKQDTD |
| | | VHYHSLTGEGNFNWRYLFPFDYLAAEEKIVISKKESMFSWDETEYKI |
| | | PARLTLQIWDADHFSADDFLGAIELDLNRFPRGAKTAKQCTMEMAT |
| | | GEVDVPLVSIFKQKRVKGWWPLLARNENDEFELTGKVEAELHLLTA |
| | | EEAEKNPVGLARNEPDPLEKPNRPDTSFIWFLNPLKSARYFLWHTY |
| | | RWLLLKLLLLLLLLLLLALFLYSVPGYLVKKILGA |
| 3 | OTOF-203 protein | MIKTEKSYPERRLRGVLEELSCGCCRFLSLADKDQGHSSRTRLDRE |
| | (NP_919304.1), | RLKSCMRELENMGQQARMLRAQVKRHTVRDKLRLCQNFLQKLRF |
| | human otoferlin | LADEPQHSIPDIFIWMMSNNKRVAYARVPSKDLLFSIVEEETGKDCA |
| | isoform d, 1230 aa | KVKTLFLKLPGKRGFGSAGWTVQAKVELYLWLGLSKQRKEFLCGL |
| | | PCGFQEVKAAQGLGLHAFPPVSLVYTKKQAFQLRAHMYQARSLFA |
| | | ADSSGLSDPFARVFFINQSQCTEVLNETLCPTWDQMLVFDNLELYG |
| | | EAHELRDDPPIIVIEIYDQDSMGKADFMGRTFAKPLVKMADEAYCPP |
| | | RFPPQLEYYQIYRGNATAGDLLAAFELLQIGPAGKADLPPINGPVDV |
| | | DRGPIMPVPMGIRPVLSKYRVEVLFWGLRDLKRVNLAQVDRPRVDI |
| | | ECAGKGVQSSLIHNYKKNPNFNTLVKWFEVDLPENELLHPPLNIRV |
| | | VDCRAFGRYTLVGSHAVSSLRRFIYRPPDRSAPSWNTTGEVVVTM |
| | | EPEVPIKKLETMVKLDATSEAVVKVDVAEEEKEKKKKKKGTAEEPE |
| | | EEEPDESMLDWWSKYFASIDTMKEQLRQQEPSGIDLEEKEEVDNT |
| | | EGLKGSMKGKEKARAAKEEKKKKTQSSGSGQGSEAPEKKKPKIDE |
| | | LKVYPKELESEFDNFEDWLHTFNLLRGKTGDDEDGSTEEERIVGRF |
| | | KGSLCVYKVPLPEDVSREAGYDSTYGMFQGIPSNDPINVLVRVYVV |
| | | RATDLHPADINGKADPYIAIRLGKTDIRDKENYISKQLNPVFGKSFDI |
| | | EASFPMESMLTVAVYDWDLVGTDDLIGETKIDLENRFYSKHRATCG |
| | | IAQTYSTHGYNIWRDPMKPSQILTRLCKDGKVDGPHFGPPGRVKVA |
| | | NRVFTGPSEIEDENGQRKPTDEHVALLALRHWEDIPRAGCRLVPEH |
| | | VETRPLLNPDKPGIEQGRLELWVDMFPMDMPAPGTPLDISPRKPKK |
| | | YELRVIIWNTDEVVLEDDDFFTGEKSSDIFVRGWLKGQQEDKQDTD |
| | | VHYHSLTGEGNFNWRYLFPFDYLAAEEKIVISKKESMFSWDETEYKI |
| | | PARLTLQIWDADHFSADDFLGAIELDLNRFPRGAKTAKQCTMEMAT |
| | | GEVDVPLVSIFKQKRVKGWWPLLARNENDEFELTGKVEAELHLLTA |
| | | EEAEKNPVGLARNEPDPLEKPNRPDTAFVWFLNPLKSIKYLICTRYK |
| | | WLIIKIVLALLGLLMLGLFLYSLPGYMVKKLLGA |
| 4 | OTOF-208 protein | MMTDTQDGPSESSQIMRSLTPLINREEAFGEAGEAGLWPSITHTPD |
| | (NP_919303.1), | SQEEGLNDIQEMIKTEKSYPERRLRGVLEELSCGCCRFLSLADKDQ |
| | human otoferlin | GHSSRTRLDRERLKSCMRELENMGQQARMLRAQVKRHTVRDKLR |
| | isoform c, 1307 aa | LCQNFLQKLRFLADEPQHSIPDIFIWMMSNNKRVAYARVPSKDLLFS |
| | | IVEEETGKDCAKVKTLFLKLPGKRGFGSAGWTVQAKVELYLWLGLS |
| | | KQRKEFLCGLPCGFQEVKAAQGLGLHAFPPVSLVYTKKQAFQLRA |
| | | HMYQARSLFAADSSGLSDPFARVFFINQSQCTEVLNETLCPTWDQ |
| | | MLVFDNLELYGEAHELRDDPPIIVIEIYDQDSMGKADFMGRTFAKPL |
| | | VKMADEAYCPPRFPPQLEYYQIYRGNATAGDLLAAFELLQIGPAGK |
| | | ADLPPINGPVDVDRGPIMPVPMGIRPVLSKYRVEVLFWGLRDLKRV |
| | | NLAQVDRPRVDIECAGKGVQSSLIHNYKKNPNFNTLVKWFEVDLPE |
| | | NELLHPPLNIRVVDCRAFGRYTLVGSHAVSSLRRFIYRPPDRSAPS |
| | | WNTTVRLLRRCRVLCNGGSSSHSTGEVVVTMEPEVPIKKLETMVK |
| | | LDATSEAVVKVDVAEEEKEKKKKKKGTAEEPEEEEPDESMLDWWS |
| | | KYFASIDTMKEQLRQQEPSGIDLEEKEEVDNTEGLKGSMKGKEKAR |
| | | AAKEEKKKKTQSSGSGQGSEAPEKKKPKIDELKVYPKELESEFDNF |
| | | EDWLHTFNLLRGKTGDDEDGSTEEERIVGRFKGSLCVYKVPLPED |
| | | VSREAGYDSTYGMFQGIPSNDPINVLVRVYVVRATDLHPADINGKA |
| | | DPYIAIRLGKTDIRDKENYISKQLNPVFGKSFDIEASFPMESMLTVAV |
| | | YDWDLVGTDDLIGETKIDLENRFYSKHRATCGIAQTYSTHGYNIWR |
| | | DPMKPSQILTRLCKDGKVDGPHFGPPGRVKVANRVFTGPSEIEDE |
| | | NGQRKPTDEHVALLALRHWEDIPRAGCRLVPEHVETRPLLNPDKP |
| | | GIEQGRLELWVDMFPMDMPAPGTPLDISPRKPKKYELRVIIWNTDE |
| | | VVLEDDDFFTGEKSSDIFVRGWLKGQQEDKQDTDVHYHSLTGEGN |
| | | FNWRYLFPFDYLAAEEKIVISKKESMFSWDETEYKIPARLTLQIWDA |
| | | DHFSADDFLGAIELDLNRFPRGAKTAKQCTMEMATGEVDVPLVSIF |
| | | KQKRVKGWWPLLARNENDEFELTGKVEAELHLLTAEEAEKNPVGL |
| | | ARNEPDPLEKPNRPDTSFIWFLNPLKSARYFLWHTYRWLLLKLLLLL |
| | | LLLLLLALFLYSVPGYLVKKILGA |
| 5 | OTOF-205 protein | MALLIHLKTVSELRGRGDRIAKVTFRGQSFYSRVLENCEDVADFDE |
| | (NP_001274418.1), | TFRWPVASSIDRNEMLEIQVFNYSKVFSNKLIGTFRMVLQKVVEES |
| | human otoferlin | HVEVTDTLIDDNNAIIKTSLCVEVRYQATDGTVGSWDDGDFLGDES |
| | isoform e, 1997 aa, | LQEEEKDSQETDGLLPGSRPSSRPPGEKSFRRAGRSVFSAMKLGK |
| | also called human | NRSHKEEPQRPDEPAVLEMEDLDHLAIRLGDGLDPDSVSLASVTAL |
| | OTOF isoform 5 | TTNVSNKRSKPDIKMEPSAGRPMDYQVSITVIEARQLVGLNMDPVV |
| | | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLQNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTAFVWFLNPLKSIKYLICTRYKWLIIKIVLAL |
| | | LGLLMLGLFLYSLPGYMVKKLLGA |
| 6 | mOTOF-201_1 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_114081.2), | VEVTDTLMDDSNAIIKTSLSMEVRYQATDGTVGPWDDGDFLGDESL |
| | mouse otoferlin | QEEKDSQETDGLLPGSRPSTRISGEKSFRRAGRSVFSAMKLGKTR |
| | isoform 2, 1997 aa | SHKEEPQRQDEPAVLEMEDLDHLAIQLGDGLDPDSVSLASVTALTS |
| | | NVSNKRSKPDIKMEPSAGRPMDYQVSITVIEARQLVGLNMDPVVCV |
| | | EVGDDKKYTSMKESTNCPYYNEYFVFDFHVSPDVMFDKIIKISVIHS |
| | | KNLLRSGTLVGSFKMDVGTVYSQPEHQFHHKWAILSDPDDISAGLK |
| | | GYVKCDVAVVGKGDNIKTPHKANETDEDDIEGNLLLPEGVPPERQ |
| | | WARFYVKIYRAEGLPRMNTSLMANVKKAFIGENKDLVDPYVQVFFA |
| | | GQKGKTSVQKSSYEPLWNEQVVFTDLFPPLCKRMKVQIRDSDKVN |
| | | DVAIGTHFIDLRKISNDGDKGFLPTLGPAWVNMYGSTRNYTLLDEH |
| | | QDLNEGLGEGVSFRARLMLGLAVEILDTSNPELTSSTEVQVEQATP |
| | | VSESCTGRMEEFFLFGAFLEASMIDRKNGDKPITFEVTIGNYGNEV |
| | | DGMSRPLRPRPRKEPGDEEEVDLIQNSSDDEGDEAGDLASVSSTP |
| | | PMRPQITDRNYFHLPYLERKPCIYIKSWWPDQRRRLYNANIMDHIA |
| | | DKLEEGLNDVQEMIKTEKSYPERRLRGVLEELSCGCHRFLSLSDKD |
| | | QGRSSRTRLDRERLKSCMRELESMGQQAKSLRAQVKRHTVRDKL |
| | | RSCQNFLQKLRFLADEPQHSIPDVFIWMMSNNKRIAYARVPSKDLL |
| | | FSIVEEELGKDCAKVKTLFLKLPGKRGFGSAGWTVQAKLELYLWLG |
| | | LSKQRKDFLCGLPCGFEEVKAAQGLGLHSFPPISLVYTKKQAFQLR |
| | | AHMYQARSLFAADSSGLSDPFARVFFINQSQCTEVLNETLCPTWD |
| | | QMLVFDNLELYGEAHELRDDPPIIVIEIYDQDSMGKADFMGRTFAKP |
| | | LVKMADEAYCPPRFPPQLEYYQIYRGSATAGDLLAAFELLQIGPSG |
| | | KADLPPINGPVDMDRGPIMPVPVGIRPVLSKYRVEVLFWGLRDLKR |
| | | VNLAQVDRPRVDIECAGKGVQSSLIHNYKKNPNFNTLVKWFEVDLP |
| | | ENELLHPPLNIRVVDCRAFGRYTLVGSHAVSSLRRFIYRPPDRSAP |
| | | NWNTTVRLLRGCHRLRNGGPSSRPTGEVVVSMEPEEPVKKLETM |
| | | VKLDATSDAVVKVDVAEDEKERKKKKKKGPSEEPEEEEPDESMLD |
| | | WWSKYFASIDTMKEQLRQHETSGTDLEEKEEMESAEGLKGPMKS |
| | | KEKSRAAKEEKKKKNQSPGPGQGSEAPEKKKAKIDELKVYPKELES |
| | | EFDSFEDWLHTFNLLRGKTGDDEDGSTEEERIVGRFKGSLCVYKV |
| | | PLPEDVSREAGYDPTYGMFQGIPSNDPINVLVRIYVVRATDLHPADI |
| | | NGKADPYIAIKLGKTDIRDKENYISKQLNPVFGKSFDIEASFPMESML |
| | | TVAVYDWDLVGTDDLIGETKIDLENRFYSKHRATCGIAQTYSIHGYN |
| | | IWRDPMKPSQILTRLCKEGKVDGPHFGPHGRVRVANRVFTGPSEIE |
| | | DENGQRKPTDEHVALSALRHWEDIPRVGCRLVPEHVETRPLLNPD |
| | | KPGIEQGRLELWVDMFPMDMPAPGTPLDISPRKPKKYELRVIVWNT |
| | | DEVVLEDDDFFTGEKSSDIFVRGWLKGQQEDKQDTDVHYHSLTGE |
| | | GNFNWRYLFPFDYLAAEEKIVMSKKESMFSWDETEYKIPARLTLQI |
| | | WDADHFSADDFLGAIELDLNRFPRGAKTAKQCTMEMATGEVDVPL |
| | | VSIFKQKRVKGWWPLLARNENDEFELTGKVEAELHLLTAEEAEKNP |
| | | VGLARNEPDPLEKPNRPDTSFIWFLNPLKSARYFLWHTYRWLLLKF |
| | | LLLFLLLLLFALFLYSLPGYLAKKILGA |
| 7 | mOTOF-201_2 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_001273350.1), | VEVTDTLMDDSNAIIKTSLSMEVRYQATDGTVGPWDDGDFLGDESL |
| | mouse otoferlin | QEEKDSQETDGLLPGSRPSTRISGEKSFRRAGRSVFSAMKLGKTR |
| | isoform 3, 1977 aa | SHKEEPQRQDEPAVLEMEDLDHLAIQLGDGLDPDSVSLASVTALTS |
| | | NVSNKRSKPDIKMEPSAGRPMDYQVSITVIEARQLVGLNMDPVVCV |
| | | EVGDDKKYTSMKESTNCPYYNEYFVFDFHVSPDVMFDKIIKISVIHS |
| | | KNLLRSGTLVGSFKMDVGTVYSQPEHQFHHKWAILSDPDDISAGLK |
| | | GYVKCDVAVVGKGDNIKTPHKANETDEDDIEGNLLLPEGVPPERQ |
| | | WARFYVKIYRAEGLPRMNTSLMANVKKAFIGENKDLVDPYVQVFFA |
| | | GQKGKTSVQKSSYEPLWNEQVVFTDLFPPLCKRMKVQIRDSDKVN |
| | | DVAIGTHFIDLRKISNDGDKGFLPTLGPAWVNMYGSTRNYTLLDEH |
| | | QDLNEGLGEGVSFRARLMLGLAVEILDTSNPELTSSTEVQVEQATP |
| | | VSESCTGRMEEFFLFGAFLEASMIDRKNGDKPITFEVTIGNYGNEV |
| | | DGMSRPLRPRPRKEPGDEEEVDLIQNSSDDEGDEAGDLASVSSTP |
| | | PMRPQITDRNYFHLPYLERKPCIYIKSWWPDQRRRLYNANIMDHIA |
| | | DKLEEGLNDVQEMIKTEKSYPERRLRGVLEELSCGCHRFLSLSDKD |
| | | QGRSSRTRLDRERLKSCMRELESMGQQAKSLRAQVKRHTVRDKL |
| | | RSCQNFLQKLRFLADEPQHSIPDVFIWMMSNNKRIAYARVPSKDLL |
| | | FSIVEEELGKDCAKVKTLFLKLPGKRGFGSAGWTVQAKLELYLWLG |
| | | LSKQRKDFLCGLPCGFEEVKAAQGLGLHSFPPISLVYTKKQAFQLR |
| | | AHMYQARSLFAADSSGLSDPFARVFFINQSQCTEVLNETLCPTWD |
| | | QMLVFDNLELYGEAHELRDDPPIIVIEIYDQDSMGKADFMGRTFAKP |
| | | LVKMADEAYCPPRFPPQLEYYQIYRGSATAGDLLAAFELLQIGPSG |
| | | KADLPPINGPVDMDRGPIMPVPVGIRPVLSKYRVEVLFWGLRDLKR |
| | | VNLAQVDRPRVDIECAGKGVQSSLIHNYKKNPNFNTLVKWFEVDLP |
| | | ENELLHPPLNIRVVDCRAFGRYTLVGSHAVSSLRRFIYRPPDRSAP |
| | | NWNTTGEVVVSMEPEEPVKKLETMVKLDATSDAVVKVDVAEDEKE |
| | | RKKKKKKGPSEEPEEEEPDESMLDWWSKYFASIDTMKEQLRQHET |
| | | SGTDLEEKEEMESAEGLKGPMKSKEKSRAAKEEKKKKNQSPGPG |
| | | QGSEAPEKKKAKIDELKVYPKELESEFDSFEDWLHTFNLLRGKTGD |
| | | DEDGSTEEERIVGRFKGSLCVYKVPLPEDVSREAGYDPTYGMFQGI |
| | | PSNDPINVLVRIYVVRATDLHPADINGKADPYIAIKLGKTDIRDKENYI |
| | | SKQLNPVFGKSFDIEASFPMESMLTVAVYDWDLVGTDDLIGETKIDL |
| | | ENRFYSKHRATCGIAQTYSIHGYNIWRDPMKPSQILTRLCKEGKVD |
| | | GPHFGPHGRVRVANRVFTGPSEIEDENGQRKPTDEHVALSALRHW |
| | | EDIPRVGCRLVPEHVETRPLLNPDKPGIEQGRLELWVDMFPMDMP |
| | | APGTPLDISPRKPKKYELRVIVWNTDEVVLEDDDFFTGEKSSDIFVR |
| | | GWLKGQQEDKQDTDVHYHSLTGEGNFNWRYLFPFDYLAAEEKIV |
| | | MSKKESMFSWDETEYKIPARLTLQIWDADHFSADDFLGAIELDLNR |
| | | FPRGAKTAKQCTMEMATGEVDVPLVSIFKQKRVKGWWPLLARNEN |
| | | DEFELTGKVEAELHLLTAEEAEKNPVGLARNEPDPLEKPNRPDTSFI |
| | | WFLNPLKSARYFLWHTYRWLLLKFLLLFLLLLLFALFLYSLPGYLAKK |
| | | ILGA |
| 8 | mOTOF-202_1 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_001093865.1), | VEVTDTLMDDSNAIIKTSLSMEVRYQATDGTVGPWDDGDFLGDESL |
| | mouse otoferlin | QEEKDSQETDGLLPGSRPSTRISGEKSFRSKGREKTKGGRDGEHK |
| | isoform 1, 1992 aa | AGRSVFSAMKLGKTRSHKEEPQRQDEPAVLEMEDLDHLAIQLGDG |
| | | LDPDSVSLASVTALTSNVSNKRSKPDIKMEPSAGRPMDYQVSITVIE |
| | | ARQLVGLNMDPVVCVEVGDDKKYTSMKESTNCPYYNEYFVFDFHV |
| | | SPDVMFDKIIKISVIHSKNLLRSGTLVGSFKMDVGTVYSQPEHQFHH |
| | | KWAILSDPDDISAGLKGYVKCDVAVVGKGDNIKTPHKANETDEDDIE |
| | | GNLLLPEGVPPERQWARFYVKIYRAEGLPRMNTSLMANVKKAFIGE |
| | | NKDLVDPYVQVFFAGQKGKTSVQKSSYEPLWNEQVVFTDLFPPLC |
| | | KRMKVQIRDSDKVNDVAIGTHFIDLRKISNDGDKGFLPTLGPAWVN |
| | | MYGSTRNYTLLDEHQDLNEGLGEGVSFRARLMLGLAVEILDTSNPE |
| | | LTSSTEVQVEQATPVSESCTGRMEEFFLFGAFLEASMIDRKNGDKP |
| | | ITFEVTIGNYGNEVDGMSRPLRPRPRKEPGDEEEVDLIQNSSDDEG |
| | | DEAGDLASVSSTPPMRPQITDRNYFHLPYLERKPCIYIKSWWPDQR |
| | | RRLYNANIMDHIADKLEEGLNDVQEMIKTEKSYPERRLRGVLEELSC |
| | | GCHRFLSLSDKDQGRSSRTRLDRERLKSCMRELESMGQQAKSLR |
| | | AQVKRHTVRDKLRSCQNFLQKLRFLADEPQHSIPDVFIWMMSNNK |
| | | RIAYARVPSKDLLFSIVEEELGKDCAKVKTLFLKLPGKRGFGSAGWT |
| | | VQAKLELYLWLGLSKQRKDFLCGLPCGFEEVKAAQGLGLHSFPPIS |
| | | LVYTKKQAFQLRAHMYQARSLFAADSSGLSDPFARVFFINQSQCTE |
| | | VLNETLCPTWDQMLVFDNLELYGEAHELRDDPPIIVIEIYDQDSMGK |
| | | ADFMGRTFAKPLVKMADEAYCPPRFPPQLEYYQIYRGSATAGDLLA |
| | | AFELLQIGPSGKADLPPINGPVDMDRGPIMPVPVGIRPVLSKYRVEV |
| | | LFWGLRDLKRVNLAQVDRPRVDIECAGKGVQSSLIHNYKKNPNFNT |
| | | LVKWFEVDLPENELLHPPLNIRVVDCRAFGRYTLVGSHAVSSLRRFI |
| | | YRPPDRSAPNWNTTGEVVVSMEPEEPVKKLETMVKLDATSDAVVK |
| | | VDVAEDEKERKKKKKKGPSEEPEEEEPDESMLDWWSKYFASIDTM |
| | | KEQLRQHETSGTDLEEKEEMESAEGLKGPMKSKEKSRAAKEEKKK |
| | | KNQSPGPGQGSEAPEKKKAKIDELKVYPKELESEFDSFEDWLHTF |
| | | NLLRGKTGDDEDGSTEEERIVGRFKGSLCVYKVPLPEDVSREAGY |
| | | DPTYGMFQGIPSNDPINVLVRIYVVRATDLHPADINGKADPYIAIKLG |
| | | KTDIRDKENYISKQLNPVFGKSFDIEASFPMESMLTVAVYDWDLVG |
| | | TDDLIGETKIDLENRFYSKHRATCGIAQTYSIHGYNIWRDPMKPSQIL |
| | | TRLCKEGKVDGPHFGPHGRVRVANRVFTGPSEIEDENGQRKPTDE |
| | | HVALSALRHWEDIPRVGCRLVPEHVETRPLLNPDKPGIEQGRLELW |
| | | VDMFPMDMPAPGTPLDISPRKPKKYELRVIVWNTDEVVLEDDDFFT |
| | | GEKSSDIFVRGWLKGQQEDKQDTDVHYHSLTGEGNFNWRYLFPF |
| | | DYLAAEEKIVMSKKESMFSWDETEYKIPARLTLQIWDADHFSADDF |
| | | LGAIELDLNRFPRGAKTAKQCTMEMATGEVDVPLVSIFKQKRVKGW |
| | | WPLLARNENDEFELTGKVEAELHLLTAEEAEKNPVGLARNEPDPLE |
| | | KPNRPDTAFVWFLNPLKSIKYLICTRYKWLIIKIVLALLGLLMLALFLY |
| | | SLPGYMVKKLLGA |
| 9 | mOTOF-202_2 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_001300696.1), | VEVTDTLMDDSNAIIKTSLSMEVRYQATDGTVGPWDDGDFLGDESL |
| | mouse otoferlin | QEEKDSQETDGLLPGSRPSTRISGEKSFRRAGRSVFSAMKLGKTR |
| | isoform 4, 1977 aa | SHKEEPQRQDEPAVLEMEDLDHLAIQLGDGLDPDSVSLASVTALTS |
| | | NVSNKRSKPDIKMEPSAGRPMDYQVSITVIEARQLVGLNMDPVVCV |
| | | EVGDDKKYTSMKESTNCPYYNEYFVFDFHVSPDVMFDKIIKISVIHS |
| | | KNLLRSGTLVGSFKMDVGTVYSQPEHQFHHKWAILSDPDDISAGLK |
| | | GYVKCDVAVVGKGDNIKTPHKANETDEDDIEGNLLLPEGVPPERQ |
| | | WARFYVKIYRAEGLPRMNTSLMANVKKAFIGENKDLVDPYVQVFFA |
| | | GQKGKTSVQKSSYEPLWNEQVVFTDLFPPLCKRMKVQIRDSDKVN |
| | | DVAIGTHFIDLRKISNDGDKGFLPTLGPAWVNMYGSTRNYTLLDEH |
| | | QDLNEGLGEGVSFRARLMLGLAVEILDTSNPELTSSTEVQVEQATP |
| | | VSESCTGRMEEFFLFGAFLEASMIDRKNGDKPITFEVTIGNYGNEV |
| | | DGMSRPLRPRPRKEPGDEEEVDLIQNSSDDEGDEAGDLASVSSTP |
| | | PMRPQITDRNYFHLPYLERKPCIYIKSWWPDQRRRLYNANIMDHIA |
| | | DKLEEGLNDVQEMIKTEKSYPERRLRGVLEELSCGCHRFLSLSDKD |
| | | QGRSSRTRLDRERLKSCMRELESMGQQAKSLRAQVKRHTVRDKL |
| | | RSCQNFLQKLRFLADEPQHSIPDVFIWMMSNNKRIAYARVPSKDLL |
| | | FSIVEEELGKDCAKVKTLFLKLPGKRGFGSAGWTVQAKLELYLWLG |
| | | LSKQRKDFLCGLPCGFEEVKAAQGLGLHSFPPISLVYTKKQAFQLR |
| | | AHMYQARSLFAADSSGLSDPFARVFFINQSQCTEVLNETLCPTWD |
| | | QMLVFDNLELYGEAHELRDDPPIIVIEIYDQDSMGKADFMGRTFAKP |
| | | LVKMADEAYCPPRFPPQLEYYQIYRGSATAGDLLAAFELLQIGPSG |
| | | KADLPPINGPVDMDRGPIMPVPVGIRPVLSKYRVEVLFWGLRDLKR |
| | | VNLAQVDRPRVDIECAGKGVQSSLIHNYKKNPNFNTLVKWFEVDLP |
| | | ENELLHPPLNIRVVDCRAFGRYTLVGSHAVSSLRRFIYRPPDRSAP |
| | | NWNTTGEVVVSMEPEEPVKKLETMVKLDATSDAVVKVDVAEDEKE |
| | | RKKKKKKGPSEEPEEEEPDESMLDWWSKYFASIDTMKEQLRQHET |
| | | SGTDLEEKEEMESAEGLKGPMKSKEKSRAAKEEKKKKNQSPGPG |
| | | QGSEAPEKKKAKIDELKVYPKELESEFDSFEDWLHTFNLLRGKTGD |
| | | DEDGSTEEERIVGRFKGSLCVYKVPLPEDVSREAGYDPTYGMFQGI |
| | | PSNDPINVLVRIYVVRATDLHPADINGKADPYIAIKLGKTDIRDKENYI |
| | | SKQLNPVFGKSFDIEASFPMESMLTVAVYDWDLVGTDDLIGETKIDL |
| | | ENRFYSKHRATCGIAQTYSIHGYNIWRDPMKPSQILTRLCKEGKVD |
| | | GPHFGPHGRVRVANRVFTGPSEIEDENGQRKPTDEHVALSALRHW |
| | | EDIPRVGCRLVPEHVETRPLLNPDKPGIEQGRLELWVDMFPMDMP |
| | | APGTPLDISPRKPKKYELRVIVWNTDEVVLEDDDFFTGEKSSDIFVR |
| | | GWLKGQQEDKQDTDVHYHSLTGEGNFNWRYLFPFDYLAAEEKIV |
| | | MSKKESMFSWDETEYKIPARLTLQIWDADHFSADDFLGAIELDLNR |
| | | FPRGAKTAKQCTMEMATGEVDVPLVSIFKQKRVKGWWPLLARNEN |
| | | DEFELTGKVEAELHLLTAEEAEKNPVGLARNEPDPLEKPNRPDTAF |
| | | VWFLNPLKSIKYLICTRYKWLIIKIVLALLGLLMLALFLYSLPGYMVKK |
| | | LLGA |
| 10 | DNA sequence | ATGGCCCTGATTGTTCACCTCAAGACTGTCTCAGAGCTCCGAGG |
| | encoding the human | CAAAGGTGACCGGATTGCCAAAGTCACTTTCCGAGGGCAGTCTT |
| | otoferlin isoform 1 | TCTACTCCCGGGTCCTGGAGAACTGCGAGGGTGTGGCTGACTT |
| | protein (SEQ ID NO: | TGATGAGACGTTCCGGTGGCCAGTGGCCAGCAGCATCGACCGG |
| | 1), 5979 bp, | AATGAAGTGTTGGAGATTCAGATTTTCAACTACAGCAAAGTCTTC |
| | corresponds to the | AGCAACAAGCTGATAGGGACCTTCTGCATGGTGCTGCAGAAAGT |
| | coding sequence | GGTGGAGGAGAATCGGGTAGAGGTGACCGACACGCTGATGGAT |
| | documented in | GACAGCAATGCTATCATCAAGACCAGCCTGAGCATGGAGGTCC |
| | NM_001100395 | GGTATCAGGCCACAGATGGCACTGTGGGCCCCTGGGATGATGG |
| | | AGACTTCCTGGGAGATGAATCCCTCCAGGAGGAGAAGGACAGC |
| | | CAGGAGACAGATGGGCTGCTACCTGGTTCCCGACCCAGCACCC |
| | | GGATATCTGGCGAGAAGAGCTTTCGCAGCAAAGGCAGAGAGAA |
| | | GACCAAGGGAGGCAGAGATGGCGAGCACAAAGCGGGAAGGAG |
| | | TGTGTTCTCGGCCATGAAACTCGGCAAAACTCGGTCCCACAAAG |
| | | AGGAGCCCCAAAGACAAGATGAGCCAGCAGTGCTGGAGATGGA |
| | | GGACCTGGACCACCTAGCCATTCAGCTGGGGGATGGGCTGGAT |
| | | CCTGACTCCGTGTCTCTAGCCTCGGTCACCGCTCTCACCAGCAA |
| | | TGTCTCCAACAAACGGTCTAAGCCAGATATTAAGATGGAGCCCA |
| | | GTGCTGGAAGGCCCATGGATTACCAGGTCAGCATCACAGTGATT |
| | | GAGGCTCGGCAGCTGGTGGGCTTGAACATGGACCCTGTGGTGT |
| | | GTGTGGAGGTGGGTGATGACAAGAAATACACGTCAATGAAGGA |
| | | GTCCACAAACTGCCCTTACTACAACGAGTACTTTGTCTTCGACTT |
| | | CCATGTCTCTCCTGATGTCATGTTTGACAAGATCATCAAGATCTC |
| | | GGTTATCCATTCTAAGAACCTGCTTCGGAGCGGCACCCTGGTGG |
| | | GTTCCTTCAAAATGGATGTGGGGACTGTGTATTCCCAGCCTGAA |
| | | CACCAGTTCCATCACAAATGGGCCATCCTGTCAGACCCCGATGA |
| | | CATCTCTGCTGGGTTGAAGGGTTATGTAAAGTGTGATGTCGCTG |
| | | TGGTGGGCAAGGGAGACAACATCAAGACACCCCACAAGGCCAA |
| | | CGAGACGGATGAGGACGACATTGAAGGGAACTTGCTGCTCCCC |
| | | GAGGGCGTGCCCCCCGAACGGCAGTGGGCACGGTTCTATGTGA |
| | | AAATTTACCGAGCAGAGGGACTGCCCCGGATGAACACAAGCCT |
| | | CATGGCCAACGTGAAGAAGGCGTTCATCGGTGAGAACAAGGAC |
| | | CTCGTCGACCCCTATGTGCAAGTCTTCTTTGCTGGACAAAAGGG |
| | | CAAAACATCAGTGCAGAAGAGCAGCTATGAGCCGCTATGGAATG |
| | | AGCAGGTCGTCTTCACAGACTTGTTCCCCCCACTCTGCAAACGC |
| | | ATGAAGGTGCAGATCCGGGACTCTGACAAGGTCAATGATGTGG |
| | | CCATCGGCACCCACTTCATCGACCTGCGCAAGATTTCCAACGAT |
| | | GGAGACAAAGGCTTCCTGCCTACCCTCGGTCCAGCCTGGGTGA |
| | | ACATGTACGGCTCCACGCGCAACTACACACTGCTGGACGAGCA |
| | | CCAGGACTTGAATGAAGGCCTGGGGGAGGGTGTGTCCTTCCGG |
| | | GCCCGCCTCATGTTGGGACTAGCTGTGGAGATCCTGGACACCT |
| | | CCAACCCAGAGCTCACCAGCTCCACGGAGGTGCAGGTGGAGCA |
| | | GGCCACGCCTGTCTCGGAGAGCTGCACAGGGAGAATGGAAGAA |
| | | TTTTTTCTATTTGGAGCCTTCTTGGAAGCCTCAATGATTGACCGG |
| | | AAAAATGGGGACAAGCCAATTACCTTTGAGGTGACCATAGGAAA |
| | | CTACGGCAATGAAGTCGATGGTATGTCCCGGCCCCTGAGGCCT |
| | | CGGCCCCGGAAAGAGCCTGGGGATGAAGAAGAGGTAGACCTGA |
| | | TTCAGAACTCCAGTGACGATGAAGGTGACGAAGCCGGGGACCT |
| | | GGCCTCGGTGTCCTCCACCCCACCTATGCGGCCCCAGATCACG |
| | | GACAGGAACTATTTCCACCTGCCCTACCTGGAGCGCAAGCCCT |
| | | GCATCTATATCAAGAGCTGGTGGCCTGACCAGAGGCGGCGCCT |
| | | CTACAATGCCAACATCATGGATCACATTGCTGACAAGCTGGAAG |
| | | AAGGCCTGAATGATGTACAGGAGATGATCAAAACGGAGAAGTCC |
| | | TACCCGGAGCGCCGCCTGCGGGGTGTGCTAGAGGAACTCAGCT |
| | | GTGGCTGCCACCGCTTCCTCTCCCTCTCGGACAAGGACCAGGG |
| | | CCGCTCGTCCCGCACCAGGCTGGATCGAGAGCGTCTTAAGTCC |
| | | TGTATGAGGGAGTTGGAGAGCATGGGACAGCAGGCCAAGAGCC |
| | | TGAGGGCTCAGGTGAAGCGGCACACTGTTCGGGACAAGCTGAG |
| | | GTCATGCCAGAACTTTCTGCAGAAGCTACGCTTCCTGGCGGATG |
| | | AGCCCCAGCACAGCATTCCTGATGTGTTCATTTGGATGATGAGC |
| | | AACAACAAACGTATCGCCTATGCCCGCGTGCCTTCCAAAGACCT |
| | | GCTCTTCTCCATCGTGGAGGAGGAACTGGGCAAGGACTGCGCC |
| | | AAAGTCAAGACCCTCTTCCTGAAGCTGCCAGGGAAGAGGGGCT |
| | | TCGGCTCGGCAGGCTGGACAGTACAGGCCAAGCTGGAGCTCTA |
| | | CCTGTGGCTGGGCCTCAGCAAGCAGCGAAAGGACTTCCTGTGT |
| | | GGTCTGCCCTGTGGCTTCGAGGAGGTCAAGGCAGCCCAAGGCC |
| | | TGGGCCTGCATTCCTTTCCGCCCATCAGCCTAGTCTACACCAAG |
| | | AAGCAAGCCTTCCAGCTCCGAGCACACATGTATCAGGCCCGAA |
| | | GCCTCTTTGCTGCTGACAGCAGTGGGCTCTCTGATCCCTTTGCC |
| | | CGTGTCTTCTTCATCAACCAGAGCCAATGCACTGAGGTTCTAAA |
| | | CGAGACACTGTGTCCCACCTGGGACCAGATGCTGGTATTTGACA |
| | | ACCTGGAGCTGTACGGTGAAGCTCACGAGTTACGAGATGATCC |
| | | CCCCATCATTGTCATTGAAATCTACGACCAGGACAGCATGGGCA |
| | | AAGCCGACTTCATGGGCCGGACCTTCGCCAAGCCCCTGGTGAA |
| | | GATGGCAGATGAAGCATACTGCCCACCTCGCTTCCCGCCGCAG |
| | | CTTGAGTACTACCAGATCTACCGAGGCAGTGCCACTGCCGGAG |
| | | ACCTACTGGCTGCCTTCGAGCTGCTGCAGATTGGGCCATCAGG |
| | | GAAGGCTGACCTGCCACCCATCAATGGCCCAGTGGACATGGAC |
| | | AGAGGGCCCATCATGCCTGTGCCCGTGGGAATCCGGCCAGTGC |
| | | TCAGCAAGTACCGAGTGGAGGTGCTGTTCTGGGGCCTGAGGGA |
| | | CCTAAAGAGGGTGAACCTGGCCCAGGTGGACCGACCACGGGTG |
| | | GACATCGAGTGTGCAGGAAAGGGGGTACAATCCTCCCTGATTCA |
| | | CAATTATAAGAAGAACCCCAACTTCAACACGCTGGTCAAGTGGT |
| | | TTGAAGTGGACCTCCCGGAGAATGAGCTCCTGCACCCACCCTT |
| | | GAACATCCGAGTGGTAGATTGCCGGGCCTTTGGACGATACACC |
| | | CTGGTGGGTTCCCACGCAGTCAGCTCACTGAGGCGCTTCATCTA |
| | | CCGACCTCCAGACCGCTCAGCCCCCAACTGGAACACCACAGGG |
| | | GAGGTTGTAGTAAGCATGGAGCCTGAGGAGCCAGTTAAGAAGC |
| | | TGGAGACCATGGTGAAACTGGATGCGACTTCTGATGCTGTGGTC |
| | | AAGGTGGATGTGGCTGAAGATGAGAAGGAAAGGAAGAAGAAGA |
| | | AAAAGAAAGGCCCGTCAGAGGAGCCAGAGGAGGAAGAGCCCG |
| | | ATGAGAGCATGCTGGATTGGTGGTCCAAGTACTTCGCCTCCATC |
| | | GACACAATGAAGGAGCAACTTCGACAACATGAGACCTCTGGAAC |
| | | TGACTTGGAAGAGAAGGAAGAGATGGAAAGCGCTGAGGGCCTG |
| | | AAGGGACCAATGAAGAGCAAGGAGAAGTCCAGAGCTGCAAAGG |
| | | AGGAGAAAAAGAAGAAAAACCAGAGCCCTGGCCCTGGCCAGGG |
| | | ATCGGAGGCTCCTGAGAAGAAGAAAGCCAAGATCGATGAGCTTA |
| | | AGGTGTACCCCAAGGAGCTGGAATCGGAGTTTGACAGCTTTGA |
| | | GGACTGGCTGCACACCTTCAACCTGTTGAGGGGCAAGACGGGA |
| | | GATGATGAGGATGGCTCCACAGAGGAGGAGCGCATAGTAGGCC |
| | | GATTCAAGGGCTCCCTCTGTGTGTACAAAGTGCCACTCCCAGAA |
| | | GATGTATCTCGAGAAGCTGGCTATGATCCCACCTATGGAATGTT |
| | | CCAGGGCATCCCAAGCAATGACCCCATCAATGTGCTGGTCCGA |
| | | ATCTATGTGGTCCGGGCCACAGACCTGCACCCGGCCGACATCA |
| | | ATGGCAAAGCTGACCCCTATATTGCCATCAAGTTAGGCAAGACC |
| | | GACATCCGAGACAAGGAGAACTACATCTCCAAGCAGCTCAACCC |
| | | TGTGTTTGGGAAGTCCTTTGACATTGAGGCCTCCTTCCCCATGG |
| | | AGTCCATGTTGACAGTGGCCGTGTACGACTGGGATCTGGTGGG |
| | | CACTGATGACCTCATCGGAGAAACCAAGATTGACCTGGAAAACC |
| | | GCTTCTACAGCAAGCATCGCGCCACCTGCGGCATCGCACAGAC |
| | | CTATTCCATACATGGCTACAATATCTGGAGGGACCCCATGAAGC |
| | | CCAGCCAGATCCTGACACGCCTCTGTAAAGAGGGCAAAGTGGA |
| | | CGGCCCCCACTTTGGTCCCCATGGGAGAGTGAGGGTTGCCAAC |
| | | CGTGTCTTCACGGGGCCTTCAGAAATAGAGGATGAGAATGGTCA |
| | | GAGGAAGCCCACAGATGAGCACGTGGCACTGTCTGCTCTGAGA |
| | | CACTGGGAGGACATCCCCCGGGTGGGCTGCCGCCTTGTGCCG |
| | | GAACACGTGGAGACCAGGCCGCTGCTCAACCCTGACAAGCCAG |
| | | GCATTGAGCAGGGCCGCCTGGAGCTGTGGGTGGACATGTTCCC |
| | | CATGGACATGCCAGCCCCTGGGACACCTCTGGATATATCCCCCA |
| | | GGAAACCCAAGAAGTACGAGCTGCGGGTCATCGTGTGGAACAC |
| | | AGACGAGGTGGTCCTGGAAGACGATGATTTCTTCACGGGAGAG |
| | | AAGTCCAGTGACATTTTTGTGAGGGGGTGGCTGAAGGGCCAGC |
| | | AGGAGGACAAACAGGACACAGATGTCCACTATCACTCCCTCACG |
| | | GGGGAGGGCAACTTCAACTGGAGATACCTCTTCCCCTTCGACTA |
| | | CCTAGCGGCCGAAGAGAAGATCGTTATGTCCAAAAAGGAGTCTA |
| | | TGTTCTCCTGGGATGAGACGGAGTACAAGATCCCTGCGCGGCT |
| | | CACCCTGCAGATCTGGGACGCTGACCACTTCTCGGCTGACGAC |
| | | TTCCTGGGGGCTATCGAGCTGGACCTGAACCGGTTCCCGAGGG |
| | | GCGCTAAGACAGCCAAGCAGTGCACCATGGAGATGGCCACCGG |
| | | GGAGGTGGACGTACCCCTGGTTTCCATCTTTAAACAGAAACGTG |
| | | TCAAAGGCTGGTGGCCCCTCCTGGCCCGCAATGAGAATGATGA |
| | | GTTTGAGCTCACAGGCAAAGTGGAGGCGGAGCTACACCTACTC |
| | | ACGGCAGAGGAGGCAGAGAAGAACCCTGTGGGCCTGGCTCGC |
| | | AATGAACCTGATCCCCTAGAAAAACCCAACCGGCCTGACACGGC |
| | | ATTCGTCTGGTTCCTGAACCCACTCAAATCTATCAAGTACCTCAT |
| | | CTGCACCCGGTACAAGTGGCTGATCATCAAGATCGTGCTGGCG |
| | | CTGCTGGGGCTGCTCATGCTGGCCCTCTTCCTTTACAGCCTCCC |
| | | AGGCTACATGGTCAAGAAGCTCCTAGGGGCCTGA |
| 11 | OTOF-202 transcript | CCGTGAGTTCTGCCCAGGCCCTGTGAGCTCACCAGAGCCACAG |
| | (NM_004802.3), | ACTCACAGCCCAGAGGTGGCTTCTTCCTTCAGGAACTGAAGAAC |
| | human otoferlin | CCCCATGAACACCAACATCTCCAGGTTCTGAGAACAGAACCTGG |
| | transcript variant 2, | GAAATTGATGACTTCCTCATGATGACCGATACTCAGGATGGCCC |
| | 4969 bp, encodes | TAGCGAGAGCTCCCAGATCATGAGGAAGAAGGCCTGAACGACA |
| | the protein of SEQ | TACAGGAGATGATCAAAACGGAGAAGTCCTACCCTGAGCGTCG |
| | ID NO: 2 | CCTGCGGGGCGTCCTGGAGGAGCTGAGCTGTGGCTGCTGCCG |
| | | CTTCCTCTCCCTCGCTGACAAGGACCAGGGCCACTCATCCCGC |
| | | ACCAGGCTTGACCGGGAGCGCCTCAAGTCCTGCATGAGGGAGC |
| | | TGGAAAACATGGGGCAGCAGGCCAGGATGCTGCGGGCCCAGG |
| | | TGAAGCGGCACACGGTGCGGGACAAGCTGAGGCTGTGCCAGAA |
| | | CTTCCTGCAGAAGCTGCGCTTCCTGGCGGACGAGCCCCAGCAC |
| | | AGCATTCCCGACATCTTCATCTGGATGATGAGCAACAACAAGCG |
| | | TGTCGCCTATGCCCGTGTGCCCTCCAAGGACCTGCTCTTCTCCA |
| | | TCGTGGAGGAGGAGACTGGCAAGGACTGCGCCAAGGTCAAGAC |
| | | GCTCTTCCTTAAGCTGCCAGGGAAGCGGGGCTTCGGCTCGGCA |
| | | GGCTGGACAGTGCAGGCCAAGGTGGAGCTGTACCTGTGGCTGG |
| | | GCCTCAGCAAACAGCGCAAGGAGTTCCTGTGCGGCCTGCCCTG |
| | | TGGCTTCCAGGAGGTCAAGGCAGCCCAGGGCCTGGGCCTGCAT |
| | | GCCTTCCCACCCGTCAGCCTGGTCTACACCAAGAAGCAGGCGT |
| | | TCCAGCTCCGAGCGCACATGTACCAGGCCCGCAGCCTCTTTGC |
| | | CGCCGACAGCAGCGGACTCTCAGACCCCTTTGCCCGCGTCTTC |
| | | TTCATCAATCAGAGTCAGTGCACAGAGGTGCTGAATGAGACCCT |
| | | GTGTCCCACCTGGGACCAGATGCTGGTGTTCGACAACCTGGAG |
| | | CTCTATGGTGAAGCTCATGAGCTGAGGGACGATCCGCCCATCAT |
| | | TGTCATTGAAATCTATGACCAGGATTCCATGGGCAAAGCTGACT |
| | | TCATGGGCCGGACCTTCGCCAAACCCCTGGTGAAGATGGCAGA |
| | | CGAGGCGTACTGCCCACCCCGCTTCCCACCTCAGCTCGAGTAC |
| | | TACCAGATCTACCGTGGCAACGCCACAGCTGGAGACCTGCTGG |
| | | CGGCCTTCGAGCTGCTGCAGATTGGACCAGCAGGGAAGGCTGA |
| | | CCTGCCCCCCATCAATGGCCCGGTGGACGTGGACCGAGGTCCC |
| | | ATCATGCCCGTGCCCATGGGCATCCGGCCCGTGCTCAGCAAGT |
| | | ACCGAGTGGAGGTGCTGTTCTGGGGCCTACGGGACCTAAAGCG |
| | | GGTGAACCTGGCCCAGGTGGACCGGCCACGGGTGGACATCGA |
| | | GTGTGCAGGGAAGGGGGTGCAGTCGTCCCTGATCCACAATTAT |
| | | AAGAAGAACCCCAACTTCAACACCCTCGTCAAGTGGTTTGAAGT |
| | | GGACCTCCCAGAGAACGAGCTGCTGCACCCGCCCTTGAACATC |
| | | CGTGTGGTGGACTGCCGGGCCTTCGGTCGCTACACACTGGTGG |
| | | GCTCCCATGCCGTCAGCTCCCTGCGACGCTTCATCTACCGGCC |
| | | CCCAGACCGCTCGGCCCCCAGCTGGAACACCACGGGGGAGGT |
| | | TGTGGTGACTATGGAGCCAGAGGTACCCATCAAGAAACTGGAG |
| | | ACCATGGTGAAGCTGGACGCGACTTCTGAAGCTGTTGTCAAGGT |
| | | GGATGTGGCTGAGGAGGAGAAGGAGAAGAAGAAGAAGAAGAAG |
| | | GGCACTGCGGAGGAGCCAGAGGAGGAGGAGCCAGACGAGAGC |
| | | ATGCTGGACTGGTGGTCCAAGTACTTTGCCTCCATTGACACCAT |
| | | GAAGGAGCAACTTCGACAACAAGAGCCCTCTGGAATTGACTTGG |
| | | AGGAGAAGGAGGAAGTGGACAATACCGAGGGCCTGAAGGGGT |
| | | CAATGAAGGGCAAGGAGAAGGCAAGGGCTGCCAAAGAGGAGAA |
| | | GAAGAAGAAAACTCAGAGCTCTGGCTCTGGCCAGGGGTCCGAG |
| | | GCCCCCGAGAAGAAGAAACCCAAGATTGATGAGCTTAAGGTATA |
| | | CCCCAAAGAGCTGGAGTCCGAGTTTGATAACTTTGAGGACTGGC |
| | | TGCACACTTTCAACTTGCTTCGGGGCAAGACCGGGGATGATGA |
| | | GGATGGCTCCACCGAGGAGGAGCGCATTGTGGGACGCTTCAAG |
| | | GGCTCCCTCTGCGTGTACAAAGTGCCACTCCCAGAGGACGTGT |
| | | CCCGGGAAGCCGGCTACGACTCCACCTACGGCATGTTCCAGGG |
| | | CATCCCGAGCAATGACCCCATCAATGTGCTGGTCCGAGTCTATG |
| | | TGGTCCGGGCCACGGACCTGCACCCTGCTGACATCAACGGCAA |
| | | AGCTGACCCCTACATCGCCATCCGGCTAGGCAAGACTGACATC |
| | | CGCGACAAGGAGAACTACATCTCCAAGCAGCTCAACCCTGTCTT |
| | | TGGGAAGTCCTTTGACATCGAGGCCTCCTTCCCCATGGAATCCA |
| | | TGCTGACGGTGGCTGTGTATGACTGGGACCTGGTGGGCACTGA |
| | | TGACCTCATTGGGGAAACCAAGATCGACCTGGAGAACCGCTTCT |
| | | ACAGCAAGCACCGCGCCACCTGCGGCATCGCCCAGACCTACTC |
| | | CACACATGGCTACAATATCTGGCGGGACCCCATGAAGCCCAGC |
| | | CAGATCCTGACCCGCCTCTGCAAAGACGGCAAAGTGGACGGCC |
| | | CCCACTTTGGGCCCCCTGGGAGAGTGAAGGTGGCCAACCGCGT |
| | | CTTCACTGGGCCCTCTGAGATTGAGGACGAGAACGGTCAGAGG |
| | | AAGCCCACAGACGAGCATGTGGCGCTGTTGGCCCTGAGGCACT |
| | | GGGAGGACATCCCCCGCGCAGGCTGCCGCCTGGTGCCAGAGC |
| | | ATGTGGAGACGAGGCCGCTGCTCAACCCCGACAAGCCGGGCAT |
| | | CGAGCAGGGCCGCCTGGAGCTGTGGGTGGACATGTTCCCCATG |
| | | GACATGCCAGCCCCTGGGACGCCTCTGGACATCTCACCTCGGA |
| | | AGCCCAAGAAGTACGAGCTGCGGGTCATCATCTGGAACACAGA |
| | | TGAGGTGGTCTTGGAGGACGACGACTTCTTCACAGGGGAGAAG |
| | | TCCAGTGACATCTTCGTGAGGGGGTGGCTGAAGGGCCAGCAGG |
| | | AGGACAAGCAGGACACAGACGTCCACTACCACTCCCTCACTGG |
| | | CGAGGGCAACTTCAACTGGCGCTACCTGTTCCCCTTCGACTACC |
| | | TGGCGGCGGAGGAGAAGATCGTCATCTCCAAGAAGGAGTCCAT |
| | | GTTCTCCTGGGACGAGACCGAGTACAAGATCCCCGCGCGGCTC |
| | | ACCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGA |
| | | GCTTCATCTGGTTCCTGAACCCTCTCAAGTCGGCTCGCTACTTC |
| | | TTGTGGCACACGTATCGCTGGCTGCTCCTCAAACTGTTGCTGCT |
| | | CCTGCTGCTGCTCCTCCTCCTCGCCCTGTTCCTCTACTCTGTGC |
| | | CTGGCTACCTGGTCAAGAAAATCCTCGGGGCCTGAGCCCAGTG |
| | | GCCTCCTGGCCGGCCCGACACGGCCTTCGTCTGGTTCCTCAAC |
| | | CCTCTCAAGTCCATCAAGTACCTCATCTGCACCCGGTACAAGTG |
| | | GCTCATCATCAAGATCGTGCTGGCGCTGTTGGGGCTGCTCATGT |
| | | TGGGGCTCTTCCTCTACAGCCTCCCTGGCTACATGGTCAAAAAG |
| | | CTCCTTGGGGCATGAAGGCCGCCAGCTCCCGCCAGCCGCTCCC |
| | | CAGCCCTGCCGCATTTCCTTTCAGTGGCTTGGACTCTTTCCCAT |
| | | CTCCCCTGGGGAGCCTGAGGAGCCCAGCGTCCACTCTTCATGC |
| | | CTTGGGCCGAGCCTGCCTCCTGCTTGCGGGGGCCGCCTGTCCT |
| | | CACTGCCCCAGGCTGCGGCTTGCCCAGTCCCGCCCCTCTGACC |
| | | CCTGCCTGTGGGCTGGGGAGCCTTGGATGGGGTGGGGACCTG |
| | | GAATGGGTCTCTCTTGCCCCACCTGGCTGAGGCGCCACCCTTC |
| | | TTCAGGCCCAGGCTCCAGAGGAAGACTCCTGAAACCCTCCCCA |
| | | GGTCTTCCAAGTACAGGATTGAAGCTTTAGTGAAATTAACCAAG |
| | | GACCATGGGTCAGTGCCCAGGGCTTTAAAAAGAATGAACGAGC |
| | | AAAAGGTATCCCCGCCGTGACCCCTGCAGATAGCACCGGTCTTT |
| | | GATCCGCAGCAGGGGCCAGACCCTGCCCACAAGTCCCAGCGC |
| | | GGCTGCTTCTGCCACTGCTGGGCTCCACTTGGCTCCTCTCACTT |
| | | CCCAGGGGGTCGCCTGTCCTGCCTGTGGGTTTCCATGGCTTCC |
| | | CAGAGCTCCCTCTGCCCCAGCCAGCGCCTCCAGGCCCAGCTGA |
| | | GGAGCTGTGAGAAGCAGCAGAGGGGACTCCCCATCCCGGGCA |
| | | CACCCTGTCCTCCCACCCCTGCCCCCTTGCCCTTCCAGCCCTTT |
| | | CAGCTGCAGCTGGGAGCTGGCCCGTCAAGTGCTGCCCCTGCCT |
| | | GTGTCTGGGTTTCTGTTGGCTGTTTTTCTTTTCTTGAGTGGTGAT |
| | | TTTTCTCTAAATAAAAGAAGTCAAGCACTGAAAAAAAAAAAAAAA |
| | | A |
| 12 | OTOF-203 transcript | CCGTGAGTTCTGCCCAGGCCCTGTGAGCTCACCAGAGCCACAG |
| | (NM_194323.2), | ACTCACAGCCCAGAGGTGGCTTCTTCCTTCAGGAACTGAAGAAC |
| | human otoferlin | CCCCATGAACACCAACATCTCCAGGTTCTGAGAACAGAACCTGG |
| | transcript variant 4, | GAAATTGATGACTTCCTCATGATGACCGATACTCAGGATGGCCC |
| | 4771 bp, encodes | TAGCGAGAGCTCCCAGATCATGAGGAAGAAGGCCTGAACGACA |
| | the protein of SEQ | TACAGGAGATGATCAAAACGGAGAAGTCCTACCCTGAGCGTCG |
| | ID NO: 3 | CCTGCGGGGCGTCCTGGAGGAGCTGAGCTGTGGCTGCTGCCG |
| | | CTTCCTCTCCCTCGCTGACAAGGACCAGGGCCACTCATCCCGC |
| | | ACCAGGCTTGACCGGGAGCGCCTCAAGTCCTGCATGAGGGAGC |
| | | TGGAAAACATGGGGCAGCAGGCCAGGATGCTGCGGGCCCAGG |
| | | TGAAGCGGCACACGGTGCGGGACAAGCTGAGGCTGTGCCAGAA |
| | | CTTCCTGCAGAAGCTGCGCTTCCTGGCGGACGAGCCCCAGCAC |
| | | AGCATTCCCGACATCTTCATCTGGATGATGAGCAACAACAAGCG |
| | | TGTCGCCTATGCCCGTGTGCCCTCCAAGGACCTGCTCTTCTCCA |
| | | TCGTGGAGGAGGAGACTGGCAAGGACTGCGCCAAGGTCAAGAC |
| | | GCTCTTCCTTAAGCTGCCAGGGAAGCGGGGCTTCGGCTCGGCA |
| | | GGCTGGACAGTGCAGGCCAAGGTGGAGCTGTACCTGTGGCTGG |
| | | GCCTCAGCAAACAGCGCAAGGAGTTCCTGTGCGGCCTGCCCTG |
| | | TGGCTTCCAGGAGGTCAAGGCAGCCCAGGGCCTGGGCCTGCAT |
| | | GCCTTCCCACCCGTCAGCCTGGTCTACACCAAGAAGCAGGCGT |
| | | TCCAGCTCCGAGCGCACATGTACCAGGCCCGCAGCCTCTTTGC |
| | | CGCCGACAGCAGCGGACTCTCAGACCCCTTTGCCCGCGTCTTC |
| | | TTCATCAATCAGAGTCAGTGCACAGAGGTGCTGAATGAGACCCT |
| | | GTGTCCCACCTGGGACCAGATGCTGGTGTTCGACAACCTGGAG |
| | | CTCTATGGTGAAGCTCATGAGCTGAGGGACGATCCGCCCATCAT |
| | | TGTCATTGAAATCTATGACCAGGATTCCATGGGCAAAGCTGACT |
| | | TCATGGGCCGGACCTTCGCCAAACCCCTGGTGAAGATGGCAGA |
| | | CGAGGCGTACTGCCCACCCCGCTTCCCACCTCAGCTCGAGTAC |
| | | TACCAGATCTACCGTGGCAACGCCACAGCTGGAGACCTGCTGG |
| | | CGGCCTTCGAGCTGCTGCAGATTGGACCAGCAGGGAAGGCTGA |
| | | CCTGCCCCCCATCAATGGCCCGGTGGACGTGGACCGAGGTCCC |
| | | ATCATGCCCGTGCCCATGGGCATCCGGCCCGTGCTCAGCAAGT |
| | | ACCGAGTGGAGGTGCTGTTCTGGGGCCTACGGGACCTAAAGCG |
| | | GGTGAACCTGGCCCAGGTGGACCGGCCACGGGTGGACATCGA |
| | | GTGTGCAGGGAAGGGGGTGCAGTCGTCCCTGATCCACAATTAT |
| | | AAGAAGAACCCCAACTTCAACACCCTCGTCAAGTGGTTTGAAGT |
| | | GGACCTCCCAGAGAACGAGCTGCTGCACCCGCCCTTGAACATC |
| | | CGTGTGGTGGACTGCCGGGCCTTCGGTCGCTACACACTGGTGG |
| | | GCTCCCATGCCGTCAGCTCCCTGCGACGCTTCATCTACCGGCC |
| | | CCCAGACCGCTCGGCCCCCAGCTGGAACACCACGGGGGAGGT |
| | | TGTGGTGACTATGGAGCCAGAGGTACCCATCAAGAAACTGGAG |
| | | ACCATGGTGAAGCTGGACGCGACTTCTGAAGCTGTTGTCAAGGT |
| | | GGATGTGGCTGAGGAGGAGAAGGAGAAGAAGAAGAAGAAGAAG |
| | | GGCACTGCGGAGGAGCCAGAGGAGGAGGAGCCAGACGAGAGC |
| | | ATGCTGGACTGGTGGTCCAAGTACTTTGCCTCCATTGACACCAT |
| | | GAAGGAGCAACTTCGACAACAAGAGCCCTCTGGAATTGACTTGG |
| | | AGGAGAAGGAGGAAGTGGACAATACCGAGGGCCTGAAGGGGT |
| | | CAATGAAGGGCAAGGAGAAGGCAAGGGCTGCCAAAGAGGAGAA |
| | | GAAGAAGAAAACTCAGAGCTCTGGCTCTGGCCAGGGGTCCGAG |
| | | GCCCCCGAGAAGAAGAAACCCAAGATTGATGAGCTTAAGGTATA |
| | | CCCCAAAGAGCTGGAGTCCGAGTTTGATAACTTTGAGGACTGGC |
| | | TGCACACTTTCAACTTGCTTCGGGGCAAGACCGGGGATGATGA |
| | | GGATGGCTCCACCGAGGAGGAGCGCATTGTGGGACGCTTCAAG |
| | | GGCTCCCTCTGCGTGTACAAAGTGCCACTCCCAGAGGACGTGT |
| | | CCCGGGAAGCCGGCTACGACTCCACCTACGGCATGTTCCAGGG |
| | | CATCCCGAGCAATGACCCCATCAATGTGCTGGTCCGAGTCTATG |
| | | TGGTCCGGGCCACGGACCTGCACCCTGCTGACATCAACGGCAA |
| | | AGCTGACCCCTACATCGCCATCCGGCTAGGCAAGACTGACATC |
| | | CGCGACAAGGAGAACTACATCTCCAAGCAGCTCAACCCTGTCTT |
| | | TGGGAAGTCCTTTGACATCGAGGCCTCCTTCCCCATGGAATCCA |
| | | TGCTGACGGTGGCTGTGTATGACTGGGACCTGGTGGGCACTGA |
| | | TGACCTCATTGGGGAAACCAAGATCGACCTGGAGAACCGCTTCT |
| | | ACAGCAAGCACCGCGCCACCTGCGGCATCGCCCAGACCTACTC |
| | | CACACATGGCTACAATATCTGGCGGGACCCCATGAAGCCCAGC |
| | | CAGATCCTGACCCGCCTCTGCAAAGACGGCAAAGTGGACGGCC |
| | | CCCACTTTGGGCCCCCTGGGAGAGTGAAGGTGGCCAACCGCGT |
| | | CTTCACTGGGCCCTCTGAGATTGAGGACGAGAACGGTCAGAGG |
| | | AAGCCCACAGACGAGCATGTGGCGCTGTTGGCCCTGAGGCACT |
| | | GGGAGGACATCCCCCGCGCAGGCTGCCGCCTGGTGCCAGAGC |
| | | ATGTGGAGACGAGGCCGCTGCTCAACCCCGACAAGCCGGGCAT |
| | | CGAGCAGGGCCGCCTGGAGCTGTGGGTGGACATGTTCCCCATG |
| | | GACATGCCAGCCCCTGGGACGCCTCTGGACATCTCACCTCGGA |
| | | AGCCCAAGAAGTACGAGCTGCGGGTCATCATCTGGAACACAGA |
| | | TGAGGTGGTCTTGGAGGACGACGACTTCTTCACAGGGGAGAAG |
| | | TCCAGTGACATCTTCGTGAGGGGGTGGCTGAAGGGCCAGCAGG |
| | | AGGACAAGCAGGACACAGACGTCCACTACCACTCCCTCACTGG |
| | | CGAGGGCAACTTCAACTGGCGCTACCTGTTCCCCTTCGACTACC |
| | | TGGCGGCGGAGGAGAAGATCGTCATCTCCAAGAAGGAGTCCAT |
| | | GTTCTCCTGGGACGAGACCGAGTACAAGATCCCCGCGCGGCTC |
| | | ACCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGG |
| | | CCTTCGTCTGGTTCCTCAACCCTCTCAAGTCCATCAAGTACCTCA |
| | | TCTGCACCCGGTACAAGTGGCTCATCATCAAGATCGTGCTGGCG |
| | | CTGTTGGGGCTGCTCATGTTGGGGCTCTTCCTCTACAGCCTCCC |
| | | TGGCTACATGGTCAAAAAGCTCCTTGGGGCATGAAGGCCGCCA |
| | | GCTCCCGCCAGCCGCTCCCCAGCCCTGCCGCATTTCCTTTCAG |
| | | TGGCTTGGACTCTTTCCCATCTCCCCTGGGGAGCCTGAGGAGC |
| | | CCAGCGTCCACTCTTCATGCCTTGGGCCGAGCCTGCCTCCTGC |
| | | TTGCGGGGGCCGCCTGTCCTCACTGCCCCAGGCTGCGGCTTGC |
| | | CCAGTCCCGCCCCTCTGACCCCTGCCTGTGGGCTGGGGAGCCT |
| | | TGGATGGGGTGGGGACCTGGAATGGGTCTCTCTTGCCCCACCT |
| | | GGCTGAGGCGCCACCCTTCTTCAGGCCCAGGCTCCAGAGGAAG |
| | | ACTCCTGAAACCCTCCCCAGGTCTTCCAAGTACAGGATTGAAGC |
| | | TTTAGTGAAATTAACCAAGGACCATGGGTCAGTGCCCAGGGCTT |
| | | TAAAAAGAATGAACGAGCAAAAGGTATCCCCGCCGTGACCCCTG |
| | | CAGATAGCACCGGTCTTTGATCCGCAGCAGGGGCCAGACCCTG |
| | | CCCACAAGTCCCAGCGCGGCTGCTTCTGCCACTGCTGGGCTCC |
| | | ACTTGGCTCCTCTCACTTCCCAGGGGGTCGCCTGTCCTGCCTGT |
| | | GGGTTTCCATGGCTTCCCAGAGCTCCCTCTGCCCCAGCCAGCG |
| | | CCTCCAGGCCCAGCTGAGGAGCTGTGAGAAGCAGCAGAGGGG |
| | | ACTCCCCATCCCGGGCACACCCTGTCCTCCCACCCCTGCCCCC |
| | | TTGCCCTTCCAGCCCTTTCAGCTGCAGCTGGGAGCTGGCCCGT |
| | | CAAGTGCTGCCCCTGCCTGTGTCTGGGTTTCTGTTGGCTGTTTT |
| | | TCTTTTCTTGAGTGGTGATTTTTCTCTAAATAAAAGAAGTCAAGC |
| | | ACTGAAAAAAAAAAAAAAAA |
| 13 | OTOF-208 transcript | CCGTGAGTTCTGCCCAGGCCCTGTGAGCTCACCAGAGCCACAG |
| | (NM_194322.2), | ACTCACAGCCCAGAGGTGGCTTCTTCCTTCAGGAACTGAAGAAC |
| | human otoferlin | CCCCATGAACACCAACATCTCCAGGTTCTGAGAACAGAACCTGG |
| | transcript variant 3, | GAAATTGATGACTTCCTCATGATGACCGATACTCAGGATGGCCC |
| | 5123 bp, encodes | TAGCGAGAGCTCCCAGATCATGAGGTCCCTCACTCCCCTGATCA |
| | the protein of SEQ | ACAGGGAGGAGGCATTTGGGGAGGCTGGGGAGGCGGGGCTGT |
| | ID NO: 4 | GGCCCAGCATCACCCACACTCCTGATTCACAGGAAGAAGGCCT |
| | | GAACGACATACAGGAGATGATCAAAACGGAGAAGTCCTACCCTG |
| | | AGCGTCGCCTGCGGGGCGTCCTGGAGGAGCTGAGCTGTGGCT |
| | | GCTGCCGCTTCCTCTCCCTCGCTGACAAGGACCAGGGCCACTC |
| | | ATCCCGCACCAGGCTTGACCGGGAGCGCCTCAAGTCCTGCATG |
| | | AGGGAGCTGGAAAACATGGGGCAGCAGGCCAGGATGCTGCGG |
| | | GCCCAGGTGAAGCGGCACACGGTGCGGGACAAGCTGAGGCTG |
| | | TGCCAGAACTTCCTGCAGAAGCTGCGCTTCCTGGCGGACGAGC |
| | | CCCAGCACAGCATTCCCGACATCTTCATCTGGATGATGAGCAAC |
| | | AACAAGCGTGTCGCCTATGCCCGTGTGCCCTCCAAGGACCTGC |
| | | TCTTCTCCATCGTGGAGGAGGAGACTGGCAAGGACTGCGCCAA |
| | | GGTCAAGACGCTCTTCCTTAAGCTGCCAGGGAAGCGGGGCTTC |
| | | GGCTCGGCAGGCTGGACAGTGCAGGCCAAGGTGGAGCTGTAC |
| | | CTGTGGCTGGGCCTCAGCAAACAGCGCAAGGAGTTCCTGTGCG |
| | | GCCTGCCCTGTGGCTTCCAGGAGGTCAAGGCAGCCCAGGGCCT |
| | | GGGCCTGCATGCCTTCCCACCCGTCAGCCTGGTCTACACCAAG |
| | | AAGCAGGCGTTCCAGCTCCGAGCGCACATGTACCAGGCCCGCA |
| | | GCCTCTTTGCCGCCGACAGCAGCGGACTCTCAGACCCCTTTGC |
| | | CCGCGTCTTCTTCATCAATCAGAGTCAGTGCACAGAGGTGCTGA |
| | | ATGAGACCCTGTGTCCCACCTGGGACCAGATGCTGGTGTTCGA |
| | | CAACCTGGAGCTCTATGGTGAAGCTCATGAGCTGAGGGACGAT |
| | | CCGCCCATCATTGTCATTGAAATCTATGACCAGGATTCCATGGG |
| | | CAAAGCTGACTTCATGGGCCGGACCTTCGCCAAACCCCTGGTG |
| | | AAGATGGCAGACGAGGCGTACTGCCCACCCCGCTTCCCACCTC |
| | | AGCTCGAGTACTACCAGATCTACCGTGGCAACGCCACAGCTGG |
| | | AGACCTGCTGGCGGCCTTCGAGCTGCTGCAGATTGGACCAGCA |
| | | GGGAAGGCTGACCTGCCCCCCATCAATGGCCCGGTGGACGTG |
| | | GACCGAGGTCCCATCATGCCCGTGCCCATGGGCATCCGGCCCG |
| | | TGCTCAGCAAGTACCGAGTGGAGGTGCTGTTCTGGGGCCTACG |
| | | GGACCTAAAGCGGGTGAACCTGGCCCAGGTGGACCGGCCACG |
| | | GGTGGACATCGAGTGTGCAGGGAAGGGGGTGCAGTCGTCCCT |
| | | GATCCACAATTATAAGAAGAACCCCAACTTCAACACCCTCGTCAA |
| | | GTGGTTTGAAGTGGACCTCCCAGAGAACGAGCTGCTGCACCCG |
| | | CCCTTGAACATCCGTGTGGTGGACTGCCGGGCCTTCGGTCGCT |
| | | ACACACTGGTGGGCTCCCATGCCGTCAGCTCCCTGCGACGCTT |
| | | CATCTACCGGCCCCCAGACCGCTCGGCCCCCAGCTGGAACACC |
| | | ACGGTCAGGCTTCTCCGGCGCTGCCGTGTGCTGTGCAATGGGG |
| | | GCTCCTCCTCTCACTCCACAGGGGAGGTTGTGGTGACTATGGA |
| | | GCCAGAGGTACCCATCAAGAAACTGGAGACCATGGTGAAGCTG |
| | | GACGCGACTTCTGAAGCTGTTGTCAAGGTGGATGTGGCTGAGG |
| | | AGGAGAAGGAGAAGAAGAAGAAGAAGAAGGGCACTGCGGAGG |
| | | AGCCAGAGGAGGAGGAGCCAGACGAGAGCATGCTGGACTGGT |
| | | GGTCCAAGTACTTTGCCTCCATTGACACCATGAAGGAGCAACTT |
| | | CGACAACAAGAGCCCTCTGGAATTGACTTGGAGGAGAAGGAGG |
| | | AAGTGGACAATACCGAGGGCCTGAAGGGGTCAATGAAGGGCAA |
| | | GGAGAAGGCAAGGGCTGCCAAAGAGGAGAAGAAGAAGAAAACT |
| | | CAGAGCTCTGGCTCTGGCCAGGGGTCCGAGGCCCCCGAGAAG |
| | | AAGAAACCCAAGATTGATGAGCTTAAGGTATACCCCAAAGAGCT |
| | | GGAGTCCGAGTTTGATAACTTTGAGGACTGGCTGCACACTTTCA |
| | | ACTTGCTTCGGGGCAAGACCGGGGATGATGAGGATGGCTCCAC |
| | | CGAGGAGGAGCGCATTGTGGGACGCTTCAAGGGCTCCCTCTGC |
| | | GTGTACAAAGTGCCACTCCCAGAGGACGTGTCCCGGGAAGCCG |
| | | GCTACGACTCCACCTACGGCATGTTCCAGGGCATCCCGAGCAA |
| | | TGACCCCATCAATGTGCTGGTCCGAGTCTATGTGGTCCGGGCC |
| | | ACGGACCTGCACCCTGCTGACATCAACGGCAAAGCTGACCCCT |
| | | ACATCGCCATCCGGCTAGGCAAGACTGACATCCGCGACAAGGA |
| | | GAACTACATCTCCAAGCAGCTCAACCCTGTCTTTGGGAAGTCCT |
| | | TTGACATCGAGGCCTCCTTCCCCATGGAATCCATGCTGACGGTG |
| | | GCTGTGTATGACTGGGACCTGGTGGGCACTGATGACCTCATTG |
| | | GGGAAACCAAGATCGACCTGGAGAACCGCTTCTACAGCAAGCA |
| | | CCGCGCCACCTGCGGCATCGCCCAGACCTACTCCACACATGGC |
| | | TACAATATCTGGCGGGACCCCATGAAGCCCAGCCAGATCCTGA |
| | | CCCGCCTCTGCAAAGACGGCAAAGTGGACGGCCCCCACTTTGG |
| | | GCCCCCTGGGAGAGTGAAGGTGGCCAACCGCGTCTTCACTGGG |
| | | CCCTCTGAGATTGAGGACGAGAACGGTCAGAGGAAGCCCACAG |
| | | ACGAGCATGTGGCGCTGTTGGCCCTGAGGCACTGGGAGGACAT |
| | | CCCCCGCGCAGGCTGCCGCCTGGTGCCAGAGCATGTGGAGAC |
| | | GAGGCCGCTGCTCAACCCCGACAAGCCGGGCATCGAGCAGGG |
| | | CCGCCTGGAGCTGTGGGTGGACATGTTCCCCATGGACATGCCA |
| | | GCCCCTGGGACGCCTCTGGACATCTCACCTCGGAAGCCCAAGA |
| | | AGTACGAGCTGCGGGTCATCATCTGGAACACAGATGAGGTGGT |
| | | CTTGGAGGACGACGACTTCTTCACAGGGGAGAAGTCCAGTGAC |
| | | ATCTTCGTGAGGGGGTGGCTGAAGGGCCAGCAGGAGGACAAG |
| | | CAGGACACAGACGTCCACTACCACTCCCTCACTGGCGAGGGCA |
| | | ACTTCAACTGGCGCTACCTGTTCCCCTTCGACTACCTGGCGGCG |
| | | GAGGAGAAGATCGTCATCTCCAAGAAGGAGTCCATGTTCTCCTG |
| | | GGACGAGACCGAGTACAAGATCCCCGCGCGGCTCACCCTGCAG |
| | | ATCTGGGATGCGGACCACTTCTCCGCTGACGACTTCCTGGGGG |
| | | CCATCGAGCTGGACCTGAACCGGTTCCCGCGGGGCGCAAAGAC |
| | | AGCCAAGCAGTGCACCATGGAGATGGCCACCGGGGAGGTGGA |
| | | CGTGCCCCTCGTGTCCATCTTCAAGCAAAAGCGCGTCAAAGGCT |
| | | GGTGGCCCCTCCTGGCCCGCAATGAGAACGATGAGTTTGAGCT |
| | | CACGGGCAAGGTGGAGGCTGAGCTGCATTTACTGACAGCAGAG |
| | | GAGGCAGAGAAGAACCCAGTGGGCCTGGCCCGCAATGAACCTG |
| | | ACCCCCTAGAGAAACCCAACCGGCCCGACACGAGCTTCATCTG |
| | | GTTCCTGAACCCTCTCAAGTCGGCTCGCTACTTCTTGTGGCACA |
| | | CGTATCGCTGGCTGCTCCTCAAACTGTTGCTGCTCCTGCTGCTG |
| | | CTCCTCCTCCTCGCCCTGTTCCTCTACTCTGTGCCTGGCTACCT |
| | | GGTCAAGAAAATCCTCGGGGCCTGAGCCCAGTGGCCTCCTGGC |
| | | CGGCCCGACACGGCCTTCGTCTGGTTCCTCAACCCTCTCAAGTC |
| | | CATCAAGTACCTCATCTGCACCCGGTACAAGTGGCTCATCATCA |
| | | AGATCGTGCTGGCGCTGTTGGGGCTGCTCATGTTGGGGCTCTT |
| | | CCTCTACAGCCTCCCTGGCTACATGGTCAAAAAGCTCCTTGGGG |
| | | CATGAAGGCCGCCAGCTCCCGCCAGCCGCTCCCCAGCCCTGCC |
| | | GCATTTCCTTTCAGTGGCTTGGACTCTTTCCCATCTCCCCTGGG |
| | | GAGCCTGAGGAGCCCAGCGTCCACTCTTCATGCCTTGGGCCGA |
| | | GCCTGCCTCCTGCTTGCGGGGGCCGCCTGTCCTCACTGCCCCA |
| | | GGCTGCGGCTTGCCCAGTCCCGCCCCTCTGACCCCTGCCTGTG |
| | | GGCTGGGGAGCCTTGGATGGGGTGGGGACCTGGAATGGGTCT |
| | | CTCTTGCCCCACCTGGCTGAGGCGCCACCCTTCTTCAGGCCCA |
| | | GGCTCCAGAGGAAGACTCCTGAAACCCTCCCCAGGTCTTCCAA |
| | | GTACAGGATTGAAGCTTTAGTGAAATTAACCAAGGACCATGGGT |
| | | CAGTGCCCAGGGCTTTAAAAAGAATGAACGAGCAAAAGGTATCC |
| | | CCGCCGTGACCCCTGCAGATAGCACCGGTCTTTGATCCGCAGC |
| | | AGGGGCCAGACCCTGCCCACAAGTCCCAGCGCGGCTGCTTCTG |
| | | CCACTGCTGGGCTCCACTTGGCTCCTCTCACTTCCCAGGGGGT |
| | | CGCCTGTCCTGCCTGTGGGTTTCCATGGCTTCCCAGAGCTCCCT |
| | | CTGCCCCAGCCAGCGCCTCCAGGCCCAGCTGAGGAGCTGTGA |
| | | GAAGCAGCAGAGGGGACTCCCCATCCCGGGCACACCCTGTCCT |
| | | CCCACCCCTGCCCCCTTGCCCTTCCAGCCCTTTCAGCTGCAGCT |
| | | GGGAGCTGGCCCGTCAAGTGCTGCCCCTGCCTGTGTCTGGGTT |
| | | TCTGTTGGCTGTTTTTCTTTTCTTGAGTGGTGATTTTTCTCTAAAT |
| | | AAAAGAAGTCAAGCACTGAAAAAAAAAAAAAAAA |
| 14 | DNA sequence | ATGGCCTTGCTCATCCACCTCAAGACAGTCTCGGAGCTGCGGG |
| | encoding the human | GCAGGGGCGACCGGATCGCCAAAGTGACTTTCCGAGGGCAATC |
| | otoferlin isoform 5 | CTTCTACTCTCGGGTCCTGGAGAACTGTGAGGATGTGGCTGACT |
| | protein (SEQ ID NO: | TTGATGAGACATTTCGGTGGCCGGTGGCCAGCAGCATCGACAG |
| | 5), 5994 bp, | AAATGAGATGCTGGAGATTCAGGTTTTCAACTACAGCAAAGTCTT |
| | corresponds to the | CAGCAACAAGCTCATCGGGACCTTCCGCATGGTGCTGCAGAAG |
| | coding sequence | GTGGTAGAGGAGAGCCATGTGGAGGTGACTGACACGCTGATTG |
| | documented in | ATGACAACAATGCTATCATCAAGACCAGCCTGTGCGTGGAGGTC |
| | NM_001287489 | CGGTATCAGGCCACTGACGGCACAGTGGGCTCCTGGGACGATG |
| | | GGGACTTCCTGGGAGATGAGTCTCTTCAAGAGGAAGAGAAGGA |
| | | CAGCCAAGAGACGGATGGACTGCTCCCAGGCTCCCGGCCCAGC |
| | | TCCCGGCCCCCAGGAGAGAAGAGCTTCCGGAGAGCCGGGAGG |
| | | AGCGTGTTCTCCGCCATGAAGCTCGGCAAAAACCGGTCTCACAA |
| | | GGAGGAGCCCCAAAGACCAGATGAACCGGCGGTGCTGGAGAT |
| | | GGAAGACCTTGACCATCTGGCCATTCGGCTAGGAGATGGACTG |
| | | GATCCCGACTCGGTGTCTCTAGCCTCAGTCACAGCTCTCACCAC |
| | | TAATGTCTCCAACAAGCGATCTAAGCCAGACATTAAGATGGAGC |
| | | CAAGTGCTGGGCGGCCCATGGATTACCAGGTCAGCATCACGGT |
| | | GATCGAGGCCCGGCAGCTGGTGGGCTTGAACATGGACCCTGTG |
| | | GTGTGCGTGGAGGTGGGTGACGACAAGAAGTACACATCCATGA |
| | | AGGAGTCCACTAACTGCCCCTATTACAACGAGTACTTCGTCTTC |
| | | GACTTCCATGTCTCTCCGGATGTCATGTTTGACAAGATCATCAAG |
| | | ATTTCGGTGATTCACTCCAAGAACCTGCTGCGCAGTGGCACCCT |
| | | GGTGGGCTCCTTCAAAATGGACGTGGGAACCGTGTACTCGCAG |
| | | CCAGAGCACCAGTTCCATCACAAGTGGGCCATCCTGTCTGACCC |
| | | CGATGACATCTCCTCGGGGCTGAAGGGCTACGTGAAGTGTGAC |
| | | GTTGCCGTGGTGGGCAAAGGGGACAACATCAAGACGCCCCACA |
| | | AGGCCAATGAGACCGACGAAGATGACATTGAGGGGAACTTGCT |
| | | GCTCCCCGAGGGGGTGCCCCCCGAACGCCAGTGGGCCCGGTT |
| | | CTATGTGAAAATTTACCGAGCAGAGGGGCTGCCCCGTATGAACA |
| | | CAAGCCTCATGGCCAATGTAAAGAAGGCTTTCATCGGTGAAAAC |
| | | AAGGACCTCGTGGACCCCTACGTGCAAGTCTTCTTTGCTGGCCA |
| | | GAAGGGCAAGACTTCAGTGCAGAAGAGCAGCTATGAGCCCCTG |
| | | TGGAATGAGCAGGTCGTCTTTACAGACCTCTTCCCCCCACTCTG |
| | | CAAACGCATGAAGGTGCAGATCCGAGACTCGGACAAGGTCAAC |
| | | GACGTGGCCATCGGCACCCACTTCATTGACCTGCGCAAGATTTC |
| | | TAATGACGGAGACAAAGGCTTCCTGCCCACACTGGGCCCAGCC |
| | | TGGGTGAACATGTACGGCTCCACACGTAACTACACGCTGCTGGA |
| | | TGAGCATCAGGACCTGAACGAGGGCCTGGGGGAGGGTGTGTC |
| | | CTTCCGGGCCCGGCTCCTGCTGGGCCTGGCTGTGGAGATCGTA |
| | | GACACCTCCAACCCTGAGCTCACCAGCTCCACAGAGGTGCAGG |
| | | TGGAGCAGGCCACGCCCATCTCGGAGAGCTGTGCAGGTAAAAT |
| | | GGAAGAATTCTTTCTCTTTGGAGCCTTCCTGGAGGCCTCAATGA |
| | | TCGACCGGAGAAACGGAGACAAGCCCATCACCTTTGAGGTCAC |
| | | CATAGGCAACTATGGGAACGAAGTTGATGGCCTGTCCCGGCCC |
| | | CAGCGGCCTCGGCCCCGGAAGGAGCCGGGGGATGAGGAAGAA |
| | | GTAGACCTGATTCAGAACGCAAGTGATGACGAGGCCGGTGATG |
| | | CCGGGGACCTGGCCTCAGTCTCCTCCACTCCACCAATGCGGCC |
| | | CCAGGTCACCGACAGGAACTACTTCCATCTGCCCTACCTGGAGC |
| | | GAAAGCCCTGCATCTACATCAAGAGCTGGTGGCCGGACCAGCG |
| | | CCGCCGCCTCTACAATGCCAACATCATGGACCACATTGCCGACA |
| | | AGCTGGAAGAAGGCCTGAACGACATACAGGAGATGATCAAAAC |
| | | GGAGAAGTCCTACCCTGAGCGTCGCCTGCGGGGCGTCCTGGA |
| | | GGAGCTGAGCTGTGGCTGCTGCCGCTTCCTCTCCCTCGCTGAC |
| | | AAGGACCAGGGCCACTCATCCCGCACCAGGCTTGACCGGGAGC |
| | | GCCTCAAGTCCTGCATGAGGGAGCTGGAAAACATGGGGCAGCA |
| | | GGCCAGGATGCTGCGGGCCCAGGTGAAGCGGCACACGGTGCG |
| | | GGACAAGCTGAGGCTGTGCCAGAACTTCCTGCAGAAGCTGCGC |
| | | TTCCTGGCGGACGAGCCCCAGCACAGCATTCCCGACATCTTCAT |
| | | CTGGATGATGAGCAACAACAAGCGTGTCGCCTATGCCCGTGTG |
| | | CCCTCCAAGGACCTGCTCTTCTCCATCGTGGAGGAGGAGACTG |
| | | GCAAGGACTGCGCCAAGGTCAAGACGCTCTTCCTTAAGCTGCC |
| | | AGGGAAGCGGGGCTTCGGCTCGGCAGGCTGGACAGTGCAGGC |
| | | CAAGGTGGAGCTGTACCTGTGGCTGGGCCTCAGCAAACAGCGC |
| | | AAGGAGTTCCTGTGCGGCCTGCCCTGTGGCTTCCAGGAGGTCA |
| | | AGGCAGCCCAGGGCCTGGGCCTGCATGCCTTCCCACCCGTCAG |
| | | CCTGGTCTACACCAAGAAGCAGGCGTTCCAGCTCCGAGCGCAC |
| | | ATGTACCAGGCCCGCAGCCTCTTTGCCGCCGACAGCAGCGGAC |
| | | TCTCAGACCCCTTTGCCCGCGTCTTCTTCATCAATCAGAGTCAG |
| | | TGCACAGAGGTGCTGAATGAGACCCTGTGTCCCACCTGGGACC |
| | | AGATGCTGGTGTTCGACAACCTGGAGCTCTATGGTGAAGCTCAT |
| | | GAGCTGAGGGACGATCCGCCCATCATTGTCATTGAAATCTATGA |
| | | CCAGGATTCCATGGGCAAAGCTGACTTCATGGGCCGGACCTTC |
| | | GCCAAACCCCTGGTGAAGATGGCAGACGAGGCGTACTGCCCAC |
| | | CCCGCTTCCCACCTCAGCTCGAGTACTACCAGATCTACCGTGGC |
| | | AACGCCACAGCTGGAGACCTGCTGGCGGCCTTCGAGCTGCTGC |
| | | AGATTGGACCAGCAGGGAAGGCTGACCTGCCCCCCATCAATGG |
| | | CCCGGTGGACGTGGACCGAGGTCCCATCATGCCCGTGCCCATG |
| | | GGCATCCGGCCCGTGCTCAGCAAGTACCGAGTGGAGGTGCTGT |
| | | TCTGGGGCCTACGGGACCTAAAGCGGGTGAACCTGGCCCAGGT |
| | | GGACCGGCCACGGGTGGACATCGAGTGTGCAGGGAAGGGGGT |
| | | GCAGTCGTCCCTGATCCACAATTATAAGAAGAACCCCAACTTCA |
| | | ACACCCTCGTCAAGTGGTTTGAAGTGGACCTCCCAGAGAACGA |
| | | GCTGCTGCACCCGCCCTTGAACATCCGTGTGGTGGACTGCCGG |
| | | GCCTTCGGTCGCTACACACTGGTGGGCTCCCATGCCGTCAGCT |
| | | CCCTGCGACGCTTCATCTACCGGCCCCCAGACCGCTCGGCCCC |
| | | CAGCTGGAACACCACGGTCAGGCTTCTCCGGCGCTGCCGTGTG |
| | | CTGTGCAATGGGGGCTCCTCCTCTCACTCCACAGGGGAGGTTG |
| | | TGGTGACTATGGAGCCAGAGGTACCCATCAAGAAACTGGAGAC |
| | | CATGGTGAAGCTGGACGCGACTTCTGAAGCTGTTGTCAAGGTG |
| | | GATGTGGCTGAGGAGGAGAAGGAGAAGAAGAAGAAGAAGAAGG |
| | | GCACTGCGGAGGAGCCAGAGGAGGAGGAGCCAGACGAGAGCA |
| | | TGCTGGACTGGTGGTCCAAGTACTTTGCCTCCATTGACACCATG |
| | | AAGGAGCAACTTCGACAACAAGAGCCCTCTGGAATTGACTTGGA |
| | | GGAGAAGGAGGAAGTGGACAATACCGAGGGCCTGAAGGGGTC |
| | | AATGAAGGGCAAGGAGAAGGCAAGGGCTGCCAAAGAGGAGAAG |
| | | AAGAAGAAAACTCAGAGCTCTGGCTCTGGCCAGGGGTCCGAGG |
| | | CCCCCGAGAAGAAGAAACCCAAGATTGATGAGCTTAAGGTATAC |
| | | CCCAAAGAGCTGGAGTCCGAGTTTGATAACTTTGAGGACTGGCT |
| | | GCACACTTTCAACTTGCTTCGGGGCAAGACCGGGGATGATGAG |
| | | GATGGCTCCACCGAGGAGGAGCGCATTGTGGGACGCTTCAAGG |
| | | GCTCCCTCTGCGTGTACAAAGTGCCACTCCCAGAGGACGTGTC |
| | | CCGGGAAGCCGGCTACGACTCCACCTACGGCATGTTCCAGGGC |
| | | ATCCCGAGCAATGACCCCATCAATGTGCTGGTCCGAGTCTATGT |
| | | GGTCCGGGCCACGGACCTGCACCCTGCTGACATCAACGGCAAA |
| | | GCTGACCCCTACATCGCCATCCGGCTAGGCAAGACTGACATCC |
| | | GCGACAAGGAGAACTACATCTCCAAGCAGCTCAACCCTGTCTTT |
| | | GGGAAGTCCTTTGACATCGAGGCCTCCTTCCCCATGGAATCCAT |
| | | GCTGACGGTGGCTGTGTATGACTGGGACCTGGTGGGCACTGAT |
| | | GACCTCATTGGGGAAACCAAGATCGACCTGGAGAACCGCTTCTA |
| | | CAGCAAGCACCGCGCCACCTGCGGCATCGCCCAGACCTACTCC |
| | | ACACATGGCTACAATATCTGGCGGGACCCCATGAAGCCCAGCC |
| | | AGATCCTGACCCGCCTCTGCAAAGACGGCAAAGTGGACGGCCC |
| | | CCACTTTGGGCCCCCTGGGAGAGTGAAGGTGGCCAACCGCGTC |
| | | TTCACTGGGCCCTCTGAGATTGAGGACGAGAACGGTCAGAGGA |
| | | AGCCCACAGACGAGCATGTGGCGCTGTTGGCCCTGAGGCACTG |
| | | GGAGGACATCCCCCGCGCAGGCTGCCGCCTGGTGCCAGAGCA |
| | | TGTGGAGACGAGGCCGCTGCTCAACCCCGACAAGCCGGGCATC |
| | | GAGCAGGGCCGCCTGGAGCTGTGGGTGGACATGTTCCCCATGG |
| | | ACATGCCAGCCCCTGGGACGCCTCTGGACATCTCACCTCGGAA |
| | | GCCCAAGAAGTACGAGCTGCGGGTCATCATCTGGAACACAGAT |
| | | GAGGTGGTCTTGGAGGACGACGACTTCTTCACAGGGGAGAAGT |
| | | CCAGTGACATCTTCGTGAGGGGGTGGCTGAAGGGCCAGCAGGA |
| | | GGACAAGCAGGACACAGACGTCCACTACCACTCCCTCACTGGC |
| | | GAGGGCAACTTCAACTGGCGCTACCTGTTCCCCTTCGACTACCT |
| | | GGCGGCGGAGGAGAAGATCGTCATCTCCAAGAAGGAGTCCATG |
| | | TTCTCCTGGGACGAGACCGAGTACAAGATCCCCGCGCGGCTCA |
| | | CCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGG |
| | | CCTTCGTCTGGTTCCTCAACCCTCTCAAGTCCATCAAGTACCTCA |
| | | TCTGCACCCGGTACAAGTGGCTCATCATCAAGATCGTGCTGGCG |
| | | CTGTTGGGGCTGCTCATGTTGGGGCTCTTCCTCTACAGCCTCCC |
| | | TGGCTACATGGTCAAAAAGCTCCTTGGGGCATGA |
| 15 | mOTOF-201_1 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_031875.2), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 2, | CGAGGCAAAGGTGACCGGATTGCCAAAGTCACTTTCCGAGGGC |
| | 7125 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 6 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGAGCGGGAAG |
| | | GAGTGTGTTCTCGGCCATGAAACTCGGCAAAACTCGGTCCCACA |
| | | AAGAGGAGCCCCAAAGACAAGATGAGCCAGCAGTGCTGGAGAT |
| | | GGAGGACCTGGACCACCTAGCCATTCAGCTGGGGGATGGGCTG |
| | | GATCCTGACTCCGTGTCTCTAGCCTCGGTCACCGCTCTCACCAG |
| | | CAATGTCTCCAACAAACGGTCTAAGCCAGATATTAAGATGGAGC |
| | | CCAGTGCTGGAAGGCCCATGGATTACCAGGTCAGCATCACAGT |
| | | GATTGAGGCTCGGCAGCTGGTGGGCTTGAACATGGACCCTGTG |
| | | GTGTGTGTGGAGGTGGGTGATGACAAGAAATACACGTCAATGAA |
| | | GGAGTCCACAAACTGCCCTTACTACAACGAGTACTTTGTCTTCG |
| | | ACTTCCATGTCTCTCCTGATGTCATGTTTGACAAGATCATCAAGA |
| | | TCTCGGTTATCCATTCTAAGAACCTGCTTCGGAGCGGCACCCTG |
| | | GTGGGTTCCTTCAAAATGGATGTGGGGACTGTGTATTCCCAGCC |
| | | TGAACACCAGTTCCATCACAAATGGGCCATCCTGTCAGACCCCG |
| | | ATGACATCTCTGCTGGGTTGAAGGGTTATGTAAAGTGTGATGTC |
| | | GCTGTGGTGGGCAAGGGAGACAACATCAAGACACCCCACAAGG |
| | | CCAACGAGACGGATGAGGACGACATTGAAGGGAACTTGCTGCT |
| | | CCCCGAGGGCGTGCCCCCCGAACGGCAGTGGGCACGGTTCTA |
| | | TGTGAAAATTTACCGAGCAGAGGGACTGCCCCGGATGAACACAA |
| | | GCCTCATGGCCAACGTGAAGAAGGCGTTCATCGGTGAGAACAA |
| | | GGACCTCGTCGACCCCTATGTGCAAGTCTTCTTTGCTGGACAAA |
| | | AGGGCAAAACATCAGTGCAGAAGAGCAGCTATGAGCCGCTATG |
| | | GAATGAGCAGGTCGTCTTCACAGACTTGTTCCCCCCACTCTGCA |
| | | AACGCATGAAGGTGCAGATCCGGGACTCTGACAAGGTCAATGAT |
| | | GTGGCCATCGGCACCCACTTCATCGACCTGCGCAAGATTTCCAA |
| | | CGATGGAGACAAAGGCTTCCTGCCTACCCTCGGTCCAGCCTGG |
| | | GTGAACATGTACGGCTCCACGCGCAACTACACACTGCTGGACG |
| | | AGCACCAGGACTTGAATGAAGGCCTGGGGGAGGGTGTGTCCTT |
| | | CCGGGCCCGCCTCATGTTGGGACTAGCTGTGGAGATCCTGGAC |
| | | ACCTCCAACCCAGAGCTCACCAGCTCCACGGAGGTGCAGGTGG |
| | | AGCAGGCCACGCCTGTCTCGGAGAGCTGCACAGGGAGAATGGA |
| | | AGAATTTTTTCTATTTGGAGCCTTCTTGGAAGCCTCAATGATTGA |
| | | CCGGAAAAATGGGGACAAGCCAATTACCTTTGAGGTGACCATAG |
| | | GAAACTACGGCAATGAAGTCGATGGTATGTCCCGGCCCCTGAG |
| | | GCCTCGGCCCCGGAAAGAGCCTGGGGATGAAGAAGAGGTAGA |
| | | CCTGATTCAGAACTCCAGTGACGATGAAGGTGACGAAGCCGGG |
| | | GACCTGGCCTCGGTGTCCTCCACCCCACCTATGCGGCCCCAGA |
| | | TCACGGACAGGAACTATTTCCACCTGCCCTACCTGGAGCGCAAG |
| | | CCCTGCATCTATATCAAGAGCTGGTGGCCTGACCAGAGGCGGC |
| | | GCCTCTACAATGCCAACATCATGGATCACATTGCTGACAAGCTG |
| | | GAAGAAGGCCTGAATGATGTACAGGAGATGATCAAAACGGAGAA |
| | | GTCCTACCCGGAGCGCCGCCTGCGGGGTGTGCTAGAGGAACTC |
| | | AGCTGTGGCTGCCACCGCTTCCTCTCCCTCTCGGACAAGGACC |
| | | AGGGCCGCTCGTCCCGCACCAGGCTGGATCGAGAGCGTCTTAA |
| | | GTCCTGTATGAGGGAGTTGGAGAGCATGGGACAGCAGGCCAAG |
| | | AGCCTGAGGGCTCAGGTGAAGCGGCACACTGTTCGGGACAAGC |
| | | TGAGGTCATGCCAGAACTTTCTGCAGAAGCTACGCTTCCTGGCG |
| | | GATGAGCCCCAGCACAGCATTCCTGATGTGTTCATTTGGATGAT |
| | | GAGCAACAACAAACGTATCGCCTATGCCCGCGTGCCTTCCAAAG |
| | | ACCTGCTCTTCTCCATCGTGGAGGAGGAACTGGGCAAGGACTG |
| | | CGCCAAAGTCAAGACCCTCTTCCTGAAGCTGCCAGGGAAGAGG |
| | | GGCTTCGGCTCGGCAGGCTGGACAGTACAGGCCAAGCTGGAG |
| | | CTCTACCTGTGGCTGGGCCTCAGCAAGCAGCGAAAGGACTTCC |
| | | TGTGTGGTCTGCCCTGTGGCTTCGAGGAGGTCAAGGCAGCCCA |
| | | AGGCCTGGGCCTGCATTCCTTTCCGCCCATCAGCCTAGTCTACA |
| | | CCAAGAAGCAAGCCTTCCAGCTCCGAGCACACATGTATCAGGC |
| | | CCGAAGCCTCTTTGCTGCTGACAGCAGTGGGCTCTCTGATCCCT |
| | | TTGCCCGTGTCTTCTTCATCAACCAGAGCCAATGCACTGAGGTT |
| | | CTAAACGAGACACTGTGTCCCACCTGGGACCAGATGCTGGTATT |
| | | TGACAACCTGGAGCTGTACGGTGAAGCTCACGAGTTACGAGAT |
| | | GATCCCCCCATCATTGTCATTGAAATCTACGACCAGGACAGCAT |
| | | GGGCAAAGCCGACTTCATGGGCCGGACCTTCGCCAAGCCCCTG |
| | | GTGAAGATGGCAGATGAAGCATACTGCCCACCTCGCTTCCCGC |
| | | CGCAGCTTGAGTACTACCAGATCTACCGAGGCAGTGCCACTGC |
| | | CGGAGACCTACTGGCTGCCTTCGAGCTGCTGCAGATTGGGCCA |
| | | TCAGGGAAGGCTGACCTGCCACCCATCAATGGCCCAGTGGACA |
| | | TGGACAGAGGGCCCATCATGCCTGTGCCCGTGGGAATCCGGCC |
| | | AGTGCTCAGCAAGTACCGAGTGGAGGTGCTGTTCTGGGGCCTG |
| | | AGGGACCTAAAGAGGGTGAACCTGGCCCAGGTGGACCGACCAC |
| | | GGGTGGACATCGAGTGTGCAGGAAAGGGGGTACAATCCTCCCT |
| | | GATTCACAATTATAAGAAGAACCCCAACTTCAACACGCTGGTCAA |
| | | GTGGTTTGAAGTGGACCTCCCGGAGAATGAGCTCCTGCACCCA |
| | | CCCTTGAACATCCGAGTGGTAGATTGCCGGGCCTTTGGACGATA |
| | | CACCCTGGTGGGTTCCCACGCAGTCAGCTCACTGAGGCGCTTC |
| | | ATCTACCGACCTCCAGACCGCTCAGCCCCCAACTGGAACACCA |
| | | CAGTCAGGCTGCTCCGGGGCTGCCACAGGCTGCGCAATGGGG |
| | | GCCCCTCTTCTCGCCCCACAGGGGAGGTTGTAGTAAGCATGGA |
| | | GCCTGAGGAGCCAGTTAAGAAGCTGGAGACCATGGTGAAACTG |
| | | GATGCGACTTCTGATGCTGTGGTCAAGGTGGATGTGGCTGAAG |
| | | ATGAGAAGGAAAGGAAGAAGAAGAAAAAGAAAGGCCCGTCAGA |
| | | GGAGCCAGAGGAGGAAGAGCCCGATGAGAGCATGCTGGATTG |
| | | GTGGTCCAAGTACTTCGCCTCCATCGACACAATGAAGGAGCAAC |
| | | TTCGACAACATGAGACCTCTGGAACTGACTTGGAAGAGAAGGAA |
| | | GAGATGGAAAGCGCTGAGGGCCTGAAGGGACCAATGAAGAGCA |
| | | AGGAGAAGTCCAGAGCTGCAAAGGAGGAGAAAAAGAAGAAAAA |
| | | CCAGAGCCCTGGCCCTGGCCAGGGATCGGAGGCTCCTGAGAA |
| | | GAAGAAAGCCAAGATCGATGAGCTTAAGGTGTACCCCAAGGAG |
| | | CTGGAATCGGAGTTTGACAGCTTTGAGGACTGGCTGCACACCTT |
| | | CAACCTGTTGAGGGGCAAGACGGGAGATGATGAGGATGGCTCC |
| | | ACAGAGGAGGAGCGCATAGTAGGCCGATTCAAGGGCTCCCTCT |
| | | GTGTGTACAAAGTGCCACTCCCAGAAGATGTATCTCGAGAAGCT |
| | | GGCTATGATCCCACCTATGGAATGTTCCAGGGCATCCCAAGCAA |
| | | TGACCCCATCAATGTGCTGGTCCGAATCTATGTGGTCCGGGCCA |
| | | CAGACCTGCACCCGGCCGACATCAATGGCAAAGCTGACCCCTA |
| | | TATTGCCATCAAGTTAGGCAAGACCGACATCCGAGACAAGGAGA |
| | | ACTACATCTCCAAGCAGCTCAACCCTGTGTTTGGGAAGTCCTTT |
| | | GACATTGAGGCCTCCTTCCCCATGGAGTCCATGTTGACAGTGGC |
| | | CGTGTACGACTGGGATCTGGTGGGCACTGATGACCTCATCGGA |
| | | GAAACCAAGATTGACCTGGAAAACCGCTTCTACAGCAAGCATCG |
| | | CGCCACCTGCGGCATCGCACAGACCTATTCCATACATGGCTACA |
| | | ATATCTGGAGGGACCCCATGAAGCCCAGCCAGATCCTGACACG |
| | | CCTCTGTAAAGAGGGCAAAGTGGACGGCCCCCACTTTGGTCCC |
| | | CATGGGAGAGTGAGGGTTGCCAACCGTGTCTTCACGGGGCCTT |
| | | CAGAAATAGAGGATGAGAATGGTCAGAGGAAGCCCACAGATGA |
| | | GCACGTGGCACTGTCTGCTCTGAGACACTGGGAGGACATCCCC |
| | | CGGGTGGGCTGCCGCCTTGTGCCGGAACACGTGGAGACCAGG |
| | | CCGCTGCTCAACCCTGACAAGCCAGGCATTGAGCAGGGCCGCC |
| | | TGGAGCTGTGGGTGGACATGTTCCCCATGGACATGCCAGCCCC |
| | | TGGGACACCTCTGGATATATCCCCCAGGAAACCCAAGAAGTACG |
| | | AGCTGCGGGTCATCGTGTGGAACACAGACGAGGTGGTCCTGGA |
| | | AGACGATGATTTCTTCACGGGAGAGAAGTCCAGTGACATTTTTG |
| | | TGAGGGGGTGGCTGAAGGGCCAGCAGGAGGACAAACAGGACA |
| | | CAGATGTCCACTATCACTCCCTCACGGGGGAGGGCAACTTCAAC |
| | | TGGAGATACCTCTTCCCCTTCGACTACCTAGCGGCCGAAGAGAA |
| | | GATCGTTATGTCCAAAAAGGAGTCTATGTTCTCCTGGGATGAGA |
| | | CGGAGTACAAGATCCCTGCGCGGCTCACCCTGCAGATCTGGGA |
| | | CGCTGACCACTTCTCGGCTGACGACTTCCTGGGGGCTATCGAG |
| | | CTGGACCTGAACCGGTTCCCGAGGGGCGCTAAGACAGCCAAGC |
| | | AGTGCACCATGGAGATGGCCACCGGGGAGGTGGACGTACCCCT |
| | | GGTTTCCATCTTTAAACAGAAACGTGTCAAAGGCTGGTGGCCCC |
| | | TCCTGGCCCGCAATGAGAATGATGAGTTTGAGCTCACAGGCAAA |
| | | GTGGAGGCGGAGCTACACCTACTCACGGCAGAGGAGGCAGAG |
| | | AAGAACCCTGTGGGCCTGGCTCGCAATGAACCTGATCCCCTAG |
| | | AAAAACCCAATCGGCCGGACACAAGCTTCATCTGGTTCTTGAAC |
| | | CCTCTCAAGTCTGCCCGCTACTTCCTGTGGCATACCTACCGCTG |
| | | GCTACTCCTCAAATTCCTGCTGCTCTTCCTCCTGCTGCTGCTCTT |
| | | CGCCCTGTTTCTCTACTCTCTGCCTGGCTACCTGGCCAAGAAGA |
| | | TCCTTGGGGCCTGAGCCCTGCAGTCGCCTAGGCCTGCCGGCCT |
| | | GACACGGCATTCGTCTGGTTCCTGAACCCACTCAAATCTATCAA |
| | | GTACCTCATCTGCACCCGGTACAAGTGGCTGATCATCAAGATCG |
| | | TGCTGGCGCTGCTGGGGCTGCTCATGCTGGCCCTCTTCCTTTAC |
| | | AGCCTCCCAGGCTACATGGTCAAGAAGCTCCTAGGGGCCTGAA |
| | | GTGTGCCCCACCCCAGCCCGCTCCAGCATCCCTCCAGGGGCTG |
| | | CTGCGTATTTTGCCTTCCCTCACCTGGACTCTCTCCCAACTCCCT |
| | | GAGGAGCCCTCCCACGCCTGCCAGCCTTGAGCAAGACACCTGC |
| | | TTGCTGGACTTCATCCCCACCCCACACCCAAACTGTTGCTTGCC |
| | | TGATCTTGTCCCAGGCCTGCCTGGGGTTTGGGGCACAGTTGGC |
| | | CTCCAAAACCAGATACCCTCTTGTCTAAAGTACCAGGTTCCTCTG |
| | | CCCAACCCCAAGAGTGGTAGTGGCCCAACCCTCCCTGTGCTTTC |
| | | CAAATCTTGTCTTAAGGCACCAGTGAAATTAACCAAGAAACGCG |
| | | GAGCAATGCCCAAGGCTCTGATGAGTAGGAACACGTGGAAAGC |
| | | ACCAGGAATGCCAGCAGAGGCGAGGCGGCACACCTCTCTGCAG |
| | | AGCATCCAGGCCGAGCGGCGGGCAGCGGCCAGCTGCTTCTGC |
| | | GCATGCTCTCCTCTTGGCTCTGCTTCTTTCTCACAGTCACAGTCA |
| | | CTTCACAGCTTAGCCTTGGGCTTCCCATCACTTCCAGGGGTGCC |
| | | TCTGCCTTGGCCAGTGTGTGTCAGCTAGTACACAAGCTCCAAGT |
| | | GTGAATCAGGTGTACTGGCCGTCCTGAAGACTGACTGCCCTGTC |
| | | CTTCCTGCCGACAGCCACACCCGAGTGTACACTTAAAGCGGTG |
| | | CCCTTCTGCCTCTGTGGGCCTGCTGGCTGCTGTTCCTTTCTTGA |
| | | GTGTGATTTTTTTTTTCTCTCCCTCAATAAAATAAATCAAACTCTG |
| | | AGAC |
| 16 | mOTOF-201_2 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_001286421.1), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 3, | CGAGGCAAAGGTGACCGGATTGCCAAAGTCACTTTCCGAGGGC |
| | 7065 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 7 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGAGCGGGAAG |
| | | GAGTGTGTTCTCGGCCATGAAACTCGGCAAAACTCGGTCCCACA |
| | | AAGAGGAGCCCCAAAGACAAGATGAGCCAGCAGTGCTGGAGAT |
| | | GGAGGACCTGGACCACCTAGCCATTCAGCTGGGGGATGGGCTG |
| | | GATCCTGACTCCGTGTCTCTAGCCTCGGTCACCGCTCTCACCAG |
| | | CAATGTCTCCAACAAACGGTCTAAGCCAGATATTAAGATGGAGC |
| | | CCAGTGCTGGAAGGCCCATGGATTACCAGGTCAGCATCACAGT |
| | | GATTGAGGCTCGGCAGCTGGTGGGCTTGAACATGGACCCTGTG |
| | | GTGTGTGTGGAGGTGGGTGATGACAAGAAATACACGTCAATGAA |
| | | GGAGTCCACAAACTGCCCTTACTACAACGAGTACTTTGTCTTCG |
| | | ACTTCCATGTCTCTCCTGATGTCATGTTTGACAAGATCATCAAGA |
| | | TCTCGGTTATCCATTCTAAGAACCTGCTTCGGAGCGGCACCCTG |
| | | GTGGGTTCCTTCAAAATGGATGTGGGGACTGTGTATTCCCAGCC |
| | | TGAACACCAGTTCCATCACAAATGGGCCATCCTGTCAGACCCCG |
| | | ATGACATCTCTGCTGGGTTGAAGGGTTATGTAAAGTGTGATGTC |
| | | GCTGTGGTGGGCAAGGGAGACAACATCAAGACACCCCACAAGG |
| | | CCAACGAGACGGATGAGGACGACATTGAAGGGAACTTGCTGCT |
| | | CCCCGAGGGCGTGCCCCCCGAACGGCAGTGGGCACGGTTCTA |
| | | TGTGAAAATTTACCGAGCAGAGGGACTGCCCCGGATGAACACAA |
| | | GCCTCATGGCCAACGTGAAGAAGGCGTTCATCGGTGAGAACAA |
| | | GGACCTCGTCGACCCCTATGTGCAAGTCTTCTTTGCTGGACAAA |
| | | AGGGCAAAACATCAGTGCAGAAGAGCAGCTATGAGCCGCTATG |
| | | GAATGAGCAGGTCGTCTTCACAGACTTGTTCCCCCCACTCTGCA |
| | | AACGCATGAAGGTGCAGATCCGGGACTCTGACAAGGTCAATGAT |
| | | GTGGCCATCGGCACCCACTTCATCGACCTGCGCAAGATTTCCAA |
| | | CGATGGAGACAAAGGCTTCCTGCCTACCCTCGGTCCAGCCTGG |
| | | GTGAACATGTACGGCTCCACGCGCAACTACACACTGCTGGACG |
| | | AGCACCAGGACTTGAATGAAGGCCTGGGGGAGGGTGTGTCCTT |
| | | CCGGGCCCGCCTCATGTTGGGACTAGCTGTGGAGATCCTGGAC |
| | | ACCTCCAACCCAGAGCTCACCAGCTCCACGGAGGTGCAGGTGG |
| | | AGCAGGCCACGCCTGTCTCGGAGAGCTGCACAGGGAGAATGGA |
| | | AGAATTTTTTCTATTTGGAGCCTTCTTGGAAGCCTCAATGATTGA |
| | | CCGGAAAAATGGGGACAAGCCAATTACCTTTGAGGTGACCATAG |
| | | GAAACTACGGCAATGAAGTCGATGGTATGTCCCGGCCCCTGAG |
| | | GCCTCGGCCCCGGAAAGAGCCTGGGGATGAAGAAGAGGTAGA |
| | | CCTGATTCAGAACTCCAGTGACGATGAAGGTGACGAAGCCGGG |
| | | GACCTGGCCTCGGTGTCCTCCACCCCACCTATGCGGCCCCAGA |
| | | TCACGGACAGGAACTATTTCCACCTGCCCTACCTGGAGCGCAAG |
| | | CCCTGCATCTATATCAAGAGCTGGTGGCCTGACCAGAGGCGGC |
| | | GCCTCTACAATGCCAACATCATGGATCACATTGCTGACAAGCTG |
| | | GAAGAAGGCCTGAATGATGTACAGGAGATGATCAAAACGGAGAA |
| | | GTCCTACCCGGAGCGCCGCCTGCGGGGTGTGCTAGAGGAACTC |
| | | AGCTGTGGCTGCCACCGCTTCCTCTCCCTCTCGGACAAGGACC |
| | | AGGGCCGCTCGTCCCGCACCAGGCTGGATCGAGAGCGTCTTAA |
| | | GTCCTGTATGAGGGAGTTGGAGAGCATGGGACAGCAGGCCAAG |
| | | AGCCTGAGGGCTCAGGTGAAGCGGCACACTGTTCGGGACAAGC |
| | | TGAGGTCATGCCAGAACTTTCTGCAGAAGCTACGCTTCCTGGCG |
| | | GATGAGCCCCAGCACAGCATTCCTGATGTGTTCATTTGGATGAT |
| | | GAGCAACAACAAACGTATCGCCTATGCCCGCGTGCCTTCCAAAG |
| | | ACCTGCTCTTCTCCATCGTGGAGGAGGAACTGGGCAAGGACTG |
| | | CGCCAAAGTCAAGACCCTCTTCCTGAAGCTGCCAGGGAAGAGG |
| | | GGCTTCGGCTCGGCAGGCTGGACAGTACAGGCCAAGCTGGAG |
| | | CTCTACCTGTGGCTGGGCCTCAGCAAGCAGCGAAAGGACTTCC |
| | | TGTGTGGTCTGCCCTGTGGCTTCGAGGAGGTCAAGGCAGCCCA |
| | | AGGCCTGGGCCTGCATTCCTTTCCGCCCATCAGCCTAGTCTACA |
| | | CCAAGAAGCAAGCCTTCCAGCTCCGAGCACACATGTATCAGGC |
| | | CCGAAGCCTCTTTGCTGCTGACAGCAGTGGGCTCTCTGATCCCT |
| | | TTGCCCGTGTCTTCTTCATCAACCAGAGCCAATGCACTGAGGTT |
| | | CTAAACGAGACACTGTGTCCCACCTGGGACCAGATGCTGGTATT |
| | | TGACAACCTGGAGCTGTACGGTGAAGCTCACGAGTTACGAGAT |
| | | GATCCCCCCATCATTGTCATTGAAATCTACGACCAGGACAGCAT |
| | | GGGCAAAGCCGACTTCATGGGCCGGACCTTCGCCAAGCCCCTG |
| | | GTGAAGATGGCAGATGAAGCATACTGCCCACCTCGCTTCCCGC |
| | | CGCAGCTTGAGTACTACCAGATCTACCGAGGCAGTGCCACTGC |
| | | CGGAGACCTACTGGCTGCCTTCGAGCTGCTGCAGATTGGGCCA |
| | | TCAGGGAAGGCTGACCTGCCACCCATCAATGGCCCAGTGGACA |
| | | TGGACAGAGGGCCCATCATGCCTGTGCCCGTGGGAATCCGGCC |
| | | AGTGCTCAGCAAGTACCGAGTGGAGGTGCTGTTCTGGGGCCTG |
| | | AGGGACCTAAAGAGGGTGAACCTGGCCCAGGTGGACCGACCAC |
| | | GGGTGGACATCGAGTGTGCAGGAAAGGGGGTACAATCCTCCCT |
| | | GATTCACAATTATAAGAAGAACCCCAACTTCAACACGCTGGTCAA |
| | | GTGGTTTGAAGTGGACCTCCCGGAGAATGAGCTCCTGCACCCA |
| | | CCCTTGAACATCCGAGTGGTAGATTGCCGGGCCTTTGGACGATA |
| | | CACCCTGGTGGGTTCCCACGCAGTCAGCTCACTGAGGCGCTTC |
| | | ATCTACCGACCTCCAGACCGCTCAGCCCCCAACTGGAACACCA |
| | | CAGGGGAGGTTGTAGTAAGCATGGAGCCTGAGGAGCCAGTTAA |
| | | GAAGCTGGAGACCATGGTGAAACTGGATGCGACTTCTGATGCT |
| | | GTGGTCAAGGTGGATGTGGCTGAAGATGAGAAGGAAAGGAAGA |
| | | AGAAGAAAAAGAAAGGCCCGTCAGAGGAGCCAGAGGAGGAAGA |
| | | GCCCGATGAGAGCATGCTGGATTGGTGGTCCAAGTACTTCGCC |
| | | TCCATCGACACAATGAAGGAGCAACTTCGACAACATGAGACCTC |
| | | TGGAACTGACTTGGAAGAGAAGGAAGAGATGGAAAGCGCTGAG |
| | | GGCCTGAAGGGACCAATGAAGAGCAAGGAGAAGTCCAGAGCTG |
| | | CAAAGGAGGAGAAAAAGAAGAAAAACCAGAGCCCTGGCCCTGG |
| | | CCAGGGATCGGAGGCTCCTGAGAAGAAGAAAGCCAAGATCGAT |
| | | GAGCTTAAGGTGTACCCCAAGGAGCTGGAATCGGAGTTTGACA |
| | | GCTTTGAGGACTGGCTGCACACCTTCAACCTGTTGAGGGGCAA |
| | | GACGGGAGATGATGAGGATGGCTCCACAGAGGAGGAGCGCATA |
| | | GTAGGCCGATTCAAGGGCTCCCTCTGTGTGTACAAAGTGCCACT |
| | | CCCAGAAGATGTATCTCGAGAAGCTGGCTATGATCCCACCTATG |
| | | GAATGTTCCAGGGCATCCCAAGCAATGACCCCATCAATGTGCTG |
| | | GTCCGAATCTATGTGGTCCGGGCCACAGACCTGCACCCGGCCG |
| | | ACATCAATGGCAAAGCTGACCCCTATATTGCCATCAAGTTAGGC |
| | | AAGACCGACATCCGAGACAAGGAGAACTACATCTCCAAGCAGCT |
| | | CAACCCTGTGTTTGGGAAGTCCTTTGACATTGAGGCCTCCTTCC |
| | | CCATGGAGTCCATGTTGACAGTGGCCGTGTACGACTGGGATCT |
| | | GGTGGGCACTGATGACCTCATCGGAGAAACCAAGATTGACCTG |
| | | GAAAACCGCTTCTACAGCAAGCATCGCGCCACCTGCGGCATCG |
| | | CACAGACCTATTCCATACATGGCTACAATATCTGGAGGGACCCC |
| | | ATGAAGCCCAGCCAGATCCTGACACGCCTCTGTAAAGAGGGCA |
| | | AAGTGGACGGCCCCCACTTTGGTCCCCATGGGAGAGTGAGGGT |
| | | TGCCAACCGTGTCTTCACGGGGCCTTCAGAAATAGAGGATGAGA |
| | | ATGGTCAGAGGAAGCCCACAGATGAGCACGTGGCACTGTCTGC |
| | | TCTGAGACACTGGGAGGACATCCCCCGGGTGGGCTGCCGCCTT |
| | | GTGCCGGAACACGTGGAGACCAGGCCGCTGCTCAACCCTGACA |
| | | AGCCAGGCATTGAGCAGGGCCGCCTGGAGCTGTGGGTGGACAT |
| | | GTTCCCCATGGACATGCCAGCCCCTGGGACACCTCTGGATATAT |
| | | CCCCCAGGAAACCCAAGAAGTACGAGCTGCGGGTCATCGTGTG |
| | | GAACACAGACGAGGTGGTCCTGGAAGACGATGATTTCTTCACG |
| | | GGAGAGAAGTCCAGTGACATTTTTGTGAGGGGGTGGCTGAAGG |
| | | GCCAGCAGGAGGACAAACAGGACACAGATGTCCACTATCACTC |
| | | CCTCACGGGGGAGGGCAACTTCAACTGGAGATACCTCTTCCCC |
| | | TTCGACTACCTAGCGGCCGAAGAGAAGATCGTTATGTCCAAAAA |
| | | GGAGTCTATGTTCTCCTGGGATGAGACGGAGTACAAGATCCCTG |
| | | CGCGGCTCACCCTGCAGATCTGGGACGCTGACCACTTCTCGGC |
| | | TGACGACTTCCTGGGGGCTATCGAGCTGGACCTGAACCGGTTC |
| | | CCGAGGGGCGCTAAGACAGCCAAGCAGTGCACCATGGAGATGG |
| | | CCACCGGGGAGGTGGACGTACCCCTGGTTTCCATCTTTAAACAG |
| | | AAACGTGTCAAAGGCTGGTGGCCCCTCCTGGCCCGCAATGAGA |
| | | ATGATGAGTTTGAGCTCACAGGCAAAGTGGAGGCGGAGCTACA |
| | | CCTACTCACGGCAGAGGAGGCAGAGAAGAACCCTGTGGGCCTG |
| | | GCTCGCAATGAACCTGATCCCCTAGAAAAACCCAATCGGCCGGA |
| | | CACAAGCTTCATCTGGTTCTTGAACCCTCTCAAGTCTGCCCGCT |
| | | ACTTCCTGTGGCATACCTACCGCTGGCTACTCCTCAAATTCCTG |
| | | CTGCTCTTCCTCCTGCTGCTGCTCTTCGCCCTGTTTCTCTACTCT |
| | | CTGCCTGGCTACCTGGCCAAGAAGATCCTTGGGGCCTGAGCCC |
| | | TGCAGTCGCCTAGGCCTGCCGGCCTGACACGGCATTCGTCTGG |
| | | TTCCTGAACCCACTCAAATCTATCAAGTACCTCATCTGCACCCG |
| | | GTACAAGTGGCTGATCATCAAGATCGTGCTGGCGCTGCTGGGG |
| | | CTGCTCATGCTGGCCCTCTTCCTTTACAGCCTCCCAGGCTACAT |
| | | GGTCAAGAAGCTCCTAGGGGCCTGAAGTGTGCCCCACCCCAGC |
| | | CCGCTCCAGCATCCCTCCAGGGGCTGCTGCGTATTTTGCCTTCC |
| | | CTCACCTGGACTCTCTCCCAACTCCCTGAGGAGCCCTCCCACG |
| | | CCTGCCAGCCTTGAGCAAGACACCTGCTTGCTGGACTTCATCCC |
| | | CACCCCACACCCAAACTGTTGCTTGCCTGATCTTGTCCCAGGCC |
| | | TGCCTGGGGTTTGGGGCACAGTTGGCCTCCAAAACCAGATACC |
| | | CTCTTGTCTAAAGTACCAGGTTCCTCTGCCCAACCCCAAGAGTG |
| | | GTAGTGGCCCAACCCTCCCTGTGCTTTCCAAATCTTGTCTTAAG |
| | | GCACCAGTGAAATTAACCAAGAAACGCGGAGCAATGCCCAAGG |
| | | CTCTGATGAGTAGGAACACGTGGAAAGCACCAGGAATGCCAGC |
| | | AGAGGCGAGGCGGCACACCTCTCTGCAGAGCATCCAGGCCGA |
| | | GCGGGGGGCAGCGGCCAGCTGCTTCTGCGCATGCTCTCCTCTT |
| | | GGCTCTGCTTCTTTCTCACAGTCACAGTCACTTCACAGCTTAGC |
| | | CTTGGGCTTCCCATCACTTCCAGGGGTGCCTCTGCCTTGGCCA |
| | | GTGTGTGTCAGCTAGTACACAAGCTCCAAGTGTGAATCAGGTGT |
| | | ACTGGCCGTCCTGAAGACTGACTGCCCTGTCCTTCCTGCCGACA |
| | | GCCACACCCGAGTGTACACTTAAAGCGGTGCCCTTCTGCCTCTG |
| | | TGGGCCTGCTGGCTGCTGTTCCTTTCTTGAGTGTGATTTTTTTTT |
| | | TCTCTCCCTCAATAAAATAAATCAAACTCTGAGAC |
| 17 | mOTOF-202_1 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_001100395.1), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 1, | CGAGGCAAAGGTGACCGGATTGCCAAAGTCACTTTCCGAGGGC |
| | 6907 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 8 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGCAAAGGCAG |
| | | AGAGAAGACCAAGGGAGGCAGAGATGGCGAGCACAAAGCGGG |
| | | AAGGAGTGTGTTCTCGGCCATGAAACTCGGCAAAACTCGGTCCC |
| | | ACAAAGAGGAGCCCCAAAGACAAGATGAGCCAGCAGTGCTGGA |
| | | GATGGAGGACCTGGACCACCTAGCCATTCAGCTGGGGGATGGG |
| | | CTGGATCCTGACTCCGTGTCTCTAGCCTCGGTCACCGCTCTCAC |
| | | CAGCAATGTCTCCAACAAACGGTCTAAGCCAGATATTAAGATGG |
| | | AGCCCAGTGCTGGAAGGCCCATGGATTACCAGGTCAGCATCAC |
| | | AGTGATTGAGGCTCGGCAGCTGGTGGGCTTGAACATGGACCCT |
| | | GTGGTGTGTGTGGAGGTGGGTGATGACAAGAAATACACGTCAAT |
| | | GAAGGAGTCCACAAACTGCCCTTACTACAACGAGTACTTTGTCT |
| | | TCGACTTCCATGTCTCTCCTGATGTCATGTTTGACAAGATCATCA |
| | | AGATCTCGGTTATCCATTCTAAGAACCTGCTTCGGAGCGGCACC |
| | | CTGGTGGGTTCCTTCAAAATGGATGTGGGGACTGTGTATTCCCA |
| | | GCCTGAACACCAGTTCCATCACAAATGGGCCATCCTGTCAGACC |
| | | CCGATGACATCTCTGCTGGGTTGAAGGGTTATGTAAAGTGTGAT |
| | | GTCGCTGTGGTGGGCAAGGGAGACAACATCAAGACACCCCACA |
| | | AGGCCAACGAGACGGATGAGGACGACATTGAAGGGAACTTGCT |
| | | GCTCCCCGAGGGCGTGCCCCCCGAACGGCAGTGGGCACGGTT |
| | | CTATGTGAAAATTTACCGAGCAGAGGGACTGCCCCGGATGAACA |
| | | CAAGCCTCATGGCCAACGTGAAGAAGGCGTTCATCGGTGAGAA |
| | | CAAGGACCTCGTCGACCCCTATGTGCAAGTCTTCTTTGCTGGAC |
| | | AAAAGGGCAAAACATCAGTGCAGAAGAGCAGCTATGAGCCGCT |
| | | ATGGAATGAGCAGGTCGTCTTCACAGACTTGTTCCCCCCACTCT |
| | | GCAAACGCATGAAGGTGCAGATCCGGGACTCTGACAAGGTCAA |
| | | TGATGTGGCCATCGGCACCCACTTCATCGACCTGCGCAAGATTT |
| | | CCAACGATGGAGACAAAGGCTTCCTGCCTACCCTCGGTCCAGC |
| | | CTGGGTGAACATGTACGGCTCCACGCGCAACTACACACTGCTG |
| | | GACGAGCACCAGGACTTGAATGAAGGCCTGGGGGAGGGTGTGT |
| | | CCTTCCGGGCCCGCCTCATGTTGGGACTAGCTGTGGAGATCCT |
| | | GGACACCTCCAACCCAGAGCTCACCAGCTCCACGGAGGTGCAG |
| | | GTGGAGCAGGCCACGCCTGTCTCGGAGAGCTGCACAGGGAGA |
| | | ATGGAAGAATTTTTTCTATTTGGAGCCTTCTTGGAAGCCTCAATG |
| | | ATTGACCGGAAAAATGGGGACAAGCCAATTACCTTTGAGGTGAC |
| | | CATAGGAAACTACGGCAATGAAGTCGATGGTATGTCCCGGCCC |
| | | CTGAGGCCTCGGCCCCGGAAAGAGCCTGGGGATGAAGAAGAG |
| | | GTAGACCTGATTCAGAACTCCAGTGACGATGAAGGTGACGAAGC |
| | | CGGGGACCTGGCCTCGGTGTCCTCCACCCCACCTATGCGGCCC |
| | | CAGATCACGGACAGGAACTATTTCCACCTGCCCTACCTGGAGCG |
| | | CAAGCCCTGCATCTATATCAAGAGCTGGTGGCCTGACCAGAGG |
| | | CGGCGCCTCTACAATGCCAACATCATGGATCACATTGCTGACAA |
| | | GCTGGAAGAAGGCCTGAATGATGTACAGGAGATGATCAAAACG |
| | | GAGAAGTCCTACCCGGAGCGCCGCCTGCGGGGTGTGCTAGAG |
| | | GAACTCAGCTGTGGCTGCCACCGCTTCCTCTCCCTCTCGGACAA |
| | | GGACCAGGGCCGCTCGTCCCGCACCAGGCTGGATCGAGAGCG |
| | | TCTTAAGTCCTGTATGAGGGAGTTGGAGAGCATGGGACAGCAG |
| | | GCCAAGAGCCTGAGGGCTCAGGTGAAGCGGCACACTGTTCGGG |
| | | ACAAGCTGAGGTCATGCCAGAACTTTCTGCAGAAGCTACGCTTC |
| | | CTGGCGGATGAGCCCCAGCACAGCATTCCTGATGTGTTCATTTG |
| | | GATGATGAGCAACAACAAACGTATCGCCTATGCCCGCGTGCCTT |
| | | CCAAAGACCTGCTCTTCTCCATCGTGGAGGAGGAACTGGGCAA |
| | | GGACTGCGCCAAAGTCAAGACCCTCTTCCTGAAGCTGCCAGGG |
| | | AAGAGGGGCTTCGGCTCGGCAGGCTGGACAGTACAGGCCAAG |
| | | CTGGAGCTCTACCTGTGGCTGGGCCTCAGCAAGCAGCGAAAGG |
| | | ACTTCCTGTGTGGTCTGCCCTGTGGCTTCGAGGAGGTCAAGGC |
| | | AGCCCAAGGCCTGGGCCTGCATTCCTTTCCGCCCATCAGCCTA |
| | | GTCTACACCAAGAAGCAAGCCTTCCAGCTCCGAGCACACATGTA |
| | | TCAGGCCCGAAGCCTCTTTGCTGCTGACAGCAGTGGGCTCTCT |
| | | GATCCCTTTGCCCGTGTCTTCTTCATCAACCAGAGCCAATGCAC |
| | | TGAGGTTCTAAACGAGACACTGTGTCCCACCTGGGACCAGATGC |
| | | TGGTATTTGACAACCTGGAGCTGTACGGTGAAGCTCACGAGTTA |
| | | CGAGATGATCCCCCCATCATTGTCATTGAAATCTACGACCAGGA |
| | | CAGCATGGGCAAAGCCGACTTCATGGGCCGGACCTTCGCCAAG |
| | | CCCCTGGTGAAGATGGCAGATGAAGCATACTGCCCACCTCGCTT |
| | | CCCGCCGCAGCTTGAGTACTACCAGATCTACCGAGGCAGTGCC |
| | | ACTGCCGGAGACCTACTGGCTGCCTTCGAGCTGCTGCAGATTG |
| | | GGCCATCAGGGAAGGCTGACCTGCCACCCATCAATGGCCCAGT |
| | | GGACATGGACAGAGGGCCCATCATGCCTGTGCCCGTGGGAATC |
| | | CGGCCAGTGCTCAGCAAGTACCGAGTGGAGGTGCTGTTCTGGG |
| | | GCCTGAGGGACCTAAAGAGGGTGAACCTGGCCCAGGTGGACC |
| | | GACCACGGGTGGACATCGAGTGTGCAGGAAAGGGGGTACAATC |
| | | CTCCCTGATTCACAATTATAAGAAGAACCCCAACTTCAACACGCT |
| | | GGTCAAGTGGTTTGAAGTGGACCTCCCGGAGAATGAGCTCCTG |
| | | CACCCACCCTTGAACATCCGAGTGGTAGATTGCCGGGCCTTTG |
| | | GACGATACACCCTGGTGGGTTCCCACGCAGTCAGCTCACTGAG |
| | | GCGCTTCATCTACCGACCTCCAGACCGCTCAGCCCCCAACTGG |
| | | AACACCACAGGGGAGGTTGTAGTAAGCATGGAGCCTGAGGAGC |
| | | CAGTTAAGAAGCTGGAGACCATGGTGAAACTGGATGCGACTTCT |
| | | GATGCTGTGGTCAAGGTGGATGTGGCTGAAGATGAGAAGGAAA |
| | | GGAAGAAGAAGAAAAAGAAAGGCCCGTCAGAGGAGCCAGAGGA |
| | | GGAAGAGCCCGATGAGAGCATGCTGGATTGGTGGTCCAAGTAC |
| | | TTCGCCTCCATCGACACAATGAAGGAGCAACTTCGACAACATGA |
| | | GACCTCTGGAACTGACTTGGAAGAGAAGGAAGAGATGGAAAGC |
| | | GCTGAGGGCCTGAAGGGACCAATGAAGAGCAAGGAGAAGTCCA |
| | | GAGCTGCAAAGGAGGAGAAAAAGAAGAAAAACCAGAGCCCTGG |
| | | CCCTGGCCAGGGATCGGAGGCTCCTGAGAAGAAGAAAGCCAAG |
| | | ATCGATGAGCTTAAGGTGTACCCCAAGGAGCTGGAATCGGAGTT |
| | | TGACAGCTTTGAGGACTGGCTGCACACCTTCAACCTGTTGAGGG |
| | | GCAAGACGGGAGATGATGAGGATGGCTCCACAGAGGAGGAGC |
| | | GCATAGTAGGCCGATTCAAGGGCTCCCTCTGTGTGTACAAAGTG |
| | | CCACTCCCAGAAGATGTATCTCGAGAAGCTGGCTATGATCCCAC |
| | | CTATGGAATGTTCCAGGGCATCCCAAGCAATGACCCCATCAATG |
| | | TGCTGGTCCGAATCTATGTGGTCCGGGCCACAGACCTGCACCC |
| | | GGCCGACATCAATGGCAAAGCTGACCCCTATATTGCCATCAAGT |
| | | TAGGCAAGACCGACATCCGAGACAAGGAGAACTACATCTCCAAG |
| | | CAGCTCAACCCTGTGTTTGGGAAGTCCTTTGACATTGAGGCCTC |
| | | CTTCCCCATGGAGTCCATGTTGACAGTGGCCGTGTACGACTGG |
| | | GATCTGGTGGGCACTGATGACCTCATCGGAGAAACCAAGATTGA |
| | | CCTGGAAAACCGCTTCTACAGCAAGCATCGCGCCACCTGCGGC |
| | | ATCGCACAGACCTATTCCATACATGGCTACAATATCTGGAGGGA |
| | | CCCCATGAAGCCCAGCCAGATCCTGACACGCCTCTGTAAAGAG |
| | | GGCAAAGTGGACGGCCCCCACTTTGGTCCCCATGGGAGAGTGA |
| | | GGGTTGCCAACCGTGTCTTCACGGGGCCTTCAGAAATAGAGGA |
| | | TGAGAATGGTCAGAGGAAGCCCACAGATGAGCACGTGGCACTG |
| | | TCTGCTCTGAGACACTGGGAGGACATCCCCCGGGTGGGCTGCC |
| | | GCCTTGTGCCGGAACACGTGGAGACCAGGCCGCTGCTCAACCC |
| | | TGACAAGCCAGGCATTGAGCAGGGCCGCCTGGAGCTGTGGGTG |
| | | GACATGTTCCCCATGGACATGCCAGCCCCTGGGACACCTCTGG |
| | | ATATATCCCCCAGGAAACCCAAGAAGTACGAGCTGCGGGTCATC |
| | | GTGTGGAACACAGACGAGGTGGTCCTGGAAGACGATGATTTCTT |
| | | CACGGGAGAGAAGTCCAGTGACATTTTTGTGAGGGGGTGGCTG |
| | | AAGGGCCAGCAGGAGGACAAACAGGACACAGATGTCCACTATC |
| | | ACTCCCTCACGGGGGAGGGCAACTTCAACTGGAGATACCTCTTC |
| | | CCCTTCGACTACCTAGCGGCCGAAGAGAAGATCGTTATGTCCAA |
| | | AAAGGAGTCTATGTTCTCCTGGGATGAGACGGAGTACAAGATCC |
| | | CTGCGCGGCTCACCCTGCAGATCTGGGACGCTGACCACTTCTC |
| | | GGCTGACGACTTCCTGGGGGCTATCGAGCTGGACCTGAACCGG |
| | | TTCCCGAGGGGCGCTAAGACAGCCAAGCAGTGCACCATGGAGA |
| | | TGGCCACCGGGGAGGTGGACGTACCCCTGGTTTCCATCTTTAAA |
| | | CAGAAACGTGTCAAAGGCTGGTGGCCCCTCCTGGCCCGCAATG |
| | | AGAATGATGAGTTTGAGCTCACAGGCAAAGTGGAGGCGGAGCT |
| | | ACACCTACTCACGGCAGAGGAGGCAGAGAAGAACCCTGTGGGC |
| | | CTGGCTCGCAATGAACCTGATCCCCTAGAAAAACCCAACCGGCC |
| | | TGACACGGCATTCGTCTGGTTCCTGAACCCACTCAAATCTATCA |
| | | AGTACCTCATCTGCACCCGGTACAAGTGGCTGATCATCAAGATC |
| | | GTGCTGGCGCTGCTGGGGCTGCTCATGCTGGCCCTCTTCCTTT |
| | | ACAGCCTCCCAGGCTACATGGTCAAGAAGCTCCTAGGGGCCTG |
| | | AAGTGTGCCCCACCCCAGCCCGCTCCAGCATCCCTCCAGGGGC |
| | | TGCTGCGTATTTTGCCTTCCCTCACCTGGACTCTCTCCCAACTC |
| | | CCTGAGGAGCCCTCCCACGCCTGCCAGCCTTGAGCAAGACACC |
| | | TGCTTGCTGGACTTCATCCCCACCCCACACCCAAACTGTTGCTT |
| | | GCCTGATCTTGTCCCAGGCCTGCCTGGGGTTTGGGGCACAGTT |
| | | GGCCTCCAAAACCAGATACCCTCTTGTCTAAAGTACCAGGTTCC |
| | | TCTGCCCAACCCCAAGAGTGGTAGTGGCCCAACCCTCCCTGTG |
| | | CTTTCCAAATCTTGTCTTAAGGCACCAGTGAAATTAACCAAGAAA |
| | | CGCGGAGCAATGCCCAAGGCTCTGATGAGTAGGAACACGTGGA |
| | | AAGCACCAGGAATGCCAGCAGAGGCGAGGCGGCACACCTCTCT |
| | | GCAGAGCATCCAGGCCGAGCGGCGGGCAGCGGCCAGCTGCTT |
| | | CTGCGCATGCTCTCCTCTTGGCTCTGCTTCTTTCTCACAGTCACA |
| | | GTCACTTCACAGCTTAGCCTTGGGCTTCCCATCACTTCCAGGGG |
| | | TGCCTCTGCCTTGGCCAGTGTGTGTCAGCTAGTACACAAGCT |
| | | CCAAGTGTGAATCAGGTGTACTGGCCGTCCTGAAGACTGACTGC |
| | | CCTGTCCTTCCTGCCGACAGCCACACCCGAGTGTACACTTAAAG |
| | | CGGTGCCCTTCTGCCTCTGTGGGCCTGCTGGCTGCTGTTCCTTT |
| | | CTTGAGTGTGATTTTTTTTTTCTCTCCCTCAATAAAATAAATCAAA |
| | | CTCTGAGAC |
| 18 | mOTOF-202_2 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_001313767.1), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 4, | CGAGGCAAAGGTGACCGGATTGCCAAAGTCACTTTCCGAGGGC |
| | 6862 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 9 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGAGCGGGAAG |
| | | GAGTGTGTTCTCGGCCATGAAACTCGGCAAAACTCGGTCCCACA |
| | | AAGAGGAGCCCCAAAGACAAGATGAGCCAGCAGTGCTGGAGAT |
| | | GGAGGACCTGGACCACCTAGCCATTCAGCTGGGGGATGGGCTG |
| | | GATCCTGACTCCGTGTCTCTAGCCTCGGTCACCGCTCTCACCAG |
| | | CAATGTCTCCAACAAACGGTCTAAGCCAGATATTAAGATGGAGC |
| | | CCAGTGCTGGAAGGCCCATGGATTACCAGGTCAGCATCACAGT |
| | | GATTGAGGCTCGGCAGCTGGTGGGCTTGAACATGGACCCTGTG |
| | | GTGTGTGTGGAGGTGGGTGATGACAAGAAATACACGTCAATGAA |
| | | GGAGTCCACAAACTGCCCTTACTACAACGAGTACTTTGTCTTCG |
| | | ACTTCCATGTCTCTCCTGATGTCATGTTTGACAAGATCATCAAGA |
| | | TCTCGGTTATCCATTCTAAGAACCTGCTTCGGAGCGGCACCCTG |
| | | GTGGGTTCCTTCAAAATGGATGTGGGGACTGTGTATTCCCAGCC |
| | | TGAACACCAGTTCCATCACAAATGGGCCATCCTGTCAGACCCCG |
| | | ATGACATCTCTGCTGGGTTGAAGGGTTATGTAAAGTGTGATGTC |
| | | GCTGTGGTGGGCAAGGGAGACAACATCAAGACACCCCACAAGG |
| | | CCAACGAGACGGATGAGGACGACATTGAAGGGAACTTGCTGCT |
| | | CCCCGAGGGCGTGCCCCCCGAACGGCAGTGGGCACGGTTCTA |
| | | TGTGAAAATTTACCGAGCAGAGGGACTGCCCCGGATGAACACAA |
| | | GCCTCATGGCCAACGTGAAGAAGGCGTTCATCGGTGAGAACAA |
| | | GGACCTCGTCGACCCCTATGTGCAAGTCTTCTTTGCTGGACAAA |
| | | AGGGCAAAACATCAGTGCAGAAGAGCAGCTATGAGCCGCTATG |
| | | GAATGAGCAGGTCGTCTTCACAGACTTGTTCCCCCCACTCTGCA |
| | | AACGCATGAAGGTGCAGATCCGGGACTCTGACAAGGTCAATGAT |
| | | GTGGCCATCGGCACCCACTTCATCGACCTGCGCAAGATTTCCAA |
| | | CGATGGAGACAAAGGCTTCCTGCCTACCCTCGGTCCAGCCTGG |
| | | GTGAACATGTACGGCTCCACGCGCAACTACACACTGCTGGACG |
| | | AGCACCAGGACTTGAATGAAGGCCTGGGGGAGGGTGTGTCCTT |
| | | CCGGGCCCGCCTCATGTTGGGACTAGCTGTGGAGATCCTGGAC |
| | | ACCTCCAACCCAGAGCTCACCAGCTCCACGGAGGTGCAGGTGG |
| | | AGCAGGCCACGCCTGTCTCGGAGAGCTGCACAGGGAGAATGGA |
| | | AGAATTTTTTCTATTTGGAGCCTTCTTGGAAGCCTCAATGATTGA |
| | | CCGGAAAAATGGGGACAAGCCAATTACCTTTGAGGTGACCATAG |
| | | GAAACTACGGCAATGAAGTCGATGGTATGTCCCGGCCCCTGAG |
| | | GCCTCGGCCCCGGAAAGAGCCTGGGGATGAAGAAGAGGTAGA |
| | | CCTGATTCAGAACTCCAGTGACGATGAAGGTGACGAAGCCGGG |
| | | GACCTGGCCTCGGTGTCCTCCACCCCACCTATGCGGCCCCAGA |
| | | TCACGGACAGGAACTATTTCCACCTGCCCTACCTGGAGCGCAAG |
| | | CCCTGCATCTATATCAAGAGCTGGTGGCCTGACCAGAGGCGGC |
| | | GCCTCTACAATGCCAACATCATGGATCACATTGCTGACAAGCTG |
| | | GAAGAAGGCCTGAATGATGTACAGGAGATGATCAAAACGGAGAA |
| | | GTCCTACCCGGAGCGCCGCCTGCGGGGTGTGCTAGAGGAACTC |
| | | AGCTGTGGCTGCCACCGCTTCCTCTCCCTCTCGGACAAGGACC |
| | | AGGGCCGCTCGTCCCGCACCAGGCTGGATCGAGAGCGTCTTAA |
| | | GTCCTGTATGAGGGAGTTGGAGAGCATGGGACAGCAGGCCAAG |
| | | AGCCTGAGGGCTCAGGTGAAGCGGCACACTGTTCGGGACAAGC |
| | | TGAGGTCATGCCAGAACTTTCTGCAGAAGCTACGCTTCCTGGCG |
| | | GATGAGCCCCAGCACAGCATTCCTGATGTGTTCATTTGGATGAT |
| | | GAGCAACAACAAACGTATCGCCTATGCCCGCGTGCCTTCCAAAG |
| | | ACCTGCTCTTCTCCATCGTGGAGGAGGAACTGGGCAAGGACTG |
| | | CGCCAAAGTCAAGACCCTCTTCCTGAAGCTGCCAGGGAAGAGG |
| | | GGCTTCGGCTCGGCAGGCTGGACAGTACAGGCCAAGCTGGAG |
| | | CTCTACCTGTGGCTGGGCCTCAGCAAGCAGCGAAAGGACTTCC |
| | | TGTGTGGTCTGCCCTGTGGCTTCGAGGAGGTCAAGGCAGCCCA |
| | | AGGCCTGGGCCTGCATTCCTTTCCGCCCATCAGCCTAGTCTACA |
| | | CCAAGAAGCAAGCCTTCCAGCTCCGAGCACACATGTATCAGGC |
| | | CCGAAGCCTCTTTGCTGCTGACAGCAGTGGGCTCTCTGATCCCT |
| | | TTGCCCGTGTCTTCTTCATCAACCAGAGCCAATGCACTGAGGTT |
| | | CTAAACGAGACACTGTGTCCCACCTGGGACCAGATGCTGGTATT |
| | | TGACAACCTGGAGCTGTACGGTGAAGCTCACGAGTTACGAGAT |
| | | GATCCCCCCATCATTGTCATTGAAATCTACGACCAGGACAGCAT |
| | | GGGCAAAGCCGACTTCATGGGCCGGACCTTCGCCAAGCCCCTG |
| | | GTGAAGATGGCAGATGAAGCATACTGCCCACCTCGCTTCCCGC |
| | | CGCAGCTTGAGTACTACCAGATCTACCGAGGCAGTGCCACTGC |
| | | CGGAGACCTACTGGCTGCCTTCGAGCTGCTGCAGATTGGGCCA |
| | | TCAGGGAAGGCTGACCTGCCACCCATCAATGGCCCAGTGGACA |
| | | TGGACAGAGGGCCCATCATGCCTGTGCCCGTGGGAATCCGGCC |
| | | AGTGCTCAGCAAGTACCGAGTGGAGGTGCTGTTCTGGGGCCTG |
| | | AGGGACCTAAAGAGGGTGAACCTGGCCCAGGTGGACCGACCAC |
| | | GGGTGGACATCGAGTGTGCAGGAAAGGGGGTACAATCCTCCCT |
| | | GATTCACAATTATAAGAAGAACCCCAACTTCAACACGCTGGTCAA |
| | | GTGGTTTGAAGTGGACCTCCCGGAGAATGAGCTCCTGCACCCA |
| | | CCCTTGAACATCCGAGTGGTAGATTGCCGGGCCTTTGGACGATA |
| | | CACCCTGGTGGGTTCCCACGCAGTCAGCTCACTGAGGCGCTTC |
| | | ATCTACCGACCTCCAGACCGCTCAGCCCCCAACTGGAACACCA |
| | | CAGGGGAGGTTGTAGTAAGCATGGAGCCTGAGGAGCCAGTTAA |
| | | GAAGCTGGAGACCATGGTGAAACTGGATGCGACTTCTGATGCT |
| | | GTGGTCAAGGTGGATGTGGCTGAAGATGAGAAGGAAAGGAAGA |
| | | AGAAGAAAAAGAAAGGCCCGTCAGAGGAGCCAGAGGAGGAAGA |
| | | GCCCGATGAGAGCATGCTGGATTGGTGGTCCAAGTACTTCGCC |
| | | TCCATCGACACAATGAAGGAGCAACTTCGACAACATGAGACCTC |
| | | TGGAACTGACTTGGAAGAGAAGGAAGAGATGGAAAGCGCTGAG |
| | | GGCCTGAAGGGACCAATGAAGAGCAAGGAGAAGTCCAGAGCTG |
| | | CAAAGGAGGAGAAAAAGAAGAAAAACCAGAGCCCTGGCCCTGG |
| | | CCAGGGATCGGAGGCTCCTGAGAAGAAGAAAGCCAAGATCGAT |
| | | GAGCTTAAGGTGTACCCCAAGGAGCTGGAATCGGAGTTTGACA |
| | | GCTTTGAGGACTGGCTGCACACCTTCAACCTGTTGAGGGGCAA |
| | | GACGGGAGATGATGAGGATGGCTCCACAGAGGAGGAGCGCATA |
| | | GTAGGCCGATTCAAGGGCTCCCTCTGTGTGTACAAAGTGCCACT |
| | | CCCAGAAGATGTATCTCGAGAAGCTGGCTATGATCCCACCTATG |
| | | GAATGTTCCAGGGCATCCCAAGCAATGACCCCATCAATGTGCTG |
| | | GTCCGAATCTATGTGGTCCGGGCCACAGACCTGCACCCGGCCG |
| | | ACATCAATGGCAAAGCTGACCCCTATATTGCCATCAAGTTAGGC |
| | | AAGACCGACATCCGAGACAAGGAGAACTACATCTCCAAGCAGCT |
| | | CAACCCTGTGTTTGGGAAGTCCTTTGACATTGAGGCCTCCTTCC |
| | | CCATGGAGTCCATGTTGACAGTGGCCGTGTACGACTGGGATCT |
| | | GGTGGGCACTGATGACCTCATCGGAGAAACCAAGATTGACCTG |
| | | GAAAACCGCTTCTACAGCAAGCATCGCGCCACCTGCGGCATCG |
| | | CACAGACCTATTCCATACATGGCTACAATATCTGGAGGGACCCC |
| | | ATGAAGCCCAGCCAGATCCTGACACGCCTCTGTAAAGAGGGCA |
| | | AAGTGGACGGCCCCCACTTTGGTCCCCATGGGAGAGTGAGGGT |
| | | TGCCAACCGTGTCTTCACGGGGCCTTCAGAAATAGAGGATGAGA |
| | | ATGGTCAGAGGAAGCCCACAGATGAGCACGTGGCACTGTCTGC |
| | | TCTGAGACACTGGGAGGACATCCCCCGGGTGGGCTGCCGCCTT |
| | | GTGCCGGAACACGTGGAGACCAGGCCGCTGCTCAACCCTGACA |
| | | AGCCAGGCATTGAGCAGGGCCGCCTGGAGCTGTGGGTGGACAT |
| | | GTTCCCCATGGACATGCCAGCCCCTGGGACACCTCTGGATATAT |
| | | CCCCCAGGAAACCCAAGAAGTACGAGCTGCGGGTCATCGTGTG |
| | | GAACACAGACGAGGTGGTCCTGGAAGACGATGATTTCTTCACG |
| | | GGAGAGAAGTCCAGTGACATTTTTGTGAGGGGGTGGCTGAAGG |
| | | GCCAGCAGGAGGACAAACAGGACACAGATGTCCACTATCACTC |
| | | CCTCACGGGGGAGGGCAACTTCAACTGGAGATACCTCTTCCCC |
| | | TTCGACTACCTAGCGGCCGAAGAGAAGATCGTTATGTCCAAAAA |
| | | GGAGTCTATGTTCTCCTGGGATGAGACGGAGTACAAGATCCCTG |
| | | CGCGGCTCACCCTGCAGATCTGGGACGCTGACCACTTCTCGGC |
| | | TGACGACTTCCTGGGGGCTATCGAGCTGGACCTGAACCGGTTC |
| | | CCGAGGGGCGCTAAGACAGCCAAGCAGTGCACCATGGAGATGG |
| | | CCACCGGGGAGGTGGACGTACCCCTGGTTTCCATCTTTAAACAG |
| | | AAACGTGTCAAAGGCTGGTGGCCCCTCCTGGCCCGCAATGAGA |
| | | ATGATGAGTTTGAGCTCACAGGCAAAGTGGAGGCGGAGCTACA |
| | | CCTACTCACGGCAGAGGAGGCAGAGAAGAACCCTGTGGGCCTG |
| | | GCTCGCAATGAACCTGATCCCCTAGAAAAACCCAACCGGCCTGA |
| | | CACGGCATTCGTCTGGTTCCTGAACCCACTCAAATCTATCAAGT |
| | | ACCTCATCTGCACCCGGTACAAGTGGCTGATCATCAAGATCGTG |
| | | CTGGCGCTGCTGGGGCTGCTCATGCTGGCCCTCTTCCTTTACA |
| | | GCCTCCCAGGCTACATGGTCAAGAAGCTCCTAGGGGCCTGAAG |
| | | TGTGCCCCACCCCAGCCCGCTCCAGCATCCCTCCAGGGGCTGC |
| | | TGCGTATTTTGCCTTCCCTCACCTGGACTCTCTCCCAACTCCCT |
| | | GAGGAGCCCTCCCACGCCTGCCAGCCTTGAGCAAGACACCTGC |
| | | TTGCTGGACTTCATCCCCACCCCACACCCAAACTGTTGCTTGCC |
| | | TGATCTTGTCCCAGGCCTGCCTGGGGTTTGGGGCACAGTTGGC |
| | | CTCCAAAACCAGATACCCTCTTGTCTAAAGTACCAGGTTCCTCTG |
| | | CCCAACCCCAAGAGTGGTAGTGGCCCAACCCTCCCTGTGCTTTC |
| | | CAAATCTTGTCTTAAGGCACCAGTGAAATTAACCAAGAAACGCG |
| | | GAGCAATGCCCAAGGCTCTGATGAGTAGGAACACGTGGAAAGC |
| | | ACCAGGAATGCCAGCAGAGGCGAGGCGGCACACCTCTCTGCAG |
| | | AGCATCCAGGCCGAGCGGCGGGCAGCGGCCAGCTGCTTCTGC |
| | | GCATGCTCTCCTCTTGGCTCTGCTTCTTTCTCACAGTCACAGTCA |
| | | CTTCACAGCTTAGCCTTGGGCTTCCCATCACTTCCAGGGGTGCC |
| | | TCTGCCTTGGCCAGTGTGTGTCAGCTAGTACACAAGCTCCAAGT |
| | | GTGAATCAGGTGTACTGGCCGTCCTGAAGACTGACTGCCCTGTC |
| | | CTTCCTGCCGACAGCCACACCCGAGTGTACACTTAAAGCGGTG |
| | | CCCTTCTGCCTCTGTGGGCCTGCTGGCTGCTGTTCCTTTCTTGA |
| | | GTGTGATTTTTTTTTTCTCTCCCTCAATAAAATAAATCAAACTCTG |
| | | AGAC |

### Expression of OTOF in Mammalian Cells

Mutations in OTOF have been linked to sensorineural hearing loss and auditory neuropathy. The compositions and methods described herein increase the expression of WT OTOF protein through administration a first nucleic acid vector that contains a polynucleotide encoding an N-terminal portion of an OTOF protein and a second nucleic acid vector that contains a polynucleotide encoding a C-terminal portion of an OTOF protein. In order to utilize nucleic acid vectors for therapeutic application in the treatment of sensorineural hearing loss and auditory neuropathy, they can be directed to the interior of the cell, and, in particular, to specific cell types. A wide array of methods has been established for the delivery of proteins to mammalian cells and for the stable expression of genes encoding proteins in mammalian cells.

### Polynucleotides Encoding OTOF

One platform that can be used to achieve therapeutically effective intracellular concentrations of OTOF in mammalian cells is via the stable expression of the gene encoding OTOF (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

OTOF can also be introduced into a mammalian cell by targeting vectors containing portions of a gene encoding an OTOF protein to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding an OTOF protein by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode an OTOF protein downstream of a mammalian promoter (e.g., a polynucleotide that encodes an N-terminal portion of an OTOF protein downstream of a mammalian promoter). Promoters that are useful for the expression of an OTOF protein in mammalian cells include ubiquitous promoters, cochlear hair cell-specific promoters, and inner hair cell-specific promoters. Ubiquitous promoters include the CAG promoter, a cytomegalovirus (CMV) promoter (e.g., the CMV immediate-early enhancer and promoter, a CMVmini promoter, a minCMV promoter, a CMV-TATA+INR promoter, or a min CMV-T6 promoter), the chicken β-actin promoter, the smCBA promoter, the CB7 promoter, the hybrid CMV enhancer/human β-actin promoter, the CASI promoter, the dihydrofolate reductase (DHFR) promoter, the human β-actin promoter, a β-globin promoter (e.g., a minimal (3-globin promoter), an HSV promoter (e.g., a minimal HSV ICP0 promoter or a truncated HSV ICP0 promoter), an SV40 promoter (e.g., an SV40 minimal promoter), the EF1a promoter, and the PGK promoter. Cochlear hair cell-specific promoters include the Myosin 15 (Myo15) promoter, the Myosin 7A (Myo7A) promoter, the Myosin 6 (Myo6) promoter, the POU4F3 promoter, the Atonal BHLH Transcription Factor 1 (ATOH1) promoter, the LIM Homeobox 3 (LHX3) promoter, the α9 acetylcholine receptor (α9AChR) promoter, and the α10 acetylcholine receptor (α10AChR) promoter. Inner hair cell-specific promoters include the FGF8 promoter, the VGLUT3 promoter, the OTOF promoter, and the calcium binding protein 2 (CABP2) promoter (described in International Patent Application Publication Number WO2021/091940, which is incorporated herein by reference). Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

Murine Myosin 15 Promoters

In some embodiments, the Myo15 promoter for use in the compositions and methods described herein includes nucleic acid sequences from regions of the murine Myo15 locus that are capable of expressing a transgene specifically in hair cells, or variants thereof, such as a nucleic acid sequences that have at least 85% sequence identity (e.g., 85%, 86%, 87%, 88%, 89%, 90%, 91%, 92%, 93%, 94%, 95%, 96%, 97%, 98%, 99%, or more, sequence identity) to regions of the murine Myo15 locus that are capable of expressing a transgene specifically in hair cells. These regions include nucleic acid sequences immediately preceding the murine Myo15 translation start site and an upstream regulatory element that is located over 5 kb from the murine Myo15 translation start site. The Myo15 promoter for use in the compositions and methods described herein can optionally include a linker operably linking the regions of the murine Myo15 locus that are capable of expressing a transgene specifically in hair cells, or the regions of the murine Myo15 locus can be joined directly without an intervening linker.

In some embodiments, the Myo15 promoter for use in the compositions and methods described herein contains a first region (an upstream regulatory element) having at least 85% sequence identity (e.g., 85%, 86%, 87%, 88%, 89%, 90%, 91%, 92%, 93%, 94%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 24) or a functional portion or derivative thereof joined (e.g., operably linked) to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence immediately preceding the murine Myo15 translation start site (nucleic acids from −1 to −1157 with respect to the murine Myo15 translation start site, the sequence of which is set forth in SEQ ID NO: 25) or a functional portion or derivative thereof. The functional portion of SEQ ID NO: 24 may have the sequence of nucleic acids from −7166 to −7091 with respect to the murine Myo15 translation start site (set forth in SEQ ID NO: 26) and/or the sequence of nucleic acids from −7077 to −6983 with respect to the murine Myo15 translation start site (set forth in SEQ ID NO: 27). The first region may contain the nucleic acid sequence of SEQ ID NO: 26 fused to the nucleic acid sequence of SEQ ID NO: 27 with no intervening nucleic acids, as set forth in SEQ ID NO: 28, or the first region may contain the nucleic acid sequence of SEQ ID NO: 27 fused to the nucleic acid sequence of SEQ ID NO: 26 with no intervening nucleic acids, as set forth in SEQ ID NO: 29. Alternatively, the first region may contain the sequences of SEQ ID NO: 26 and SEQ ID NO: 27 joined by the endogenous intervening nucleic acid sequence (e.g., the first region may have or include the sequence of nucleic acids from −7166 to −6983 with respect to the murine Myo15 translation start site, as set forth in SEQ ID NO: 30 and SEQ ID NO: 50) or a nucleic acid linker. In a murine Myo15 promoter in which the first region contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26). The functional portion of SEQ ID NO: 25 may have the sequence of nucleic acids from −590 to −509 with respect to the murine Myo15 translation start site (set forth in SEQ ID NO: 31) and/or the sequence of nucleic acids from −266 to −161 with respect to the murine Myo15 translation start site (set forth in SEQ ID NO: 32). In some embodiments, the sequence containing SEQ ID NO: 31 has the sequence of SEQ ID NO: 51. In some embodiments, the sequence containing SEQ ID NO: 32 has the sequence of SEQ ID NO: 52. The second region may contain the nucleic acid sequence of SEQ ID NO: 31 fused to the nucleic acid sequence of SEQ ID NO: 32 with no intervening nucleic acids, as set forth in SEQ ID NO: 33, or the second region may contain the nucleic acid sequence of SEQ ID NO: 32 fused to the nucleic acid sequence of SEQ ID NO: 31 with no intervening nucleic acids, as set forth in SEQ ID NO: 34. The second region may contain the nucleic acid sequence of SEQ ID NO: 51 fused to the nucleic acid sequence of SEQ ID NO: 52 with no intervening nucleic acids, as set forth in SEQ ID NO: 55, or the second region may contain the nucleic acid sequence of SEQ ID NO: 52 fused to the nucleic acid sequence of SEQ ID NO: 51 with no intervening nucleic acids. Alternatively, the second region may contain the sequences of SEQ ID NO: 31 and SEQ ID NO: 32 joined by the endogenous intervening nucleic acid sequence (e.g., the second region may have the sequence of nucleic acids from −590 to −161 with respect to the murine Myo15 translation start site, as set forth in SEQ ID NO: 35) or a nucleic acid linker. In a murine Myo15 promoter in which the second region contains both SEQ ID NO: 31 and SEQ ID NO: 32, the two sequences can be included in any order (e.g., SEQ ID NO: 31 may be joined to (e.g., precede) SEQ ID NO: 32, or SEQ ID NO: 32 may be joined to (e.g., precede) SEQ ID NO: 31).

The first region and the second region of the murine Myo15 promoter can be joined directly or can be joined by a nucleic acid linker. For example, the murine Myo15 promoter can contain the sequence of SEQ ID NO: 24 or a functional portion or derivative thereof (e.g., any one or more of SEQ ID NOs: 26-30 and 50, e.g., SEQ ID NOs 26 and 27) fused to the sequence of SEQ ID NO: 25 or a functional portion or derivative thereof (e.g., any one or more of SEQ ID NOs: 31-35, 51, 52, and 55, e.g., SEQ ID NOs 31 and 32) with no intervening nucleic acids. For example, the nucleic acid sequence of the murine Myo15 promoter that results from direct fusion of SEQ ID NO: 24 to SEQ ID NO: 25 is set forth in SEQ ID NO: 36. Alternatively, a linker can be used to join the sequence of SEQ ID NO: 24 or a functional portion or derivative thereof (e.g., any one or more of SEQ ID NOs: 26-30 and 50, e.g., SEQ ID NOs 26 and 27) to the sequence of SEQ ID NO: 25 or a functional portion or derivative thereof (e.g., any one or more of SEQ ID NOs: 31-35, 51, 52, and 55, e.g., SEQ ID NOs 31 and 32). Exemplary Myo15 promoters containing functional portions of both SEQ ID NO: 24 and SEQ ID NO: 25 are provided in SEQ ID NOs: 38, 39, 53, 54, 59, and 60.

The length of a nucleic acid linker for use in a murine Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the murine Myo15 promoter described herein do not disrupt the ability of the murine Myo15 promoter of the invention to induce transgene expression in hair cells.

In some embodiments, the sequence of SEQ ID NO: 24 or a functional portion or derivative thereof (e.g., any one or more of SEQ ID NOs: 26-30 and 50, e.g., SEQ ID NOs 26 and 27) is joined (e.g., operably linked) to the sequence of SEQ ID NO: 25 or a functional portion or derivative thereof (e.g., any one or more of SEQ ID NOs: 31-35, 51, 52, and 55, e.g., SEQ ID NOs 31 and 32), and, in some embodiments, the order of the regions is reversed (e.g., the sequence of SEQ ID NO: 25 or a functional portion or derivative thereof (e.g., any one or more of SEQ ID NOs: 31-35, 51, 52, and 55, e.g., SEQ ID NOs 31 and 32) is joined (e.g., operably linked) to the sequence of SEQ ID NO: 24 or a functional portion or derivative thereof (e.g., any one or more of SEQ ID NOs: 26-30 and 50, e.g., SEQ ID NOs 26 and 27)). For example, the nucleic acid sequence of a murine Myo15 promoter that results from direct fusion of SEQ ID NO: 25 to SEQ ID NO: 24 is set forth in SEQ ID NO: 37. An example of a murine Myo15 promoter in which a functional portion or derivative of SEQ ID NO: 25 precedes a functional portion or derivative of SEQ ID NO: 24 is provided in SEQ ID NO: 58. Regardless of order, the sequence of SEQ ID NO: 24 or a functional portion or derivative thereof and the sequence of SEQ ID NO: 25 or a functional portion or derivative thereof can be joined by direct fusion or a nucleic acid linker, as described above.

In some embodiments, the murine Myo15 promoter for use in the compositions and methods described herein contains a region having at least 85% sequence identity (e.g., 85%, 86%, 87%, 88%, 89%, 90%, 91%, 92%, 93%, 94%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 24) or a functional portion or derivative thereof. The functional portion of SEQ ID NO: 24 may have the sequence of nucleic acids from −7166 to −7091 with respect to the murine Myo15 translation start site (set forth in SEQ ID NO: 26) and/or the sequence of nucleic acids from −7077 to −6983 with respect to the murine Myo15 translation start site (set forth in SEQ ID NO: 27). The murine Myo15 promoter may contain the nucleic acid sequence of SEQ ID NO: 26 fused to the nucleic acid sequence of SEQ ID NO: 27 with no intervening nucleic acids, as set forth in SEQ ID NO: 28, or the murine Myo15 promoter may contain the nucleic acid sequence of SEQ ID NO: 27 fused to the nucleic acid sequence of SEQ ID NO: 26 with no intervening nucleic acids, as set forth in SEQ ID NO: 29. Alternatively, the murine Myo15 promoter may contain the sequences of SEQ ID NO: 26 and SEQ ID NO: 27 joined by the endogenous intervening nucleic acid sequence (e.g., the first region may have or include the sequence of nucleic acids from −7166 to −6983 with respect to the murine Myo15 translation start site, as set forth in SEQ ID NO: 30 and SEQ ID NO: 50) or a nucleic acid linker. In a murine Myo15 promoter that contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26).

In some embodiments, the murine Myo15 promoter for use in the compositions and methods described herein contains a region having at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence immediately upstream of the murine Myo15 translation start site (nucleic acids from −1 to −1157 with respect to the murine Myo15 translation start site, the sequence of which is set forth in SEQ ID NO: 25) or a functional portion or derivative thereof. The functional portion of SEQ ID NO: 25 may have the sequence of nucleic acids from −590 to −509 with respect to the murine Myo15 translation start site (set forth in SEQ ID NO: 31) and/or the sequence of nucleic acids from −266 to −161 with respect to the murine Myo15 translation start site (set forth in SEQ ID NO: 32). In some embodiments, the sequence containing SEQ ID NO: 31 has the sequence of SEQ ID NO: 51. In some embodiments, the sequence containing SEQ ID NO: 32 has the sequence of SEQ ID NO: 52. The murine Myo15 promoter may contain the nucleic acid sequence of SEQ ID NO: 31 fused to the nucleic acid sequence of SEQ ID NO: 32 with no intervening nucleic acids, as set forth in SEQ ID NO: 33, or the murine Myo15 promoter may contain the nucleic acid sequence of SEQ ID NO: 32 fused to the nucleic acid sequence of SEQ ID NO: 31 with no intervening nucleic acids, as set forth in SEQ ID NO: 34. The murine Myo15 promoter may contain the nucleic acid sequence of SEQ ID NO: 51 fused to the nucleic acid sequence of SEQ ID NO: 52 with no intervening nucleic acids, as set forth in SEQ ID NO: 55, or the murine Myo15 promoter may contain the nucleic acid sequence of SEQ ID NO: 52 fused to the nucleic acid sequence of SEQ ID NO: 51 with no intervening nucleic acids. Alternatively, the murine Myo15 promoter may contain the sequences of SEQ ID NO: 31 and SEQ ID NO: 32 joined by the endogenous intervening nucleic acid sequence (e.g., the second region may have the sequence of nucleic acids from −590 to −161 with respect to the murine Myo15 translation start site, as set forth in SEQ ID NO: 35) or a nucleic acid linker. In a murine Myo15 promoter that contains both SEQ ID NO: 31 and SEQ ID NO: 32, the two sequences can be included in any order (e.g., SEQ ID NO: 31 may be joined to (e.g., precede) SEQ ID NO: 32, or SEQ ID NO: 32 may be joined to (e.g., precede) SEQ ID NO: 31).

In some embodiments, the murine Myo15 promoter for use in the compositions and methods described herein contains a functional portion or derivative of a region having at least 85% sequence identity (e.g., 85%, 86%, 87%, 88%, 89%, 90%, 91%, 92%, 93%, 94%, 95%, 96%, 97%, 98%, 99%, or more, sequence identity) to a region containing the first non-coding exon of the Myo15 gene (nucleic acids from −6755 to −7209 with respect to the murine Myo15 translation start site, the sequence of which is set forth in SEQ ID NO: 24) flanked on both sides by a functional portion or derivative of a region having at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence immediately upstream of the murine Myo15 translation start site (nucleic acids from −1 to −1157 with respect to the murine Myo15 translation start site, the sequence of which is set forth in SEQ ID NO: 25). For example, a functional portion or derivative of SEQ ID NO: 25, such as SEQ ID NO: 31 or 51 may be directly fused or joined by a nucleic acid linker to a portion of SEQ ID NO: 24, such as any one of SEQ ID NOs: 26-30 and 50, which is directly fused or joined by a nucleic acid linker to a different functional portion of SEQ ID NO: 25, such as SEQ ID NO: 32 or 52. In other embodiments, a functional portion or derivative of SEQ ID NO: 25, such as SEQ ID NO: 32 or 52 may be directly fused or joined by a nucleic acid linker to a portion of SEQ ID NO: 24, such as any one of SEQ ID NOs: 26-30 and 50, which is directly fused or joined by a nucleic acid linker to a different functional portion of SEQ ID NO: 25, such as SEQ ID NO: 31 or 51. For example, polynucleotides having at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence of SEQ ID NOs: 51, 50, and 52 can be fused to produce a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence of SEQ ID NO: 56. In some embodiments, polynucleotides having at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence of SEQ ID NOs: 52, 50, and 51 can be fused to produce a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequence of SEQ ID NO: 57.

Human Myosin 15 Promoters

In some embodiments, the Myo15 promoter for use in the compositions and methods described herein includes nucleic acid sequences from regions of the human Myo15 locus that are capable of expressing a transgene specifically in hair cells, or variants thereof, such as a nucleic acid sequences that have at least 85% sequence identity (e.g., 85%, 86%, 87%, 88%, 89%, 90%, 91%, 92%, 93%, 94%, 95%, 96%, 97%, 98%, 99%, or more, sequence identity) to regions of the human Myo15 locus that are capable of expressing a transgene specifically in hair cells. The Myo15 promoter for use in the compositions and methods described herein can optionally include a linker operably linking the regions of the human Myo15 locus that are capable of expressing a transgene specifically in hair cells, or the regions of the human Myo15 locus can be joined directly without an intervening linker.

In some embodiments, the Myo15 promoter for use in the compositions and methods described herein contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to the sequence set forth in SEQ ID NO: 40 or a functional portion or derivative thereof joined (e.g., operably linked) to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to the sequence set forth in SEQ ID NO: 41 or a functional portion or derivative thereof. The functional portion of SEQ ID NO: 40 may have the sequence set forth in SEQ ID NO: 42. The functional portion of SEQ ID NO: 41 may have the sequence set forth in SEQ ID NO: 43 and/or the sequence set forth in SEQ ID NO: 44. The second region may contain the nucleic acid sequence of SEQ ID NO: 43 fused to the nucleic acid sequence of SEQ ID NO: 44 with no intervening nucleic acids, as set forth in SEQ ID NO: 45, or the second region may contain the nucleic acid sequence of SEQ ID NO: 44 fused to the nucleic acid sequence of SEQ ID NO: 43 with no intervening nucleic acids, as set forth in SEQ ID NO: 46. Alternatively, the second region may contain the sequences of SEQ ID NO: 43 and SEQ ID NO: 44 joined by the endogenous intervening nucleic acid sequence (as set forth in SEQ ID NO: 47) or a nucleic acid linker. In a human Myo15 promoter in which the second region contains both SEQ ID NO: 43 and SEQ ID NO: 44, the two sequences can be included in any order (e.g., SEQ ID NO: 43 may be joined to (e.g., precede) SEQ ID NO: 44, or SEQ ID NO: 44 may be joined to (e.g., precede) SEQ ID NO: 43).

The first region and the second region of the human Myo15 promoter can be joined directly or can be joined by a nucleic acid linker. For example, the human Myo15 promoter can contain the sequence of SEQ ID NO: 40 or a functional portion or derivative thereof (e.g., SEQ ID NO: 42) fused to the sequence of SEQ ID NO: 41 or a functional portion or derivative thereof (e.g., any one or more of SEQ ID NOs: 43-47, e.g., SEQ ID NOs: 43 and/or 44) with no intervening nucleic acids. Alternatively, a linker can be used to join the sequence of SEQ ID NO: 40 or a functional portion or derivative thereof (e.g., SEQ ID NO: 42) to the sequence of SEQ ID NO: 41 or a functional portion or derivative thereof (e.g., any one or more of SEQ ID NOs: 43-47, e.g., SEQ ID NOs: 43 and/or 44). Exemplary human Myo15 promoters containing functional portions of both SEQ ID NO: 40 and SEQ ID NO: 41 are provided in SEQ ID NOs: 48 and 49.

In some embodiments, the sequence of SEQ ID NO: 40 or a functional portion or derivative thereof (e.g., SEQ ID NO: 42) is joined (e.g., operably linked) to the sequence of SEQ ID NO: 41 or a functional portion or derivative thereof (e.g., any one or more of SEQ ID NOs: 43-47, e.g., SEQ ID NOs: 43 and 44), and, in some embodiments, the order of the regions is reversed (e.g., the sequence of SEQ ID NO: 41 or a functional portion or derivative thereof (e.g., any one or more of SEQ ID NOs: 43-47, e.g., SEQ ID NOs: 43 and/or 44) is joined (e.g., operably linked) to the sequence of SEQ ID NO: 40 or a functional portion or derivative thereof (e.g., SEQ ID NO: 42). Regardless of order, the sequence of SEQ ID NO: 40 or a functional portion or derivative thereof and the sequence of SEQ ID NO: 41 or a functional portion or derivative thereof can be joined by direct fusion or a nucleic acid linker, as described above.

In some embodiments, the human Myo15 promoter for use in the compositions and methods described herein contains a region having at least 85% sequence identity (e.g., 85%, 86%, 87%, 88%, 89%, 90%, 91%, 92%, 93%, 94%, 95%, 96%, 97%, 98%, 99%, or more, sequence identity) to a region containing the sequence set forth in SEQ ID NO: 40 or a functional portion or derivative thereof. The functional portion of SEQ ID NO: 40 may have the sequence of nucleic acids set forth in SEQ ID NO: 42.

In some embodiments, the human Myo15 promoter for use in the compositions and methods described herein contains a region having at least 85% sequence identity (e.g., 85%, 86%, 87%, 88%, 89%, 90%, 91%, 92%, 93%, 94%, 95%, 96%, 97%, 98%, 99%, or more, sequence identity) to the sequence set forth in SEQ ID NO: 41 or a functional portion or derivative thereof. The functional portion of SEQ ID NO: 41 may have the sequence set forth in SEQ ID NO: 43 and/or the sequence set forth in SEQ ID NO: 44. The human Myo15 promoter may contain the nucleic acid sequence of SEQ ID NO: 43 fused to the nucleic acid sequence of SEQ ID NO: 44 with no intervening nucleic acids, as set forth in SEQ ID NO: 45, or the human Myo15 promoter may contain the nucleic acid sequence of SEQ ID NO: 44 fused to the nucleic acid sequence of SEQ ID NO: 43 with no intervening nucleic acids, as set forth in SEQ ID NO: 46. Alternatively, the human Myo15 promoter may contain the sequences of SEQ ID NO: 43 and SEQ ID NO: 44 joined by the endogenous intervening nucleic acid sequence (e.g., as set forth in SEQ ID NO: 47) or a nucleic acid linker. In a human Myo15 promoter that contains both SEQ ID NO: 43 and SEQ ID NO: 44, the two sequences can be included in any order (e.g., SEQ ID NO: 43 may be joined to (e.g., precede) SEQ ID NO: 44, or SEQ ID NO: 44 may be joined to (e.g., precede) SEQ ID NO: 43).

The length of a nucleic acid linker for use in a human Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the human Myo15 promoters described herein do not disrupt the ability of the human Myo15 promoter of the invention to induce transgene expression in hair cells.

The foregoing Myo15 promoter sequences are summarized in Table 3, below.

TABLE 3
Exemplary nucleotide sequences for use
in the Myo15 promoter described herein

| SEQ | | |
|---|---|---|
| ID | Description of nucleic | Nucleic Acid |
| NO. | acid sequence | Sequence |
| 24 | Region containing non- | CTGCAGCTCAGCCTACTAC |
| | coding exon 1 of Myo15 | TTGCTTTCCAGGCTGTTCC |
| | (−6755 to −7209) | TAGTTCCCATGTCAGCTGC |
| | | TTGTGCTTTCCAGAGACAA |
| | | AACAGGAATAATAGATGTC |
| | | ATTAAATATACATTGGGCC |
| | | CCAGGCGGTCAATGTGGCA |
| | | GCCTGAGCCTCCTTTCCAT |
| | | CTCTGTGGAGGCAGACATA |
| | | GGACCCCCAACAAACAGCA |
| | | TGCAGGTTGGGAGCCAGCC |
| | | ACAGGACCCAGGTAAGGGG |
| | | CCCTGGGTCCTTAAGCTTC |
| | | TGCCACTGGCTCCGGCATT |
| | | GCAGAGAGAAGAGAAGGGG |
| | | CGGCAGAGCTGAACCTTAG |
| | | CCTTGCCTTCCTGGGTACC |
| | | CTTCTGAGCCTCACTGTCT |
| | | TCTGTGAGATGGGCAAAGT |
| | | GCGGGTGTGACTCCTTGGC |
| | | AACGGTGTTACACCAGGGC |
| | | AGGTAAAGTTGTAGTTATT |
| | | TGTGGGGTACACCAGGACT |
| | | GTTAAAGGTGTAACTAT |
| 25 | Region immediately | GGTCTCACCCAGCATTTTC |
| | preceding the | ACTTCTAATAAGTTCAAAT |
| | translation | GTGATACGGCACCTTTCTA |
| | start site of | AAAATTAGTTTTCAGGGAA |
| | Myo15 (−1 to −1157) | ATAGGGTTCAAAACTGGTA |
| | | GTGGTAGGGTCCATTCTCA |
| | | CGACCCCCAGGCCTGCTAA |
| | | CCCTGACCAAGCTACCTAT |
| | | TACTTACCCTCCTCTTTCT |
| | | CCTCCTCCTCTTTCTCCTT |
| | | CTCCTGCTTCCCCTCTTCC |
| | | TTCTCCCTCCCTTCCTCTC |
| | | CCTCCTCCCCCTCCTTGGC |
| | | TGTGATCAGATCCAGAGCC |
| | | TGAATGAGCCTCCTGACCC |
| | | CACACCCCCACTAGCATGG |
| | | GCCTGCAAGTGCCCAGAAG |
| | | TCCCTCCTGCCTCCTAAAC |
| | | TGCCCAGCCGATCCATTAG |
| | | CTCTTCCTTCTTCCCAGTG |
| | | AAAGAAGCAGGCACAGCCT |
| | | GTCCCTCCCGTTCTACAGA |
| | | AAGGAAGCTACAGCACAGG |
| | | GAGGGCCAAAGGCCTTCCT |
| | | GGGACTAGACAGTTGATCA |
| | | ACAGCAGGACTGGAGAGCT |
| | | GGGCTCCATTTTTGTTCCT |
| | | TGGTGCCCTGCCCCTCCCC |
| | | ATGACCTGCAGAGACATTC |
| | | AGCCTGCCAGGCTTTATGA |
| | | GGTGGGAGCTGGGCTCTCC |
| | | CTGATGTATTATTCAGCTC |
| | | CCTGGAGTTGGCCAGCTCC |
| | | TGTTACACTGGCCACAGCC |
| | | CTGGGCATCCGCTTCTCAC |
| | | TTCTAGTTTCCCCTCCAAG |
| | | GTAATGTGGTGGGTCATGA |
| | | TCATTCTATCCTGGCTTCA |
| | | GGGACCTGACTCCACTTTG |
| | | GGGCCATTCGAGGGGTCTA |
| | | GGGTAGATGATGTCCCCCT |
| | | GTGGGGATTAATGTCCTGC |
| | | TCTGTAAAACTGAGCTAGC |
| | | TGAGATCCAGGAGGGCTTG |
| | | GCCAGAGACAGCAAGTTGT |
| | | TGCCATGGTGACTTTAAAG |
| | | CCAGGTTGCTGCCCCAGCA |
| | | CAGGCCTCCCAGTCTACCC |
| | | TCACTAGAAAACAACACCC |
| | | AGGCACTTTCCACCACCTC |
| | | TCAAAGGTGAAACCCAAGG |
| | | CTGGTCTAGAGAATGAATT |
| | | ATGGATCCTCGCTGTCCGT |
| | | GCCACCCAGCTAGTCCCAG |
| | | CGGCTCAGACACTGAGGAG |
| | | AGACTGTAGGTTCAGCTAC |
| | | AAGCAAAAAGACCTAGCTG |
| | | GTCTCCAAGCAGTGTCTCC |
| | | AAGTCCCTGAACCTGTGAC |
| | | ACCTGCCCCAGGCATCATC |
| | | AGGCACAGAGGGCCACC |
| 26 | Portion of SEQ ID NO: | CCCATGTCAGCTGCTTGTG |
| | 24 (−7166 to −7091) | CTTTCCAGAGACAAAACAG |
| | | GAATAATAGATGTCATTAA |
| | | ATATACATTGGGCCCCAGG |
| 27 | Portion of SEQ ID NO: | AGCCTGAGCCTCCTTTCCA |
| | 24 (−7077 to −6983) | TCTCTGTGGAGGCAGACAT |
| | | AGGACCCCCAACAAACAGC |
| | | ATGCAGGTTGGGAGCCAGC |
| | | CACAGGACCCAGGTAAGGG |
| 28 | Portion of SEQ ID NO: | CCCATGTCAGCTGCTTGTG |
| | 24 (SEQ ID NO: 26 | CTTTCCAGAGACAAAACAG |
| | fused to SEQ ID NO: | GAATAATAGATGTCATTAA |
| | 27) | ATATACATTGGGCCCCAGG |
| | | AGCCTGAGCCTCCTTTCCA |
| | | TCTCTGTGGAGGCAGACAT |
| | | AGGACCCCCAACAAACAGC |
| | | ATGCAGGTTGGGAGCCAGC |
| | | CACAGGACCCAGGTAAGGG |
| 29 | Portion of SEQ ID NO: | AGCCTGAGCCTCCTTTCCA |
| | 24 (SEQ ID NO: 27 | TCTCTGTGGAGGCAGACAT |
| | fused to SEQ ID NO: | AGGACCCCCAACAAACAGC |
| | 26) | ATGCAGGTTGGGAGCCAGC |
| | | CACAGGACCCAGGTAAGGG |
| | | CCCATGTCAGCTGCTTGTG |
| | | CTTTCCAGAGACAAAACAG |
| | | GAATAATAGATGTCATTAA |
| | | ATATACATTGGGCCCCAGG |
| 30 | Portion of SEQ ID NO: | CCCATGTCAGCTGCTTGTG |
| | 24 | CTTTCCAGAGACAAAACAG |
| | (−7166 to −6983) | GAATAATAGATGTCATTAA |
| | | ATATACATTGGGCCCCAGG |
| | | CGGTCAATGTGGCAGCCTG |
| | | AGCCTCCTTTCCATCTCTG |
| | | TGGAGGCAGACATAGGACC |
| | | CCCAACAAACAGCATGCAG |
| | | GTTGGGAGCCAGCCACAGG |
| | | ACCCAGGTAAGGG |
| 31 | Portion of SEQ ID NO: | TGAGGTGGGAGCTGGGCTC |
| | 25 (−590 to −509) | TCCCTGATGTATTATTCAG |
| | | CTCCCTGGAGTTGGCCAGC |
| | | TCCTGTTACACTGGCCACA |
| | | GCCCTG |
| 32 | Portion of SEQ ID NO: | CACAGGCCTCCCAGTCTAC |
| | 25 (−266 to −161) | CCTCACTAGAAAACAACAC |
| | | CCAGGCACTTTCCACCACC |
| | | TCTCAAAGGTGAAACCCAA |
| | | GGCTGGTCTAGAGAATGAA |
| | | TTATGGATCCT |
| 33 | Portion of SEQ ID NO: | TGAGGTGGGAGCTGGGCTC |
| | 25 | TCCCTGATGTATTATTCAG |
| | (SEQ ID NO: 31 fused | CTCCCTGGAGTTGGCCAGC |
| | to SEQ ID NO: 32) | TCCTGTTACACTGGCCACA |
| | | GCCCTGCACAGGCCTCCCA |
| | | GTCTACCCTCACTAGAAAA |
| | | CAACACCCAGGCACTTTCC |
| | | ACCACCTCTCAAAGGTGAA |
| | | ACCCAAGGCTGGTCTAGAG |
| | | AATGAATTATGGATCCT |
| 34 | Portion of SEQ ID NO: | CACAGGCCTCCCAGTCTAC |
| | 25 | CCTCACTAGAAAACAACAC |
| | (SEQ ID NO: 32 fused | CCAGGCACTTTCCACCACC |
| | to SEQ ID NO: 31) | TCTCAAAGGTGAAACCCAA |
| | | GGCTGGTCTAGAGAATGAA |
| | | TTATGGATCCTTGAGGTGG |
| | | GAGCTGGGCTCTCCCTGAT |
| | | GTATTATTCAGCTCCCTGG |
| | | AGTTGGCCAGCTCCTGTTA |
| | | CACTGGCCACAGCCCTG |
| 35 | Portion of SEQ ID NO: | TGAGGTGGGAGCTGGGCTC |
| | 25 | TCCCTGATGTATTATTCAG |
| | (−590 to −161) | CTCCCTGGAGTTGGCCAGC |
| | | TCCTGTTACACTGGCCACA |
| | | GCCCTGGGCATCCGCTTCT |
| | | CACTTCTAGTTTCCCCTCC |
| | | AAGGTAATGTGGTGGGTCA |
| | | TGATCATTCTATCCTGGCT |
| | | TCAGGGACCTGACTCCACT |
| | | TTGGGGCCATTCGAGGGGT |
| | | CTAGGGTAGATGATGTCCC |
| | | CCTGTGGGGATTAATGTCC |
| | | TGCTCTGTAAAACTGAGCT |
| | | AGCTGAGATCCAGGAGGGC |
| | | TTGGCCAGAGACAGCAAGT |
| | | TGTTGCCATGGTGACTTTA |
| | | AAGCCAGGTTGCTGCCCCA |
| | | GCACAGGCCTCCCAGTCTA |
| | | CCCTCACTAGAAAACAACA |
| | | CCCAGGCACTTTCCACCAC |
| | | CTCTCAAAGGTGAAACCCA |
| | | AGGCTGGTCTAGAGAATGA |
| | | ATTATGGATCCT |
| 36 | SEQ ID NO: 24 fused | CTGCAGCTCAGCCTACTAC |
| | to SEQ ID NO: 25 | TTGCTTTCCAGGCTGTTCC |
| | | TAGTTCCCATGTCAGCTGC |
| | | TTGTGCTTTCCAGAGACAA |
| | | AACAGGAATAATAGATGTC |
| | | ATTAAATATACATTGGGCC |
| | | CCAGGCGGTCAATGTGGCA |
| | | GCCTGAGCCTCCTTTCCAT |
| | | CTCTGTGGAGGCAGACATA |
| | | GGACCCCCAACAAACAGCA |
| | | TGCAGGTTGGGAGCCAGCC |
| | | ACAGGACCCAGGTAAGGGG |
| | | CCCTGGGTCCTTAAGCTTC |
| | | TGCCACTGGCTCCGGCATT |
| | | GCAGAGAGAAGAGAAGGGG |
| | | CGGCAGAGCTGAACCTTAG |
| | | CCTTGCCTTCCTGGGTACC |
| | | CTTCTGAGCCTCACTGTCT |
| | | TCTGTGAGATGGGCAAAGT |
| | | GCGGGTGTGACTCCTTGGC |
| | | AACGGTGTTACACCAGGGC |
| | | AGGTAAAGTTGTAGTTATT |
| | | TGTGGGGTACACCAGGACT |
| | | GTTAAAGGTGTAACTATGG |
| | | TCTCACCCAGCATTTTCAC |
| | | TTCTAATAAGTTCAAATGT |
| | | GATACGGCACCTTTCTAAA |
| | | AATTAGTTTTCAGGGAAAT |
| | | AGGGTTCAAAACTGGTAGT |
| | | GGTAGGGTCCATTCTCACG |
| | | ACCCCCAGGCCTGCTAACC |
| | | CTGACCAAGCTACCTATTA |
| | | CTTACCCTCCTCTTTCTCC |
| | | TCCTCCTCTTTCTCCTTCT |
| | | CCTGCTTCCCCTCTTCCTT |
| | | CTCCCTCCCTTCCTCTCCC |
| | | TCCTCCCCCTCCTTGGCTG |
| | | TGATCAGATCCAGAGCCTG |
| | | AATGAGCCTCCTGACCCCA |
| | | CACCCCCACTAGCATGGGC |
| | | CTGCAAGTGCCCAGAAGTC |
| | | CCTCCTGCCTCCTAAACTG |
| | | CCCAGCCGATCCATTAGCT |
| | | CTTCCTTCTTCCCAGTGAA |
| | | AGAAGCAGGCACAGCCTGT |
| | | CCCTCCCGTTCTACAGAAA |
| | | GGAAGCTACAGCACAGGGA |
| | | GGGCCAAAGGCCTTCCTGG |
| | | GACTAGACAGTTGATCAAC |
| | | AGCAGGACTGGAGAGCTGG |
| | | GCTCCATTTTTGTTCCTTG |
| | | GTGCCCTGCCCCTCCCCAT |
| | | GACCTGCAGAGACATTCAG |
| | | CCTGCCAGGCTTTATGAGG |
| | | TGGGAGCTGGGCTCTCCCT |
| | | GATGTATTATTCAGCTCCC |
| | | TGGAGTTGGCCAGCTCCTG |
| | | TTACACTGGCCACAGCCCT |
| | | GGGCATCCGCTTCTCACTT |
| | | CTAGTTTCCCCTCCAAGGT |
| | | AATGTGGTGGGTCATGATC |
| | | ATTCTATCCTGGCTTCAGG |
| | | GACCTGACTCCACTTTGGG |
| | | GCCATTCGAGGGGTCTAGG |
| | | GTAGATGATGTCCCCCTGT |
| | | GGGGATTAATGTCCTGCTC |
| | | TGTAAAACTGAGCTAGCTG |
| | | AGATCCAGGAGGGCTTGGC |
| | | CAGAGACAGCAAGTTGTTG |
| | | CCATGGTGACTTTAAAGCC |
| | | AGGTTGCTGCCCCAGCACA |
| | | GGCCTCCCAGTCTACCCTC |
| | | ACTAGAAAACAACACCCAG |
| | | GCACTTTCCACCACCTCTC |
| | | AAAGGTGAAACCCAAGGCT |
| | | GGTCTAGAGAATGAATTAT |
| | | GGATCCTCGCTGTCCGTGC |
| | | CACCCAGCTAGTCCCAGCG |
| | | GCTCAGACACTGAGGAGAG |
| | | ACTGTAGGTTCAGCTACAA |
| | | GCAAAAAGACCTAGCTGGT |
| | | CTCCAAGCAGTGTCTCCAA |
| | | GTCCCTGAACCTGTGACAC |
| | | CTGCCCCAGGCATCATCAG |
| | | GCACAGAGGGCCACC |
| 37 | SEQ ID NO: 25 fused | GGTCTCACCCAGCATTTTC |
| | to SEQ ID NO: 24 | ACTTCTAATAAGTTCAAAT |
| | | GTGATACGGCACCTTTCTA |
| | | AAAATTAGTTTTCAGGGAA |
| | | ATAGGGTTCAAAACTGGTA |
| | | GTGGTAGGGTCCATTCTCA |
| | | CGACCCCCAGGCCTGCTAA |
| | | CCCTGACCAAGCTACCTAT |
| | | TACTTACCCTCCTCTTTCT |
| | | CCTCCTCCTCTTTCTCCTT |
| | | CTCCTGCTTCCCCTCTTCC |
| | | TTCTCCCTCCCTTCCTCTC |
| | | CCTCCTCCCCCTCCTTGGC |
| | | TGTGATCAGATCCAGAGCC |
| | | TGAATGAGCCTCCTGACCC |
| | | CACACCCCCACTAGCATGG |
| | | GCCTGCAAGTGCCCAGAAG |
| | | TCCCTCCTGCCTCCTAAAC |
| | | TGCCCAGCCGATCCATTAG |
| | | CTCTTCCTTCTTCCCAGTG |
| | | AAAGAAGCAGGCACAGCCT |
| | | GTCCCCCCGTTCTACAGAA |
| | | AGGAAGCTACAGCACAGGG |
| | | AGGGCCAAAGGCCTTCCTG |
| | | GGACTAGACAGTTGATCAA |
| | | CAGCAGGACTGGAGAGCTG |
| | | GGCTCCATTTTTGTTCCTT |
| | | GGTGCCCTGCCCCTCCCCA |
| | | TGACCTGCAGAGACATTCA |
| | | GCCTGCCAGGCTTTATGAG |
| | | GTGGGAGCTGGGCTCTCCC |
| | | TGATGTATTATTCAGCTCC |
| | | CTGGAGTTGGCCAGCTCCT |
| | | GTTACACTGGCCACAGCCC |
| | | TGGGCATCCGCTTCTCACT |
| | | TCTAGTTTCCCCTCCAAGG |
| | | TAATGTGGTGGGTCATGAT |
| | | CATTCTATCCTGGCTTCAG |
| | | GGACCTGACTCCACTTTGG |
| | | GGCCATTCGAGGGGTCTAG |
| | | GGTAGATGATGTCCCCCTG |
| | | TGGGGATTAATGTCCTGCT |
| | | CTGTAAAACTGAGCTAGCT |
| | | GAGATCCAGGAGGGCTTGG |
| | | CCAGAGACAGCAAGTTGTT |
| | | GCCATGGTGACTTTAAAGC |
| | | CAGGTTGCTGCCCCAGCAC |
| | | AGGCCTCCCAGTCTACCCT |
| | | CACTAGAAAACAACACCCA |
| | | GGCACTTTCCACCACCTCT |
| | | CAAAGGTGAAACCCAAGGC |
| | | TGGTCTAGAGAATGAATTA |
| | | TGGATCCTCGCTGTCCGTG |
| | | CCACCCAGCTAGTCCCAGC |
| | | GGCTCAGACACTGAGGAGA |
| | | GACTGTAGGTTCAGCTACA |
| | | AGCAAAAAGACCTAGCTGG |
| | | TCTCCAAGCAGTGTCTCCA |
| | | AGTCCCTGAACCTGTGACA |
| | | CCTGCCCCAGGCATCATCA |
| | | GGCACAGAGGGCCACCCTG |
| | | CAGCTCAGCCTAC |
| 38 | Portion of SEQ ID NO: | TACTTGCTTTCCAGGCTGT |
| | 24 that contains SEQ | TCCTAGTTCCCATGTCAGC |
| | ID NO: 26 and SEQ ID | TGCTTGTGCTTTCCAGAGA |
| | NO: 27 fused to portion | CAAAACAGGAATAATAGAT |
| | of SEQ ID NO: 25 that | GTCATTAAATATACATTGG |
| | contains SEQ ID NO: | GCCCCAGGCGGTCAATGTG |
| | 31 and SEQ ID NO: 32 | GCAGCCTGAGCCTCCTTTC |
| | | CATCTCTGTGGAGGCAGAC |
| | | ATAGGACCCCCAACAAACA |
| | | GCATGCAGGTTGGGAGCCA |
| | | GCCACAGGACCCAGGTAAG |
| | | GGGCCCTGGGTCCTTAAGC |
| | | TTCTGCCACTGGCTCCGGC |
| | | ATTGCAGAGAGAAGAGAAG |
| | | GGGCGGCAGAGCTGAACCT |
| | | TAGCCTTGCCTTCCTGGGT |
| | | ACCCTTCTGAGCCTCACTG |
| | | TCTTCTGTGAGATGGGCAA |
| | | AGTGCGGGTGTGACTCCTT |
| | | GGCAACGGTGTTACACCAG |
| | | GGCAGGTAAAGTTGTAGTT |
| | | ATTTGTGGGGTACACCAGG |
| | | ACTGTTAAAGGTGTAACTA |
| | | TCTGCAGCTCAGCCTACTA |
| | | CTTGCTTTCCAGGCTGTTC |
| | | CTAGTTCCCATGTCAGCTG |
| | | CTTGTGCTTTCCAGAGACA |
| | | AAACAGGAATAATAGATGT |
| | | CATTAAATATACATTGGGC |
| | | CCCAGGCGGTCAATGTGGC |
| | | AGCCTGAGCCTCCTTTCCA |
| | | TCTCTGTGGAGGCAGACAT |
| | | AGGACCCCCAACAAACAGC |
| | | ATGCAGGTTGGGAGCCAGC |
| | | CACAGGACCCAGGTAAGGG |
| | | GCCCTGGGTCCTTAAGCTT |
| | | CTGCCACTGGCTCCGGCAT |
| | | TGCAGAGAGAAGAGAAGGG |
| | | GCGGCAGACTGGAGAGCTG |
| | | GGCTCCATTTTTGTTCCTT |
| | | GGTGCCCTGCCCCTCCCCA |
| | | TGACCTGCAGAGACATTCA |
| | | GCCTGCCAGGCTTTATGAG |
| | | GTGGGAGCTGGGCTCTCCC |
| | | TGATGTATTATTCAGCTCC |
| | | CTGGAGTTGGCCAGCTCCT |
| | | GTTACACTGGCCACAGCCC |
| | | TGGGCATCCGCTTCTCACT |
| | | TCTAGTTTCCCCTCCAAGG |
| | | TAATGTGGTGGGTCATGAT |
| | | CATTCTATCCTGGCTTCAG |
| | | GGACCTGACTCCACTTTGG |
| | | GGCCATTCGAGGGGTCTAG |
| | | GGTAGATGATGTCCCCCTG |
| | | TGGGGATTAATGTCCTGCT |
| | | CTGTAAAACTGAGCTAGCT |
| | | GAGATCCAGGAGGGCTTGG |
| | | CCAGAGACAGCAAGTTGTT |
| | | GCCATGGTGACTTTAAAGC |
| | | CAGGTTGCTGCCCCAGCAC |
| | | AGGCCTCCCAGTCTACCCT |
| | | CACTAGAAAACAACACCCA |
| | | GGCACTTTCCACCACCTCT |
| | | CAAAGGTGAAACCCAAGGC |
| | | TGGTCTAGAGAATGAATTA |
| | | TGGATCCTCGCTGTCCGTG |
| | | CCACCCAGCTAGTCCCAGC |
| | | GGCTCAGACACTGAGGAGA |
| | | GACTGTAGGTTCAGCTACA |
| | | AGCAAAAAGACCTAGCTGG |
| | | TCTCCAAGCAGTGTCTCCA |
| | | AGTCCCTGAACCTGTGACA |
| | | CCTGCCCCAGGCATCATCA |
| | | GGCACAGAGGGCCACC |
| 39 | Portion of SEQ ID NO: | CTGCAGCTCAGCCTACTAC |
| | 24 that contains SEQ | TTGCTTTCCAGGCTGTTCC |
| | ID NO: 26 and SEQ ID | TAGTTCCCATGTCAGCTGC |
| | NO: 27 fused to portion | TTGTGCTTTCCAGAGACAA |
| | of SEQ ID NO: 25 that | AACAGGAATAATAGATGTC |
| | contains SEQ ID NO: | ATTAAATATACATTGGGCC |
| | 31 and SEQ ID NO: 32 | CCAGGCGGTCAATGTGGCA |
| | | GCCTGAGCCTCCTTTCCAT |
| | | CTCTGTGGAGGCAGACATA |
| | | GGACCCCCAACAAACAGCA |
| | | TGCAGGTTGGGAGCCAGCC |
| | | ACAGGACCCAGGTAAGGGG |
| | | CCCTGGGTCCTTTTTATGA |
| | | GGTGGGAGCTGGGCTCTCC |
| | | CTGATGTATTATTCAGCTC |
| | | CCTGGAGTTGGCCAGCTCC |
| | | TGTTACACTGGCCACAGCC |
| | | CTGGGCATCCGCTGCCATG |
| | | GTGACTTTAAAGCCAGGTT |
| | | GCTGCCCCAGCACAGGCCT |
| | | CCCAGTCTACCCTCACTAG |
| | | AAAACAACACCCAGGCACT |
| | | TTCCACCACCTCTCAAAGG |
| | | TGAAACCCAAGGCTGGTCT |
| | | AGAGAATGAATTATGGATC |
| | | CTCGCTGTCCGTGCCACCC |
| | | AGCTAGTCCCAGCGGCTCA |
| | | GACACTG |
| 40 | Region 1 of the human | GTATGCCTTTTGAGATGGA |
| | Myo15 promoter | TGCAGCAGGTTCTGTGAGG |
| | | CTGCCAGGAGGGGTAGAGT |
| | | TCCCGGGGGCCTCGGGCCC |
| | | CGCTGGAGTGTGGAGCAGG |
| | | CCCATGCTCAGCTCTCCAG |
| | | GCTGTTCGTGGCTCCCCTG |
| | | TCAGCTGCTCACTCCTTTC |
| | | CAGAGACAAAACAGGAATA |
| | | ATAGACATCATTAAATATA |
| | | CATAGGGCCCCAGGCGGTC |
| | | GGCGTGGTGGGCTGGGCCT |
| | | CCCTTCC |
| 41 | Region 2 of human | TGCCCTGCCTTCTGAGCCG |
| | Myo15 promoter | GCAGCCTGGCTCCCCACCC |
| | | CATGTATTATTCAGCTCCT |
| | | GAGAGCCAGCCAGCTCCTG |
| | | TTACACTGACCGCAGCCCA |
| | | GCACCTGCTCTGCCCATTC |
| | | CCCTCCTCCCTTGCCTAGG |
| | | ACCTAGAGGGTTCAAAGTT |
| | | CTCCTCCAAGATGACTTGG |
| | | TGGGCTTTGGCCATCCCAC |
| | | CCTAGGCCCCACTTCTGGC |
| | | CCAGTGCAGGTGTGCTGGT |
| | | GATTTAGGGCAGGTGGCAT |
| | | TCCATCTCTGTGGCTCAAT |
| | | GTCTTCCTCTGTGAAGCCG |
| | | AAGTGACCCAAGGGCTCCC |
| | | TTCATGGGGTTGAGCCAGC |
| | | TGTGGCCCAGGGAGGGCCT |
| | | AACCAGGATGAGCACTGAT |
| | | GTTGCCATGACGACTCCGA |
| | | GGCCAGAATGTCTCCCCCA |
| | | GCACAGGCCTCATAGGCAG |
| | | GCTTCCCCATCCTGGTAAA |
| | | CAACACCCACACACTTTCT |
| | | ACTACTGCTCTAGGGTGAA |
| | | ACCCAAGGCGCTCTAGAGG |
| | | AGATGAATTATGGATCCGC |
| | | CCTCCCGGAATCCTGGCTC |
| | | GGCCCTCCCCACGCCACCC |
| | | AGGGCCAGTCGGGTCTGCT |
| | | CACAGCCCGAGGAGGCCGC |
| | | GTGTCCAGCCGCGGGCAAG |
| | | AGACAGAGCAGGTCCCTGT |
| | | GTCTCCAAGTCCCTGAGCC |
| | | CGTGACACCGGCCCCAGGC |
| | | CCTGTAGAGAGCAGGCAGC |
| | | CACC |
| 42 | Portion of SEQ ID NO: | CCCCTGTCAGCTGCTCACT |
| | 40 | CCTTTCCAGAGACAAAACA |
| | | GGAATAATAGACATCATTA |
| | | AATATACATAGGGCCCCAG |
| | | G |
| 43 | Portion of SEQ ID NO: | TGAGCCGGCAGCCTGGCTC |
| | 41 | CCCACCCCATGTATTATTC |
| | | AGCTCCTGAGAGCCAGCCA |
| | | GCTCCTGTTACACTGACCG |
| | | CAGCCC |
| 44 | Portion of SEQ ID NO: | CACAGGCCTCATAGGCAGG |
| | 41 | CTTCCCCATCCTGGTAAAC |
| | | AACACCCACACACTTTCTA |
| | | CTACTGCTCTAGGGTGAAA |
| | | CCCAAGGCGCTCTAGAGGA |
| | | GATGAATTATGGATCC |
| 45 | Portion of SEQ ID NO: | TGAGCCGGCAGCCTGGCTC |
| | 41 | CCCACCCCATGTATTATTC |
| | (SEQ ID NO: 43 fused | AGCTCCTGAGAGCCAGCCA |
| | to SEQ ID NO: 44) | GCTCCTGTTACACTGACCG |
| | | CAGCCCCACAGGCCTCATA |
| | | GGCAGGCTTCCCCATCCTG |
| | | GTAAACAACACCCACACAC |
| | | TTTCTACTACTGCTCTAGG |
| | | GTGAAACCCAAGGCGCTCT |
| | | AGAGGAGATGAATTATGGA |
| | | TCC |
| 46 | Portion of SEQ ID NO: | CACAGGCCTCATAGGCAGG |
| | 41 | CTTCCCCATCCTGGTAAAC |
| | (SEQ ID NO: 44 fused | AACACCCACACACTTTCTA |
| | to SEQ ID NO: 43) | CTACTGCTCTAGGGTGAAA |
| | | CCCAAGGCGCTCTAGAGGA |
| | | GATGAATTATGGATCCTGA |
| | | GCCGGCAGCCTGGCTCCCC |
| | | ACCCCATGTATTATTCAGC |
| | | TCCTGAGAGCCAGCCAGCT |
| | | CCTGTTACACTGACCGCAG |
| | | CCC |
| 47 | Portion of SEQ ID NO: | TGAGCCGGCAGCCTGGCTC |
| | 41 (contiguous | CCCACCCCATGTATTATTC |
| | sequence including | AGCTCCTGAGAGCCAGCCA |
| | SEQ ID NO: 43 and | GCTCCTGTTACACTGACCG |
| | SEQ ID NO: 44) | CAGCCCAGCACCTGCTCTG |
| | | CCCATTCCCCTCCTCCCTT |
| | | GCCTAGGACCTAGAGGGTT |
| | | CAAAGTTCTCCTCCAAGAT |
| | | GACTTGGTGGGCTTTGGCC |
| | | ATCCCACCCTAGGCCCCAC |
| | | TTCTGGCCCAGTGCAGGTG |
| | | TGCTGGTGATTTAGGGCAG |
| | | GTGGCATTCCATCTCTGTG |
| | | GCTCAATGTCTTCCTCTGT |
| | | GAAGCCGAAGTGACCCAAG |
| | | GGCTCCCTTCATGGGGTTG |
| | | AGCCAGCTGTGGCCCAGGG |
| | | AGGGCCTAACCAGGATGAG |
| | | CACTGATGTTGCCATGACG |
| | | ACTCCGAGGCCAGAATGTC |
| | | TCCCCCAGCACAGGCCTCA |
| | | TAGGCAGGCTTCCCCATCC |
| | | TGGTAAACAACACCCACAC |
| | | ACTTTCTACTACTGCTCTA |
| | | GGGTGAAACCCAAGGCGCT |
| | | CTAGAGGAGATGAATTATG |
| | | GATCC |
| 48 | Polynucleotide | GTATGCCTTTTGAGATGGA |
| | containing SEQ ID NO: | TGCAGCAGGTTCTGTGAGG |
| | 40 and SEQ ID NO: 41 | CTGCCAGGAGGGGTAGAGT |
| | | TCCCGGGGGCCTCGGGCCC |
| | | CGCTGGAGTGTGGAGCAGG |
| | | CCCATGCTCAGCTCTCCAG |
| | | GCTGTTCGTGGCTCCCCTG |
| | | TCAGCTGCTCACTCCTTTC |
| | | CAGAGACAAAACAGGAATA |
| | | ATAGACATCATTAAATATA |
| | | CATAGGGCCCCAGGCGGTC |
| | | GGCGTGGTGGGCTGGGCCT |
| | | CCCTTCCCCATAACACTGA |
| | | GCTGCTCTGCTGGGCCAAC |
| | | CGTGCTCCTGGGCCAGCCA |
| | | GAGGACCCCCATGAGGCGG |
| | | CATGCAGGCGGGGAGCAGG |
| | | CCACAGAACGCAGGTAAGG |
| | | AGACCTTAGCCTAGAGTCC |
| | | TTGGGGTCTGTCACTGGCC |
| | | ACCCTCGCATCCCAGGCTG |
| | | CAGGAAACTGAGGCCCAGA |
| | | GAGGACAAGGACTTTCCTG |
| | | GACCCACACAGCCAGTCAG |
| | | TGACAGAGCCTAGGGTCTG |
| | | AGCCAGGCCTGACCCAACC |
| | | TCCATTTCTGCCTCTCTAC |
| | | CCCTGCCCCCGCCCCAACA |
| | | CACACACACACACAAGTGG |
| | | AGTTCCACTGAAACGCCCC |
| | | TCCTTGCCCTGCCTTCTGA |
| | | GCCGGCAGCCTGGCTCCCC |
| | | ACCCCATGTATTATTCAGC |
| | | TCCTGAGAGCCAGCCAGCT |
| | | CCTGTTACACTGACCGCAG |
| | | CCCAGCACCTGCTCTGCCC |
| | | ATTCCCCTCCTCCCTTGCC |
| | | TAGGACCTAGAGGGTTCAA |
| | | AGTTCTCCTCCAAGATGAC |
| | | TTGGTGGGCTTTGGCCATC |
| | | CCACCCTAGGCCCCACTTC |
| | | TGGCCCAGTGCAGGTGTGC |
| | | TGGTGATTTAGGGCAGGTG |
| | | GCATTCCATCTCTGTGGCT |
| | | CAATGTCTTCCTCTGTGAA |
| | | GCCGAAGTGACCCAAGGGC |
| | | TCCCTTCATGGGGTTGAGC |
| | | CAGCTGTGGCCCAGGGAGG |
| | | GCCTAACCAGGATGAGCAC |
| | | TGATGTTGCCATGACGACT |
| | | CCGAGGCCAGAATGTCTCC |
| | | CCCAGCACAGGCCTCATAG |
| | | GCAGGCTTCCCCATCCTGG |
| | | TAAACAACACCCACACACT |
| | | TTCTACTACTGCTCTAGGG |
| | | TGAAACCCAAGGCGCTCTA |
| | | GAGGAGATGAATTATGGAT |
| | | CCGCCCTCCCGGAATCCTG |
| | | GCTCGGCCCTCCCCACGCC |
| | | ACCCAGGGCCAGTCGGGTC |
| | | TGCTCACAGCCCGAGGAGG |
| | | CCGCGTGTCCAGCCGCGGG |
| | | CAAGAGACAGAGCAGGTCC |
| | | CTGTGTCTCCAAGTCCCTG |
| | | AGCCCGTGACACCGGCCCC |
| | | AGGCCCTGTAGAGAGCAGG |
| | | CAGCCACC |
| 49 | Polynucleotide | GCAGGCCCATGCTCAGCTC |
| | containing SEQ ID NO: | TCCAGGCTGTTCGTGGCTC |
| | 42, SEQ ID NO: 43, | CCCTGTCAGCTGCTCACTC |
| | and SEQ ID NO: 44 | CTTTCCAGAGACAAAACAG |
| | | GAATAATAGACATCATTAA |
| | | ATATACATAGGGCCCCAGG |
| | | CGGTCGGCGTGGTGGGCTG |
| | | GGCCTCCCTTCCCCATAAC |
| | | ACTGAGCTGCTCTGCTGGG |
| | | CCAACCGTGCTCCTGGGCC |
| | | AGCCAGAGGACCCCCATGA |
| | | GGCGGCATGCAGGCGGGGA |
| | | GCAGGCCACAGAACGCAGG |
| | | TAAGGAGACCTTGCCTTCT |
| | | GAGCCGGCAGCCTGGCTCC |
| | | CCACCCCATGTATTATTCA |
| | | GCTCCTGAGAGCCAGCCAG |
| | | CTCCTGTTACACTGACCGC |
| | | AGCCCAGCACCTGCTCTGC |
| | | CCATTCCCCTCCTCCCTTG |
| | | CCTAGGACCTAGAGGGTTC |
| | | AAAGTTCTCCTCCAAGATG |
| | | ACTTGGTGGGCTTTGGCCA |
| | | TCGGGCCTAACCAGGATGA |
| | | GCACTGATGTTGCCATGAC |
| | | GACTCCGAGGCCAGAATGT |
| | | CTCCCCCAGCACAGGCCTC |
| | | ATAGGCAGGCTTCCCCATC |
| | | CTGGTAAACAACACCCACA |
| | | CACTTTCTACTACTGCTCT |
| | | AGGGTGAAACCCAAGGCGC |
| | | TCTAGAGGAGATGAATTAT |
| | | GGATCCGCCCTCCCGGAAT |
| | | CCTGGCTCGGCCCTCCCCA |
| | | CGC |
| 50 | Portion of SEQ ID NO: | CTGCAGCTCAGCCTACTAC |
| | 24 that contains SEQ | TTGCTTTCCAGGCTGTTCC |
| | ID NO: 26 and SEQ ID | TAGTTCCCATGTCAGCTGC |
| | NO: 27 | TTGTGCTTTCCAGAGACAA |
| | | AACAGGAATAATAGATGTC |
| | | ATTAAATATACATTGGGCC |
| | | CCAGGCGGTCAATGTGGCA |
| | | GCCTGAGCCTCCTTTCCAT |
| | | CTCTGTGGAGGCAGACATA |
| | | GGACCCCCAACAAACAGCA |
| | | TGCAGGTTGGGAGCCAGCC |
| | | ACAGGACCCAGGTAAGGGG |
| | | CCCTGGGTCCTT |
| 51 | Portion of SEQ ID NO: | TTTATGAGGTGGGAGCTGG |
| | 25 that contains SEQ | GCTCTCCCTGATGTATTAT |
| | ID NO: 31 | TCAGCTCCCTGGAGTTGGC |
| | | CAGCTCCTGTTACACTGGC |
| | | CACAGCCCTGGGCATCCGC |
| 52 | Portion of SEQ ID NO: | TGCCATGGTGACTTTAAAG |
| | 25 that contains SEQ | CCAGGTTGCTGCCCCAGCA |
| | ID NO: 32 | CAGGCCTCCCAGTCTACCC |
| | | TCACTAGAAAACAACACCC |
| | | AGGCACTTTCCACCACCTC |
| | | TCAAAGGTGAAACCCAAGG |
| | | CTGGTCTAGAGAATGAATT |
| | | ATGGATCCTCGCTGTCCGT |
| | | GCCACCCAGCTAGTCCCAG |
| | | CGGCTCAGACACTG |
| 53 | SEQ ID NO: 50 fused | CTGCAGCTCAGCCTACTAC |
| | to SEQ ID NO: 51 | TTGCTTTCCAGGCTGTTCC |
| | | TAGTTCCCATGTCAGCTGC |
| | | TTGTGCTTTCCAGAGACAA |
| | | AACAGGAATAATAGATGTC |
| | | ATTAAATATACATTGGGCC |
| | | CCAGGCGGTCAATGTGGCA |
| | | GCCTGAGCCTCCTTTCCAT |
| | | CTCTGTGGAGGCAGACATA |
| | | GGACCCCCAACAAACAGCA |
| | | TGCAGGTTGGGAGCCAGCC |
| | | ACAGGACCCAGGTAAGGGG |
| | | CCCTGGGTCCTTTTTATGA |
| | | GGTGGGAGCTGGGCTCTCC |
| | | CTGATGTATTATTCAGCTC |
| | | CCTGGAGTTGGCCAGCTCC |
| | | TGTTACACTGGCCACAGCC |
| | | CTGGGCATCCGC |
| 54 | SEQ ID NO: 50 fused | CTGCAGCTCAGCCTACTAC |
| | to SEQ ID NO: 52 | TTGCTTTCCAGGCTGTTCC |
| | | TAGTTCCCATGTCAGCTGC |
| | | TTGTGCTTTCCAGAGACAA |
| | | AACAGGAATAATAGATGTC |
| | | ATTAAATATACATTGGGCC |
| | | CCAGGCGGTCAATGTGGCA |
| | | GCCTGAGCCTCCTTTCCAT |
| | | CTCTGTGGAGGCAGACATA |
| | | GGACCCCCAACAAACAGCA |
| | | TGCAGGTTGGGAGCCAGCC |
| | | ACAGGACCCAGGTAAGGGG |
| | | CCCTGGGTCCTTTGCCATG |
| | | GTGACTTTAAAGCCAGGTT |
| | | GCTGCCCCAGCACAGGCCT |
| | | CCCAGTCTACCCTCACTAG |
| | | AAAACAACACCCAGGCACT |
| | | TTCCACCACCTCTCAAAGG |
| | | TGAAACCCAAGGCTGGTCT |
| | | AGAGAATGAATTATGGATC |
| | | CTCGCTGTCCGTGCCACCC |
| | | AGCTAGTCCCAGCGGCTCA |
| | | GACACTG |
| 55 | SEQ ID NO: 51 fused | TTTATGAGGTGGGAGCTGG |
| | to SEQ ID NO: 52 | GCTCTCCCTGATGTATTAT |
| | | TCAGCTCCCTGGAGTTGGC |
| | | CAGCTCCTGTTACACTGGC |
| | | CACAGCCCTGGGCATCCGC |
| | | TGCCATGGTGACTTTAAAG |
| | | CCAGGTTGCTGCCCCAGCA |
| | | CAGGCCTCCCAGTCTACCC |
| | | TCACTAGAAAACAACACCC |
| | | AGGCACTTTCCACCACCTC |
| | | TCAAAGGTGAAACCCAAGG |
| | | CTGGTCTAGAGAATGAATT |
| | | ATGGATCCTCGCTGTCCGT |
| | | GCCACCCAGCTAGTCCCAG |
| | | CGGCTCAGACACTG |
| 56 | SEQ ID NO: 51 fused | TTTATGAGGTGGGAGCTGG |
| | to SEQ ID NO: 50, | GCTCTCCCTGATGTATTAT |
| | which is fused to SEQ | TCAGCTCCCTGGAGTTGGC |
| | ID NO: 52 | CAGCTCCTGTTACACTGGC |
| | | CACAGCCCTGGGCATCCGC |
| | | CTGCAGCTCAGCCTACTAC |
| | | TTGCTTTCCAGGCTGTTCC |
| | | TAGTTCCCATGTCAGCTGC |
| | | TTGTGCTTTCCAGAGACAA |
| | | AACAGGAATAATAGATGTC |
| | | ATTAAATATACATTGGGCC |
| | | CCAGGCGGTCAATGTGGCA |
| | | GCCTGAGCCTCCTTTCCAT |
| | | CTCTGTGGAGGCAGACATA |
| | | GGACCCCCAACAAACAGCA |
| | | TGCAGGTTGGGAGCCAGCC |
| | | ACAGGACCCAGGTAAGGGG |
| | | CCCTGGGTCCTTTGCCATG |
| | | GTGACTTTAAAGCCAGGTT |
| | | GCTGCCCCAGCACAGGCCT |
| | | CCCAGTCTACCCTCACTAG |
| | | AAAACAACACCCAGGCACT |
| | | TTCCACCACCTCTCAAAGG |
| | | TGAAACCCAAGGCTGGTCT |
| | | AGAGAATGAATTATGGATC |
| | | CTCGCTGTCCGTGCCACCC |
| | | AGCTAGTCCCAGCGGCTCA |
| | | GACACTG |
| 57 | SEQ ID NO: 52 fused | TGCCATGGTGACTTTAAAG |
| | to SEQ ID NO: 50, | CCAGGTTGCTGCCCCAGCA |
| | which is fused to SEQ | CAGGCCTCCCAGTCTACCC |
| | ID NO: 51 | TCACTAGAAAACAACACCC |
| | | AGGCACTTTCCACCACCTC |
| | | TCAAAGGTGAAACCCAAGG |
| | | CTGGTCTAGAGAATGAATT |
| | | ATGGATCCTCGCTGTCCGT |
| | | GCCACCCAGCTAGTCCCAG |
| | | CGGCTCAGACACTGCTGCA |
| | | GCTCAGCCTACTACTTGCT |
| | | TTCCAGGCTGTTCCTAGTT |
| | | CCCATGTCAGCTGCTTGTG |
| | | CTTTCCAGAGACAAAACAG |
| | | GAATAATAGATGTCATTAA |
| | | ATATACATTGGGCCCCAGG |
| | | CGGTCAATGTGGCAGCCTG |
| | | AGCCTCCTTTCCATCTCTG |
| | | TGGAGGCAGACATAGGACC |
| | | CCCAACAAACAGCATGCAG |
| | | GTTGGGAGCCAGCCACAGG |
| | | ACCCAGGTAAGGGGCCCTG |
| | | GGTCCTTTTTATGAGGTGG |
| | | GAGCTGGGCTCTCCCTGAT |
| | | GTATTATTCAGCTCCCTGG |
| | | AGTTGGCCAGCTCCTGTTA |
| | | CACTGGCCACAGCCCTGGG |
| | | CATCCGC |
| 58 | SEQ ID NO: 51 fused | TTTATGAGGTGGGAGCTGG |
| | to SEQ ID NO: 52, | GCTCTCCCTGATGTATTAT |
| | which is fused to SEQ | TCAGCTCCCTGGAGTTGGC |
| | ID NO: 50 | CAGCTCCTGTTACACTGGC |
| | | CACAGCCCTGGGCATCCGC |
| | | TGCCATGGTGACTTTAAAG |
| | | CCAGGTTGCTGCCCCAGCA |
| | | CAGGCCTOCCAGTCTACCC |
| | | TCACTAGAAAACAACACCC |
| | | AGGCACTTTCCACCACCTC |
| | | TCAAAGGTGAAACCCAAGG |
| | | CTGGTCTAGAGAATGAATT |
| | | ATGGATCCTCGCTGTCCGT |
| | | GCCACCCAGCTAGTCCCAG |
| | | CGGCTCAGACACTGCTGCA |
| | | GCTCAGCCTACTACTTGCT |
| | | TTCCAGGCTGTTCCTAGTT |
| | | CCCATGTCAGCTGCTTGTG |
| | | CTTTCCAGAGACAAAACAG |
| | | GAATAATAGATGTCATTAA |
| | | ATATACATTGGGCCCCAGG |
| | | CGGTCAATGTGGCAGCCTG |
| | | AGCCTCCTTTCCATCTCTG |
| | | TGGAGGCAGACATAGGACC |
| | | CCCAACAAACAGCATGCAG |
| | | GTTGGGAGCCAGCCACAGG |
| | | ACCCAGGTAAGGGGCCCTG |
| | | GGTCCTT |
| 59 | Portion of SEQ ID NO: | TGCAGCTCAGCCTACTACT |
| | 24 that contains SEQ | TGCTTTCCAGGCTGTTCCT |
| | ID NO: 26 and SEQ ID | AGTTCCCATGTCAGCTGCT |
| | NO: 27 fused to portion | TGTGCTTTCCAGAGACAAA |
| | of SEQ ID NO: 25 that | ACAGGAATAATAGATGTCA |
| | contains SEQ ID NO: | TTAAATATACATTGGGCCC |
| | 31 and SEQ ID NO: 32 | CAGGCGGTCAATGTGGCAG |
| | | CCTGAGCCTCCTTTCCATC |
| | | TCTGTGGAGGCAGACATAG |
| | | GACCCCCAACAAACAGCAT |
| | | GCAGGTTGGGAGCCAGCCA |
| | | CAGGACCCAGGTAAGGGGC |
| | | CCTGGGTCCTTAAGCTTCT |
| | | GCCACTGGCTCCGGCATTG |
| | | CAGAGAGAAGAGAAGGGGC |
| | | GGCAGACTGGAGAGCTGGG |
| | | CTCCATTTTTGTTCCTTGG |
| | | TGCCCTGCCCCTCCCCATG |
| | | ACCTGCAGAGACATTCAGC |
| | | CTGCCAGGCTTTATGAGGT |
| | | GGGAGCTGGGCTCTCCCTG |
| | | ATGTATTATTCAGCTCCCT |
| | | GGAGTTGGCCAGCTCCTGT |
| | | TACACTGGCCACAGCCCTG |
| | | GGCATCCGCTTCTCACTTC |
| | | TAGTTTCCCCTCCAAGGTA |
| | | ATGTGGTGGGTCATGATCA |
| | | TTCTATCCTGGCTTCAGGG |
| | | ACCTGACTCCACTTTGGGG |
| | | CCATTCGAGGGGTCTAGGG |
| | | TAGATGATGTCCCCCTGTG |
| | | GGGATTAATGTCCTGCTCT |
| | | GTAAAACTGAGCTAGCTGA |
| | | GATCCAGGAGGGCTTGGCC |
| | | AGAGACAGCAAGTTGTTGC |
| | | CATGGTGACTTTAAAGCCA |
| | | GGTTGCTGCCCCAGCACAG |
| | | GCCTCCCAGTCTACCCTCA |
| | | CTAGAAAACAACACCCAGG |
| | | CACTTTCCACCACCTCTCA |
| | | AAGGTGAAACCCAAGGCTG |
| | | GTCTAGAGAATGAATTATG |
| | | GATCCTCGCTGTCCGTGCC |
| | | ACCCAGCTAGTCCCAGCGG |
| | | CTCAGACACTGAGGAGAGA |
| | | CTGTAGGTTCAGCTACAAG |
| | | CAAAAAGACCTAGCTGGTC |
| | | TCCAAGCAGTGTCTCCAAG |
| | | TCCCTGAACCTGTGACACC |
| | | TGCCCCAGGCATCATCAGG |
| | | CACAGAGGGCCACC |
| 60 | Portion of SEQ ID NO: | TGCAGCTCAGCCTACTACT |
| | 24 that contains SEQ | TGCTTTCCAGGCTGTTCCT |
| | ID NO: 26 and SEQ ID | AGTTCCCATGTCAGCTGCT |
| | NO: 27 fused to portion | TGTGCTTTCCAGAGACAAA |
| | of SEQ ID NO: 25 that | ACAGGAATAATAGATGTCA |
| | contains SEQ ID NO: | TTAAATATACATTGGGCCC |
| | 31 and SEQ ID NO: 32 | CAGGCGGTCAATGTGGCAG |
| | | CCTGAGCCTCCTTTCCATC |
| | | TCTGTGGAGGCAGACATAG |
| | | GACCCCCAACAAACAGCAT |
| | | GCAGGTTGGGAGCCAGCCA |
| | | CAGGACCCAGGTAAGGGGC |
| | | CCTGGGTCCTTTTTATGAG |
| | | GTGGGAGCTGGGCTCTCCC |
| | | TGATGTATTATTCAGCTCC |
| | | CTGGAGTTGGCCAGCTCCT |
| | | GTTACACTGGCCACAGCCC |
| | | TGGGCATCCGCTGCCATGG |
| | | TGACTTTAAAGCCAGGTTG |
| | | CTGCCCCAGCACAGGCCTC |
| | | CCAGTCTACCCTCACTAGA |
| | | AAACAACACCCAGGCACTT |
| | | TCCACCACCTCTCAAAGGT |
| | | GAAACCCAAGGCTGGTCTA |
| | | GAGAATGAATTATGGATCC |
| | | TCGCTGTCCGTGCCACCCA |
| | | GCTAGTCCCAGCGGCTCAG |
| | | ACACTG |

Additional Myo15 promoters useful in conjunction with the compositions and methods described herein include nucleic acid molecules that have at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequences set forth in Table 3, as well as functional portions or derivatives of the nucleic acid sequences set forth in Table 3. The Myo15 promoters listed in Table 3 are characterized in International Application Publication Nos. WO2019210181A1 and WO2020163761A1, which are incorporated herein by reference.

In embodiments in which an smCBA promoter is included in a dual vector system described herein (e.g., in the first vector in a dual vector system), the smCBA promoter may have the sequence of the smCBA promoter described in U.S. Pat. No. 8,298,818, which is incorporated herein by reference. In some embodiments, the smCBA promoter has the sequence of:

(SEQ ID NO: 70)
GGTACCTAGTTATTAATAGTAATCAATTACGGGGTCATTAGTTCAT
AGCCCATATATGGAGTTCCGCGTTACATAACTTACGGTAAATGGC
CCGCCTGGCTGACCGCCCAACGACCCCCGCCCATTGACGTCAATA
ATGACGTATGTTCCCATAGTAACGCCAATAGGGACTTTCCATTGA
CGTCAATGGGTGGAGTATTTACGGTAAACTGCCCACTTGGCAGTA
CATCAAGTGTATCATATGCCAAGTACGCCCCCTATTGACGTCAAT
GACGGTAAATGGCCCGCCTGGCATTATGCCCAGTACATGACCTTA
TGGGACTTTCCTACTTGGCAGTACATCTACGTATTAGTCATCGCT
ATTACCATGGTCGAGGTGAGCCCCACGTTCTGCTTCACTCTCCCC
ATCTCCCCCCCCTCCCCACCCCCAATTTTGTATTTATTTATTTTT
TAATTATTTTGTGCAGCGATGGGGGGGGGGGGGGGGGGGGGGGGC
GCGCCAGGGGGGCGGGGGGGGGCGAGGGGGGGGGGGGGCGAGGCG
GAGAGGTGCGGCGGCAGCCAATCAGAGCGGCGCGCTCCGAAAGTT
TCCTTTTATGGCGAGGGGGGGGCGGCGGCGGCCCTATAAAAAGCG
AAGCGCGCGGGGGGCGGGAGTCGCTGCGCGCTGCCTTCGCCCCGT
GCCCCGCTCCGCCGCCGCCTCGCGCCGCCCGCCCCGGCTCTGACT
GACCGCGTTACTCCCACAGGTGAGCGGGCGGGACGGCCCTTCTCC
TCCGGGCTGTAATTAGCGCTTGGTTTAATGACGGCTTGTTTCTTT
TCTGTGGCTGCGTGAAAGCCTTGAGGGGCTCCGGGAGCTAGAGCC
TCTGCTAACCATGTTCATGCCTTCTTCTTTTTCCTACAGCTCCTG
GGCAACGTGCTGGTTATTGTGCTGTCTCATCATTTTGGCA.

In some embodiments, the smCBA promoter has the sequence of:

(SEQ ID NO: 84)
AATTCGGTACCCTAGTTATTAATAGTAATCAATTACGGGGTCATTAGTTC
ATAGCCCATATATGGAGTTCCGCGTTACATAACTTACGGTAAATGGCCCG
CCTGGCTGACCGCCCAACGACCCCCGCCCATTGACGTCAATAATGACGTA
TGTTCCCATAGTAACGCCAATAGGGACTTTCCATTGACGTCAATGGGTGG
ACTATTTACGGTAAACTGCCCACTTGGCAGTACATCAAGTGTATCATATG
CCAAGTACGCCCCCTATTGACGTCAATGACGGTAAATGGCCCGCCTGGCA
TTATGCCCAGTACATGACCTTATGGGACTTTCCTACTTGGCAGTACATCT
ACGTATTAGTCATCGCTATTACCATGGTCGAGGTGAGCCCCACGTTCTGC
TTCACTCTCCCCATCTCCCCCCCCTCCCCACCCCCAATTTTGTATTTATT
TATTTTTTAATTATTTTGTGCAGCGATGGGGGGGGGGGGGGGGGGGGGGG
GCGCGCCAGGCGGGGGGGGGGGGGGCGAGGGGGGGGGGGGGCGAGGCGGA
GAGGTGCGGCGGCAGCCAATCAGAGCGGCGCGCTCCGAAAGTTTCCTTTT
ATGGCGAGGGGGGGGCGGCGGCGGCCCTATAAAAAGCGAAGCGGGGGGGG
GGGGGAGTCGCTGCGACGCTGCCTTCGCCCCGTGCCCCGCTCCGCCGCCG
CCTCGCGCCGCCCGCCCCGGCTCTGACTGACCGCGTTACTCCCACAGGTG
AGCGGGGGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCC
GGGAGCTAGAGCCTCTGCTAACCATGTTCATGCCTTCTTCTTTTTCCTAC
AGCTCCTGGGCAACGTGCTGGTTATTGTGCTGTCTCATCATTTTGGCAAA
G.

Once a polynucleotide encoding OTOF has been incorporated into the nuclear DNA of a mammalian cell or stabilized in an episomal monomer or concatemer, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in the nucleic acid vectors for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode an OTOF protein and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv, et al., Nature 297:17 (1982). An enhancer may be spliced into a vector containing a polynucleotide encoding an OTOF protein, for example, at a position 5′ or 3′ to this gene. In a preferred orientation, the enhancer is positioned at the 5′ side of the promoter, which in turn is located 5′ relative to the polynucleotide encoding an OTOF protein.

The nucleic acid vectors described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo. The WPRE can be located in the second nucleic acid vector between the polynucleotide encoding a C-terminal portion of an OTOF protein and the poly(A) sequence. In some embodiments of the compositions and methods described herein, the WPRE has the sequence:

(SEQ ID NO: 23)
GATCCAATCAACCTCTGGATTACAAAATTTGTGAAAGATTGACTGGTATT
CTTAACTATGTTGCTCCTTTTACGCTATGTGGATACGCTGCTTTAATGCC
TTTGTATCATGCTATTGCTTCCCGTATGGCTTTCATTTTCTCCTCCTTGT
ATAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTTG
GGGCATTGCCACCACCTGTCAGCTCCTTTCCGGGACTTTCGCTTTCCCCC
TCCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGAA
ATCATCGTCCTTTCCTTGGCTGCTCGCCTGTGTTGCCACCTGGATTCTGC
GCGGGACGTCCTTCTGCTACGTCCCTTCGGCCCTCAATCCAGCGGACCTT
CCTTCCCGCGGCCTGCTGCCGGCTCTGCGGCCTCTTCCGCGTCTTCGA.

In other embodiments, the WPRE has the sequence:

(SEQ ID NO: 61)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTAGTTCTTGCCACGGCGGAACTCATCGCCGCCTGCCTTGCCCG
CTGCTGGACAGGGGCTCGGCTGTTGGGCACTGACAATTCCGTGGTGTTTA
TTTGTGAAATTTGTGATGCTATTGCTTTATTTGTAACCATCTAGCTTTAT
TTGTGAAATTTGTGATGCTATTGCTTTATTTGTAACCATTATAAGCTGCA
ATAAACAAGTTAACAACAACAATTGCATTCATTTTATGTTTCAGGTTCAG
GGGGAGATGTGGGAGGTTTTTTAAA.

In some embodiments, the nucleic acid vectors for use in the compositions and methods described herein include a reporter sequence, which can be useful in verifying OTOF gene expression, for example, in specific cells and tissues (e.g., in cochlear hair cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Overlapping Dual Vectors

One approach for expressing large proteins in mammalian cells involves the use of overlapping dual vectors. This approach is based on the use of two nucleic acid vectors, each of which contains a portion of a polynucleotide that encodes a protein of interest and has a defined region of sequence overlap with the other polynucleotide. Homologous recombination can occur at the region of overlap and lead to the formation of a single nucleic acid molecule that encodes the full-length protein of interest.

Overlapping dual vectors for use in the methods and compositions described herein contain at least one kilobase (kb) of overlapping sequence (e.g., 1 kb, 1.5 kb, 2 kb, 2.5 kb, 3 kb or more of overlapping sequence). The nucleic acid vectors are designed such that the overlapping region is centered at an OTOF exon boundary, with an equal amount of overlap on either side of the boundary. The boundaries are chosen based on the size of the promoter and the locations of the portions of the polynucleotide that encode OTOF C2 domains. Overlapping regions are centered on exon boundaries that occur outside of the portion of the polynucleotide that encodes the C2C domain (e.g., after the portion of the polynucleotide that encodes the C2C domain). Exon boundaries within the portion of the polynucleotide that encodes the C2D domain can be selected as the center of the overlapping region, or exon boundaries located after the portion of the polynucleotide that encodes the C2D domain and before the portion of the polynucleotide that encodes the C2E domain can serve as the center of an overlapping region. The nucleic acid vectors for use in the methods and compositions described herein are also designed such that approximately half of the OTOF gene is contained within each vector (e.g., each vector contains a polynucleotide that encodes approximately half of the OTOF protein).

One exemplary overlapping dual vector system includes a first nucleic acid vector containing a CAG promoter operably linked to exons 1-28 and the 500 base pairs (bp) immediately 3′ of the exon 28/29 boundary of a polynucleotide encoding an OTOF protein (e.g., human OTOF, e.g., SEQ ID NO: 1 or SEQ ID NO: 5, or mouse OTOF, e.g., SEQ ID NO: 6); and a second nucleic acid vector containing the 500 bp immediately 5′ of the exon 28/29 boundary and the remaining exons (e.g., exons 29-48 for mouse OTOF, exons 29-45 and 47 or exons 29-46 for human OTOF) of the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bovine growth hormone (bGH) poly(A) signal sequence). In this overlapping dual vector system, the overlapping sequence is centered at the exon 28/29 boundary, which is after the portion of the polynucleotide that encodes the C2D domain. Another exemplary overlapping dual vector system includes a first nucleic acid vector containing a CAG promoter operably linked to exons 1-24 and the 500 bp immediately 3′ of the exon 24/25 boundary of a polynucleotide encoding an OTOF protein (e.g., human OTOF, e.g., SEQ ID NO: 1 or SEQ ID NO: 5, or mouse OTOF, e.g., SEQ ID NO: 6); and a second nucleic acid vector containing the 500 bp immediately 5′ of the exon 24/25 boundary and the remaining exons (e.g., exons 25-48 for mouse OTOF, exons 25-45 and 47 or exons 25-46 for human OTOF) the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence). In this overlapping dual vector system, the overlapping sequence is centered at the exon 24/25 boundary, which is within the portion of the polynucleotide that encodes the C2D domain. The two exon boundaries described above can be used with any promoter that is a similar size to the CAG promoter (e.g., the CMV promoter or smCBA promoter), such as promoters that are 1 kb or shorter (e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter). For example, in either of the foregoing dual vector systems, the CMV promoter or the smCBA promoter, can be used in the place of the CAG promoter. A Myo15 promoter having a sequence that is 1 kb or shorter (e.g., a Myo15 promoter described hereinabove, e.g., a Myo15 promoter having the sequence of any one of SEQ ID NOs: 38, 39, or 49-60) can also be used in place of the CAG promoter. Alternatively, a different exon boundary can be chosen that is within or after the portion of the polynucleotide that encodes the C2D domain and before the portion of the polynucleotide that encodes the C2E domain. The nucleic acid vectors containing promoters of this size can optionally contain OTOF UTRs. For example, in the foregoing overlapping dual vector system in which the overlapping region is centered at the exon 28/29 boundary of OTOF, the second nucleic acid vector can contain the full length OTOF 3′ UTR (e.g., the 1035 bp human OTOF 3′ UTR in dual vector systems encoding human OTOF, or the 1001 bp mouse OTOF 3′ UTR in dual vector systems encoding mouse OTOF). In the foregoing overlapping dual vector system in which the overlapping region is centered at the exon 24/25 boundary of OTOF, neither the first nor the second nucleic acid vector contains an OTOF UTR.

In some embodiments, the first nucleic acid vector in the overlapping dual vector system contains a long promoter (e.g., a promoter that is longer than 1 kb, e.g., 1.1 kb, 1.25 kb, 1.5 kb, 1.75 kb, 2 kb, 2.5 kb, 3 kb or longer). In such overlapping dual vector systems, the overlapping region can be centered at an exon boundary that is located after the portion of the polynucleotide that encodes the C2C domain and before the portion of the polynucleotide that encodes the C2D domain. For example, an overlapping dual vector system for use in the methods and compositions described herein includes a first nucleic acid vector containing a Myo15 promoter that is longer than 1 kb (e.g., SEQ ID NO: 36) operably linked exons 1-21 and the 500 bp immediately 3′ of the exon 21/22 boundary of a polynucleotide encoding an OTOF protein (e.g., human OTOF, e.g., SEQ ID NO: 1 or SEQ ID NO: 5, or mouse OTOF, e.g., SEQ ID NO: 6); and a second nucleic acid vector containing the 500 bp immediately 5′ of the exon 21/22 boundary and the remaining exons (e.g., exons 29-48 for mouse OTOF, exons 22-45 and 47 or exons 22-46 for human OTOF) of the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence). The exon 20/21 boundary can also be selected as the center of the overlapping region. In such overlapping dual vector systems, neither the first nor the second nucleic acid vector may include an OTOF UTR. A short promoter (e.g., a CMV promoter, CAG promoter, smCBA promoter, or a Myo15 promoter having a sequence that is 1 kb or shorter, e.g., a Myo15 promoter having the sequence of any one of SEQ ID NOs: 38, 39, or 49-60) can also be used in these dual vector systems (e.g., a dual vector system in which the overlapping region is centered at the exon 21/22 or exon 20/21 boundary). If a short promoter is used, additional elements, such as a 5′ OTOF UTR, can be included in the first vector (e.g., the vector containing exons 1-21 and the 500 bp immediately 3′ of the exon 21/22 boundary or exons 1-20 and the 500 bp immediately 3′ of the exon 20/21 boundary of a polynucleotide encoding an OTOF protein).

### Trans-Splicing Dual Vectors

A second approach for expressing large proteins in mammalian cells involves the use of trans-splicing dual vectors. In this approach, two nucleic acid vectors are used that contain distinct nucleic acid sequences, and the polynucleotide encoding the N-terminal portion of the protein of interest and the polynucleotide encoding the C-terminal portion of the protein of interest do not overlap. Instead, the first nucleic acid vector includes a splice donor sequence 3′ of the polynucleotide encoding the N-terminal portion of the protein of interest, and the second nucleic acid vector includes a splice acceptor sequence 5′ of the polynucleotide encoding the C-terminal portion of the protein of interest. When the first and second nucleic acids are present in the same cell, their ITRs can concatemerize, forming a single nucleic acid structure in which the concatemerized ITRs are positioned between the splice donor and splice acceptor. Trans-splicing then occurs during transcription, producing a nucleic acid molecule in which the polynucleotides encoding the N-terminal and C-terminal portions of the protein of interest are contiguous, thereby forming the full-length coding sequence.

Trans-splicing dual vectors for use in the methods and compositions described herein are designed such that approximately half of the OTOF gene is contained within each vector (e.g., each vector contains a polynucleotide that encodes approximately half of the OTOF protein). The determination of how to split the polynucleotide sequence between the two nucleic acid vectors is made based on the size of the promoter and the locations of the portions of the polynucleotide that encode the OTOF C2 domains. When a short promoter is used in the trans-splicing dual vector system (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter), such as a CAG promoter, a CMV promoter, a smCBA promoter, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60) the OTOF polynucleotide sequence can be divided between the two nucleic acid vectors at an exon boundary that occurs after the portion of the polynucleotide that encodes the C2D domain and before the portion of the polynucleotide that encodes the C2E domain, for example, the exon 26/27 boundary. The nucleic acid vectors containing promoters of this size can optionally contain OTOF UTRs (e.g., both the 5′ and 3′ OTOF UTRs, e.g., full-length UTRs). When a long promoter is used in the trans-splicing dual vector system (e.g., a promoter that is longer than 1 kb, e.g., 1.1 kb, 1.25 kb, 1.5 kb, 1.75 kb, 2 kb, 2.5 kb, 3 kb or longer), such as a Myo15 promoter that is longer than 1 kb (e.g., SEQ ID NO: 36), the OTOF polynucleotide sequence can be divided between the two nucleic acid vectors at an exon boundary that occurs after the portion of the polynucleotide that encodes the C2C domain, and either before the portion of the polynucleotide that encodes the C2D domain, such as the exon 19/20 boundary, the exon 20/21 boundary, or the exon 21/22 boundary, or within the portion of the polynucleotide that encodes the C2D domain, such as the exon 25/26 boundary. A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the dual vector systems designed for large promoters, in which case additional elements (e.g., OTOF UTR sequences) may be included in the first vector (e.g., the vector containing the portion of the polynucleotide the encodes the C2C domain).

One exemplary trans-splicing dual vector system that uses a short promoter includes a first nucleic acid vector containing a CAG promoter operably linked to exons 1-26 of a polynucleotide encoding an OTOF protein (e.g., human OTOF, e.g., SEQ ID NO: 1 or SEQ ID NO: 5, or mouse OTOF, e.g., SEQ ID NO: 6) and a splice donor sequence 3′ of the polynucleotide sequence; and a second nucleic acid vector containing a splice acceptor sequence 5′ of the remaining exons (e.g., exons 27-48 for mouse OTOF, or exons 27-45 and 47 or exons 27-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence). An alternative trans-splicing dual vector system includes a first nucleic acid vector containing a CAG promoter operably linked to exons 1-28 of a polynucleotide encoding an OTOF protein (e.g., human OTOF, e.g., SEQ ID NO: 1 or SEQ ID NO: 5, or mouse OTOF, e.g., SEQ ID NO: 6) and a splice donor sequence 3′ of the polynucleotide sequence; and a second nucleic acid vector containing a splice acceptor sequence 5′ of the remaining exons (e.g., exons 29-48 of mouse OTOF, or exons 29-45 and 47 or exons 29-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence). The CMV promoter, smCBA promoter, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60) can be used in place of the CAG promoter either of the foregoing dual vector systems. These nucleic acid vectors can also contain full length 5′ and 3′ OTOF UTRs in the first and second nucleic acid vectors, respectively (e.g., the first nucleic acid vector can contain the 5′ human OTOF UTR (127 bp) in dual vector systems encoding human OTOF, or the 5′ mouse UTR (134 bp) in dual vector systems encoding mouse OTOF; and the second nucleic acid vector can contain the 3′ human OTOF UTR (1035 bp) in dual vector systems encoding human OTOF, or the 3′ mouse OTOF UTR (1001 bp) in dual vector systems encoding mouse OTOF).

An exemplary trans-splicing dual vector system that uses a long promoter includes a first nucleic acid vector containing a Myo15 promoter that is longer than 1 kb (e.g., SEQ ID NO: 36) operably linked to exons 1-19 of a polynucleotide encoding an OTOF protein (e.g., human OTOF, e.g., SEQ ID NO: 1 or SEQ ID NO: 5, or mouse OTOF, e.g., SEQ ID NO: 6) and a splice donor sequence 3′ of the polynucleotide sequence; and a second nucleic acid vector containing a splice acceptor sequence 5′ of the remaining exons (e.g., exons 20-48 of mouse OTOF, or exons 20-45 and 47 or exons 20-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence). Alternatively, the trans-splicing dual vector system can include a first nucleic acid vector containing a Myo15 promoter that is longer than 1 kb (e.g., SEQ ID NO: 36) operably linked to exons 1-20 of a polynucleotide encoding an OTOF protein (e.g., human OTOF, e.g., SEQ ID NO: 1 or SEQ ID NO: 5, or mouse OTOF, e.g., SEQ ID NO: 6) and a splice donor sequence 3′ of the polynucleotide sequence; and a second nucleic acid vector containing a splice acceptor sequence 5′ of the remaining exons (e.g., exons 21-48 of mouse OTOF, or exons 21-45 and 47 or exons 21-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence). Neither the first nor the second nucleic acid vector in either of the foregoing Myo15 promoter trans-splicing dual vector systems contains an OTOF UTR. A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include a 5′ OTOF UTR or another element of a similar size in the first vector.

To accommodate an OTOF UTR, the OTOF coding sequence can be divided in a different position. For example, in a trans-splicing dual vector system in which the first nucleic acid vector contains a Myo15 promoter that is longer than 1 kb (e.g., SEQ ID NO: 36) operably linked to exons 1-25 of a polynucleotide encoding an OTOF protein (e.g., human OTOF, e.g., SEQ ID NO: 1 or SEQ ID NO: 5, or mouse OTOF, e.g., SEQ ID NO: 6) and a splice donor sequence 3′ of the polynucleotide sequence; and the second nucleic acid vector contains a splice acceptor sequence 5′ of the remaining exons (e.g., exons 26-48 of mouse OTOF, or exons 26-45 and 47 or 26-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence), the second nucleic acid can also contain a full length OTOF 3′ UTR (e.g., the 1035 bp human OTOF 3′ UTR). For mouse OTOF, the trans-splicing dual vector system can also contain a 3′ UTR if the first nucleic acid vector contains a Myo15 promoter that is longer than 1 kb (e.g., SEQ ID NO: 36) operably linked to exons 1-24 of a polynucleotide encoding an OTOF protein (e.g., mouse OTOF, e.g., SEQ ID NO: 6) and a splice donor sequence 3′ of the polynucleotide sequence; and the second nucleic acid vector contains a splice acceptor sequence 5′ of exons 25-48 of a polynucleotide encoding an OTOF protein (e.g., mouse OTOF, e.g., SEQ ID NO: 6) and a poly(A) sequence (e.g., a bGH poly(A) signal sequence). In this dual vector system, the second nucleic acid can also contain a full length OTOF 3′ UTR (e.g., the 1001 bp mouse OTOF 3′ UTR). A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include a 5′ OTOF UTR in the first vector.

### Dual Hybrid Vectors

A third approach for expressing large proteins in mammalian cells involves the use of dual hybrid vectors. This approach combines elements of the overlapping dual vector strategy and the trans-splicing strategy in that it features both an overlapping region at which homologous recombination can occur and splice donor and splice acceptor sequences. In dual hybrid vector systems, the overlapping region is a recombinogenic region that is contained in both the first and second nucleic acid vectors, rather than a portion of the polynucleotide sequence encoding the protein of interest—the polynucleotide encoding the N-terminal portion of the protein of interest and the polynucleotide encoding the C-terminal portion of the protein of interest do not overlap in this approach. The recombinogenic region is 3′ of the splice donor sequence in the first nucleic acid vector and 5′ of the splice acceptor sequence in the second nucleic acid vector. The first and second polynucleotide sequences can then join to form a single sequence based on one of two mechanisms: 1) recombination at the overlapping region, or 2) concatemerization of the ITRs. The remaining recombinogenic region(s) and/or the concatemerized ITRs can be removed by splicing, leading to the formation of a contiguous polynucleotide sequence that encodes the full-length protein of interest.

Recombinogenic regions that can be used in the compositions and methods described herein include the F1 phage AK gene having a sequence of: GGGATTTTGCCGATTTCGGCCTATTGGTTAA AAAATGAGCTGATTTAACAAAAATTTAACGCGAATTTTAACAAAAT (SEQ ID NO: 19) and alkaline phosphatase (AP) gene fragments as described in U.S. Pat. No. 8,236,557, which are incorporated herein by reference. In some embodiments, the AP gene fragment has the sequence of:

(SEQ ID NO: 62)
CCCCGGGTGCGCGGCGTCGGTGGTGCCGGGGGGGGCGCCAGGTCGCAGGC
GGTGTAGGGCTCCAGGCAGGCGGCGAAGGCCATGACGTGCGCTATGAAGG
TCTGCTCCTGCACGCCGTGAACCAGGTGCGCCTGCGGGCCGCGCGCGAAC
ACCGCCACGTCCTCGCCTGCGTGGGTCTCTTCGTCCAGGGGCACTGCTGA
CTGCTGCCGATACTCGGGGCTCCCGCTCTCGCTCTCGGTAACATCCGGCC
GGGCGCCGTCCTTGAGCACATAGCCTGGACCGTTTCCGTATAGGAGGACC
GTGTAGGCCTTCCTGTCCCGGGCCTTGCCAGCGGCCAGCCCGATGAAGGA
GCTCCCTCGCAGGGGGTAGCCTCCGAAGGAGAAGACGTGGGAGTGGTCGG
CAGTGACGAGGCTCAGCGTGTCCTCCTCGCTGGTGAGCTGGCCCGCCCTC
TCAATGGCGTCGTCGAACATGATCGTCTCAGTCAGTGCCCGGTAAGCCCT
GCTTTCATGATGACCATGGTCGATGCGACCACCCTCCACGAAGAGGAAGA
AGCCGCGGGGGTGTCTGCTCAGCAGGCGCAGGGCAGCCTCTGTCATCTCC
ATCAGGGAGGGGTCCAGTGTGGAGTCTCGGTGGATCTCGTATTTCATGTC
TCCAGGCTCAAAGAGACCCATGAGATGGGTCACAGACGGGTCCAGGGAAG
CCTGCATGAGCTCAGTGCGGTTCCACACGTACCGGGCACCCTGGCGTTCG
CCGAGCCATTCCTGCACCAGATTCTTCCCGTCCAGCCTGGTCCCACCTTG
GCTGTAGTCATCTGGGTACTCAGGGTCTGGGGTTCCCATGCGAAACATGT
ACTTTCGGCCTCCA.

In some embodiments, the AP gene fragment has the sequence of:

(SEQ ID NO: 63)
CCCCGGGTGCGCGGCGTCGGTGGTGCCGGGGGGGGCGCCAGGTCGCAGGC
GGTGTAGGGCTCCAGGCAGGCGGCGAAGGCCATGACGTGCGCTATGAAGG
TCTGCTCCTGCACGCCGTGAACCAGGTGCGCCTGCGGGCCGCGCGCGAAC
ACCGCCACGTCCTCGCCTGCGTGGGTCTCTTCGTCCAGGGGCACTGCTGA
CTGCTGCCGATACTCGGGGCTCCCGCTCTCGCTCTCGGTAACATCCGGCC
GGGCGCCGTCCTTGAGCACATAGCCTGGACCGTTTCCGTATAGGAGGACC
GTGTAGGCCTTCCTGTCCCGGGCCTTGCCAGCGGCCAGCCCGATGAAGGA
GCTCCCTCGCAGGGGGTAGCCTCCGAAGGAGAAGACGTGGGAGTGGTCGG
CAGTGACGAGGCTCAGCGTGTCCTCCTCG CTGGTGA.

In some embodiments, the AP gene fragment has the sequence of:

(SEQ ID NO: 64)
GCTGGCCCGCCCTCTCAATGGCGTCGTCGAACATGATCGTCTCAGTCAGT
GCCCGGTAAGCCCTGCTTTCATGATGACCATGGTCGATGCGACCACCCTC
CACGAAGAGGAAGAAGCCGCGGGGGTGTCTGCTCAGCAGGCGCAGGGCAG
CCTCTGTCATCTCCATCAGGGAGGGGTCCAGTGTGGAGTCTCGGTGGATC
TCGTATTTCATGTCTCCAGGCTCAAAGAGACCCATGAGATGGGTCACAGA
CGGGTCCAGGGAAGCCTGCATGAGCTCAGTGCGGTTCCACACGTACCGGG
CACCCTGGCGTTCGCCGAGCCATTCCTGCACCAGATTCTTCCCGTCCAGC
CTGGTCCCACCTTGGCTGTAGTCATCTGGGTACTCAGGGTCTGGGGTTCC
CATGCGAAACATGTACTTTCGGCCTCCA.

In some embodiments, the AP gene fragment has the sequence of:

(SEQ ID NO: 65)
CCCCGGGTGCGCGGCGTCGGTGGTGCCGGGGGGGGCGCCAGGTCGCAGGC
GGTGTAGGGCTCCAGGCAGGCGGCGAAGGCCATGACGTGCGCTATGAAGG
TCTGCTCCTGCACGCCGTGAACCAGGTGCGCCTGCGGGCCGCGCGCGAAC
ACCGCCACGTCCTCGCCTGCGTGGGTCTCTTCGTCCAGGGGCACTGCTGA
CTGCTGCCGATACTCGGGGCTCCCGCTCTCGCTCTCGGTAACATCCGGCC
GGGCGCCGTCCTTGAGCACATAGCCTGGACCGTTTC.

In some embodiments, the AP gene fragment has the sequence of:

(SEQ ID NO: 66)
CGTATAGGAGGACCGTGTAGGCCTTCCTGTCCCGGGCCTTGCCAGCGGCC
AGCCCGATGAAGGAGCTCCCTCGCAGGGGGTAGCCTCCGAAGGAGAAGAC
GTGGGAGTGGTCGGCAGTGACGAGGCTCAGCGTGTCCTCCTCGCTGGTGA
GCTGGCCCGCCCTCTCAATGGCGTCGTCGAACATGATCGTCTCAGTCAGT
GCCCGGTAAGCCCTGCTTTCATGATGACCATGGTCGATGCGACCACCCTC
CACGAAGAGGAAGAAGCCGCGGGGGTGTCTGCTCAGCAGG.

In some embodiments, the AP gene fragment has the sequence of:

(SEQ ID NO: 67)
CGCAGGGCAGCCTCTGTCATCTCCATCAGGGAGGGGTCCAGTGTGGAGTC
TCGGTGGATCTCGTATTTCATGTCTCCAGGCTCAAAGAGACCCATGAGAT
GGGTCACAGACGGGTCCAGGGAAGCCTGCATGAGCTCAGTGCGGTTCCAC
ACGTACCGGGCACCCTGGCGTTCGCCGAGCCATTCCTGCACCAGATTCTT
CCCGTCCAGCCTGGTCCCACCTTGGCTGTAGTCATCTGGGTACTCAGGGT
CTGGGGTTCCCATGCGAAACATGTACTTTCGGCCTCCA.

An exemplary splice donor sequence for use in the methods and compositions described herein (e.g., in trans-splicing and dual hybrid approaches) has the sequence: GTAAGTATCAAGGTTACAAGAC AGGTTTAAGGAGACCAATAGAAACTGGGCTTGTCGAGACAGAGAAGACTCTTGCGTTTCT (SEQ ID NO: 20). An exemplary splice acceptor sequence for use in the methods and compositions described herein (e.g., in trans-splicing and dual hybrid approaches) has the sequence: GATAGGCACCTATTGG TCTTACTGACATCCACTTTGCCTTTCTCTCCACAG (SEQ ID NO: 21). The splice donor sequence GTAAGTATCAAGGTTACAAGACAGGTTTAAGGAGACCAATAGAAACTGGGCTTGTCGAGACAGAGAA GACTCTTGCGTTTCTGA (SEQ ID NO: 68) and the splice acceptor sequence TAGGCACCTATTGGTCTTACTGACATCCACTTTGCCTTTCTCTCCACAG (SEQ ID NO: 69) can also be used in the methods and compositions described herein. Additional examples of splice donor and splice acceptor sequences are known in the art.

Dual hybrid vectors for use in the methods and compositions described herein are designed such that approximately half of the OTOF gene is contained within each vector (e.g., each vector contains a polynucleotide that encodes approximately half of the OTOF protein). The determination of how to split the polynucleotide sequence between the two nucleic acid vectors is made based on the size of the promoter and the locations of the portions of the polynucleotide that encode the OTOF C2 domains. When a short promoter is used in the dual hybrid vector system (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter), such as CAG, CMV, smCBA, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60), the OTOF polynucleotide sequence is divided between the two nucleic acid vectors at an exon boundary that occurs after the portion of the polynucleotide that encodes the C2D domain and before the portion of the polynucleotide that encodes C2E domain, for example, the exon 26/27 boundary. The nucleic acid vectors containing promoters of this size can optionally contain OTOF UTRs (e.g., full-length 5′ and 3′ UTRs). When a long promoter is used in the dual hybrid vector system (e.g., a promoter that is longer than 1 kb, e.g., 1.1 kb, 1.25 kb, 1.5 kb, 1.75 kb, 2 kb, 2.5 kb, 3 kb or longer), such as a Myo15 promoter that is longer than 1 kb (e.g., SEQ ID NO: 36), the OTOF polynucleotide sequence will be divided between the two nucleic acid vectors at an exon boundary that occurs after the portion of the polynucleotide that encodes the C2C domain, and either before the portion of the polynucleotide that encodes the C2D domain, such as the exon 19/20 boundary, the exon 20/21 boundary, or the exon 21/22 boundary, or within the portion of the polynucleotide that encodes the C2D domain, such as the exon 25/26 boundary. A short promoter (e.g., a CMV promoter, CAG promoter, smCBA promoter, or a Myo15 promoter having a sequence that is 1 kb or shorter, e.g., a Myo15 promoter having the sequence of any one of SEQ ID NOs: 38, 39, or 49-60) can also be used in the dual vector systems designed for large promoters, in which case additional elements (e.g., OTOF UTR sequences) may be included in the first vector (e.g., the vector containing the portion of the polynucleotide the encodes the C2C domain).

One exemplary dual hybrid vector system that uses a short promoter includes a first nucleic acid vector containing a CAG promoter operably linked to exons 1-26 of a polynucleotide encoding an OTOF protein (e.g., human OTOF, e.g., SEQ ID NO: 1 or SEQ ID NO: 5, or mouse OTOF, e.g., SEQ ID NO: 6), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains the remaining exons (e.g., exons 27-48 of mouse OTOF, or exons 27-45 and 47 or exons 27-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6), and a poly(A) sequence (e.g., a bGH poly(A) signal sequence). The first and second nucleic acid vectors can also contain the full length 5′ and 3′ OTOF UTRs, respectively (e.g., the 127 bp human OTOF 5′ UTR can be included in the first nucleic acid vector, and the 1035 bp human OTOF 3′ UTR can be included in the second nucleic acid vector). Another exemplary dual hybrid vector system that uses a short promoter includes a first nucleic acid vector containing a CAG promoter operably linked to exons 1-28 of a polynucleotide encoding an OTOF protein (e.g., human OTOF, e.g., SEQ ID NO: 1 or SEQ ID NO: 5, or mouse OTOF, e.g., SEQ ID NO: 6), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains the remaining exons (e.g., 29-48 for mouse OTOF, or exons 29-45 and 47 or exons 29-46 for human OTOF) of the polynucleotide encoding the OTOF protein (e.g., human OTOF, e.g., SEQ ID NO: 1 or SEQ ID NO: 5, or mouse OTOF, e.g., SEQ ID NO: 6), and a poly(A) sequence (e.g., a bGH poly(A) signal sequence). The first and second nucleic acid vectors can also contain the full length 5′ and 3′ OTOF UTRs, respectively (e.g., the 134 bp mouse OTOF 5′ UTR can be included in the first nucleic acid vector, and the 1001 bp mouse OTOF 3′ UTR can be included in the second nucleic acid vector). The CMV promoter, smCBA promoter, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60) can be used in place of the CAG promoter either of the foregoing dual vector systems.

An exemplary dual hybrid vector system that uses a long promoter includes a first nucleic acid vector containing a Myo15 promoter that is longer than 1 kb (e.g., SEQ ID NO: 36) operably linked to exons 1-19 of a polynucleotide encoding an OTOF protein (e.g., human OTOF, e.g., SEQ ID NO: 1 or SEQ ID NO: 5, or mouse OTOF, e.g., SEQ ID NO: 6), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains the remaining exons (e.g., 20-48 exons of mouse OTOF, or exons 20-45 and 47 or 20-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6), and a poly(A) sequence (e.g., a bGH poly(A) signal sequence). Another exemplary dual hybrid vector system that uses a long promoter includes a first nucleic acid vector containing a Myo15 promoter that is longer than 1 kb (e.g., SEQ ID NO: 36) operably linked to exons 1-20 of a polynucleotide encoding an OTOF protein (e.g., mouse OTOF, e.g., SEQ ID NO: 6, or human OTOF, e.g., SEQ ID NO: 1 or SEQ ID NO: 5), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains the remaining exons (e.g., exons 21-48 of mouse OTOF, or exons 21-45 and 47 or 21-46 of human OTOF) of a polynucleotide encoding an OTOF protein (e.g., mouse OTOF, e.g., SEQ ID NO: 6, or human OTOF, e.g., SEQ ID NO: 1 or SEQ ID NO: 5), and a poly(A) sequence (e.g., a bGH poly(A) signal sequence). Neither the first nor the second nucleic acid vector in either of the foregoing Myo15 promoter dual hybrid vector systems contains an OTOF UTR. A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include an additional element (e.g., a 5′ OTOF UTR) in the first vector.

To accommodate an OTOF UTR, the OTOF coding sequence can be divided in a different position. For example, in a dual hybrid vector system in which the first nucleic acid vector contains a Myo15 promoter that is longer than 1 kb (e.g., SEQ ID NO: 36) operably linked to exons 1-25 of a polynucleotide encoding an OTOF protein (e.g., human OTOF, e.g., SEQ ID NO: 1 or SEQ ID NO: 5, or mouse OTOF, e.g., SEQ ID NO: 6), a splice donor sequence 3′ of the polynucleotide sequence, and a recombinogenic region 3′ of the splice donor sequence; and the second nucleic acid vector contains a recombinogenic region, a splice acceptor sequence 3′ of the recombinogenic region, a polynucleotide 3′ of the splice acceptor sequence that contains the remaining exons (e.g., exons 26-48 of mouse OTOF, or exons 26-45 and 47 or exons 26-46 of human OTOF) of the polynucleotide encoding the OTOF protein (e.g., human OTOF, e.g., SEQ ID NO: 1 or SEQ ID NO: 5, or mouse OTOF, e.g., SEQ ID NO: 6), and a poly(A) sequence (e.g., a bGH poly(A) signal sequence), the second nucleic acid can also contain a full-length OTOF 3′ UTR (e.g., the 1035 bp human OTOF UTR). For mouse OTOF, the dual hybrid vector system can contain a 3′ UTR if the first nucleic acid vector contains a Myo15 promoter that is longer than 1 kb (e.g., SEQ ID NO: 36) operably linked to exons 1-24 of a polynucleotide encoding an OTOF protein (e.g., mouse OTOF, e.g., SEQ ID NO: 6), a splice donor sequence 3′ of the polynucleotide sequence, and a recombinogenic region 3′ of the splice donor sequence; and the second nucleic acid vector contains a recombinogenic region, a splice acceptor sequence 3′ of the recombinogenic region, a polynucleotide 3′ of the splice acceptor sequence that contains exons 25-48 of the polynucleotide encoding the OTOF protein (e.g., mouse OTOF, e.g., SEQ ID NO: 6), and a poly(A) sequence (e.g., a bGH poly(A) signal sequence). In this dual hybrid vector system, the second nucleic acid can also contain a full-length OTOF 3′ UTR (e.g., the 1001 bp mouse OTOF UTR). A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include an additional element (e.g., a 5′ OTOF UTR) in the first vector.

The dual hybrid vectors used in the methods and compositions described herein can optionally include a degradation signal sequence in both the first and second nucleic acid vectors. The degradation signal sequence can be included to prevent or reduce the expression of portions of the OTOF protein from polynucleotides that failed to recombine and/or undergo splicing. The degradation signal sequence is positioned 3′ of the recombinogenic region in the first nucleic acid vector and is positioned between the recombinogenic region and the splice acceptor in the second nucleic acid vector. A degradation signal sequence that can be used in the compositions and methods described herein has the sequence of:

(SEQ ID NO: 22)
GCCTGCAAGAACTGGTTCAGCAGCCTGAGCCACTTCGTGATCCACCTG.

Exemplary pairs of overlapping, trans-splicing, and dual hybrid vectors are described in Table 4 below.

TABLE 4
Exemplary pairs of overlapping, trans-splicing, and hybrid dual
vectors for use in the methods and compositions described herein

| Vector Pair | Vector | |
|---|---|---|
| Number | Type | Vector Pair |
| 1 | Overlapping | First nucleic acid vector contains: CAG promoter operably linked to exons |
| | | 1-24 and the 500 bp 3′ of the exon 24/25 boundary of a polynucleotide |
| | | encoding a human OTOF protein |
| | | Second nucleic acid vector contains: the 500 bp 5′ of the exon 24/25 |
| | | boundary and the remaining exons of the polynucleotide encoding the |
| | | human OTOF protein and a bGH poly(A) sequence |
| 2 | Overlapping | First nucleic acid vector contains: CAG promoter operably linked to exons |
| | | 1-28 and the 500 bp 3′ of the exon 28/29 boundary of a polynucleotide |
| | | encoding a human OTOF protein |
| | | Second nucleic acid vector contains: the 500 bp 5′ of the exon 28/29 |
| | | boundary and the remaining exons of the polynucleotide encoding the |
| | | human OTOF protein and a bGH poly(A) sequence |
| 3 | Overlapping | First nucleic acid vector contains: Myo15 promoter operably linked to |
| | | exons 1-21 and the 500 bp 3′ of the exon 21/22 boundary of a |
| | | polynucleotide encoding a human OTOF protein |
| | | Second nucleic acid vector contains: the 500 bp 5′ of the exon 21/22 |
| | | boundary and the remaining exons of the polynucleotide encoding the |
| | | human OTOF protein and a bGH poly(A) sequence |
| 4 | Overlapping | First nucleic acid vector contains: Myo15 promoter operably linked to |
| | | exons 1-20 and the 500 bp 3′ of the exon 20/21 boundary of a |
| | | polynucleotide encoding a human OTOF protein |
| | | Second nucleic acid vector contains: the 500 bp 5′ of the exon 20/21 |
| | | boundary and the remaining exons of the polynucleotide encoding the |
| | | human OTOF protein and a bGH poly(A) sequence |
| 5 | Trans- | First nucleic acid vector contains: CAG promoter operably linked to exons |
| | splicing | 1-26 of a polynucleotide encoding a human OTOF protein and a splice |
| | | donor sequence 3′ of the polynucleotide |
| | | Second nucleic acid vector contains: a splice acceptor sequence 5′ of the |
| | | remaining exons of the polynucleotide encoding the human OTOF protein |
| | | and a bGH poly(A) sequence |
| 6 | Trans- | First nucleic acid vector contains: Myo15 promoter operably linked to |
| | splicing | exons 1-19 of a polynucleotide encoding a human OTOF protein and a |
| | | splice donor sequence 3′ of the polynucleotide |
| | | Second nucleic acid vector contains: a splice acceptor sequence 5′ of the |
| | | remaining exons of the polynucleotide encoding the human OTOF protein |
| | | and a bGH poly(A) sequence |
| 7 | Trans- | First nucleic acid vector contains: Myo15 promoter operably linked to |
| | splicing | exons 1-25 of a polynucleotide encoding a human OTOF protein and a |
| | | splice donor sequence 3′ of the polynucleotide |
| | | Second nucleic acid vector contains: a splice acceptor sequence 5′ of the |
| | | remaining exons of the polynucleotide encoding the human OTOF protein |
| | | and a bGH poly(A) sequence |
| 8 | Trans- | First nucleic acid vector contains: Myo15 promoter operably linked to |
| | splicing | exons 1-20 of a polynucleotide encoding a human OTOF protein and a |
| | | splice donor sequence 3′ of the polynucleotide |
| | | Second nucleic acid vector contains: a splice acceptor sequence 5′ of the |
| | | remaining exons of the polynucleotide encoding the human OTOF protein |
| | | and a bGH poly(A) sequence |
| 9 | Hybrid | First nucleic acid vector contains: CAG promoter operably linked to exons |
| | | 1-26 of a polynucleotide encoding a human OTOF protein, a splice donor |
| | | sequence 3′ of the polynucleotide, and a recombinogenic region 3′ of the |
| | | splice donor sequence |
| | | Second nucleic acid vector contains: a recombinogenic region, a splice |
| | | acceptor sequence 3′ of the recombinogenic region, the remaining exons |
| | | of the polynucleotide encoding the human OTOF protein 3′ of the splice |
| | | acceptor sequence, and a bGH poly(A) sequence |
| 10 | Hybrid | First nucleic acid vector contains: Myo15 promoter operably linked to |
| | | exons 1-19 of a polynucleotide encoding a human OTOF protein, a splice |
| | | donor sequence 3′ of the polynucleotide, and a recombinogenic region 3′ |
| | | of the splice donor sequence |
| | | Second nucleic acid vector contains: a recombinogenic region, a splice |
| | | acceptor sequence 3′ of the recombinogenic region, the remaining exons |
| | | of the polynucleotide encoding the human OTOF protein 3′ of the splice |
| | | acceptor sequence, and a bGH poly(A) sequence |
| 11 | Hybrid | First nucleic acid vector contains: Myo15 promoter operably linked to |
| | | exons 1-25 of a polynucleotide encoding a human OTOF protein, a splice |
| | | donor sequence 3′ of the polynucleotide, and a recombinogenic region 3′ |
| | | of the splice donor sequence |
| | | Second nucleic acid vector contains: a recombinogenic region, a splice |
| | | acceptor sequence 3′ of the recombinogenic region, the remaining exons |
| | | of the polynucleotide encoding the human OTOF protein 3′ of the splice |
| | | acceptor sequence, and a bGH poly(A) sequence |
| 12 | Hybrid | First nucleic acid vector contains: Myo15 promoter operably linked to |
| | | exons 1-20 of a polynucleotide encoding a human OTOF protein, a splice |
| | | donor sequence 3′ of the polynucleotide, and a recombinogenic region 3′ |
| | | of the splice donor sequence |
| | | Second nucleic acid vector contains: a recombinogenic region, a splice |
| | | acceptor sequence 3′ of the recombinogenic region, the remaining exons |
| | | of the polynucleotide encoding the human OTOF protein 3′ of the splice |
| | | acceptor sequence, and a bGH poly(A) sequence |

In some embodiments, the polynucleotide sequence encoding an OTOF protein is a cDNA sequence (e.g., a sequence that does not include introns). In some embodiments, the first and/or the second nucleic acid vector in the dual vector system can include intronic sequence. The intronic sequence may be included between one or more exons in the OTOF coding sequence, or the intronic sequence can be included between an exon of the coding sequence and another component of the nucleic acid vector (e.g., between an exon of the OTOF coding sequence and the splice donor sequence in the first nucleic acid vector or between an exon of the OTOF coding sequence and the splice acceptor sequence in the second nucleic acid vector).

In some embodiments, the polynucleotide encoding the OTOF protein is divided between the first and second nucleic acid vectors (e.g., AAV vectors) in the dual vector system at the exon 20/21 boundary. When the polynucleotide encoding the OTOF protein encodes OTOF isoform 5 and is divided between the first and second nucleic acid vectors (e.g., AAV vectors) at the exon 20/21 boundary, the polynucleotide sequence encoding the N-terminal portion of OTOF has the sequence of:

(SEQ ID NO: 71)
ATGGCCTTGCTCATCCACCTCAAGACAGTCTCGGAGCTGCGGGGCAGGGGCGACC
GGATCGCCAAAGTGACTTTCCGAGGGCAATCCTTCTACTCTCGGGTCCTGGAGAAC
TGTGAGGATGTGGCTGACTTTGATGAGACATTTCGGTGGCCGGTGGCCAGCAGCAT
CGACAGAAATGAGATGCTGGAGATTCAGGTTTTCAACTACAGCAAAGTCTTCAGCAA
CAAGCTCATCGGGACCTTCCGCATGGTGCTGCAGAAGGTGGTAGAGGAGAGCCAT
GTGGAGGTGACTGACACGCTGATTGATGACAACAATGCTATCATCAAGACCAGCCT
GTGCGTGGAGGTCCGGTATCAGGCCACTGACGGCACAGTGGGCTCCTGGGACGAT
GGGGACTTCCTGGGAGATGAGTCTCTTCAAGAGGAAGAGAAGGACAGCCAAGAGA
CGGATGGACTGCTCCCAGGCTCCCGGCCCAGCTCCCGGCCCCCAGGAGAGAAGA
GCTTCCGGAGAGCCGGGAGGAGCGTGTTCTCCGCCATGAAGCTCGGCAAAAACCG
GTCTCACAAGGAGGAGCCCCAAAGACCAGATGAACCGGCGGTGCTGGAGATGGAA
GACCTTGACCATCTGGCCATTCGGCTAGGAGATGGACTGGATCCCGACTCGGTGTC
TCTAGCCTCAGTCACAGCTCTCACCACTAATGTCTCCAACAAGCGATCTAAGCCAGA
CATTAAGATGGAGCCAAGTGCTGGGGGGCCCATGGATTACCAGGTCAGCATCACGG
TGATCGAGGCCCGGCAGCTGGTGGGCTTGAACATGGACCCTGTGGTGTGCGTGGA
GGTGGGTGACGACAAGAAGTACACATCCATGAAGGAGTCCACTAACTGCCCCTATT
ACAACGAGTACTTCGTCTTCGACTTCCATGTCTCTCCGGATGTCATGTTTGACAAGA
TCATCAAGATTTCGGTGATTCACTCCAAGAACCTGCTGCGCAGTGGCACCCTGGTG
GGCTCCTTCAAAATGGACGTGGGAACCGTGTACTCGCAGCCAGAGCACCAGTTCCA
TCACAAGTGGGCCATCCTGTCTGACCCCGATGACATCTCCTCGGGGCTGAAGGGCT
ACGTGAAGTGTGACGTTGCCGTGGTGGGCAAAGGGGACAACATCAAGACGCCCCA
CAAGGCCAATGAGACCGACGAAGATGACATTGAGGGGAACTTGCTGCTCCCCGAG
GGGGTGCCCCCCGAACGCCAGTGGGCCCGGTTCTATGTGAAAATTTACCGAGCAG
AGGGGCTGCCCCGTATGAACACAAGCCTCATGGCCAATGTAAAGAAGGCTTTCATC
GGTGAAAACAAGGACCTCGTGGACCCCTACGTGCAAGTCTTCTTTGCTGGCCAGAA
GGGCAAGACTTCAGTGCAGAAGAGCAGCTATGAGCCCCTGTGGAATGAGCAGGTC
GTCTTTACAGACCTCTTCCCCCCACTCTGCAAACGCATGAAGGTGCAGATCCGAGA
CTCGGACAAGGTCAACGACGTGGCCATCGGCACCCACTTCATTGACCTGCGCAAGA
TTTCTAATGACGGAGACAAAGGCTTCCTGCCCACACTGGGCCCAGCCTGGGTGAAC
ATGTACGGCTCCACACGTAACTACACGCTGCTGGATGAGCATCAGGACCTGAACGA
GGGCCTGGGGGAGGGTGTGTCCTTCCGGGCCCGGCTCCTGCTGGGCCTGGCTGT
GGAGATCGTAGACACCTCCAACCCTGAGCTCACCAGCTCCACAGAGGTGCAGGTG
GAGCAGGCCACGCCCATCTCGGAGAGCTGTGCAGGTAAAATGGAAGAATTCTTTCT
CTTTGGAGCCTTCCTGGAGGCCTCAATGATCGACCGGAGAAACGGAGACAAGCCCA
TCACCTTTGAGGTCACCATAGGCAACTATGGGAACGAAGTTGATGGCCTGTCCCGG
CCCCAGCGGCCTCGGCCCCGGAAGGAGCCGGGGGATGAGGAAGAAGTAGACCTG
ATTCAGAACGCAAGTGATGACGAGGCCGGTGATGCCGGGGACCTGGCCTCAGTCT
CCTCCACTCCACCAATGCGGCCCCAGGTCACCGACAGGAACTACTTCCATCTGCCC
TACCTGGAGCGAAAGCCCTGCATCTACATCAAGAGCTGGTGGCCGGACCAGCGCC
GCCGCCTCTACAATGCCAACATCATGGACCACATTGCCGACAAGCTGGAAGAAGGC
CTGAACGACATACAGGAGATGATCAAAACGGAGAAGTCCTACCCTGAGCGTCGCCT
GCGGGGCGTCCTGGAGGAGCTGAGCTGTGGCTGCTGCCGCTTCCTCTCCCTCGCT
GACAAGGACCAGGGCCACTCATCCCGCACCAGGCTTGACCGGGAGCGCCTCAAGT
CCTGCATGAGGGAGCTG.

The above sequence also corresponds to exons 1-20 of OTOF isoform 1.

When the polynucleotide encoding the OTOF protein encodes OTOF isoform 5 and is divided between the first and second nucleic acid vectors (e.g., AAV vectors) at the exon 20/21 boundary, the polynucleotide sequence encoding the C-terminal portion of OTOF has the sequence of:

(SEQ ID NO: 72)
GAAAACATGGGGCAGCAGGCCAGGATGCTGCGGGCCCAGGTGAAGCGGCACACG
GTGCGGGACAAGCTGAGGCTGTGCCAGAACTTCCTGCAGAAGCTGCGCTTCCTGG
CGGACGAGCCCCAGCACAGCATTCCCGACATCTTCATCTGGATGATGAGCAACAAC
AAGCGTGTCGCCTATGCCCGTGTGCCCTCCAAGGACCTGCTCTTCTCCATCGTGGA
GGAGGAGACTGGCAAGGACTGCGCCAAGGTCAAGACGCTCTTCCTTAAGCTGCCA
GGGAAGCGGGGCTTCGGCTCGGCAGGCTGGACAGTGCAGGCCAAGGTGGAGCTG
TACCTGTGGCTGGGCCTCAGCAAACAGCGCAAGGAGTTCCTGTGCGGCCTGCCCT
GTGGCTTCCAGGAGGTCAAGGCAGCCCAGGGCCTGGGCCTGCATGCCTTCCCACC
CGTCAGCCTGGTCTACACCAAGAAGCAGGCGTTCCAGCTCCGAGCGCACATGTACC
AGGCCCGCAGCCTCTTTGCCGCCGACAGCAGCGGACTCTCAGACCCCTTTGCCCG
CGTCTTCTTCATCAATCAGAGTCAGTGCACAGAGGTGCTGAATGAGACCCTGTGTCC
CACCTGGGACCAGATGCTGGTGTTCGACAACCTGGAGCTCTATGGTGAAGCTCATG
AGCTGAGGGACGATCCGCCCATCATTGTCATTGAAATCTATGACCAGGATTCCATGG
GCAAAGCTGACTTCATGGGCCGGACCTTCGCCAAACCCCTGGTGAAGATGGCAGAC
GAGGCGTACTGCCCACCCCGCTTCCCACCTCAGCTCGAGTACTACCAGATCTACCG
TGGCAACGCCACAGCTGGAGACCTGCTGGCGGCCTTCGAGCTGCTGCAGATTGGA
CCAGCAGGGAAGGCTGACCTGCCCCCCATCAATGGCCCGGTGGACGTGGACCGAG
GTCCCATCATGCCCGTGCCCATGGGCATCCGGCCCGTGCTCAGCAAGTACCGAGT
GGAGGTGCTGTTCTGGGGCCTACGGGACCTAAAGCGGGTGAACCTGGCCCAGGTG
GACCGGCCACGGGTGGACATCGAGTGTGCAGGGAAGGGGGTGCAGTCGTCCCTG
ATCCACAATTATAAGAAGAACCCCAACTTCAACACCCTCGTCAAGTGGTTTGAAGTG
GACCTCCCAGAGAACGAGCTGCTGCACCCGCCCTTGAACATCCGTGTGGTGGACT
GCCGGGCCTTCGGTCGCTACACACTGGTGGGCTCCCATGCCGTCAGCTCCCTGCG
ACGCTTCATCTACCGGCCCCCAGACCGCTCGGCCCCCAGCTGGAACACCACGGTC
AGGCTTCTCCGGCGCTGCCGTGTGCTGTGCAATGGGGGCTCCTCCTCTCACTCCAC
AGGGGAGGTTGTGGTGACTATGGAGCCAGAGGTACCCATCAAGAAACTGGAGACC
ATGGTGAAGCTGGACGCGACTTCTGAAGCTGTTGTCAAGGTGGATGTGGCTGAGGA
GGAGAAGGAGAAGAAGAAGAAGAAGAAGGGCACTGCGGAGGAGCCAGAGGAGGA
GGAGCCAGACGAGAGCATGCTGGACTGGTGGTCCAAGTACTTTGCCTCCATTGACA
CCATGAAGGAGCAACTTCGACAACAAGAGCCCTCTGGAATTGACTTGGAGGAGAAG
GAGGAAGTGGACAATACCGAGGGCCTGAAGGGGTCAATGAAGGGCAAGGAGAAGG
CAAGGGCTGCCAAAGAGGAGAAGAAGAAGAAAACTCAGAGCTCTGGCTCTGGCCA
GGGGTCCGAGGCCCCCGAGAAGAAGAAACCCAAGATTGATGAGCTTAAGGTATACC
CCAAAGAGCTGGAGTCCGAGTTTGATAACTTTGAGGACTGGCTGCACACTTTCAACT
TGCTTCGGGGCAAGACCGGGGATGATGAGGATGGCTCCACCGAGGAGGAGCGCAT
TGTGGGACGCTTCAAGGGCTCCCTCTGCGTGTACAAAGTGCCACTCCCAGAGGACG
TGTCCCGGGAAGCCGGCTACGACTCCACCTACGGCATGTTCCAGGGCATCCCGAG
CAATGACCCCATCAATGTGCTGGTCCGAGTCTATGTGGTCCGGGCCACGGACCTGC
ACCCTGCTGACATCAACGGCAAAGCTGACCCCTACATCGCCATCCGGCTAGGCAAG
ACTGACATCCGCGACAAGGAGAACTACATCTCCAAGCAGCTCAACCCTGTCTTTGG
GAAGTCCTTTGACATCGAGGCCTCCTTCCCCATGGAATCCATGCTGACGGTGGCTG
TGTATGACTGGGACCTGGTGGGCACTGATGACCTCATTGGGGAAACCAAGATCGAC
CTGGAGAACCGCTTCTACAGCAAGCACCGCGCCACCTGCGGCATCGCCCAGACCT
ACTCCACACATGGCTACAATATCTGGCGGGACCCCATGAAGCCCAGCCAGATCCTG
ACCCGCCTCTGCAAAGACGGCAAAGTGGACGGCCCCCACTTTGGGCCCCCTGGGA
GAGTGAAGGTGGCCAACCGCGTCTTCACTGGGCCCTCTGAGATTGAGGACGAGAA
CGGTCAGAGGAAGCCCACAGACGAGCATGTGGCGCTGTTGGCCCTGAGGCACTGG
GAGGACATCCCCCGCGCAGGCTGCCGCCTGGTGCCAGAGCATGTGGAGACGAGG
CCGCTGCTCAACCCCGACAAGCCGGGCATCGAGCAGGGCCGCCTGGAGCTGTGG
GTGGACATGTTCCCCATGGACATGCCAGCCCCTGGGACGCCTCTGGACATCTCACC
TCGGAAGCCCAAGAAGTACGAGCTGCGGGTCATCATCTGGAACACAGATGAGGTG
GTCTTGGAGGACGACGACTTCTTCACAGGGGAGAAGTCCAGTGACATCTTCGTGAG
GGGGTGGCTGAAGGGCCAGCAGGAGGACAAGCAGGACACAGACGTCCACTACCAC
TCCCTCACTGGCGAGGGCAACTTCAACTGGCGCTACCTGTTCCCCTTCGACTACCT
GGCGGCGGAGGAGAAGATCGTCATCTCCAAGAAGGAGTCCATGTTCTCCTGGGAC
GAGACCGAGTACAAGATCCCCGCGCGGCTCACCCTGCAGATCTGGGATGCGGACC
ACTTCTCCGCTGACGACTTCCTGGGGGCCATCGAGCTGGACCTGAACCGGTTCCCG
CGGGGCGCAAAGACAGCCAAGCAGTGCACCATGGAGATGGCCACCGGGGAGGTG
GACGTGCCCCTCGTGTCCATCTTCAAGCAAAAGCGCGTCAAAGGCTGGTGGCCCCT
CCTGGCCCGCAATGAGAACGATGAGTTTGAGCTCACGGGCAAGGTGGAGGCTGAG
CTGCATTTACTGACAGCAGAGGAGGCAGAGAAGAACCCAGTGGGCCTGGCCCGCA
ATGAACCTGACCCCCTAGAGAAACCCAACCGGCCCGACACGGCCTTCGTCTGGTTC
CTCAACCCTCTCAAGTCCATCAAGTACCTCATCTGCACCCGGTACAAGTGGCTCATC
ATCAAGATCGTGCTGGCGCTGTTGGGGCTGCTCATGTTGGGGCTCTTCCTCTACAG
CCTCCCTGGCTACATGGTCAAAAAGCTCCTTGGGGCATGA.

In embodiments in which the polynucleotide encodes OTOF isoform 5 and is divided between the first and second nucleic acid vectors (e.g., AAV vectors) at the exon 20/21 boundary, the N-terminal portion of the OTOF polypeptide has the sequence of:

(SEQ ID NO: 73)
MALLIHLKTVSELRGRGDRIAKVTFRGQSFYSRVLENCEDVADFDETFRW
PVASSIDRNEMLEIQVFNYSKVFSNKLIGTFRMVLQKVVEESHVEVTDTL
IDDNNAIIKTSLCVEVRYQATDGTVGSWDDGDFLGDESLQEEEKDSQETD
GLLPGSRPSSRPPGEKSFRRAGRSVFSAMKLGKNRSHKEEPQRPDEPAVL
EMEDLDHLAIRLGDGLDPDSVSLASVTALTTNVSNKRSKPDIKMEPSAGR
PMDYQVSITVIEARQLVGLNMDPVVCVEVGDDKKYTSMKESTNCPYYNEY
FVFDFHVSPDVMFDKIIKISVIHSKNLLRSGTLVGSFKMDVGTVYSQPEH
QFHHKWAILSDPDDISSGLKGYVKCDVAVVGKGDNIKTPHKANETDEDDI
EGNLLLPEGVPPERQWARFYVKIYRAEGLPRMNTSLMANVKKAFIGENKD
LVDPYVQVFFAGQKGKTSVQKSSYEPLWNEQVVFTDLFPPLCKRMKVQIR
DSDKVNDVAIGTHFIDLRKISNDGDKGFLPTLGPAWVNMYGSTRNYTLLD
EHQDLNEGLGEGVSFRARLLLGLAVEIVDTSNPELTSSTEVQVEQATPIS
ESCAGKMEEFFLFGAFLEASMIDRRNGDKPITFEVTIGNYGNEVDGLSRP
QRPRPRKEPGDEEEVDLIQNASDDEAGDAGDLASVSSTPPMRPQVTDRNY
FHLPYLERKPCIYIKSWWPDQRRRLYNANIMDHIADKLEEGLNDIQEMIK
TEKSYPERRLRGVLEELSCGCCRFLSLADKDQGHSSRTRLDRERLKSCMR
EL.

The above sequence also corresponds to the N-terminal portion of the OTOF isoform 1 protein encoded by exons 1-20.

In embodiments in which the polynucleotide encodes OTOF isoform 5 and is divided between the first and second nucleic acid vectors (e.g., AAV vectors) at the exon 20/21 boundary, the C-terminal portion of the OTOF polypeptide has the sequence of:

(SEQ ID NO: 74)
ENMGQQARMLRAQVKRHTVRDKLRLCQNFLQKLRFLADEPQHSIPDIFIW
MMSNNKRVAYARVPSKDLLFSIVEEETGKDCAKVKTLFLKLPGKRGFGSA
GWTVQAKVELYLWLGLSKQRKEFLCGLPCGFQEVKAAQGLGLHAFPPVSL
VYTKKQAFQLRAHMYQARSLFAADSSGLSDPFARVFFINQSQCTEVLNET
LCPTWDQMLVFDNLELYGEAHELRDDPPIIVIEIYDQDSMGKADFMGRTF
AKPLVKMADEAYCPPRFPPQLEYYQIYRGNATAGDLLAAFELLQIGPAGK
ADLPPINGPVDVDRGPIMPVPMGIRPVLSKYRVEVLFWGLRDLKRVNLAQ
VDRPRVDIECAGKGVQSSLIHNYKKNPNFNTLVKWFEVDLPENELLHPPL
NIRVVDCRAFGRYTLVGSHAVSSLRRFIYRPPDRSAPSWNTTVRLLRRCR
VLCNGGSSSHSTGEVVVTMEPEVPIKKLETMVKLDATSEAVVKVDVAEEE
KEKKKKKKGTAEEPEEEEPDESMLDWWSKYFASIDTMKEQLRQQEPSGID
LEEKEEVDNTEGLKGSMKGKEKARAAKEEKKKKTQSSGSGQGSEAPEKKK
PKIDELKVYPKELESEFDNFEDWLHTFNLLRGKTGDDEDGSTEEERIVGR
FKGSLCVYKVPLPEDVSREAGYDSTYGMFQGIPSNDPINVLVRVYVVRAT
DLHPADINGKADPYIAIRLGKTDIRDKENYISKQLNPVFGKSFDIEASFP
MESMLTVAVYDWDLVGTDDLIGETKIDLENRFYSKHRATCGIAQTYSTHG
YNIWRDPMKPSQILTRLCKDGKVDGPHFGPPGRVKVANRVFTGPSEIEDE
NGQRKPTDEHVALLALRHWEDIPRAGCRLVPEHVETRPLLNPDKPGIEQG
RLELWVDMFPMDMPAPGTPLDISPRKPKKYELRVIIWNTDEVVLEDDDFF
TGEKSSDIFVRGWLKGQQEDKQDTDVHYHSLTGEGNFNWRYLFPFDYLAA
EEKIVISKKESMFSWDETEYKIPARLTLQIWDADHFSADDFLGAIELDLN
RFPRGAKTAKQCTMEMATGEVDVPLVSIFKQKRVKGWWPLLARNENDEFE
LTGKVEAELHLLTAEEAEKNPVGLARNEPDPLEKPNRPDTAFVWFLNPLK
SIKYLICTRYKWLIIKIVLALLGLLMLGLFLYSLPGYMVKKLLGA.

Transfer plasmids that may be used to produce the nucleic acid vectors for use in the compositions and methods described herein are provided in Table 5. These transfer plasmids are designed for the expression of OTOF isoform 5. A transfer plasmid (e.g., a plasmid containing a DNA sequence to be delivered by a nucleic acid vector, e.g., to be delivered by an AAV) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. Nucleic acid vectors (e.g., a nucleic acid vector (e.g., an AAV vector) containing a polynucleotide encoding an N-terminal portion of OTOF and a nucleic acid vector (e.g., an AAV vector) containing a polynucleotide encoding a C-terminal portion of OTOF) can be combined (e.g., in a single formulation) prior to administration. The following transfer plasmids are designed to produce nucleic acid vectors (e.g., AAV vectors) for co-formulation or co-administration (e.g., administration simultaneously or sequentially) in a dual hybrid vector system: SEQ ID NO: 75 and SEQ ID NO: 76; SEQ ID NO: 77 and SEQ ID NO: 78; SEQ ID NO: 79 and SEQ ID NO: 76; SEQ ID NO: 80 and SEQ ID NO: 78; SEQ ID NO: 81 and SEQ ID NO: 82; and SEQ ID NO: 83 and SEQ ID NO: 82.

TABLE 5
Transfer plasmids for the production of dual hybrid vector systems

| SEQ | | |
|---|---|---|
| ID | | |
| NO. | Description | Plasmid Sequence |
| 75 | 5′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda (Biologically inert and | TTTCCATCATTCCAGTCGAACTCACACACAACACCAAA |
| | inactivated DNA derived from | TGCATTTAAGTCGCTTGAAATTGCTATAAGCAGAGCAT |
| | bacteriophage lambda to | GTTGCGCCAGCATGATTAATACAGCATTTAATACAGAG |
| | reduce off-target DNA | CCGTGTTTATTGAGTCGGTATTCAGAGTCTGACCAGAA |
| | encapsidation) at positions 53- | ATTATTAATCTGGTGAAGTTATTCCTCTGTCATTACGTC |
| | 2027 | ATGGTCGATTTCAATTTCTATTGATGCTTTCCAGTCGTA |
| | ITR at positions 2049-2178 | ATCAATGATGTATTTTTTGATGTTTGACCTCTGTTCATA |
| | Myo15 promoter at positions | TCCTCACAGATAAAAAATCGCCCTCACACTGGAGGGC |
| | 2272-3236 | AAAGAAGATTTCCAATAATCAGAACAAGTCGGCTCCTG |
| | Kozak sequence (Site to initiate | TTTAGTTACGAGCGACATTGCTCCGTGTATTCACTCGT |
| | protein translation) at positions | TGGAATGAATACACAGTGCAGTGTTTATTCTGTTATTTA |
| | 3253-3262 | TGCCAAAAATTAAGGCCACTATCAGGCAGCTTTGTTGT |
| | N-terminal portion of human | TCTGTTTACCAAGTTCTCTGGCAATCATTGCCGTCGTT |
| | OTOF isoform 5 at positions | CGTATTGCCCATTTATCGACATATTTCCCATCTTCCTAT |
| | 3259-5664 | ACAGGAAACATTTCTTCAGGCTTAACCATGCATTCCGA |
| | Splice donor (APSD) sequence | TTGCAGCTTGCATCCATTGCATCGCTTGAATTGTCCAC |
| | at positions 5665-5748 | ACCATTGATTTTTATCAATAGTCGTAGTTTAACGGATAG |
| | AP head sequence | TCCTGGTATTGTTCCATCACATCCTGAGGATGCCCTTC |
| | (recombinogenic region) at | GAACTCTTCAAATTCTTCTTCCTAATATCACCTTAAATA |
| | positions 5755-6041 | GTGGATTGCGGTAGTAAAGATTGTGCCTGTCTTTTAAC |
| | ITR at positions 6135-6264 | CACATCAGGCTCGGTGGTTCTCGTGTACCCCTACAGC |
| | Lambda at positions 6275-8287 | GAGAAATCGGATAAACTATTACAACCCCTACAGTTTGT |
| | Ori (origin of replication) at | AGAGTATAGAAAATGATCCACTCGTTATTCTCGGACGA |
| | positions 8344-8932 | GTGTTCAGTAATGAACCTCTGGAGAGAACCATCTATAT |
| | KanR (antibiotic resistance | GATCGTTATCTGGGTTTGACTTCTGCTTTTAAGCCCAG |
| | gene) at positions 9110-9919 | ATAACTTGCCTGAATATGTTAATGAGAGAATCGGTATT |
| | Transgene to be transferred | CCTCATGTGTGGCATGTTTTCGTCTTTGCTCTTGCATTT |
| | into vector in dual vector | TCACTAGCAATTAATGTGCATCGATTATCAGCTATTGC |
| | system at positions 2049-6264 | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGCGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCCAATTC |
| | | TGCAGCTCAGCCTACTACTTGCTTTCCAGGCTGTTCCT |
| | | AGTTCCCATGTCAGCTGCTTGTGCTTTCCAGAGACAAA |
| | | ACAGGAATAATAGATGTCATTAAATATACATTGGGCCC |
| | | CAGGCGGTCAATGTGGCAGCCTGAGCCTCCTTTCCAT |
| | | CTCTGTGGAGGCAGACATAGGACCCCCAACAAACAGC |
| | | ATGCAGGTTGGGAGCCAGCCACAGGACCCAGGTAAG |
| | | GGGCCCTGGGTCCTTAAGCTTCTGCCACTGGCTCCGG |
| | | CATTGCAGAGAGAAGAGAAGGGGCGGCAGACTGGAG |
| | | AGCTGGGCTCCATTTTTGTTCCTTGGTGCCCTGCCCCT |
| | | CCCCATGACCTGCAGAGACATTCAGCCTGCCAGGCTT |
| | | TATGAGGTGGGAGCTGGGCTCTCCCTGATGTATTATTC |
| | | AGCTCCCTGGAGTTGGCCAGCTCCTGTTACACTGGCC |
| | | ACAGCCCTGGGCATCCGCTTCTCACTTCTAGTTTCCCC |
| | | TCCAAGGTAATGTGGTGGGTCATGATCATTCTATCCTG |
| | | GCTTCAGGGACCTGACTCCACTTTGGGGCCATTCGAG |
| | | GGGTCTAGGGTAGATGATGTCCCCCTGTGGGGATTAA |
| | | TGTCCTGCTCTGTAAAACTGAGCTAGCTGAGATCCAG |
| | | GAGGGCTTGGCCAGAGACAGCAAGTTGTTGCCATGGT |
| | | GACTTTAAAGCCAGGTTGCTGCCCCAGCACAGGCCTC |
| | | CCAGTCTACCCTCACTAGAAAACAACACCCAGGCACTT |
| | | TCCACCACCTCTCAAAGGTGAAACCCAAGGCTGGTCT |
| | | AGAGAATGAATTATGGATCCTCGCTGTCCGTGCCACC |
| | | CAGCTAGTCCCAGCGGCTCAGACACTGAGGAGAGACT |
| | | GTAGGTTCAGCTACAAGCAAAAAGACCTAGCTGGTCT |
| | | CCAAGCAGTGTCTCCAAGTCCCTGAACCTGTGACACC |
| | | TGCCCCAGGCATCATCAGGCACAGAGGGCCACCAAGA |
| | | ATTCTAGCGGCCGCCACCATGGCCTTGCTCATCCACC |
| | | TCAAGACAGTCTCGGAGCTGCGGGGCAGGGGCGACC |
| | | GGATCGCCAAAGTGACTTTCCGAGGGCAATCCTTCTA |
| | | CTCTCGGGTCCTGGAGAACTGTGAGGATGTGGCTGAC |
| | | TTTGATGAGACATTTCGGTGGCCGGTGGCCAGCAGCA |
| | | TCGACAGAAATGAGATGCTGGAGATTCAGGTTTTCAAC |
| | | TACAGCAAAGTCTTCAGCAACAAGCTCATCGGGACCTT |
| | | CCGCATGGTGCTGCAGAAGGTGGTAGAGGAGAGCCA |
| | | TGTGGAGGTGACTGACACGCTGATTGATGACAACAAT |
| | | GCTATCATCAAGACCAGCCTGTGCGTGGAGGTCCGGT |
| | | ATCAGGCCACTGACGGCACAGTGGGCTCCTGGGACG |
| | | ATGGGGACTTCCTGGGAGATGAGTCTCTTCAAGAGGA |
| | | AGAGAAGGACAGCCAAGAGACGGATGGACTGCTCCCA |
| | | GGCTCCCGGCCCAGCTCCCGGCCCCCAGGAGAGAAG |
| | | AGCTTCCGGAGAGCCGGGAGGAGCGTGTTCTCCGCC |
| | | ATGAAGCTCGGCAAAAACCGGTCTCACAAGGAGGAGC |
| | | CCCAAAGACCAGATGAACCGGCGGTGCTGGAGATGG |
| | | AAGACCTTGACCATCTGGCCATTCGGCTAGGAGATGG |
| | | ACTGGATCCCGACTCGGTGTCTCTAGCCTCAGTCACA |
| | | GCTCTCACCACTAATGTCTCCAACAAGCGATCTAAGCC |
| | | AGACATTAAGATGGAGCCAAGTGCTGGGGGGCCCATG |
| | | GATTACCAGGTCAGCATCACGGTGATCGAGGCCCGGC |
| | | AGCTGGTGGGCTTGAACATGGACCCTGTGGTGTGCGT |
| | | GGAGGTGGGTGACGACAAGAAGTACACATCCATGAAG |
| | | GAGTCCACTAACTGCCCCTATTACAACGAGTACTTCGT |
| | | CTTCGACTTCCATGTCTCTCCGGATGTCATGTTTGACA |
| | | AGATCATCAAGATTTCGGTGATTCACTCCAAGAACCTG |
| | | CTGCGCAGTGGCACCCTGGTGGGCTCCTTCAAAATGG |
| | | ACGTGGGAACCGTGTACTCGCAGCCAGAGCACCAGTT |
| | | CCATCACAAGTGGGCCATCCTGTCTGACCCCGATGAC |
| | | ATCTCCTCGGGGCTGAAGGGCTACGTGAAGTGTGACG |
| | | TTGCCGTGGTGGGCAAAGGGGACAACATCAAGACGCC |
| | | CCACAAGGCCAATGAGACCGACGAAGATGACATTGAG |
| | | GGGAACTTGCTGCTCCCCGAGGGGGTGCCCCCCGAA |
| | | CGCCAGTGGGCCCGGTTCTATGTGAAAATTTACCGAG |
| | | CAGAGGGGCTGCCCCGTATGAACACAAGCCTCATGGC |
| | | CAATGTAAAGAAGGCTTTCATCGGTGAAAACAAGGACC |
| | | TCGTGGACCCCTACGTGCAAGTCTTCTTTGCTGGCCA |
| | | GAAGGGCAAGACTTCAGTGCAGAAGAGCAGCTATGAG |
| | | CCCCTGTGGAATGAGCAGGTCGTCTTTACAGACCTCTT |
| | | CCCCCCACTCTGCAAACGCATGAAGGTGCAGATCCGA |
| | | GACTCGGACAAGGTCAACGACGTGGCCATCGGCACC |
| | | CACTTCATTGACCTGCGCAAGATTTCTAATGACGGAGA |
| | | CAAAGGCTTCCTGCCCACACTGGGCCCAGCCTGGGTG |
| | | AACATGTACGGCTCCACACGTAACTACACGCTGCTGG |
| | | ATGAGCATCAGGACCTGAACGAGGGCCTGGGGGAGG |
| | | GTGTGTCCTTCCGGGCCCGGCTCCTGCTGGGCCTGG |
| | | CTGTGGAGATCGTAGACACCTCCAACCCTGAGCTCAC |
| | | CAGCTCCACAGAGGTGCAGGTGGAGCAGGCCACGCC |
| | | CATCTCGGAGAGCTGTGCAGGTAAAATGGAAGAATTC |
| | | TTTCTCTTTGGAGCCTTCCTGGAGGCCTCAATGATCGA |
| | | CCGGAGAAACGGAGACAAGCCCATCACCTTTGAGGTC |
| | | ACCATAGGCAACTATGGGAACGAAGTTGATGGCCTGT |
| | | CCCGGCCCCAGCGGCCTCGGCCCCGGAAGGAGCCG |
| | | GGGGATGAGGAAGAAGTAGACCTGATTCAGAACGCAA |
| | | GTGATGACGAGGCCGGTGATGCCGGGGACCTGGCCT |
| | | CAGTCTCCTCCACTCCACCAATGCGGCCCCAGGTCAC |
| | | CGACAGGAACTACTTCCATCTGCCCTACCTGGAGCGA |
| | | AAGCCCTGCATCTACATCAAGAGCTGGTGGCCGGACC |
| | | AGCGCCGCCGCCTCTACAATGCCAACATCATGGACCA |
| | | CATTGCCGACAAGCTGGAAGAAGGCCTGAACGACATA |
| | | CAGGAGATGATCAAAACGGAGAAGTCCTACCCTGAGC |
| | | GTCGCCTGCGGGGCGTCCTGGAGGAGCTGAGCTGTG |
| | | GCTGCTGCCGCTTCCTCTCCCTCGCTGACAAGGACCA |
| | | GGGCCACTCATCCCGCACCAGGCTTGACCGGGAGCG |
| | | CCTCAAGTCCTGCATGAGGGAGCTGGTAAGTATCAAG |
| | | GTTACAAGACAGGTTTAAGGAGACCAATAGAAACTGG |
| | | GCTTGTCGAGACAGAGAAGACTCTTGCGTTTCTGAGC |
| | | TAGCCCCCGGGTGCGCGGCGTCGGTGGTGCCGGCG |
| | | GGGGGCGCCAGGTCGCAGGCGGTGTAGGGCTCCAG |
| | | GCAGGCGGCGAAGGCCATGACGTGCGCTATGAAGGT |
| | | CTGCTCCTGCACGCCGTGAACCAGGTGCGCCTGCGG |
| | | GCCGCGCGCGAACACCGCCACGTCCTCGCCTGCGTG |
| | | GGTCTCTTCGTCCAGGGGCACTGCTGACTGCTGCCGA |
| | | TACTCGGGGCTCCCGCTCTCGCTCTCGGTAACATCCG |
| | | GCCGGGCGCCGTCCTTGAGCACATAGCCTGGACCGTT |
| | | TCGTCGACCTCGAGTTAAGGGCGAATTCCCGATAAGG |
| | | ATCTTCCTAGAGCATGGCTACGTAGATAAGTAGCATGG |
| | | CGGGTTAATCATTAACTACAAGGAACCCCTAGTGATGG |
| | | AGTTGGCCACTCCCTCTCTGCGCGCTCGCTCGCTCAC |
| | | TGAGGCCGGGCGACCAAAGGTCGCCCGACGCCCGGG |
| | | CTTTGCCCGGGGGGCCTCAGTGAGCGAGCGAGCGCG |
| | | CAGCCTTAATTAAATCCACATCTGTATGTTTTTTATATT |
| | | AATTTATTTTTTGCAGGGGGGCATTGTTTGGTAGGTGA |
| | | GAGTTCTGAATTGCTATGTTTAGTGAGTTGTATCTATTT |
| | | ATTTTTCAATAAATACAATTAGTTATGTGTTTTGGGGGC |
| | | GATCGTGAGGCAAAGAAAACCCGGCGCTGAGGCCGG |
| | | GTTATTCTTGTTCTCTGGTCAAATTATATAGTTGGAAAA |
| | | CAAGGATGCATATATGAATGAACGATGCAGAGGCAAT |
| | | GCCGATGGCGATAGTGGGTATCAGGTAGCCGCTTATG |
| | | CTGGAAAGAAGCAATAACCCGCAGAAAAACAAAGCTC |
| | | CAAGCTCAACAAAACTAAGGGCATAGACAATAACTACC |
| | | TATGTCATATACCCATACTCTCTAATCTTGGCCAGTCG |
| | | GCGCGTTCTGCTTCCGATTAGAAACGTCAAGGCAGCA |
| | | ATCAGGATTGCAATCTTGGTTCCTGCATAGGATGACAA |
| | | TGTCGCCCCAAGACCATCTCTATGAGCTGAAAAAGAAA |
| | | CACAAGGAATGTAGTGGCGGAAAAGGAGATAGCAAAT |
| | | GCTTACGATAACGTAAGGAATTATTACTATGTAAACAC |
| | | CAGGCAAGATTCTGTTCCGTATAATTACTCCTGATAATT |
| | | AATCCTTAACTTTGCCCACCTGCCTTTTAAAACATTCCA |
| | | GTATATCACTTTTCATTCTTGCGTAGCAATATGCCCTCT |
| | | CTTCAGCTATCTCAGCATTGGTGACCTTGTTCAGAGGC |
| | | GCTGAGAGATGGCCTTTTTCTGATAGATAATGTTCTGT |
| | | TAAAATATCTCCGGCCTCATCTTTTGCCCGCAGGCTAA |
| | | TGTCTGAAAATTGAGGTGACGGGTTAAAAATAATATCC |
| | | TTGGCAACCTTTTTTATATCCCTTTTAAATTTTGGCTTA |
| | | ATGACTATATCCAATGAGTCAAAAAGCTCCCCTTCAAT |
| | | ATCTGTTGCCCCTAAGACCTTTAATATATCGCCAAATA |
| | | CAGGTAGCTTGGCTTCTACCTTCACCGTTGTTCTGCCG |
| | | ATGAAATGCTAATGCATAACATCGTCTTTGGTGGTTCC |
| | | CCTCATCAGTGGCTCTATCTGAACGCGCTCTCCACTG |
| | | CTTAATGACATTCCTTTCCCGATTAAAAAATCTGTCAGA |
| | | TCGGATGTGGTCGGCCCGAAAACAGTTCTGGCAAAAC |
| | | CAATGGTGTCGCCTTCAACAAACAAAAAAGATGGGAAT |
| | | CCCAATGATTCGTCATCTGCGAGGCTGTTCTTAATATC |
| | | TTCAACTGTAGCTTTAGAGCGATTTATCTTCTGAACCA |
| | | GACTCTTGTCATTTGTTTTGGTAAAGAGAAAAGTTTTTC |
| | | CATCGATTTTATGAATATACAAATAATTGGAGCCAACCT |
| | | TCAGGTGATGATTATCAGCCAGCAGAGAATTAAGGAAA |
| | | ACAGACAGGTTTATTGAGCACTTATCTTTCCCTTTATTT |
| | | TTGCTGCGGTAAGTCGCATAAAAACCATTCTTCACAAT |
| | | TCAATCCATTTACTATGTTATGTTCTGAGGGGAGTGAA |
| | | AATTCCCCTAATTCGATGAAGATTCTTGCTAAATTGTTA |
| | | TCAGCTATGCGCCGACCAGAACACCTTGCCGATCAGC |
| | | CAAACGTCTAATCAGGCCACTGACTAGCGATAACTTTC |
| | | CCCACAACGGAACAACTCTCATTGCATGGGATAATTGG |
| | | GTACTGTGGGTTTAGTGGTTGTAAAAACACCTGACCGC |
| | | TATCCCTGATCAGTTTCTTGAAGGTAAACTCATCACCC |
| | | CCAAGTCTGGCTATACAGAAATCACCTGGCTCAACAG |
| | | CCTGCTCAGGGTCAACGAGAATTTACATTCCGTCAGG |
| | | ATAGCTTGGCTTGGAGCCTGTTGGTGCGGTCACGGAA |
| | | TTACCTTCAACCTCAAGCCAGAATGCAGAATCACTGGC |
| | | TTTTTTGGTTGTGCTTACCCATCTCTCCGCATCACCTTT |
| | | GGTAAAGGTTCTAAGCTAAGGTGAGAACATCCCTGCC |
| | | TGAACATGAGAAAAAACAGGGTACTCATACTCACTTAT |
| | | TAGTGACGGCTATGAGCAAAAGGCCAGCAAAAGGCCA |
| | | GGAACCGTAAAAAGGCCGCGTTGCTGGCGTTTTTCCA |
| | | TAGGCTCCGCCCCCCTGACGAGCATCACAAAAATCGA |
| | | CGCTCAAGTCAGAGGTGGCGAAACCCGACAGGACTAT |
| | | AAAGATACCAGGCGTTTCCCCCTGGAAGCTCCCTCGT |
| | | GCGCTCTCCTGTTCCGACCCTGCCGCTTACCGGATAC |
| | | CTGTCCGCCTTTCTCCCTTCGGGAAGCGTGGCGCTTT |
| | | CTCATAGCTCACGCTGTAGGTATCTCAGTTCGGTGTAG |
| | | GTCGTTCGCTCCAAGCTGGGCTGTGTGCACGAACCCC |
| | | CCGTTCAGCCCGACCGCTGCGCCTTATCCGGTAACTA |
| | | TCGTCTTGAGTCCAACCCGGTAAGACACGACTTATCG |
| | | CCACTGGCAGCAGCCACTGGTAACAGGATTAGCAGAG |
| | | CGAGGTATGTAGGCGGTGCTACAGAGTTCTTGAAGTG |
| | | GTGGCCTAACTACGGCTACACTAGAAGAACAGTATTTG |
| | | GTATCTGCGCTCTGCTGAAGCCAGTTACCTTCGGAAAA |
| | | AGAGTTGGTAGCTCTTGATCCGGCAAACAAACCACCG |
| | | CTGGTAGCGGTGGTTTTTTTGTTTGCAAGCAGCAGATT |
| | | ACGCGCAGAAAAAAAGGATCTCAAGAAGATCCTTTGAT |
| | | CTTTTCTACGGGGTCTGACGCTCAGTGGAACGAAAAC |
| | | TCACGTTAAGGGATTTTGGTCATGAGATTATCAAAAAG |
| | | GATCTTCACCTAGATCCTTTTAAATTAAAAATGAAGTTT |
| | | TAAATCAAGCCCAATCTGAATAATGTTACAACCAATTAA |
| | | CCAATTCTGATTAGAAAAACTCATCGAGCATCAAATGA |
| | | AACTGCAATTTATTCATATCAGGATTATCAATACCATAT |
| | | TTTTGAAAAAGCCGTTTCTGTAATGAAGGAGAAAACTC |
| | | ACCGAGGCAGTTCCATAGGATGGCAAGATCCTGGTAT |
| | | CGGTCTGCGATTCCGACTCGTCCAACATCAATACAACC |
| | | TATTAATTTCCCCTCGTCAAAAATAAGGTTATCAAGTGA |
| | | GAAATCACCATGAGTGACGACTGAATCCGGTGAGAAT |
| | | GGCAAAAGTTTATGCATTTCTTTCCAGACTTGTTCAACA |
| | | GGCCAGCCATTACGCTCGTCATCAAAATCACTCGCATC |
| | | AACCAAACCGTTATTCATTCGTGATTGCGCCTGAGCAA |
| | | GACGAAATACGCGATCGCTGTTAAAAGGACAATTACAA |
| | | ACAGGAATCGAATGCAACCGGCGCAGGAACACTGCCA |
| | | GCGCATCAACAATATTTTCACCTGAATCAGGATATTCTT |
| | | CTAATACCTGGAATGCTGTTTTTCCGGGGATCGCAGTG |
| | | GTGAGTAACCATGCATCATCAGGAGTACGGATAAAATG |
| | | CTTGATGGTCGGAAGAGGCATAAATTCCGTCAGCCAG |
| | | TTTAGTCTGACCATCTCATCTGTAACATCATTGGCAAC |
| | | GCTACCTTTGCCATGTTTCAGAAACAACTCTGGCGCAT |
| | | CGGGCTTCCCATACAAGCGATAGATTGTCGCACCTGA |
| | | TTGCCCGACATTATCGCGAGCCCATTTATACCCATATA |
| | | AATCAGCATCCATGTTGGAATTTAATCGCGGCCTCGAC |
| | | GTTTCCCGTTGAATATGGCTCATAACACCCCTTGTATT |
| | | ACTGTTTATGTAAGCAGACAGTTTTATTGTTCATGATGA |
| | | TATATTTTTATCTTGTGCAATGTAACATCAGAGATTTTG |
| | | AGACACGGGCCAGAGCTGCA |
| 76 | 3′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda at positions 53-2027 | TTTCCATCATTCCAGTCGAACTCACACACAACACCAAA |
| | ITR at positions 2049-2178 | TGCATTTAAGTCGCTTGAAATTGCTATAAGCAGAGCAT |
| | AP head sequence at positions | GTTGCGCCAGCATGATTAATACAGCATTTAATACAGAG |
| | 2267-2553 | CCGTGTTTATTGAGTCGGTATTCAGAGTCTGACCAGAA |
| | Splice acceptor sequence at | ATTATTAATCTGGTGAAGTTATTCCTCTGTCATTACGTC |
| | positions 2576-2624 | ATGGTCGATTTCAATTTCTATTGATGCTTTCCAGTCGTA |
| | C-terminal portion of human | ATCAATGATGTATTTTTTGATGTTTGACCTCTGTTCATA |
| | OTOF isoform 5 at positions | TCCTCACAGATAAAAAATCGCCCTCACACTGGAGGGC |
| | 2625-6212 | AAAGAAGATTTCCAATAATCAGAACAAGTCGGCTCCTG |
| | bGH poly(A) sequence at | TTTAGTTACGAGCGACATTGCTCCGTGTATTCACTCGT |
| | positions 6255-6476 | TGGAATGAATACACAGTGCAGTGTTTATTCTGTTATTTA |
| | ITR at positions 6564-6693 | TGCCAAAAATTAAGGCCACTATCAGGCAGCTTTGTTGT |
| | Lambda at positions 6704-8716 | TCTGTTTACCAAGTTCTCTGGCAATCATTGCCGTCGTT |
| | Ori at positions 8773-9361 | CGTATTGCCCATTTATCGACATATTTCCCATCTTCCTAT |
| | KanR at positions 9539-10,348 | ACAGGAAACATTTCTTCAGGCTTAACCATGCATTCCGA |
| | Transgene to be transferred | TTGCAGCTTGCATCCATTGCATCGCTTGAATTGTCCAC |
| | into vector in dual vector | ACCATTGATTTTTATCAATAGTCGTAGTTTAACGGATAG |
| | system at positions 2049-6693 | TCCTGGTATTGTTCCATCACATCCTGAGGATGCCCTTC |
| | | GAACTCTTCAAATTCTTCTTCCTAATATCACCTTAAATA |
| | | GTGGATTGCGGTAGTAAAGATTGTGCCTGTCTTTTAAC |
| | | CACATCAGGCTCGGTGGTTCTCGTGTACCCCTACAGC |
| | | GAGAAATCGGATAAACTATTACAACCCCTACAGTTTGT |
| | | AGAGTATAGAAAATGATCCACTCGTTATTCTCGGACGA |
| | | GTGTTCAGTAATGAACCTCTGGAGAGAACCATCTATAT |
| | | GATCGTTATCTGGGTTTGACTTCTGCTTTTAAGCCCAG |
| | | ATAACTTGCCTGAATATGTTAATGAGAGAATCGGTATT |
| | | CCTCATGTGTGGCATGTTTTCGTCTTTGCTCTTGCATTT |
| | | TCACTAGCAATTAATGTGCATCGATTATCAGCTATTGC |
| | | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGCGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCCCCCG |
| | | GGTGCGCGGCGTCGGTGGTGCCGGCGGGGGGCGCC |
| | | AGGTCGCAGGCGGTGTAGGGCTCCAGGCAGGCGGCG |
| | | AAGGCCATGACGTGCGCTATGAAGGTCTGCTCCTGCA |
| | | CGCCGTGAACCAGGTGCGCCTGCGGGCCGCGCGCGA |
| | | ACACCGCCACGTCCTCGCCTGCGTGGGTCTCTTCGTC |
| | | CAGGGGCACTGCTGACTGCTGCCGATACTCGGGGCT |
| | | CCCGCTCTCGCTCTCGGTAACATCCGGCCGGGCGCC |
| | | GTCCTTGAGCACATAGCCTGGACCGTTTCCTTAAGCG |
| | | ACGCATGCTCGCGATAGGCACCTATTGGTCTTACTGA |
| | | CATCCACTTTGCCTTTCTCTCCACAGGAAAACATGGGG |
| | | CAGCAGGCCAGGATGCTGCGGGCCCAGGTGAAGCGG |
| | | CACACGGTGCGGGACAAGCTGAGGCTGTGCCAGAAC |
| | | TTCCTGCAGAAGCTGCGCTTCCTGGCGGACGAGCCCC |
| | | AGCACAGCATTCCCGACATCTTCATCTGGATGATGAGC |
| | | AACAACAAGCGTGTCGCCTATGCCCGTGTGCCCTCCA |
| | | AGGACCTGCTCTTCTCCATCGTGGAGGAGGAGACTGG |
| | | CAAGGACTGCGCCAAGGTCAAGACGCTCTTCCTTAAG |
| | | CTGCCAGGGAAGCGGGGCTTCGGCTCGGCAGGCTGG |
| | | ACAGTGCAGGCCAAGGTGGAGCTGTACCTGTGGCTG |
| | | GGCCTCAGCAAACAGCGCAAGGAGTTCCTGTGCGGC |
| | | CTGCCCTGTGGCTTCCAGGAGGTCAAGGCAGCCCAG |
| | | GGCCTGGGCCTGCATGCCTTCCCACCCGTCAGCCTG |
| | | GTCTACACCAAGAAGCAGGCGTTCCAGCTCCGAGCGC |
| | | ACATGTACCAGGCCCGCAGCCTCTTTGCCGCCGACAG |
| | | CAGCGGACTCTCAGACCCCTTTGCCCGCGTCTTCTTC |
| | | ATCAATCAGAGTCAGTGCACAGAGGTGCTGAATGAGA |
| | | CCCTGTGTCCCACCTGGGACCAGATGCTGGTGTTCGA |
| | | CAACCTGGAGCTCTATGGTGAAGCTCATGAGCTGAGG |
| | | GACGATCCGCCCATCATTGTCATTGAAATCTATGACCA |
| | | GGATTCCATGGGCAAAGCTGACTTCATGGGCCGGACC |
| | | TTCGCCAAACCCCTGGTGAAGATGGCAGACGAGGCGT |
| | | ACTGCCCACCCCGCTTCCCACCTCAGCTCGAGTACTA |
| | | CCAGATCTACCGTGGCAACGCCACAGCTGGAGACCTG |
| | | CTGGCGGCCTTCGAGCTGCTGCAGATTGGACCAGCAG |
| | | GGAAGGCTGACCTGCCCCCCATCAATGGCCCGGTGG |
| | | ACGTGGACCGAGGTCCCATCATGCCCGTGCCCATGG |
| | | GCATCCGGCCCGTGCTCAGCAAGTACCGAGTGGAGG |
| | | TGCTGTTCTGGGGCCTACGGGACCTAAAGCGGGTGAA |
| | | CCTGGCCCAGGTGGACCGGCCACGGGTGGACATCGA |
| | | GTGTGCAGGGAAGGGGGTGCAGTCGTCCCTGATCCA |
| | | CAATTATAAGAAGAACCCCAACTTCAACACCCTCGTCA |
| | | AGTGGTTTGAAGTGGACCTCCCAGAGAACGAGCTGCT |
| | | GCACCCGCCCTTGAACATCCGTGTGGTGGACTGCCGG |
| | | GCCTTCGGTCGCTACACACTGGTGGGCTCCCATGCCG |
| | | TCAGCTCCCTGCGACGCTTCATCTACCGGCCCCCAGA |
| | | CCGCTCGGCCCCCAGCTGGAACACCACGGTCAGGCT |
| | | TCTCCGGCGCTGCCGTGTGCTGTGCAATGGGGGCTC |
| | | CTCCTCTCACTCCACAGGGGAGGTTGTGGTGACTATG |
| | | GAGCCAGAGGTACCCATCAAGAAACTGGAGACCATGG |
| | | TGAAGCTGGACGCGACTTCTGAAGCTGTTGTCAAGGT |
| | | GGATGTGGCTGAGGAGGAGAAGGAGAAGAAGAAGAA |
| | | GAAGAAGGGCACTGCGGAGGAGCCAGAGGAGGAGGA |
| | | GCCAGACGAGAGCATGCTGGACTGGTGGTCCAAGTAC |
| | | TTTGCCTCCATTGACACCATGAAGGAGCAACTTCGACA |
| | | ACAAGAGCCCTCTGGAATTGACTTGGAGGAGAAGGAG |
| | | GAAGTGGACAATACCGAGGGCCTGAAGGGGTCAATGA |
| | | AGGGCAAGGAGAAGGCAAGGGCTGCCAAAGAGGAGA |
| | | AGAAGAAGAAAACTCAGAGCTCTGGCTCTGGCCAGGG |
| | | GTCCGAGGCCCCCGAGAAGAAGAAACCCAAGATTGAT |
| | | GAGCTTAAGGTATACCCCAAAGAGCTGGAGTCCGAGT |
| | | TTGATAACTTTGAGGACTGGCTGCACACTTTCAACTTG |
| | | CTTCGGGGCAAGACCGGGGATGATGAGGATGGCTCC |
| | | ACCGAGGAGGAGCGCATTGTGGGACGCTTCAAGGGC |
| | | TCCCTCTGCGTGTACAAAGTGCCACTCCCAGAGGACG |
| | | TGTCCCGGGAAGCCGGCTACGACTCCACCTACGGCAT |
| | | GTTCCAGGGCATCCCGAGCAATGACCCCATCAATGTG |
| | | CTGGTCCGAGTCTATGTGGTCCGGGCCACGGACCTGC |
| | | ACCCTGCTGACATCAACGGCAAAGCTGACCCCTACAT |
| | | CGCCATCCGGCTAGGCAAGACTGACATCCGCGACAAG |
| | | GAGAACTACATCTCCAAGCAGCTCAACCCTGTCTTTGG |
| | | GAAGTCCTTTGACATCGAGGCCTCCTTCCCCATGGAAT |
| | | CCATGCTGACGGTGGCTGTGTATGACTGGGACCTGGT |
| | | GGGCACTGATGACCTCATTGGGGAAACCAAGATCGAC |
| | | CTGGAGAACCGCTTCTACAGCAAGCACCGCGCCACCT |
| | | GCGGCATCGCCCAGACCTACTCCACACATGGCTACAA |
| | | TATCTGGCGGGACCCCATGAAGCCCAGCCAGATCCTG |
| | | ACCCGCCTCTGCAAAGACGGCAAAGTGGACGGCCCC |
| | | CACTTTGGGCCCCCTGGGAGAGTGAAGGTGGCCAAC |
| | | CGCGTCTTCACTGGGCCCTCTGAGATTGAGGACGAGA |
| | | ACGGTCAGAGGAAGCCCACAGACGAGCATGTGGCGC |
| | | TGTTGGCCCTGAGGCACTGGGAGGACATCCCCCGCG |
| | | CAGGCTGCCGCCTGGTGCCAGAGCATGTGGAGACGA |
| | | GGCCGCTGCTCAACCCCGACAAGCCGGGCATCGAGC |
| | | AGGGCCGCCTGGAGCTGTGGGTGGACATGTTCCCCAT |
| | | GGACATGCCAGCCCCTGGGACGCCTCTGGACATCTCA |
| | | CCTCGGAAGCCCAAGAAGTACGAGCTGCGGGTCATCA |
| | | TCTGGAACACAGATGAGGTGGTCTTGGAGGACGACGA |
| | | CTTCTTCACAGGGGAGAAGTCCAGTGACATCTTCGTG |
| | | AGGGGGTGGCTGAAGGGCCAGCAGGAGGACAAGCAG |
| | | GACACAGACGTCCACTACCACTCCCTCACTGGCGAGG |
| | | GCAACTTCAACTGGCGCTACCTGTTCCCCTTCGACTAC |
| | | CTGGCGGCGGAGGAGAAGATCGTCATCTCCAAGAAG |
| | | GAGTCCATGTTCTCCTGGGACGAGACCGAGTACAAGA |
| | | TCCCCGCGCGGCTCACCCTGCAGATCTGGGATGCGG |
| | | ACCACTTCTCCGCTGACGACTTCCTGGGGGCCATCGA |
| | | GCTGGACCTGAACCGGTTCCCGCGGGGCGCAAAGAC |
| | | AGCCAAGCAGTGCACCATGGAGATGGCCACCGGGGA |
| | | GGTGGACGTGCCCCTCGTGTCCATCTTCAAGCAAAAG |
| | | CGCGTCAAAGGCTGGTGGCCCCTCCTGGCCCGCAAT |
| | | GAGAACGATGAGTTTGAGCTCACGGGCAAGGTGGAG |
| | | GCTGAGCTGCATTTACTGACAGCAGAGGAGGCAGAGA |
| | | AGAACCCAGTGGGCCTGGCCCGCAATGAACCTGACCC |
| | | CCTAGAGAAACCCAACCGGCCCGACACGGCCTTCGTC |
| | | TGGTTCCTCAACCCTCTCAAGTCCATCAAGTACCTCAT |
| | | CTGCACCCGGTACAAGTGGCTCATCATCAAGATCGTG |
| | | CTGGCGCTGTTGGGGCTGCTCATGTTGGGGCTCTTCC |
| | | TCTACAGCCTCCCTGGCTACATGGTCAAAAAGCTCCTT |
| | | GGGGCATGAACGGCCGCTATGCTAGCTTGGTACCAAG |
| | | GGCGGATCCTGCATAGAGCTCGCTGATCAGCCTCGAC |
| | | TGTGCCTTCTAGTTGCCAGCCATCTGTTGTTTGCCCCT |
| | | CCCCCGTGCCTTCCTTGACCCTGGAAGGTGCCACTCC |
| | | CACTGTCCTTTCCTAATAAAATGAGGAAATTGCATCGC |
| | | ATTGTCTGAGTAGGTGTCATTCTATTCTGGGGGGTGG |
| | | GGTGGGGCAGGACAGCAAGGGGGAGGATTGGGAAGA |
| | | CAATAGCAGGCATCTCGAGTTAAGGGCGAATTCCCGA |
| | | TAAGGATCTTCCTAGAGCATGGCTACGTAGATAAGTAG |
| | | CATGGGGGGTTAATCATTAACTACAAGGAACCCCTAGT |
| | | GATGGAGTTGGCCACTCCCTCTCTGCGCGCTCGCTCG |
| | | CTCACTGAGGCCGGGCGACCAAAGGTCGCCCGACGC |
| | | CCGGGCTTTGCCCGGGGGGCCTCAGTGAGCGAGCGA |
| | | GCGCGCAGCCTTAATTAAATCCACATCTGTATGTTTTTT |
| | | ATATTAATTTATTTTTTGCAGGGGGGCATTGTTTGGTAG |
| | | GTGAGAGTTCTGAATTGCTATGTTTAGTGAGTTGTATC |
| | | TATTTATTTTTCAATAAATACAATTAGTTATGTGTTTTGG |
| | | GGGCGATCGTGAGGCAAAGAAAACCCGGCGCTGAGG |
| | | CCGGGTTATTCTTGTTCTCTGGTCAAATTATATAGTTG |
| | | GAAAACAAGGATGCATATATGAATGAACGATGCAGAG |
| | | GCAATGCCGATGGCGATAGTGGGTATCAGGTAGCCGC |
| | | TTATGCTGGAAAGAAGCAATAACCCGCAGAAAAACAAA |
| | | GCTCCAAGCTCAACAAAACTAAGGGCATAGACAATAAC |
| | | TACCTATGTCATATACCCATACTCTCTAATCTTGGCCA |
| | | GTCGGCGCGTTCTGCTTCCGATTAGAAACGTCAAGGC |
| | | AGCAATCAGGATTGCAATCTTGGTTCCTGCATAGGATG |
| | | ACAATGTCGCCCCAAGACCATCTCTATGAGCTGAAAAA |
| | | GAAACACAAGGAATGTAGTGGCGGAAAAGGAGATAGC |
| | | AAATGCTTACGATAACGTAAGGAATTATTACTATGTAAA |
| | | CACCAGGCAAGATTCTGTTCCGTATAATTACTCCTGAT |
| | | AATTAATCCTTAACTTTGCCCACCTGCCTTTTAAAACAT |
| | | TCCAGTATATCACTTTTCATTCTTGCGTAGCAATATGCC |
| | | CTCTCTTCAGCTATCTCAGCATTGGTGACCTTGTTCAG |
| | | AGGCGCTGAGAGATGGCCTTTTTCTGATAGATAATGTT |
| | | CTGTTAAAATATCTCCGGCCTCATCTTTTGCCCGCAGG |
| | | CTAATGTCTGAAAATTGAGGTGACGGGTTAAAAATAAT |
| | | ATCCTTGGCAACCTTTTTTATATCCCTTTTAAATTTTGG |
| | | CTTAATGACTATATCCAATGAGTCAAAAAGCTCCCCTT |
| | | CAATATCTGTTGCCCCTAAGACCTTTAATATATCGCCA |
| | | AATACAGGTAGCTTGGCTTCTACCTTCACCGTTGTTCT |
| | | GCCGATGAAATGCTAATGCATAACATCGTCTTTGGTGG |
| | | TTCCCCTCATCAGTGGCTCTATCTGAACGCGCTCTCCA |
| | | CTGCTTAATGACATTCCTTTCCCGATTAAAAAATCTGTC |
| | | AGATCGGATGTGGTCGGCCCGAAAACAGTTCTGGCAA |
| | | AACCAATGGTGTCGCCTTCAACAAACAAAAAAGATGGG |
| | | AATCCCAATGATTCGTCATCTGCGAGGCTGTTCTTAAT |
| | | ATCTTCAACTGTAGCTTTAGAGCGATTTATCTTCTGAAC |
| | | CAGACTCTTGTCATTTGTTTTGGTAAAGAGAAAAGTTTT |
| | | TCCATCGATTTTATGAATATACAAATAATTGGAGCCAAC |
| | | CTTCAGGTGATGATTATCAGCCAGCAGAGAATTAAGGA |
| | | AAACAGACAGGTTTATTGAGCACTTATCTTTCCCTTTAT |
| | | TTTTGCTGCGGTAAGTCGCATAAAAACCATTCTTCACA |
| | | ATTCAATCCATTTACTATGTTATGTTCTGAGGGGAGTG |
| | | AAAATTCCCCTAATTCGATGAAGATTCTTGCTAAATTGT |
| | | TATCAGCTATGCGCCGACCAGAACACCTTGCCGATCA |
| | | GCCAAACGTCTAATCAGGCCACTGACTAGCGATAACTT |
| | | TCCCCACAACGGAACAACTCTCATTGCATGGGATAATT |
| | | GGGTACTGTGGGTTTAGTGGTTGTAAAAACACCTGAC |
| | | CGCTATCCCTGATCAGTTTCTTGAAGGTAAACTCATCA |
| | | CCCCCAAGTCTGGCTATACAGAAATCACCTGGCTCAA |
| | | CAGCCTGCTCAGGGTCAACGAGAATTTACATTCCGTCA |
| | | GGATAGCTTGGCTTGGAGCCTGTTGGTGCGGTCACGG |
| | | AATTACCTTCAACCTCAAGCCAGAATGCAGAATCACTG |
| | | GCTTTTTTGGTTGTGCTTACCCATCTCTCCGCATCACC |
| | | TTTGGTAAAGGTTCTAAGCTAAGGTGAGAACATCCCTG |
| | | CCTGAACATGAGAAAAAACAGGGTACTCATACTCACTT |
| | | ATTAGTGACGGCTATGAGCAAAAGGCCAGCAAAAGGC |
| | | CAGGAACCGTAAAAAGGCCGCGTTGCTGGCGTTTTTC |
| | | CATAGGCTCCGCCCCCCTGACGAGCATCACAAAAATC |
| | | GACGCTCAAGTCAGAGGTGGCGAAACCCGACAGGACT |
| | | ATAAAGATACCAGGCGTTTCCCCCTGGAAGCTCCCTC |
| | | GTGCGCTCTCCTGTTCCGACCCTGCCGCTTACCGGAT |
| | | ACCTGTCCGCCTTTCTCCCTTCGGGAAGCGTGGCGCT |
| | | TTCTCATAGCTCACGCTGTAGGTATCTCAGTTCGGTGT |
| | | AGGTCGTTCGCTCCAAGCTGGGCTGTGTGCACGAACC |
| | | CCCCGTTCAGCCCGACCGCTGCGCCTTATCCGGTAAC |
| | | TATCGTCTTGAGTCCAACCCGGTAAGACACGACTTATC |
| | | GCCACTGGCAGCAGCCACTGGTAACAGGATTAGCAGA |
| | | GCGAGGTATGTAGGCGGTGCTACAGAGTTCTTGAAGT |
| | | GGTGGCCTAACTACGGCTACACTAGAAGAACAGTATTT |
| | | GGTATCTGCGCTCTGCTGAAGCCAGTTACCTTCGGAA |
| | | AAAGAGTTGGTAGCTCTTGATCCGGCAAACAAACCAC |
| | | CGCTGGTAGCGGTGGTTTTTTTGTTTGCAAGCAGCAG |
| | | ATTACGCGCAGAAAAAAAGGATCTCAAGAAGATCCTTT |
| | | GATCTTTTCTACGGGGTCTGACGCTCAGTGGAACGAA |
| | | AACTCACGTTAAGGGATTTTGGTCATGAGATTATCAAA |
| | | AAGGATCTTCACCTAGATCCTTTTAAATTAAAAATGAAG |
| | | TTTTAAATCAAGCCCAATCTGAATAATGTTACAACCAAT |
| | | TAACCAATTCTGATTAGAAAAACTCATCGAGCATCAAAT |
| | | GAAACTGCAATTTATTCATATCAGGATTATCAATACCAT |
| | | ATTTTTGAAAAAGCCGTTTCTGTAATGAAGGAGAAAAC |
| | | TCACCGAGGCAGTTCCATAGGATGGCAAGATCCTGGT |
| | | ATCGGTCTGCGATTCCGACTCGTCCAACATCAATACAA |
| | | CCTATTAATTTCCCCTCGTCAAAAATAAGGTTATCAAGT |
| | | GAGAAATCACCATGAGTGACGACTGAATCCGGTGAGA |
| | | ATGGCAAAAGTTTATGCATTTCTTTCCAGACTTGTTCAA |
| | | CAGGCCAGCCATTACGCTCGTCATCAAAATCACTCGC |
| | | ATCAACCAAACCGTTATTCATTCGTGATTGCGCCTGAG |
| | | CAAGACGAAATACGCGATCGCTGTTAAAAGGACAATTA |
| | | CAAACAGGAATCGAATGCAACCGGCGCAGGAACACTG |
| | | CCAGCGCATCAACAATATTTTCACCTGAATCAGGATAT |
| | | TCTTCTAATACCTGGAATGCTGTTTTTCCGGGGATCGC |
| | | AGTGGTGAGTAACCATGCATCATCAGGAGTACGGATA |
| | | AAATGCTTGATGGTCGGAAGAGGCATAAATTCCGTCA |
| | | GCCAGTTTAGTCTGACCATCTCATCTGTAACATCATTG |
| | | GCAACGCTACCTTTGCCATGTTTCAGAAACAACTCTGG |
| | | CGCATCGGGCTTCCCATACAAGCGATAGATTGTCGCA |
| | | CCTGATTGCCCGACATTATCGCGAGCCCATTTATACCC |
| | | ATATAAATCAGCATCCATGTTGGAATTTAATCGCGGCC |
| | | TCGACGTTTCCCGTTGAATATGGCTCATAACACCCCTT |
| | | GTATTACTGTTTATGTAAGCAGACAGTTTTATTGTTCAT |
| | | GATGATATATTTTTATCTTGTGCAATGTAACATCAGAGA |
| | | TTTTGAGACACGGGCCAGAGCTGCA |
| 77 | 5′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGCGGCCTC |
| | Myo15 promoter at positions | AGTGAGCGAGCGAGCGCGCAGAGAGGGAGTGGCCAA |
| | 182-1146 | CTCCATCACTAGGGGTTCCTCAGATCTGAATTCGGTAC |
| | N-terminal portion of human | CTGCAGCTCAGCCTACTACTTGCTTTCCAGGCTGTTCC |
| | OTOF isoform 5 at positions | TAGTTCCCATGTCAGCTGCTTGTGCTTTCCAGAGACAA |
| | 1167-3572 | AACAGGAATAATAGATGTCATTAAATATACATTGGGCC |
| | Splice donor sequence at | CCAGGCGGTCAATGTGGCAGCCTGAGCCTCCTTTCCA |
| | positions 3573-3656 | TCTCTGTGGAGGCAGACATAGGACCCCCAACAAACAG |
| | AP head sequence at positions | CATGCAGGTTGGGAGCCAGCCACAGGACCCAGGTAA |
| | 3663-3949 | GGGGCCCTGGGTCCTTAAGCTTCTGCCACTGGCTCCG |
| | ITR at positions 3973-4115 | GCATTGCAGAGAGAAGAGAAGGGGCGGCAGACTGGA |
| | KanR at positions 4641-5435 | GAGCTGGGCTCCATTTTTGTTCCTTGGTGCCCTGCCC |
| | pUC ori at positions 5821-6491 | CTCCCCATGACCTGCAGAGACATTCAGCCTGCCAGGC |
| | Transgene to be transferred | TTTATGAGGTGGGAGCTGGGCTCTCCCTGATGTATTAT |
| | into vector in dual vector | TCAGCTCCCTGGAGTTGGCCAGCTCCTGTTACACTGG |
| | system at positions 19-4115 | CCACAGCCCTGGGCATCCGCTTCTCACTTCTAGTTTCC |
| | | CCTCCAAGGTAATGTGGTGGGTCATGATCATTCTATCC |
| | | TGGCTTCAGGGACCTGACTCCACTTTGGGGCCATTCG |
| | | AGGGGTCTAGGGTAGATGATGTCCCCCTGTGGGGATT |
| | | AATGTCCTGCTCTGTAAAACTGAGCTAGCTGAGATCCA |
| | | GGAGGGCTTGGCCAGAGACAGCAAGTTGTTGCCATGG |
| | | TGACTTTAAAGCCAGGTTGCTGCCCCAGCACAGGCCT |
| | | CCCAGTCTACCCTCACTAGAAAACAACACCCAGGCAC |
| | | TTTCCACCACCTCTCAAAGGTGAAACCCAAGGCTGGT |
| | | CTAGAGAATGAATTATGGATCCTCGCTGTCCGTGCCAC |
| | | CCAGCTAGTCCCAGCGGCTCAGACACTGAGGAGAGAC |
| | | TGTAGGTTCAGCTACAAGCAAAAAGACCTAGCTGGTCT |
| | | CCAAGCAGTGTCTCCAAGTCCCTGAACCTGTGACACC |
| | | TGCCCCAGGCATCATCAGGCACAGAGGGCCACCGAAT |
| | | TCTAGCGGCCGCCACCATGGCCTTGCTCATCCACCTC |
| | | AAGACAGTCTCGGAGCTGCGGGGCAGGGGCGACCGG |
| | | ATCGCCAAAGTGACTTTCCGAGGGCAATCCTTCTACTC |
| | | TCGGGTCCTGGAGAACTGTGAGGATGTGGCTGACTTT |
| | | GATGAGACATTTCGGTGGCCGGTGGCCAGCAGCATCG |
| | | ACAGAAATGAGATGCTGGAGATTCAGGTTTTCAACTAC |
| | | AGCAAAGTCTTCAGCAACAAGCTCATCGGGACCTTCC |
| | | GCATGGTGCTGCAGAAGGTGGTAGAGGAGAGCCATGT |
| | | GGAGGTGACTGACACGCTGATTGATGACAACAATGCT |
| | | ATCATCAAGACCAGCCTGTGCGTGGAGGTCCGGTATC |
| | | AGGCCACTGACGGCACAGTGGGCTCCTGGGACGATG |
| | | GGGACTTCCTGGGAGATGAGTCTCTTCAAGAGGAAGA |
| | | GAAGGACAGCCAAGAGACGGATGGACTGCTCCCAGG |
| | | CTCCCGGCCCAGCTCCCGGCCCCCAGGAGAGAAGAG |
| | | CTTCCGGAGAGCCGGGAGGAGCGTGTTCTCCGCCAT |
| | | GAAGCTCGGCAAAAACCGGTCTCACAAGGAGGAGCCC |
| | | CAAAGACCAGATGAACCGGCGGTGCTGGAGATGGAA |
| | | GACCTTGACCATCTGGCCATTCGGCTAGGAGATGGAC |
| | | TGGATCCCGACTCGGTGTCTCTAGCCTCAGTCACAGC |
| | | TCTCACCACTAATGTCTCCAACAAGCGATCTAAGCCAG |
| | | ACATTAAGATGGAGCCAAGTGCTGGGCGGCCCATGGA |
| | | TTACCAGGTCAGCATCACGGTGATCGAGGCCCGGCAG |
| | | CTGGTGGGCTTGAACATGGACCCTGTGGTGTGCGTGG |
| | | AGGTGGGTGACGACAAGAAGTACACATCCATGAAGGA |
| | | GTCCACTAACTGCCCCTATTACAACGAGTACTTCGTCT |
| | | TCGACTTCCATGTCTCTCCGGATGTCATGTTTGACAAG |
| | | ATCATCAAGATTTCGGTGATTCACTCCAAGAACCTGCT |
| | | GCGCAGTGGCACCCTGGTGGGCTCCTTCAAAATGGAC |
| | | GTGGGAACCGTGTACTCGCAGCCAGAGCACCAGTTCC |
| | | ATCACAAGTGGGCCATCCTGTCTGACCCCGATGACAT |
| | | CTCCTCGGGGCTGAAGGGCTACGTGAAGTGTGACGTT |
| | | GCCGTGGTGGGCAAAGGGGACAACATCAAGACGCCC |
| | | CACAAGGCCAATGAGACCGACGAAGATGACATTGAGG |
| | | GGAACTTGCTGCTCCCCGAGGGGGTGCCCCCCGAAC |
| | | GCCAGTGGGCCCGGTTCTATGTGAAAATTTACCGAGC |
| | | AGAGGGGCTGCCCCGTATGAACACAAGCCTCATGGCC |
| | | AATGTAAAGAAGGCTTTCATCGGTGAAAACAAGGACCT |
| | | CGTGGACCCCTACGTGCAAGTCTTCTTTGCTGGCCAG |
| | | AAGGGCAAGACTTCAGTGCAGAAGAGCAGCTATGAGC |
| | | CCCTGTGGAATGAGCAGGTCGTCTTTACAGACCTCTTC |
| | | CCCCCACTCTGCAAACGCATGAAGGTGCAGATCCGAG |
| | | ACTCGGACAAGGTCAACGACGTGGCCATCGGCACCCA |
| | | CTTCATTGACCTGCGCAAGATTTCTAATGACGGAGACA |
| | | AAGGCTTCCTGCCCACACTGGGCCCAGCCTGGGTGAA |
| | | CATGTACGGCTCCACACGTAACTACACGCTGCTGGAT |
| | | GAGCATCAGGACCTGAACGAGGGCCTGGGGGAGGGT |
| | | GTGTCCTTCCGGGCCCGGCTCCTGCTGGGCCTGGCT |
| | | GTGGAGATCGTAGACACCTCCAACCCTGAGCTCACCA |
| | | GCTCCACAGAGGTGCAGGTGGAGCAGGCCACGCCCA |
| | | TCTCGGAGAGCTGTGCAGGTAAAATGGAAGAATTCTTT |
| | | CTCTTTGGAGCCTTCCTGGAGGCCTCAATGATCGACC |
| | | GGAGAAACGGAGACAAGCCCATCACCTTTGAGGTCAC |
| | | CATAGGCAACTATGGGAACGAAGTTGATGGCCTGTCC |
| | | CGGCCCCAGCGGCCTCGGCCCCGGAAGGAGCCGGG |
| | | GGATGAGGAAGAAGTAGACCTGATTCAGAACGCAAGT |
| | | GATGACGAGGCCGGTGATGCCGGGGACCTGGCCTCA |
| | | GTCTCCTCCACTCCACCAATGCGGCCCCAGGTCACCG |
| | | ACAGGAACTACTTCCATCTGCCCTACCTGGAGCGAAA |
| | | GCCCTGCATCTACATCAAGAGCTGGTGGCCGGACCAG |
| | | CGCCGCCGCCTCTACAATGCCAACATCATGGACCACA |
| | | TTGCCGACAAGCTGGAAGAAGGCCTGAACGACATACA |
| | | GGAGATGATCAAAACGGAGAAGTCCTACCCTGAGCGT |
| | | CGCCTGCGGGGCGTCCTGGAGGAGCTGAGCTGTGGC |
| | | TGCTGCCGCTTCCTCTCCCTCGCTGACAAGGACCAGG |
| | | GCCACTCATCCCGCACCAGGCTTGACCGGGAGCGCC |
| | | TCAAGTCCTGCATGAGGGAGCTGGTAAGTATCAAGGT |
| | | TACAAGACAGGTTTAAGGAGACCAATAGAAACTGGGC |
| | | TTGTCGAGACAGAGAAGACTCTTGCGTTTCTGAGCTAG |
| | | CCCCCGGGTGCGCGGCGTCGGTGGTGCCGGCGGGG |
| | | GGCGCCAGGTCGCAGGCGGTGTAGGGCTCCAGGCAG |
| | | GCGGCGAAGGCCATGACGTGCGCTATGAAGGTCTGCT |
| | | CCTGCACGCCGTGAACCAGGTGCGCCTGCGGGCCGC |
| | | GCGCGAACACCGCCACGTCCTCGCCTGCGTGGGTCT |
| | | CTTCGTCCAGGGGCACTGCTGACTGCTGCCGATACTC |
| | | GGGGCTCCCGCTCTCGCTCTCGGTAACATCCGGCCG |
| | | GGCGCCGTCCTTGAGCACATAGCCTGGACCGTTTCGT |
| | | CGACTGGGGAGAGATCTGAGGAACCCCTAGTGATGGA |
| | | GTTGGCCACTCCCTCTCTGCGCGCTCGCTCGCTCACT |
| | | GAGGCCGCCCGGGCAAAGCCCGGGCGTCGGGCGAC |
| | | CTTTGGTCGCCCGGCCTCAGTGAGCGAGCGAGCGCG |
| | | CAGAGAGGGAGTGGCCAACCCCCCCCCCCCCCCCCC |
| | | TGCAGCCTGGCGTAATAGCGAAGAGGCCCGCACCGAT |
| | | CGCCCTTCCCAACAGTTGCGTAGCCTGAATGGCGAAT |
| | | GGCGCGACGCGCCCTGTAGCGGCGCATTAAGCGCGG |
| | | CGGGTGTGGTGGTTACGCGCAGCGTGACCGCTACACT |
| | | TGCCAGCGCCCTAGCGCCCGCTCCTTTCGCTTTCTTC |
| | | CCTTCCTTTCTCGCCACGTTCGCCGGCTTTCCCCGTCA |
| | | AGCTCTAAATCGGGGGCTCCCTTTAGGGTTCCGATTTA |
| | | GTGCTTTACGGCACCTCGACCCCAAAAAACTTGATTAG |
| | | GGTGATGGTTCACGTAGTGGGCCATCGCCCTGATAGA |
| | | CGGTTTTTCGCCCTTTGACGTTGGAGTCCACGTTCTTT |
| | | AATAGTGGACTCTTGTTCCAAACTGGAACAACACTCAA |
| | | CCCTATCGCGGTCTATTCTTTTGATTTATAAGGGATGTT |
| | | GCCGATTTCGGCCTATTGGTTAAAAAATGAGCTGATTT |
| | | AACAAAAATTTTAACAAAATTCAGAAGAACTCGTCAAGA |
| | | AGGCGATAGAAGGCGATGCGCTGCGAATCGGGAGCG |
| | | GCGATACCGTAAAGCACGAGGAAGCGGTCAGCCCATT |
| | | CGCCGCCAAGCTCTTCAGCAATATCACGGGTAGCCAA |
| | | CGCTATGTCCTGATAGCGGTCCGCCACACCCAGCCGG |
| | | CCACAGTCGATGAATCCAGAAAAGCGGCCATTTTCCA |
| | | CCATGATATTCGGCAAGCAGGCATCGCCATGGGTCAC |
| | | GACGAGATCCTCGCCGTCGGGCATGCTCGCCTTGAGC |
| | | CTGGCGAACAGTTCGGCTGGCGCGAGCCCCTGATGC |
| | | TCTTCGTCCAGATCATCCTGATCGACAAGACCGGCTTC |
| | | CATCCGAGTACGTGCTCGCTCGATGCGATGTTTCGCT |
| | | TGGTGGTCGAATGGGCAGGTAGCCGGATCAAGCGTAT |
| | | GCAGCCGCCGCATTGCATCAGCCATGATGGATACTTT |
| | | CTCGGCAGGAGCAAGGTGAGATGACAGGAGATCCTG |
| | | CCCCGGCACTTCGCCCAATAGCAGCCAGTCCCTTCCC |
| | | GCTTCAGTGACAACGTCGAGCACAGCTGCGCAAGGAA |
| | | CGCCCGTCGTGGCCAGCCACGATAGCCGCGCTGCCT |
| | | CGTCTTGCAGTTCATTCAGGGCACCGGACAGGTCGGT |
| | | CTTGACAAAAAGAACCGGGCGCCCCTGCGCTGACAGC |
| | | CGGAACACGGCGGCATCAGAGCAGCCGATTGTCTGTT |
| | | GTGCCCAGTCATAGCCGAATAGCCTCTCCACCCAAGC |
| | | GGCCGGAGAACCTGCGTGCAATCCATCTTGTTCAATC |
| | | ATGCGAAACGATCCTCATCCTGTCTCTTGATCAGATCT |
| | | TGATCCCCTGCGCCATCAGATCCTTGGCGGCGAGAAA |
| | | GCCATCCAGTTTACTTTGCAGGGCTTCCCAACCTTACC |
| | | AGAGGGCGCCCCAGCTGGCAATTCCGGTTCGCTTGCT |
| | | GTCCATAAAACCGCCCAGTCTAGCTATCGCCATGTAAG |
| | | CCCACTGCAAGCTACCTGCTTTCTCTTTGCGCTTGCGT |
| | | TTTCCCTTGTCCAGATAGCCCAGTAGCTGACATTCATC |
| | | CGGGGTCAGCACCGTTTCTGCGGACTGGCTTTCTACG |
| | | TGAAAAGGATCTAGGTGAAGATCCTTTTTGATAATCTC |
| | | ATGACCAAAATCCCTTAACGTGAGTTTTCGTTCCACTG |
| | | AGCGTCAGACCCCGTAGAAAAGATCAAAGGATCTTCTT |
| | | GAGATCCTTTTTTTCTGCGCGTAATCTGCTGCTTGCAA |
| | | ACAAAAAAACCACCGCTACCAGCGGTGGTTTGTTTGC |
| | | CGGATCAAGAGCTACCAACTCTTTTTCCGAAGGTAACT |
| | | GGCTTCAGCAGAGCGCAGATACCAAATACTGTCCTTCT |
| | | AGTGTAGCCGTAGTTAGGCCACCACTTCAAGAACTCT |
| | | GTAGCACCGCCTACATACCTCGCTCTGCTAATCCTGTT |
| | | ACCAGTGGCTGCTGCCAGTGGCGATAAGTCGTGTCTT |
| | | ACCGGGTTGGACTCAAGACGATAGTTACCGGATAAGG |
| | | CGCAGCGGTCGGGCTGAACGGGGGGTTCGTGCACAC |
| | | AGCCCAGCTTGGAGCGAACGACCTACACCGAACTGAG |
| | | ATACCTACAGCGTGAGCTATGAGAAAGCGCCACGCTT |
| | | CCCGAAGGGAGAAAGGCGGACAGGTATCCGGTAAGC |
| | | GGCAGGGTCGGAACAGGAGAGCGCACGAGGGAGCTT |
| | | CCAGGGGGAAACGCCTGGTATCTTTATAGTCCTGTCG |
| | | GGTTTCGCCACCTCTGACTTGAGCGTCGATTTTTGTGA |
| | | TGCTCGTCAGGGGGGCGGAGCCTATGGAAAAACGCC |
| | | AGCAACGCGGCCTTTTTACGGTTCCTGGGCTTTTGCT |
| | | GGCCTTTTGCTCACATGTTCTTTCCTGCGTTATCCCCT |
| | | GATTCTGTGGATAACCGTATTACCGCCTTTGAGTGAGC |
| | | TGATACCGCTCGCCGCAGCCGAACGACCGAGCGCAG |
| | | CGAGTCAGTGAGCGAGGAAGCGGAAGAGCGCCCAAT |
| | | ACGCAAACCGCCTCTCCCCGCGCGTTGGCCGATTCAT |
| | | TAATGCAGGGCTGCA |
| 78 | 3′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGCGGCCTC |
| | AP head sequence at positions | AGTGAGCGAGCGAGCGCGCAGAGAGGGAGTGGCCAA |
| | 187-473 | CTCCATCACTAGGGGTTCCTCAGATCTGAATTCTAGCG |
| | Splice acceptor sequence at | GCCGCCCCCGGGTGCGCGGCGTCGGTGGTGCCGGC |
| | positions 496-544 | GGGGGGCGCCAGGTCGCAGGCGGTGTAGGGCTCCA |
| | C-terminal portion of human | GGCAGGCGGCGAAGGCCATGACGTGCGCTATGAAGG |
| | OTOF isoform 5 at positions | TCTGCTCCTGCACGCCGTGAACCAGGTGCGCCTGCG |
| | 545-4132 | GGCCGCGCGCGAACACCGCCACGTCCTCGCCTGCGT |
| | bGH poly(A) sequence at | GGGTCTCTTCGTCCAGGGGCACTGCTGACTGCTGCCG |
| | positions 4175-4396 | ATACTCGGGGCTCCCGCTCTCGCTCTCGGTAACATCC |
| | ITR at positions 4447-4589 | GGCCGGGCGCCGTCCTTGAGCACATAGCCTGGACCG |
| | KanR at positions 5115-5909 | TTTCCTTAAGCGACGCATGCTCGCGATAGGCACCTATT |
| | pUC ori at positions 6295-6965 | GGTCTTACTGACATCCACTTTGCCTTTCTCTCCACAGG |
| | Transgene to be transferred | AAAACATGGGGCAGCAGGCCAGGATGCTGCGGGCCC |
| | into vector in dual vector | AGGTGAAGCGGCACACGGTGCGGGACAAGCTGAGGC |
| | system at positions 19-4589 | TGTGCCAGAACTTCCTGCAGAAGCTGCGCTTCCTGGC |
| | | GGACGAGCCCCAGCACAGCATTCCCGACATCTTCATC |
| | | TGGATGATGAGCAACAACAAGCGTGTCGCCTATGCCC |
| | | GTGTGCCCTCCAAGGACCTGCTCTTCTCCATCGTGGA |
| | | GGAGGAGACTGGCAAGGACTGCGCCAAGGTCAAGAC |
| | | GCTCTTCCTTAAGCTGCCAGGGAAGCGGGGCTTCGGC |
| | | TCGGCAGGCTGGACAGTGCAGGCCAAGGTGGAGCTG |
| | | TACCTGTGGCTGGGCCTCAGCAAACAGCGCAAGGAGT |
| | | TCCTGTGCGGCCTGCCCTGTGGCTTCCAGGAGGTCAA |
| | | GGCAGCCCAGGGCCTGGGCCTGCATGCCTTCCCACC |
| | | CGTCAGCCTGGTCTACACCAAGAAGCAGGCGTTCCAG |
| | | CTCCGAGCGCACATGTACCAGGCCCGCAGCCTCTTTG |
| | | CCGCCGACAGCAGCGGACTCTCAGACCCCTTTGCCCG |
| | | CGTCTTCTTCATCAATCAGAGTCAGTGCACAGAGGTGC |
| | | TGAATGAGACCCTGTGTCCCACCTGGGACCAGATGCT |
| | | GGTGTTCGACAACCTGGAGCTCTATGGTGAAGCTCAT |
| | | GAGCTGAGGGACGATCCGCCCATCATTGTCATTGAAA |
| | | TCTATGACCAGGATTCCATGGGCAAAGCTGACTTCATG |
| | | GGCCGGACCTTCGCCAAACCCCTGGTGAAGATGGCA |
| | | GACGAGGCGTACTGCCCACCCCGCTTCCCACCTCAGC |
| | | TCGAGTACTACCAGATCTACCGTGGCAACGCCACAGC |
| | | TGGAGACCTGCTGGCGGCCTTCGAGCTGCTGCAGATT |
| | | GGACCAGCAGGGAAGGCTGACCTGCCCCCCATCAAT |
| | | GGCCCGGTGGACGTGGACCGAGGTCCCATCATGCCC |
| | | GTGCCCATGGGCATCCGGCCCGTGCTCAGCAAGTACC |
| | | GAGTGGAGGTGCTGTTCTGGGGCCTACGGGACCTAAA |
| | | GCGGGTGAACCTGGCCCAGGTGGACCGGCCACGGGT |
| | | GGACATCGAGTGTGCAGGGAAGGGGGTGCAGTCGTC |
| | | CCTGATCCACAATTATAAGAAGAACCCCAACTTCAACA |
| | | CCCTCGTCAAGTGGTTTGAAGTGGACCTCCCAGAGAA |
| | | CGAGCTGCTGCACCCGCCCTTGAACATCCGTGTGGTG |
| | | GACTGCCGGGCCTTCGGTCGCTACACACTGGTGGGCT |
| | | CCCATGCCGTCAGCTCCCTGCGACGCTTCATCTACCG |
| | | GCCCCCAGACCGCTCGGCCCCCAGCTGGAACACCAC |
| | | GGTCAGGCTTCTCCGGCGCTGCCGTGTGCTGTGCAAT |
| | | GGGGGCTCCTCCTCTCACTCCACAGGGGAGGTTGTG |
| | | GTGACTATGGAGCCAGAGGTACCCATCAAGAAACTGG |
| | | AGACCATGGTGAAGCTGGACGCGACTTCTGAAGCTGT |
| | | TGTCAAGGTGGATGTGGCTGAGGAGGAGAAGGAGAA |
| | | GAAGAAGAAGAAGAAGGGCACTGCGGAGGAGCCAGA |
| | | GGAGGAGGAGCCAGACGAGAGCATGCTGGACTGGTG |
| | | GTCCAAGTACTTTGCCTCCATTGACACCATGAAGGAGC |
| | | AACTTCGACAACAAGAGCCCTCTGGAATTGACTTGGA |
| | | GGAGAAGGAGGAAGTGGACAATACCGAGGGCCTGAA |
| | | GGGGTCAATGAAGGGCAAGGAGAAGGCAAGGGCTGC |
| | | CAAAGAGGAGAAGAAGAAGAAAACTCAGAGCTCTGGC |
| | | TCTGGCCAGGGGTCCGAGGCCCCCGAGAAGAAGAAA |
| | | CCCAAGATTGATGAGCTTAAGGTATACCCCAAAGAGCT |
| | | GGAGTCCGAGTTTGATAACTTTGAGGACTGGCTGCAC |
| | | ACTTTCAACTTGCTTCGGGGCAAGACCGGGGATGATG |
| | | AGGATGGCTCCACCGAGGAGGAGCGCATTGTGGGAC |
| | | GCTTCAAGGGCTCCCTCTGCGTGTACAAAGTGCCACT |
| | | CCCAGAGGACGTGTCCCGGGAAGCCGGCTACGACTC |
| | | CACCTACGGCATGTTCCAGGGCATCCCGAGCAATGAC |
| | | CCCATCAATGTGCTGGTCCGAGTCTATGTGGTCCGGG |
| | | CCACGGACCTGCACCCTGCTGACATCAACGGCAAAGC |
| | | TGACCCCTACATCGCCATCCGGCTAGGCAAGACTGAC |
| | | ATCCGCGACAAGGAGAACTACATCTCCAAGCAGCTCA |
| | | ACCCTGTCTTTGGGAAGTCCTTTGACATCGAGGCCTC |
| | | CTTCCCCATGGAATCCATGCTGACGGTGGCTGTGTAT |
| | | GACTGGGACCTGGTGGGCACTGATGACCTCATTGGGG |
| | | AAACCAAGATCGACCTGGAGAACCGCTTCTACAGCAA |
| | | GCACCGCGCCACCTGCGGCATCGCCCAGACCTACTC |
| | | CACACATGGCTACAATATCTGGCGGGACCCCATGAAG |
| | | CCCAGCCAGATCCTGACCCGCCTCTGCAAAGACGGCA |
| | | AAGTGGACGGCCCCCACTTTGGGCCCCCTGGGAGAG |
| | | TGAAGGTGGCCAACCGCGTCTTCACTGGGCCCTCTGA |
| | | GATTGAGGACGAGAACGGTCAGAGGAAGCCCACAGA |
| | | CGAGCATGTGGCGCTGTTGGCCCTGAGGCACTGGGA |
| | | GGACATCCCCCGCGCAGGCTGCCGCCTGGTGCCAGA |
| | | GCATGTGGAGACGAGGCCGCTGCTCAACCCCGACAA |
| | | GCCGGGCATCGAGCAGGGCCGCCTGGAGCTGTGGGT |
| | | GGACATGTTCCCCATGGACATGCCAGCCCCTGGGACG |
| | | CCTCTGGACATCTCACCTCGGAAGCCCAAGAAGTACG |
| | | AGCTGCGGGTCATCATCTGGAACACAGATGAGGTGGT |
| | | CTTGGAGGACGACGACTTCTTCACAGGGGAGAAGTCC |
| | | AGTGACATCTTCGTGAGGGGGTGGCTGAAGGGCCAG |
| | | CAGGAGGACAAGCAGGACACAGACGTCCACTACCACT |
| | | CCCTCACTGGCGAGGGCAACTTCAACTGGCGCTACCT |
| | | GTTCCCCTTCGACTACCTGGCGGCGGAGGAGAAGATC |
| | | GTCATCTCCAAGAAGGAGTCCATGTTCTCCTGGGACG |
| | | AGACCGAGTACAAGATCCCCGCGCGGCTCACCCTGCA |
| | | GATCTGGGATGCGGACCACTTCTCCGCTGACGACTTC |
| | | CTGGGGGCCATCGAGCTGGACCTGAACCGGTTCCCG |
| | | CGGGGCGCAAAGACAGCCAAGCAGTGCACCATGGAG |
| | | ATGGCCACCGGGGAGGTGGACGTGCCCCTCGTGTCC |
| | | ATCTTCAAGCAAAAGCGCGTCAAAGGCTGGTGGCCCC |
| | | TCCTGGCCCGCAATGAGAACGATGAGTTTGAGCTCAC |
| | | GGGCAAGGTGGAGGCTGAGCTGCATTTACTGACAGCA |
| | | GAGGAGGCAGAGAAGAACCCAGTGGGCCTGGCCCGC |
| | | AATGAACCTGACCCCCTAGAGAAACCCAACCGGCCCG |
| | | ACACGGCCTTCGTCTGGTTCCTCAACCCTCTCAAGTCC |
| | | ATCAAGTACCTCATCTGCACCCGGTACAAGTGGCTCAT |
| | | CATCAAGATCGTGCTGGCGCTGTTGGGGCTGCTCATG |
| | | TTGGGGCTCTTCCTCTACAGCCTCCCTGGCTACATGG |
| | | TCAAAAAGCTCCTTGGGGCATGAACGGCCGCTATGCT |
| | | AGCTTGGTACCAAGGGCGGATCCTGCATAGAGCTCGC |
| | | TGATCAGCCTCGACTGTGCCTTCTAGTTGCCAGCCATC |
| | | TGTTGTTTGCCCCTCCCCCGTGCCTTCCTTGACCCTG |
| | | GAAGGTGCCACTCCCACTGTCCTTTCCTAATAAAATGA |
| | | GGAAATTGCATCGCATTGTCTGAGTAGGTGTCATTCTA |
| | | TTCTGGGGGGTGGGGTGGGGCAGGACAGCAAGGGG |
| | | GAGGATTGGGAAGACAATAGCAGGCATGCTGGGGAG |
| | | AGATCTGAGGACTAGTCCGTCGACTGGGGAGAGATCT |
| | | GAGGAACCCCTAGTGATGGAGTTGGCCACTCCCTCTC |
| | | TGCGCGCTCGCTCGCTCACTGAGGCCGCCCGGGCAA |
| | | AGCCCGGGCGTCGGGCGACCTTTGGTCGCCCGGCCT |
| | | CAGTGAGCGAGCGAGCGCGCAGAGAGGGAGTGGCCA |
| | | ACCCCCCCCCCCCCCCCCCTGCAGCCTGGCGTAATAG |
| | | CGAAGAGGCCCGCACCGATCGCCCTTCCCAACAGTTG |
| | | CGTAGCCTGAATGGCGAATGGCGCGACGCGCCCTGT |
| | | AGCGGCGCATTAAGCGCGGCGGGTGTGGTGGTTACG |
| | | CGCAGCGTGACCGCTACACTTGCCAGCGCCCTAGCG |
| | | CCCGCTCCTTTCGCTTTCTTCCCTTCCTTTCTCGCCAC |
| | | GTTCGCCGGCTTTCCCCGTCAAGCTCTAAATCGGGGG |
| | | CTCCCTTTAGGGTTCCGATTTAGTGCTTTACGGCACCT |
| | | CGACCCCAAAAAACTTGATTAGGGTGATGGTTCACGTA |
| | | GTGGGCCATCGCCCTGATAGACGGTTTTTCGCCCTTT |
| | | GACGTTGGAGTCCACGTTCTTTAATAGTGGACTCTTGT |
| | | TCCAAACTGGAACAACACTCAACCCTATCGCGGTCTAT |
| | | TCTTTTGATTTATAAGGGATGTTGCCGATTTCGGCCTA |
| | | TTGGTTAAAAAATGAGCTGATTTAACAAAAATTTTAACA |
| | | AAATTCAGAAGAACTCGTCAAGAAGGCGATAGAAGGC |
| | | GATGCGCTGCGAATCGGGAGCGGCGATACCGTAAAG |
| | | CACGAGGAAGCGGTCAGCCCATTCGCCGCCAAGCTCT |
| | | TCAGCAATATCACGGGTAGCCAACGCTATGTCCTGATA |
| | | GCGGTCCGCCACACCCAGCCGGCCACAGTCGATGAA |
| | | TCCAGAAAAGCGGCCATTTTCCACCATGATATTCGGCA |
| | | AGCAGGCATCGCCATGGGTCACGACGAGATCCTCGCC |
| | | GTCGGGCATGCTCGCCTTGAGCCTGGCGAACAGTTCG |
| | | GCTGGCGCGAGCCCCTGATGCTCTTCGTCCAGATCAT |
| | | CCTGATCGACAAGACCGGCTTCCATCCGAGTACGTGC |
| | | TCGCTCGATGCGATGTTTCGCTTGGTGGTCGAATGGG |
| | | CAGGTAGCCGGATCAAGCGTATGCAGCCGCCGCATTG |
| | | CATCAGCCATGATGGATACTTTCTCGGCAGGAGCAAG |
| | | GTGAGATGACAGGAGATCCTGCCCCGGCACTTCGCCC |
| | | AATAGCAGCCAGTCCCTTCCCGCTTCAGTGACAACGT |
| | | CGAGCACAGCTGCGCAAGGAACGCCCGTCGTGGCCA |
| | | GCCACGATAGCCGCGCTGCCTCGTCTTGCAGTTCATT |
| | | CAGGGCACCGGACAGGTCGGTCTTGACAAAAAGAACC |
| | | GGGCGCCCCTGCGCTGACAGCCGGAACACGGCGGCA |
| | | TCAGAGCAGCCGATTGTCTGTTGTGCCCAGTCATAGC |
| | | CGAATAGCCTCTCCACCCAAGCGGCCGGAGAACCTGC |
| | | GTGCAATCCATCTTGTTCAATCATGCGAAACGATCCTC |
| | | ATCCTGTCTCTTGATCAGATCTTGATCCCCTGCGCCAT |
| | | CAGATCCTTGGCGGCGAGAAAGCCATCCAGTTTACTTT |
| | | GCAGGGCTTCCCAACCTTACCAGAGGGCGCCCCAGCT |
| | | GGCAATTCCGGTTCGCTTGCTGTCCATAAAACCGCCC |
| | | AGTCTAGCTATCGCCATGTAAGCCCACTGCAAGCTAC |
| | | CTGCTTTCTCTTTGCGCTTGCGTTTTCCCTTGTCCAGA |
| | | TAGCCCAGTAGCTGACATTCATCCGGGGTCAGCACCG |
| | | TTTCTGCGGACTGGCTTTCTACGTGAAAAGGATCTAGG |
| | | TGAAGATCCTTTTTGATAATCTCATGACCAAAATCCCTT |
| | | AACGTGAGTTTTCGTTCCACTGAGCGTCAGACCCCGT |
| | | AGAAAAGATCAAAGGATCTTCTTGAGATCCTTTTTTTCT |
| | | GCGCGTAATCTGCTGCTTGCAAACAAAAAAACCACCG |
| | | CTACCAGCGGTGGTTTGTTTGCCGGATCAAGAGCTAC |
| | | CAACTCTTTTTCCGAAGGTAACTGGCTTCAGCAGAGCG |
| | | CAGATACCAAATACTGTCCTTCTAGTGTAGCCGTAGTT |
| | | AGGCCACCACTTCAAGAACTCTGTAGCACCGCCTACA |
| | | TACCTCGCTCTGCTAATCCTGTTACCAGTGGCTGCTGC |
| | | CAGTGGCGATAAGTCGTGTCTTACCGGGTTGGACTCA |
| | | AGACGATAGTTACCGGATAAGGCGCAGCGGTCGGGCT |
| | | GAACGGGGGGTTCGTGCACACAGCCCAGCTTGGAGC |
| | | GAACGACCTACACCGAACTGAGATACCTACAGCGTGA |
| | | GCTATGAGAAAGCGCCACGCTTCCCGAAGGGAGAAAG |
| | | GCGGACAGGTATCCGGTAAGCGGCAGGGTCGGAACA |
| | | GGAGAGCGCACGAGGGAGCTTCCAGGGGGAAACGCC |
| | | TGGTATCTTTATAGTCCTGTCGGGTTTCGCCACCTCTG |
| | | ACTTGAGCGTCGATTTTTGTGATGCTCGTCAGGGGGG |
| | | CGGAGCCTATGGAAAAACGCCAGCAACGCGGCCTTTT |
| | | TACGGTTCCTGGGCTTTTGCTGGCCTTTTGCTCACATG |
| | | TTCTTTCCTGCGTTATCCCCTGATTCTGTGGATAACCG |
| | | TATTACCGCCTTTGAGTGAGCTGATACCGCTCGCCGC |
| | | AGCCGAACGACCGAGCGCAGCGAGTCAGTGAGCGAG |
| | | GAAGCGGAAGAGCGCCCAATACGCAAACCGCCTCTCC |
| | | CCGCGCGTTGGCCGATTCATTAATGCAGGGCTGCA |
| 79 | 5′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda at positions 53-2027 | TTTCCATCATTCCAGTCGAACTCACACACAACACCAAA |
| | ITR at positions 2049-2178 | TGCATTTAAGTCGCTTGAAATTGCTATAAGCAGAGCAT |
| | CMV i.e enhancer at positions | GTTGCGCCAGCATGATTAATACAGCATTTAATACAGAG |
| | 2267-2636 (part of smCBA | CCGTGTTTATTGAGTCGGTATTCAGAGTCTGACCAGAA |
| | promoter) | ATTATTAATCTGGTGAAGTTATTCCTCTGTCATTACGTC |
| | Chicken β-actin promoter at | ATGGTCGATTTCAATTTCTATTGATGCTTTCCAGTCGTA |
| | positions 2633-2915 (part of | ATCAATGATGTATTTTTTGATGTTTGACCTCTGTTCATA |
| | smCBA promoter) | TCCTCACAGATAAAAAATCGCCCTCACACTGGAGGGC |
| | Exon1 at positions 2916-3008 | AAAGAAGATTTCCAATAATCAGAACAAGTCGGCTCCTG |
| | (part of smCBA promoter) | TTTAGTTACGAGCGACATTGCTCCGTGTATTCACTCGT |
| | Chimeric intron at positions | TGGAATGAATACACAGTGCAGTGTTTATTCTGTTATTTA |
| | 3008-3209 (part of smCBA | TGCCAAAAATTAAGGCCACTATCAGGCAGCTTTGTTGT |
| | promoter) | TCTGTTTACCAAGTTCTCTGGCAATCATTGCCGTCGTT |
| | Kozak sequence at positions | CGTATTGCCCATTTATCGACATATTTCCCATCTTCCTAT |
| | 3226-3235 | ACAGGAAACATTTCTTCAGGCTTAACCATGCATTCCGA |
| | N-terminal portion of human | TTGCAGCTTGCATCCATTGCATCGCTTGAATTGTCCAC |
| | OTOF isoform 5 at positions | ACCATTGATTTTTATCAATAGTCGTAGTTTAACGGATAG |
| | 3232-5637 | TCCTGGTATTGTTCCATCACATCCTGAGGATGCCCTTC |
| | Splice donor sequence at | GAACTCTTCAAATTCTTCTTCCTAATATCACCTTAAATA |
| | positions 5638-5721 | GTGGATTGCGGTAGTAAAGATTGTGCCTGTCTTTTAAC |
| | AP head sequence at positions | CACATCAGGCTCGGTGGTTCTCGTGTACCCCTACAGC |
| | 5728-6014 | GAGAAATCGGATAAACTATTACAACCCCTACAGTTTGT |
| | ITR at positions 6108-6237 | AGAGTATAGAAAATGATCCACTCGTTATTCTCGGACGA |
| | Lambda at positions 6248-8260 | GTGTTCAGTAATGAACCTCTGGAGAGAACCATCTATAT |
| | Ori at positions 8317-8905 | GATCGTTATCTGGGTTTGACTTCTGCTTTTAAGCCCAG |
| | KanR at positions 9083-9892 | ATAACTTGCCTGAATATGTTAATGAGAGAATCGGTATT |
| | Transgene to be transferred | CCTCATGTGTGGCATGTTTTCGTCTTTGCTCTTGCATTT |
| | into vector in dual vector | TCACTAGCAATTAATGTGCATCGATTATCAGCTATTGC |
| | system at positions 2049-6237 | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGGGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCGGTACC |
| | | TAGTTATTAATAGTAATCAATTACGGGGTCATTAGTTCA |
| | | TAGCCCATATATGGAGTTCCGCGTTACATAACTTACGG |
| | | TAAATGGCCCGCCTGGCTGACCGCCCAACGACCCCC |
| | | GCCCATTGACGTCAATAATGACGTATGTTCCCATAGTA |
| | | ACGCCAATAGGGACTTTCCATTGACGTCAATGGGTGG |
| | | AGTATTTACGGTAAACTGCCCACTTGGCAGTACATCAA |
| | | GTGTATCATATGCCAAGTACGCCCCCTATTGACGTCAA |
| | | TGACGGTAAATGGCCCGCCTGGCATTATGCCCAGTAC |
| | | ATGACCTTATGGGACTTTCCTACTTGGCAGTACATCTA |
| | | CGTATTAGTCATCGCTATTACCATGGTCGAGGTGAGCC |
| | | CCACGTTCTGCTTCACTCTCCCCATCTCCCCCCCCTCC |
| | | CCACCCCCAATTTTGTATTTATTTATTTTTTAATTATTTT |
| | | GTGCAGCGATGGGGGCGGGGGGGGGGGGGGGGGCG |
| | | CGCGCCAGGCGGGGCGGGGCGGGGCGAGGGGCGG |
| | | GGCGGGGCGAGGCGGAGAGGTGCGGCGGCAGCCAA |
| | | TCAGAGCGGCGCGCTCCGAAAGTTTCCTTTTATGGCG |
| | | AGGCGGCGGCGGCGGCGGCCCTATAAAAAGCGAAGC |
| | | GCGCGGGGGGCGGGAGTCGCTGCGCGCTGCCTTCGC |
| | | CCCGTGCCCCGCTCCGCCGCCGCCTCGCGCCGCCCG |
| | | CCCCGGCTCTGACTGACCGCGTTACTCCCACAGGTGA |
| | | GCGGGCGGGACGGCCCTTCTCCTCCGGGCTGTAATTA |
| | | GCGCTTGGTTTAATGACGGCTTGTTTCTTTTCTGTGGC |
| | | TGCGTGAAAGCCTTGAGGGGCTCCGGGAGCTAGAGC |
| | | CTCTGCTAACCATGTTCATGCCTTCTTCTTTTTCCTACA |
| | | GCTCCTGGGCAACGTGCTGGTTATTGTGCTGTCTCAT |
| | | CATTTTGGCAAAGAATTCTAGCGGCCGCCACCATGGC |
| | | CTTGCTCATCCACCTCAAGACAGTCTCGGAGCTGCGG |
| | | GGCAGGGGCGACCGGATCGCCAAAGTGACTTTCCGA |
| | | GGGCAATCCTTCTACTCTCGGGTCCTGGAGAACTGTG |
| | | AGGATGTGGCTGACTTTGATGAGACATTTCGGTGGCC |
| | | GGTGGCCAGCAGCATCGACAGAAATGAGATGCTGGAG |
| | | ATTCAGGTTTTCAACTACAGCAAAGTCTTCAGCAACAA |
| | | GCTCATCGGGACCTTCCGCATGGTGCTGCAGAAGGTG |
| | | GTAGAGGAGAGCCATGTGGAGGTGACTGACACGCTGA |
| | | TTGATGACAACAATGCTATCATCAAGACCAGCCTGTGC |
| | | GTGGAGGTCCGGTATCAGGCCACTGACGGCACAGTG |
| | | GGCTCCTGGGACGATGGGGACTTCCTGGGAGATGAG |
| | | TCTCTTCAAGAGGAAGAGAAGGACAGCCAAGAGACGG |
| | | ATGGACTGCTCCCAGGCTCCCGGCCCAGCTCCCGGC |
| | | CCCCAGGAGAGAAGAGCTTCCGGAGAGCCGGGAGGA |
| | | GCGTGTTCTCCGCCATGAAGCTCGGCAAAAACCGGTC |
| | | TCACAAGGAGGAGCCCCAAAGACCAGATGAACCGGC |
| | | GGTGCTGGAGATGGAAGACCTTGACCATCTGGCCATT |
| | | CGGCTAGGAGATGGACTGGATCCCGACTCGGTGTCTC |
| | | TAGCCTCAGTCACAGCTCTCACCACTAATGTCTCCAAC |
| | | AAGCGATCTAAGCCAGACATTAAGATGGAGCCAAGTG |
| | | CTGGGCGGCCCATGGATTACCAGGTCAGCATCACGGT |
| | | GATCGAGGCCCGGCAGCTGGTGGGCTTGAACATGGA |
| | | CCCTGTGGTGTGCGTGGAGGTGGGTGACGACAAGAA |
| | | GTACACATCCATGAAGGAGTCCACTAACTGCCCCTATT |
| | | ACAACGAGTACTTCGTCTTCGACTTCCATGTCTCTCCG |
| | | GATGTCATGTTTGACAAGATCATCAAGATTTCGGTGAT |
| | | TCACTCCAAGAACCTGCTGCGCAGTGGCACCCTGGTG |
| | | GGCTCCTTCAAAATGGACGTGGGAACCGTGTACTCGC |
| | | AGCCAGAGCACCAGTTCCATCACAAGTGGGCCATCCT |
| | | GTCTGACCCCGATGACATCTCCTCGGGGCTGAAGGGC |
| | | TACGTGAAGTGTGACGTTGCCGTGGTGGGCAAAGGG |
| | | GACAACATCAAGACGCCCCACAAGGCCAATGAGACCG |
| | | ACGAAGATGACATTGAGGGGAACTTGCTGCTCCCCGA |
| | | GGGGGTGCCCCCCGAACGCCAGTGGGCCCGGTTCTA |
| | | TGTGAAAATTTACCGAGCAGAGGGGCTGCCCCGTATG |
| | | AACACAAGCCTCATGGCCAATGTAAAGAAGGCTTTCAT |
| | | CGGTGAAAACAAGGACCTCGTGGACCCCTACGTGCAA |
| | | GTCTTCTTTGCTGGCCAGAAGGGCAAGACTTCAGTGC |
| | | AGAAGAGCAGCTATGAGCCCCTGTGGAATGAGCAGGT |
| | | CGTCTTTACAGACCTCTTCCCCCCACTCTGCAAACGCA |
| | | TGAAGGTGCAGATCCGAGACTCGGACAAGGTCAACGA |
| | | CGTGGCCATCGGCACCCACTTCATTGACCTGCGCAAG |
| | | ATTTCTAATGACGGAGACAAAGGCTTCCTGCCCACACT |
| | | GGGCCCAGCCTGGGTGAACATGTACGGCTCCACACGT |
| | | AACTACACGCTGCTGGATGAGCATCAGGACCTGAACG |
| | | AGGGCCTGGGGGAGGGTGTGTCCTTCCGGGCCCGGC |
| | | TCCTGCTGGGCCTGGCTGTGGAGATCGTAGACACCTC |
| | | CAACCCTGAGCTCACCAGCTCCACAGAGGTGCAGGTG |
| | | GAGCAGGCCACGCCCATCTCGGAGAGCTGTGCAGGT |
| | | AAAATGGAAGAATTCTTTCTCTTTGGAGCCTTCCTGGA |
| | | GGCCTCAATGATCGACCGGAGAAACGGAGACAAGCCC |
| | | ATCACCTTTGAGGTCACCATAGGCAACTATGGGAACG |
| | | AAGTTGATGGCCTGTCCCGGCCCCAGCGGCCTCGGC |
| | | CCCGGAAGGAGCCGGGGGATGAGGAAGAAGTAGACC |
| | | TGATTCAGAACGCAAGTGATGACGAGGCCGGTGATGC |
| | | CGGGGACCTGGCCTCAGTCTCCTCCACTCCACCAATG |
| | | CGGCCCCAGGTCACCGACAGGAACTACTTCCATCTGC |
| | | CCTACCTGGAGCGAAAGCCCTGCATCTACATCAAGAG |
| | | CTGGTGGCCGGACCAGCGCCGCCGCCTCTACAATGC |
| | | CAACATCATGGACCACATTGCCGACAAGCTGGAAGAA |
| | | GGCCTGAACGACATACAGGAGATGATCAAAACGGAGA |
| | | AGTCCTACCCTGAGCGTCGCCTGCGGGGCGTCCTGG |
| | | AGGAGCTGAGCTGTGGCTGCTGCCGCTTCCTCTCCCT |
| | | CGCTGACAAGGACCAGGGCCACTCATCCCGCACCAG |
| | | GCTTGACCGGGAGCGCCTCAAGTCCTGCATGAGGGA |
| | | GCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACCTCGAGTTAAGGGCGA |
| | | ATTCCCGATAAGGATCTTCCTAGAGCATGGCTACGTAG |
| | | ATAAGTAGCATGGGGGTTAATCATTAACTACAAGGAA |
| | | CCCCTAGTGATGGAGTTGGCCACTCCCTCTCTGCGCG |
| | | CTCGCTCGCTCACTGAGGCCGGGCGACCAAAGGTCG |
| | | CCCGACGCCCGGGCTTTGCCCGGGGGGCCTCAGTGA |
| | | GCGAGCGAGCGCGCAGCCTTAATTAAATCCACATCTG |
| | | TATGTTTTTTATATTAATTTATTTTTTGCAGGGGGGCAT |
| | | TGTTTGGTAGGTGAGAGTTCTGAATTGCTATGTTTAGT |
| | | GAGTTGTATCTATTTATTTTTCAATAAATACAATTAGTTA |
| | | TGTGTTTTGGGGGCGATCGTGAGGCAAAGAAAACCCG |
| | | GCGCTGAGGCCGGGTTATTCTTGTTCTCTGGTCAAATT |
| | | ATATAGTTGGAAAACAAGGATGCATATATGAATGAACG |
| | | ATGCAGAGGCAATGCCGATGGCGATAGTGGGTATCAG |
| | | GTAGCCGCTTATGCTGGAAAGAAGCAATAACCCGCAG |
| | | AAAAACAAAGCTCCAAGCTCAACAAAACTAAGGGCATA |
| | | GACAATAACTACCTATGTCATATACCCATACTCTCTAAT |
| | | CTTGGCCAGTCGGCGCGTTCTGCTTCCGATTAGAAAC |
| | | GTCAAGGCAGCAATCAGGATTGCAATCTTGGTTCCTG |
| | | CATAGGATGACAATGTCGCCCCAAGACCATCTCTATGA |
| | | GCTGAAAAAGAAACACAAGGAATGTAGTGGCGGAAAA |
| | | GGAGATAGCAAATGCTTACGATAACGTAAGGAATTATT |
| | | ACTATGTAAACACCAGGCAAGATTCTGTTCCGTATAAT |
| | | TACTCCTGATAATTAATCCTTAACTTTGCCCACCTGCCT |
| | | TTTAAAACATTCCAGTATATCACTTTTCATTCTTGCGTA |
| | | GCAATATGCCCTCTCTTCAGCTATCTCAGCATTGGTGA |
| | | CCTTGTTCAGAGGCGCTGAGAGATGGCCTTTTTCTGAT |
| | | AGATAATGTTCTGTTAAAATATCTCCGGCCTCATCTTTT |
| | | GCCCGCAGGCTAATGTCTGAAAATTGAGGTGACGGGT |
| | | TAAAAATAATATCCTTGGCAACCTTTTTTATATCCCTTTT |
| | | AAATTTTGGCTTAATGACTATATCCAATGAGTCAAAAAG |
| | | CTCCCCTTCAATATCTGTTGCCCCTAAGACCTTTAATAT |
| | | ATCGCCAAATACAGGTAGCTTGGCTTCTACCTTCACCG |
| | | TTGTTCTGCCGATGAAATGCTAATGCATAACATCGTCT |
| | | TTGGTGGTTCCCCTCATCAGTGGCTCTATCTGAACGC |
| | | GCTCTCCACTGCTTAATGACATTCCTTTCCCGATTAAA |
| | | AAATCTGTCAGATCGGATGTGGTCGGCCCGAAAACAG |
| | | TTCTGGCAAAACCAATGGTGTCGCCTTCAACAAACAAA |
| | | AAAGATGGGAATCCCAATGATTCGTCATCTGCGAGGC |
| | | TGTTCTTAATATCTTCAACTGTAGCTTTAGAGCGATTTA |
| | | TCTTCTGAACCAGACTCTTGTCATTTGTTTTGGTAAAGA |
| | | GAAAAGTTTTTCCATCGATTTTATGAATATACAAATAAT |
| | | TGGAGCCAACCTTCAGGTGATGATTATCAGCCAGCAG |
| | | AGAATTAAGGAAAACAGACAGGTTTATTGAGCACTTAT |
| | | CTTTCCCTTTATTTTTGCTGCGGTAAGTCGCATAAAAAC |
| | | CATTCTTCACAATTCAATCCATTTACTATGTTATGTTCT |
| | | GAGGGGAGTGAAAATTCCCCTAATTCGATGAAGATTCT |
| | | TGCTAAATTGTTATCAGCTATGCGCCGACCAGAACACC |
| | | TTGCCGATCAGCCAAACGTCTAATCAGGCCACTGACTA |
| | | GCGATAACTTTCCCCACAACGGAACAACTCTCATTGCA |
| | | TGGGATAATTGGGTACTGTGGGTTTAGTGGTTGTAAAA |
| | | ACACCTGACCGCTATCCCTGATCAGTTTCTTGAAGGTA |
| | | AACTCATCACCCCCAAGTCTGGCTATACAGAAATCACC |
| | | TGGCTCAACAGCCTGCTCAGGGTCAACGAGAATTTAC |
| | | ATTCCGTCAGGATAGCTTGGCTTGGAGCCTGTTGGTG |
| | | CGGTCACGGAATTACCTTCAACCTCAAGCCAGAATGC |
| | | AGAATCACTGGCTTTTTTGGTTGTGCTTACCCATCTCT |
| | | CCGCATCACCTTTGGTAAAGGTTCTAAGCTAAGGTGAG |
| | | AACATCCCTGCCTGAACATGAGAAAAAACAGGGTACTC |
| | | ATACTCACTTATTAGTGACGGCTATGAGCAAAAGGCCA |
| | | GCAAAAGGCCAGGAACCGTAAAAAGGCCGCGTTGCTG |
| | | GCGTTTTTCCATAGGCTCCGCCCCCCTGACGAGCATC |
| | | ACAAAAATCGACGCTCAAGTCAGAGGTGGCGAAACCC |
| | | GACAGGACTATAAAGATACCAGGCGTTTCCCCCTGGA |
| | | AGCTCCCTCGTGCGCTCTCCTGTTCCGACCCTGCCGC |
| | | TTACCGGATACCTGTCCGCCTTTCTCCCTTCGGGAAG |
| | | CGTGGCGCTTTCTCATAGCTCACGCTGTAGGTATCTCA |
| | | GTTCGGTGTAGGTCGTTCGCTCCAAGCTGGGCTGTGT |
| | | GCACGAACCCCCCGTTCAGCCCGACCGCTGCGCCTTA |
| | | TCCGGTAACTATCGTCTTGAGTCCAACCCGGTAAGACA |
| | | CGACTTATCGCCACTGGCAGCAGCCACTGGTAACAGG |
| | | ATTAGCAGAGCGAGGTATGTAGGCGGTGCTACAGAGT |
| | | TCTTGAAGTGGTGGCCTAACTACGGCTACACTAGAAG |
| | | AACAGTATTTGGTATCTGCGCTCTGCTGAAGCCAGTTA |
| | | CCTTCGGAAAAAGAGTTGGTAGCTCTTGATCCGGCAA |
| | | ACAAACCACCGCTGGTAGCGGTGGTTTTTTTGTTTGCA |
| | | AGCAGCAGATTACGCGCAGAAAAAAAGGATCTCAAGA |
| | | AGATCCTTTGATCTTTTCTACGGGGTCTGACGCTCAGT |
| | | GGAACGAAAACTCACGTTAAGGGATTTTGGTCATGAGA |
| | | TTATCAAAAAGGATCTTCACCTAGATCCTTTTAAATTAA |
| | | AAATGAAGTTTTAAATCAAGCCCAATCTGAATAATGTTA |
| | | CAACCAATTAACCAATTCTGATTAGAAAAACTCATCGA |
| | | GCATCAAATGAAACTGCAATTTATTCATATCAGGATTAT |
| | | CAATACCATATTTTTGAAAAAGCCGTTTCTGTAATGAAG |
| | | GAGAAAACTCACCGAGGCAGTTCCATAGGATGGCAAG |
| | | ATCCTGGTATCGGTCTGCGATTCCGACTCGTCCAACAT |
| | | CAATACAACCTATTAATTTCCCCTCGTCAAAAATAAGGT |
| | | TATCAAGTGAGAAATCACCATGAGTGACGACTGAATCC |
| | | GGTGAGAATGGCAAAAGTTTATGCATTTCTTTCCAGAC |
| | | TTGTTCAACAGGCCAGCCATTACGCTCGTCATCAAAAT |
| | | CACTCGCATCAACCAAACCGTTATTCATTCGTGATTGC |
| | | GCCTGAGCAAGACGAAATACGCGATCGCTGTTAAAAG |
| | | GACAATTACAAACAGGAATCGAATGCAACCGGCGCAG |
| | | GAACACTGCCAGCGCATCAACAATATTTTCACCTGAAT |
| | | CAGGATATTCTTCTAATACCTGGAATGCTGTTTTTCCG |
| | | GGGATCGCAGTGGTGAGTAACCATGCATCATCAGGAG |
| | | TACGGATAAAATGCTTGATGGTCGGAAGAGGCATAAAT |
| | | TCCGTCAGCCAGTTTAGTCTGACCATCTCATCTGTAAC |
| | | ATCATTGGCAACGCTACCTTTGCCATGTTTCAGAAACA |
| | | ACTCTGGCGCATCGGGCTTCCCATACAAGCGATAGAT |
| | | TGTCGCACCTGATTGCCCGACATTATCGCGAGCCCAT |
| | | TTATACCCATATAAATCAGCATCCATGTTGGAATTTAAT |
| | | CGCGGCCTCGACGTTTCCCGTTGAATATGGCTCATAA |
| | | CACCCCTTGTATTACTGTTTATGTAAGCAGACAGTTTTA |
| | | TTGTTCATGATGATATATTTTTATCTTGTGCAATGTAAC |
| | | ATCAGAGATTTTGAGACACGGGCCAGAGCTGCA |
| 80 | 5′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGGGGCCTC |
| | CMV enhancer at positions | AGTGAGCGAGCGAGCGCGCAGAGAGGGAGTGGCCAA |
| | 177-546 (part of smCBA | CTCCATCACTAGGGGTTCCTCAGATCTGAATTCGGTAC |
| | promoter) | CTAGTTATTAATAGTAATCAATTACGGGGTCATTAGTTC |
| | Chicken β-actin promoter at | ATAGCCCATATATGGAGTTCCGCGTTACATAACTTACG |
| | positions 548-825 (part of | GTAAATGGCCCGCCTGGCTGACCGCCCAACGACCCC |
| | smCBA promoter) | CGCCCATTGACGTCAATAATGACGTATGTTCCCATAGT |
| | Exon1 at positions 826-918 | AACGCCAATAGGGACTTTCCATTGACGTCAATGGGTG |
| | (part of smCBA promoter) | GAGTATTTACGGTAAACTGCCCACTTGGCAGTACATCA |
| | Chimeric intron at positions | AGTGTATCATATGCCAAGTACGCCCCCTATTGACGTCA |
| | 918-1119 (part of smCBA | ATGACGGTAAATGGCCCGCCTGGCATTATGCCCAGTA |
| | promoter) | CATGACCTTATGGGACTTTCCTACTTGGCAGTACATCT |
| | N-terminal portion of human | ACGTATTAGTCATCGCTATTACCATGGTCGAGGTGAGC |
| | OTOF isoform 5 at positions | CCCACGTTCTGCTTCACTCTCCCCATCTCCCCCCCCTC |
| | 1142-3547 | CCCACCCCCAATTTTGTATTTATTTATTTTTTAATTATTT |
| | Splice donor sequence at | TGTGCAGCGATGGGGCGGGGGGGGGGGGGGGGGGGCG |
| | positions 3548-3631 | CGCGCCAGGCGGGGCGGGGCGGGGCGAGGGGCGG |
| | AP head sequence at positions | GGGGGGCGAGGCGGAGAGGTGCGGCGGCAGCCAA |
| | 3638-3924 | TCAGAGCGGCGCGCTCCGAAAGTTTCCTTTTATGGCG |
| | ITR at positions 3948-4090 | AGGCGGCGGCGGCGGCGGCCCTATAAAAAGCGAAGC |
| | KanR at positions 4616-5410 | GCGCGGCGGGGGGGAGTCGCTGCGCGCTGCCTTCGC |
| | pUC ori at positions 5796-6466 | CCCGTGCCCCGCTCCGCCGCCGCCTCGCGCCGCCCG |
| | Transgene to be transferred | CCCCGGCTCTGACTGACCGCGTTACTCCCACAGGTGA |
| | into vector in dual vector | GCGGGCGGGACGGCCCTTCTCCTCCGGGCTGTAATTA |
| | system at positions 19-4090 | GCGCTTGGTTTAATGACGGCTTGTTTCTTTTCTGTGGC |
| | | TGCGTGAAAGCCTTGAGGGGCTCCGGGAGCTAGAGC |
| | | CTCTGCTAACCATGTTCATGCCTTCTTCTTTTTCCTACA |
| | | GCTCCTGGGCAACGTGCTGGTTATTGTGCTGTCTCAT |
| | | CATTTTGGCAAAGAATTCTAGCGGCCGCCACCATGGC |
| | | CTTGCTCATCCACCTCAAGACAGTCTCGGAGCTGCGG |
| | | GGCAGGGGCGACCGGATCGCCAAAGTGACTTTCCGA |
| | | GGGCAATCCTTCTACTCTCGGGTCCTGGAGAACTGTG |
| | | AGGATGTGGCTGACTTTGATGAGACATTTCGGTGGCC |
| | | GGTGGCCAGCAGCATCGACAGAAATGAGATGCTGGAG |
| | | ATTCAGGTTTTCAACTACAGCAAAGTCTTCAGCAACAA |
| | | GCTCATCGGGACCTTCCGCATGGTGCTGCAGAAGGTG |
| | | GTAGAGGAGAGCCATGTGGAGGTGACTGACACGCTGA |
| | | TTGATGACAACAATGCTATCATCAAGACCAGCCTGTGC |
| | | GTGGAGGTCCGGTATCAGGCCACTGACGGCACAGTG |
| | | GGCTCCTGGGACGATGGGGACTTCCTGGGAGATGAG |
| | | TCTCTTCAAGAGGAAGAGAAGGACAGCCAAGAGACGG |
| | | ATGGACTGCTCCCAGGCTCCCGGCCCAGCTCCCGGC |
| | | CCCCAGGAGAGAAGAGCTTCCGGAGAGCCGGGAGGA |
| | | GCGTGTTCTCCGCCATGAAGCTCGGCAAAAACCGGTC |
| | | TCACAAGGAGGAGCCCCAAAGACCAGATGAACCGGC |
| | | GGTGCTGGAGATGGAAGACCTTGACCATCTGGCCATT |
| | | CGGCTAGGAGATGGACTGGATCCCGACTCGGTGTCTC |
| | | TAGCCTCAGTCACAGCTCTCACCACTAATGTCTCCAAC |
| | | AAGCGATCTAAGCCAGACATTAAGATGGAGCCAAGTG |
| | | CTGGGCGGCCCATGGATTACCAGGTCAGCATCACGGT |
| | | GATCGAGGCCCGGCAGCTGGTGGGCTTGAACATGGA |
| | | CCCTGTGGTGTGCGTGGAGGTGGGTGACGACAAGAA |
| | | GTACACATCCATGAAGGAGTCCACTAACTGCCCCTATT |
| | | ACAACGAGTACTTCGTCTTCGACTTCCATGTCTCTCCG |
| | | GATGTCATGTTTGACAAGATCATCAAGATTTCGGTGAT |
| | | TCACTCCAAGAACCTGCTGCGCAGTGGCACCCTGGTG |
| | | GGCTCCTTCAAAATGGACGTGGGAACCGTGTACTCGC |
| | | AGCCAGAGCACCAGTTCCATCACAAGTGGGCCATCCT |
| | | GTCTGACCCCGATGACATCTCCTCGGGGCTGAAGGGC |
| | | TACGTGAAGTGTGACGTTGCCGTGGTGGGCAAAGGG |
| | | GACAACATCAAGACGCCCCACAAGGCCAATGAGACCG |
| | | ACGAAGATGACATTGAGGGGAACTTGCTGCTCCCCGA |
| | | GGGGGTGCCCCCCGAACGCCAGTGGGCCCGGTTCTA |
| | | TGTGAAAATTTACCGAGCAGAGGGGCTGCCCCGTATG |
| | | AACACAAGCCTCATGGCCAATGTAAAGAAGGCTTTCAT |
| | | CGGTGAAAACAAGGACCTCGTGGACCCCTACGTGCAA |
| | | GTCTTCTTTGCTGGCCAGAAGGGCAAGACTTCAGTGC |
| | | AGAAGAGCAGCTATGAGCCCCTGTGGAATGAGCAGGT |
| | | CGTCTTTACAGACCTCTTCCCCCCACTCTGCAAACGCA |
| | | TGAAGGTGCAGATCCGAGACTCGGACAAGGTCAACGA |
| | | CGTGGCCATCGGCACCCACTTCATTGACCTGCGCAAG |
| | | ATTTCTAATGACGGAGACAAAGGCTTCCTGCCCACACT |
| | | GGGCCCAGCCTGGGTGAACATGTACGGCTCCACACGT |
| | | AACTACACGCTGCTGGATGAGCATCAGGACCTGAACG |
| | | AGGGCCTGGGGGAGGGTGTGTCCTTCCGGGCCCGGC |
| | | TCCTGCTGGGCCTGGCTGTGGAGATCGTAGACACCTC |
| | | CAACCCTGAGCTCACCAGCTCCACAGAGGTGCAGGTG |
| | | GAGCAGGCCACGCCCATCTCGGAGAGCTGTGCAGGT |
| | | AAAATGGAAGAATTCTTTCTCTTTGGAGCCTTCCTGGA |
| | | GGCCTCAATGATCGACCGGAGAAACGGAGACAAGCCC |
| | | ATCACCTTTGAGGTCACCATAGGCAACTATGGGAACG |
| | | AAGTTGATGGCCTGTCCCGGCCCCAGCGGCCTCGGC |
| | | CCCGGAAGGAGCCGGGGGATGAGGAAGAAGTAGACC |
| | | TGATTCAGAACGCAAGTGATGACGAGGCCGGTGATGC |
| | | CGGGGACCTGGCCTCAGTCTCCTCCACTCCACCAATG |
| | | CGGCCCCAGGTCACCGACAGGAACTACTTCCATCTGC |
| | | CCTACCTGGAGCGAAAGCCCTGCATCTACATCAAGAG |
| | | CTGGTGGCCGGACCAGCGCCGCCGCCTCTACAATGC |
| | | CAACATCATGGACCACATTGCCGACAAGCTGGAAGAA |
| | | GGCCTGAACGACATACAGGAGATGATCAAAACGGAGA |
| | | AGTCCTACCCTGAGCGTCGCCTGCGGGGCGTCCTGG |
| | | AGGAGCTGAGCTGTGGCTGCTGCCGCTTCCTCTCCCT |
| | | CGCTGACAAGGACCAGGGCCACTCATCCCGCACCAG |
| | | GCTTGACCGGGAGCGCCTCAAGTCCTGCATGAGGGA |
| | | GCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACTGGGGAGAGATCTGAG |
| | | GAACCCCTAGTGATGGAGTTGGCCACTCCCTCTCTGC |
| | | GCGCTCGCTCGCTCACTGAGGCCGCCCGGGCAAAGC |
| | | CCGGGCGTCGGGCGACCTTTGGTCGCCCGGCCTCAG |
| | | TGAGCGAGCGAGCGCGCAGAGAGGGAGTGGCCAACC |
| | | CCCCCCCCCCCCCCCCTGCAGCCTGGCGTAATAGCG |
| | | AAGAGGCCCGCACCGATCGCCCTTCCCAACAGTTGCG |
| | | TAGCCTGAATGGCGAATGGCGCGACGCGCCCTGTAG |
| | | CGGCGCATTAAGCGCGGCGGGTGTGGTGGTTACGCG |
| | | CAGCGTGACCGCTACACTTGCCAGCGCCCTAGCGCCC |
| | | GCTCCTTTCGCTTTCTTCCCTTCCTTTCTCGCCACGTT |
| | | CGCCGGCTTTCCCCGTCAAGCTCTAAATCGGGGGCTC |
| | | CCTTTAGGGTTCCGATTTAGTGCTTTACGGCACCTCGA |
| | | CCCCAAAAAACTTGATTAGGGTGATGGTTCACGTAGTG |
| | | GGCCATCGCCCTGATAGACGGTTTTTCGCCCTTTGAC |
| | | GTTGGAGTCCACGTTCTTTAATAGTGGACTCTTGTTCC |
| | | AAACTGGAACAACACTCAACCCTATCGCGGTCTATTCT |
| | | TTTGATTTATAAGGGATGTTGCCGATTTCGGCCTATTG |
| | | GTTAAAAAATGAGCTGATTTAACAAAAATTTTAACAAAA |
| | | TTCAGAAGAACTCGTCAAGAAGGCGATAGAAGGCGAT |
| | | GCGCTGCGAATCGGGAGCGGCGATACCGTAAAGCAC |
| | | GAGGAAGCGGTCAGCCCATTCGCCGCCAAGCTCTTCA |
| | | GCAATATCACGGGTAGCCAACGCTATGTCCTGATAGC |
| | | GGTCCGCCACACCCAGCCGGCCACAGTCGATGAATCC |
| | | AGAAAAGCGGCCATTTTCCACCATGATATTCGGCAAGC |
| | | AGGCATCGCCATGGGTCACGACGAGATCCTCGCCGTC |
| | | GGGCATGCTCGCCTTGAGCCTGGCGAACAGTTCGGCT |
| | | GGCGCGAGCCCCTGATGCTCTTCGTCCAGATCATCCT |
| | | GATCGACAAGACCGGCTTCCATCCGAGTACGTGCTCG |
| | | CTCGATGCGATGTTTCGCTTGGTGGTCGAATGGGCAG |
| | | GTAGCCGGATCAAGCGTATGCAGCCGCCGCATTGCAT |
| | | CAGCCATGATGGATACTTTCTCGGCAGGAGCAAGGTG |
| | | AGATGACAGGAGATCCTGCCCCGGCACTTCGCCCAAT |
| | | AGCAGCCAGTCCCTTCCCGCTTCAGTGACAACGTCGA |
| | | GCACAGCTGCGCAAGGAACGCCCGTCGTGGCCAGCC |
| | | ACGATAGCCGCGCTGCCTCGTCTTGCAGTTCATTCAG |
| | | GGCACCGGACAGGTCGGTCTTGACAAAAAGAACCGG |
| | | GCGCCCCTGCGCTGACAGCCGGAACACGGCGGCATC |
| | | AGAGCAGCCGATTGTCTGTTGTGCCCAGTCATAGCCG |
| | | AATAGCCTCTCCACCCAAGCGGCCGGAGAACCTGCGT |
| | | GCAATCCATCTTGTTCAATCATGCGAAACGATCCTCAT |
| | | CCTGTCTCTTGATCAGATCTTGATCCCCTGCGCCATCA |
| | | GATCCTTGGCGGCGAGAAAGCCATCCAGTTTACTTTG |
| | | CAGGGCTTCCCAACCTTACCAGAGGGCGCCCCAGCTG |
| | | GCAATTCCGGTTCGCTTGCTGTCCATAAAACCGCCCA |
| | | GTCTAGCTATCGCCATGTAAGCCCACTGCAAGCTACCT |
| | | GCTTTCTCTTTGCGCTTGCGTTTTCCCTTGTCCAGATA |
| | | GCCCAGTAGCTGACATTCATCCGGGGTCAGCACCGTT |
| | | TCTGCGGACTGGCTTTCTACGTGAAAAGGATCTAGGT |
| | | GAAGATCCTTTTTGATAATCTCATGACCAAAATCCCTTA |
| | | ACGTGAGTTTTCGTTCCACTGAGCGTCAGACCCCGTA |
| | | GAAAAGATCAAAGGATCTTCTTGAGATCCTTTTTTTCTG |
| | | CGCGTAATCTGCTGCTTGCAAACAAAAAAACCACCGCT |
| | | ACCAGCGGTGGTTTGTTTGCCGGATCAAGAGCTACCA |
| | | ACTCTTTTTCCGAAGGTAACTGGCTTCAGCAGAGCGCA |
| | | GATACCAAATACTGTCCTTCTAGTGTAGCCGTAGTTAG |
| | | GCCACCACTTCAAGAACTCTGTAGCACCGCCTACATAC |
| | | CTCGCTCTGCTAATCCTGTTACCAGTGGCTGCTGCCA |
| | | GTGGCGATAAGTCGTGTCTTACCGGGTTGGACTCAAG |
| | | ACGATAGTTACCGGATAAGGCGCAGCGGTCGGGCTGA |
| | | ACGGGGGGTTCGTGCACACAGCCCAGCTTGGAGCGA |
| | | ACGACCTACACCGAACTGAGATACCTACAGCGTGAGC |
| | | TATGAGAAAGCGCCACGCTTCCCGAAGGGAGAAAGGC |
| | | GGACAGGTATCCGGTAAGCGGCAGGGTCGGAACAGG |
| | | AGAGCGCACGAGGGAGCTTCCAGGGGGAAACGCCTG |
| | | GTATCTTTATAGTCCTGTCGGGTTTCGCCACCTCTGAC |
| | | TTGAGCGTCGATTTTTGTGATGCTCGTCAGGGGGGCG |
| | | GAGCCTATGGAAAAACGCCAGCAACGCGGCCTTTTTA |
| | | CGGTTCCTGGGCTTTTGCTGGCCTTTTGCTCACATGTT |
| | | CTTTCCTGCGTTATCCCCTGATTCTGTGGATAACCGTA |
| | | TTACCGCCTTTGAGTGAGCTGATACCGCTCGCCGCAG |
| | | CCGAACGACCGAGCGCAGCGAGTCAGTGAGCGAGGA |
| | | AGCGGAAGAGCGCCCAATACGCAAACCGCCTCTCCCC |
| | | GCGCGTTGGCCGATTCATTAATGCAGGGCTGCA |
| 81 | 5′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | Myo15 promoter at positions | GGGAGTGGCCAACTCCATCACTAGGGGTTCCTTGTAG |
| | 235-1199 | TTAATGATTAACCCGCCATGCTACTTATCTACGTAGCC |
| | Kozak sequence at positions | ATGCTCTAGGAAGATCGGAATTCGCCCTTAAGCTAGC |
| | 1216-1225 | GGCGCGCCCAATTCTGCAGCTCAGCCTACTACTTGCT |
| | N-terminal portion of human | TTCCAGGCTGTTCCTAGTTCCCATGTCAGCTGCTTGTG |
| | OTOF isoform 5 at positions | CTTTCCAGAGACAAAACAGGAATAATAGATGTCATTAA |
| | 1222-3627 | ATATACATTGGGCCCCAGGCGGTCAATGTGGCAGCCT |
| | Splice donor sequence at | GAGCCTCCTTTCCATCTCTGTGGAGGCAGACATAGGA |
| | positions 3628-3711 | CCCCCAACAAACAGCATGCAGGTTGGGAGCCAGCCAC |
| | AP head sequence at positions | AGGACCCAGGTAAGGGGCCCTGGGTCCTTAAGCTTCT |
| | 3718-4004 | GCCACTGGCTCCGGCATTGCAGAGAGAAGAGAAGGG |
| | ITR at positions 4098-4227 | GCGGCAGACTGGAGAGCTGGGCTCCATTTTTGTTCCT |
| | M13 fwd at positions 4246-4262 | TGGTGCCCTGCCCCTCCCCATGACCTGCAGAGACATT |
| | f1 ori at positions 4404-4859 | CAGCCTGCCAGGCTTTATGAGGTGGGAGCTGGGCTCT |
| | AmpR promoter at positions | CCCTGATGTATTATTCAGCTCCCTGGAGTTGGCCAGCT |
| | 4885-4989 | CCTGTTACACTGGCCACAGCCCTGGGCATCCGCTTCT |
| | KanR at positions 4990-5799 | CACTTCTAGTTTCCCCTCCAAGGTAATGTGGTGGGTCA |
| | mutBsmBI at positions 5430- | TGATCATTCTATCCTGGCTTCAGGGACCTGACTCCACT |
| | 5430 | TTGGGGCCATTCGAGGGGTCTAGGGTAGATGATGTCC |
| | ori at positions 5970-6558 | CCCTGTGGGGATTAATGTCCTGCTCTGTAAAACTGAGC |
| | CAP binding site at positions | TAGCTGAGATCCAGGAGGGCTTGGCCAGAGACAGCAA |
| | 6846-6867 | GTTGTTGCCATGGTGACTTTAAAGCCAGGTTGCTGCC |
| | lac promoter at positions 6882- | CCAGCACAGGCCTCCCAGTCTACCCTCACTAGAAAAC |
| | 6912 | AACACCCAGGCACTTTCCACCACCTCTCAAAGGTGAAA |
| | lac operator at positions 6920- | CCCAAGGCTGGTCTAGAGAATGAATTATGGATCCTCG |
| | 6936 | CTGTCCGTGCCACCCAGCTAGTCCCAGCGGCTCAGAC |
| | M13 rev at positions 6944-6960 | ACTGAGGAGAGACTGTAGGTTCAGCTACAAGCAAAAA |
| | Transgene to be transferred | GACCTAGCTGGTCTCCAAGCAGTGTCTCCAAGTCCCT |
| | into vector in dual vector | GAACCTGTGACACCTGCCCCAGGCATCATCAGGCACA |
| | system at positions 12-4227 | GAGGGCCACCAAGAATTCTAGCGGCCGCCACCATGG |
| | | CCTTGCTCATCCACCTCAAGACAGTCTCGGAGCTGCG |
| | | GGGCAGGGGCGACCGGATCGCCAAAGTGACTTTCCG |
| | | AGGGCAATCCTTCTACTCTCGGGTCCTGGAGAACTGT |
| | | GAGGATGTGGCTGACTTTGATGAGACATTTCGGTGGC |
| | | CGGTGGCCAGCAGCATCGACAGAAATGAGATGCTGGA |
| | | GATTCAGGTTTTCAACTACAGCAAAGTCTTCAGCAACA |
| | | AGCTCATCGGGACCTTCCGCATGGTGCTGCAGAAGGT |
| | | GGTAGAGGAGAGCCATGTGGAGGTGACTGACACGCT |
| | | GATTGATGACAACAATGCTATCATCAAGACCAGCCTGT |
| | | GCGTGGAGGTCCGGTATCAGGCCACTGACGGCACAG |
| | | TGGGCTCCTGGGACGATGGGGACTTCCTGGGAGATG |
| | | AGTCTCTTCAAGAGGAAGAGAAGGACAGCCAAGAGAC |
| | | GGATGGACTGCTCCCAGGCTCCCGGCCCAGCTCCCG |
| | | GCCCCCAGGAGAGAAGAGCTTCCGGAGAGCCGGGAG |
| | | GAGCGTGTTCTCCGCCATGAAGCTCGGCAAAAACCGG |
| | | TCTCACAAGGAGGAGCCCCAAAGACCAGATGAACCGG |
| | | CGGTGCTGGAGATGGAAGACCTTGACCATCTGGCCAT |
| | | TCGGCTAGGAGATGGACTGGATCCCGACTCGGTGTCT |
| | | CTAGCCTCAGTCACAGCTCTCACCACTAATGTCTCCAA |
| | | CAAGCGATCTAAGCCAGACATTAAGATGGAGCCAAGT |
| | | GCTGGGCGGCCCATGGATTACCAGGTCAGCATCACG |
| | | GTGATCGAGGCCCGGCAGCTGGTGGGCTTGAACATG |
| | | GACCCTGTGGTGTGCGTGGAGGTGGGTGACGACAAG |
| | | AAGTACACATCCATGAAGGAGTCCACTAACTGCCCCTA |
| | | TTACAACGAGTACTTCGTCTTCGACTTCCATGTCTCTC |
| | | CGGATGTCATGTTTGACAAGATCATCAAGATTTCGGTG |
| | | ATTCACTCCAAGAACCTGCTGCGCAGTGGCACCCTGG |
| | | TGGGCTCCTTCAAAATGGACGTGGGAACCGTGTACTC |
| | | GCAGCCAGAGCACCAGTTCCATCACAAGTGGGCCATC |
| | | CTGTCTGACCCCGATGACATCTCCTCGGGGCTGAAGG |
| | | GCTACGTGAAGTGTGACGTTGCCGTGGTGGGCAAAGG |
| | | GGACAACATCAAGACGCCCCACAAGGCCAATGAGACC |
| | | GACGAAGATGACATTGAGGGGAACTTGCTGCTCCCCG |
| | | AGGGGGTGCCCCCCGAACGCCAGTGGGCCCGGTTCT |
| | | ATGTGAAAATTTACCGAGCAGAGGGGCTGCCCCGTAT |
| | | GAACACAAGCCTCATGGCCAATGTAAAGAAGGCTTTCA |
| | | TCGGTGAAAACAAGGACCTCGTGGACCCCTACGTGCA |
| | | AGTCTTCTTTGCTGGCCAGAAGGGCAAGACTTCAGTG |
| | | CAGAAGAGCAGCTATGAGCCCCTGTGGAATGAGCAGG |
| | | TCGTCTTTACAGACCTCTTCCCCCCACTCTGCAAACGC |
| | | ATGAAGGTGCAGATCCGAGACTCGGACAAGGTCAACG |
| | | ACGTGGCCATCGGCACCCACTTCATTGACCTGCGCAA |
| | | GATTTCTAATGACGGAGACAAAGGCTTCCTGCCCACA |
| | | CTGGGCCCAGCCTGGGTGAACATGTACGGCTCCACAC |
| | | GTAACTACACGCTGCTGGATGAGCATCAGGACCTGAA |
| | | CGAGGGCCTGGGGGAGGGTGTGTCCTTCCGGGCCCG |
| | | GCTCCTGCTGGGCCTGGCTGTGGAGATCGTAGACACC |
| | | TCCAACCCTGAGCTCACCAGCTCCACAGAGGTGCAGG |
| | | TGGAGCAGGCCACGCCCATCTCGGAGAGCTGTGCAG |
| | | GTAAAATGGAAGAATTCTTTCTCTTTGGAGCCTTCCTG |
| | | GAGGCCTCAATGATCGACCGGAGAAACGGAGACAAGC |
| | | CCATCACCTTTGAGGTCACCATAGGCAACTATGGGAA |
| | | CGAAGTTGATGGCCTGTCCCGGCCCCAGCGGCCTCG |
| | | GCCCCGGAAGGAGCCGGGGGATGAGGAAGAAGTAGA |
| | | CCTGATTCAGAACGCAAGTGATGACGAGGCCGGTGAT |
| | | GCCGGGGACCTGGCCTCAGTCTCCTCCACTCCACCAA |
| | | TGCGGCCCCAGGTCACCGACAGGAACTACTTCCATCT |
| | | GCCCTACCTGGAGCGAAAGCCCTGCATCTACATCAAG |
| | | AGCTGGTGGCCGGACCAGCGCCGCCGCCTCTACAAT |
| | | GCCAACATCATGGACCACATTGCCGACAAGCTGGAAG |
| | | AAGGCCTGAACGACATACAGGAGATGATCAAAACGGA |
| | | GAAGTCCTACCCTGAGCGTCGCCTGCGGGGCGTCCT |
| | | GGAGGAGCTGAGCTGTGGCTGCTGCCGCTTCCTCTCC |
| | | CTCGCTGACAAGGACCAGGGCCACTCATCCCGCACCA |
| | | GGCTTGACCGGGAGCGCCTCAAGTCCTGCATGAGGG |
| | | AGCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACCTCGAGTTAAGGGCGA |
| | | ATTCCCGATAAGGATCTTCCTAGAGCATGGCTACGTAG |
| | | ATAAGTAGCATGGCGGGTTAATCATTAACTACAAGGAA |
| | | CCCCTAGTGATGGAGTTGGCCACTCCCTCTCTGCGCG |
| | | CTCGCTCGCTCACTGAGGCCGGGCGACCAAAGGTCG |
| | | CCCGACGCCCGGGCTTTGCCCGGGGGGCCTCAGTGA |
| | | GCGAGCGAGCGCGCAGCCTTAATTAACCTAATTCACT |
| | | GGCCGTCGTTTTACAACGTCGTGACTGGGAAAACCCT |
| | | GGCGTTACCCAACTTAATCGCCTTGCAGCACATCCCC |
| | | CTTTCGCCAGCTGGCGTAATAGCGAAGAGGCCCGCAC |
| | | CGATCGCCCTTCCCAACAGTTGCGCAGCCTGAATGGC |
| | | GAATGGGACGCGCCCTGTAGCGGCGCATTAAGCGCG |
| | | GCGGGTGTGGTGGTTACGCGCAGCGTGACCGCTACA |
| | | CTTGCCAGCGCCCTAGCGCCCGCTCCTTTCGCTTTCT |
| | | TCCCTTCCTTTCTCGCCACGTTCGCCGGCTTTCCCCGT |
| | | CAAGCTCTAAATCGGGGGCTCCCTTTAGGGTTCCGAT |
| | | TTAGTGCTTTACGGCACCTCGACCCCAAAAAACTTGAT |
| | | TAGGGTGATGGTTCACGTAGTGGGCCATCGCCCTGAT |
| | | AGACGGTTTTTCGCCCTTTGACGTTGGAGTCCACGTTC |
| | | TTTAATAGTGGACTCTTGTTCCAAACTGGAACAACACT |
| | | CAACCCTATCTCGGTCTATTCTTTTGATTTATAAGGGAT |
| | | TTTGCCGATTTCGGCCTATTGGTTAAAAAATGAGCTGA |
| | | TTTAACAAAAATTTAACGCGAATTTTAACAAAATATTAA |
| | | CGCTTACAATTTAGGTGGCACTTTTCGGGGAAATGTGC |
| | | GCGGAACCCCTATTTGTTTATTTTTCTAAATACATTCAA |
| | | ATATGTATCCGCTCATGAGACAATAACCCTGATAAATG |
| | | CTTCAATAATATTGAAAAAGGAAGAGTATGAGCCATAT |
| | | TCAACGGGAAACGTCGAGGCCGCGATTAAATTCCAAC |
| | | ATGGATGCTGATTTATATGGGTATAAATGGGCTCGCGA |
| | | TAATGTCGGGCAATCAGGTGCGACAATCTATCGCTTGT |
| | | ATGGGAAGCCCGATGCGCCAGAGTTGTTTCTGAAACA |
| | | TGGCAAAGGTAGCGTTGCCAATGATGTTACAGATGAG |
| | | ATGGTCAGACTAAACTGGCTGACGGAATTTATGCCTCT |
| | | TCCGACCATCAAGCATTTTATCCGTACTCCTGATGATG |
| | | CATGGTTACTCACCACTGCGATCCCCGGAAAAACAGC |
| | | ATTCCAGGTATTAGAAGAATATCCTGATTCAGGTGAAA |
| | | ATATTGTTGATGCGCTGGCAGTGTTCCTGCGCCGGTT |
| | | GCATTCGATTCCTGTTTGTAATTGTCCTTTTAACAGCGA |
| | | TCGCGTATTTCGTCTTGCTCAGGCGCAATCACGAATGA |
| | | ATAACGGTTTGGTTGATGCGAGTGATTTTGATGACGAG |
| | | CGTAATGGCTGGCCTGTTGAACAAGTCTGGAAAGAAA |
| | | TGCATAAACTTTTGCCATTCTCACCGGATTCAGTCGTC |
| | | ACTCATGGTGATTTCTCACTTGATAACCTTATTTTTGAC |
| | | GAGGGGAAATTAATAGGTTGTATTGATGTTGGACGAGT |
| | | CGGAATCGCAGACCGATACCAGGATCTTGCCATCCTA |
| | | TGGAACTGCCTCGGTGAGTTTTCTCCTTCATTACAGAA |
| | | ACGGCTTTTTCAAAAATATGGTATTGATAATCCTGATAT |
| | | GAATAAATTGCAGTTTCATTTGATGCTCGATGAGTTTTT |
| | | CTAACTGTCAGACCAAGTTTACTCATATATACTTTAGAT |
| | | TGATTTAAAACTTCATTTTTAATTTAAAAGGATCTAGGT |
| | | GAAGATCCTTTTTGATAATCTCATGACCAAAATCCCTTA |
| | | ACGTGAGTTTTCGTTCCACTGAGCGTCAGACCCCGTA |
| | | GAAAAGATCAAAGGATCTTCTTGAGATCCTTTTTTTCTG |
| | | CGCGTAATCTGCTGCTTGCAAACAAAAAAACCACCGCT |
| | | ACCAGCGGTGGTTTGTTTGCCGGATCAAGAGCTACCA |
| | | ACTCTTTTTCCGAAGGTAACTGGCTTCAGCAGAGCGCA |
| | | GATACCAAATACTGTTCTTCTAGTGTAGCCGTAGTTAG |
| | | GCCACCACTTCAAGAACTCTGTAGCACCGCCTACATAC |
| | | CTCGCTCTGCTAATCCTGTTACCAGTGGCTGCTGCCA |
| | | GTGGCGATAAGTCGTGTCTTACCGGGTTGGACTCAAG |
| | | ACGATAGTTACCGGATAAGGCGCAGCGGTCGGGCTGA |
| | | ACGGGGGGTTCGTGCACACAGCCCAGCTTGGAGCGA |
| | | ACGACCTACACCGAACTGAGATACCTACAGCGTGAGC |
| | | TATGAGAAAGCGCCACGCTTCCCGAAGGGAGAAAGGC |
| | | GGACAGGTATCCGGTAAGCGGCAGGGTCGGAACAGG |
| | | AGAGCGCACGAGGGAGCTTCCAGGGGGAAACGCCTG |
| | | GTATCTTTATAGTCCTGTCGGGTTTCGCCACCTCTGAC |
| | | TTGAGCGTCGATTTTTGTGATGCTCGTCAGGGGGGCG |
| | | GAGCCTATGGAAAAACGCCAGCAACGCGGCCTTTTTA |
| | | CGGTTCCTGGCCTTTTGCTGGCCTTTTGCTCACATGTT |
| | | CTTTCCTGCGTTATCCCCTGATTCTGTGGATAACCGTA |
| | | TTACCGCCTTTGAGTGAGCTGATACCGCTCGCCGCAG |
| | | CCGAACGACCGAGCGCAGCGAGTCAGTGAGCGAGGA |
| | | AGCGGAAGAGCGCCCAATACGCAAACCGCCTCTCCCC |
| | | GCGCGTTGGCCGATTCATTAATGCAGCTGGCACGACA |
| | | GGTTTCCCGACTGGAAAGCGGGCAGTGAGCGCAACG |
| | | CAATTAATGTGAGTTAGCTCACTCATTAGGCACCCCAG |
| | | GCTTTACACTTTATGCTTCCGGCTCGTATGTTGTGTGG |
| | | AATTGTGAGCGGATAACAATTTCACACAGGAAACAGCT |
| | | ATGACCATGATTACGCCAGATTTAATTAAGG |
| 82 | 3′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | AP head sequence at positions | GGGAGTGGCCAACTCCATCACTAGGGGTTCCTTGTAG |
| | 229-515 | TTAATGATTAACCCGCCATGCTACTTATCTACGTAGCC |
| | Splice acceptor sequence at | ATGCTCTAGGAAGATCGGAATTCGCCCTTAAGCTAGC |
| | positions 538-586 | GGCGCGCCCCCGGGTGCGCGGCGTCGGTGGTGCCG |
| | C-terminal portion of human | GCGGGGGGCGCCAGGTCGCAGGCGGTGTAGGGCTC |
| | OTOF isoform 5 at positions | CAGGCAGGCGGCGAAGGCCATGACGTGCGCTATGAA |
| | 587-4174 | GGTCTGCTCCTGCACGCCGTGAACCAGGTGCGCCTG |
| | bGH poly(A) sequence at | CGGGCCGCGCGCGAACACCGCCACGTCCTCGCCTGC |
| | positions 4217-4438 | GTGGGTCTCTTCGTCCAGGGGCACTGCTGACTGCTGC |
| | ITR at positions 4526-4655 | CGATACTCGGGGCTCCCGCTCTCGCTCTCGGTAACAT |
| | M13 fwd at positions 4674-4690 | CCGGCCGGGCGCCGTCCTTGAGCACATAGCCTGGAC |
| | f1 ori at positions 4832-5287 | CGTTTCCTTAAGCGACGCATGCTCGCGATAGGCACCT |
| | AmpR promoter at positions | ATTGGTCTTACTGACATCCACTTTGCCTTTCTCTCCACA |
| | 5313-5417 | GGAAAACATGGGGCAGCAGGCCAGGATGCTGCGGGC |
| | KanR at positions 5418-6227 | CCAGGTGAAGCGGCACACGGTGCGGGACAAGCTGAG |
| | mutBsmBI at positions 5858- | GCTGTGCCAGAACTTCCTGCAGAAGCTGCGCTTCCTG |
| | 5858 | GCGGACGAGCCCCAGCACAGCATTCCCGACATCTTCA |
| | ori at positions 6398-6986 | TCTGGATGATGAGCAACAACAAGCGTGTCGCCTATGC |
| | CAP binding site at positions | CCGTGTGCCCTCCAAGGACCTGCTCTTCTCCATCGTG |
| | 7274-7295 | GAGGAGGAGACTGGCAAGGACTGCGCCAAGGTCAAG |
| | lac promoter at positions 7310- | ACGCTCTTCCTTAAGCTGCCAGGGAAGCGGGGCTTCG |
| | 7340 | GCTCGGCAGGCTGGACAGTGCAGGCCAAGGTGGAGC |
| | lac operator at positions 7348- | TGTACCTGTGGCTGGGCCTCAGCAAACAGCGCAAGGA |
| | 7364 | GTTCCTGTGCGGCCTGCCCTGTGGCTTCCAGGAGGTC |
| | M13 rev at positions 7372-7388 | AAGGCAGCCCAGGGCCTGGGCCTGCATGCCTTCCCA |
| | Transgene to be transferred | CCCGTCAGCCTGGTCTACACCAAGAAGCAGGCGTTCC |
| | into vector in dual vector | AGCTCCGAGCGCACATGTACCAGGCCCGCAGCCTCTT |
| | system at positions 12-4655 | TGCCGCCGACAGCAGCGGACTCTCAGACCCCTTTGCC |
| | | CGCGTCTTCTTCATCAATCAGAGTCAGTGCACAGAGGT |
| | | GCTGAATGAGACCCTGTGTCCCACCTGGGACCAGATG |
| | | CTGGTGTTCGACAACCTGGAGCTCTATGGTGAAGCTC |
| | | ATGAGCTGAGGGACGATCCGCCCATCATTGTCATTGA |
| | | AATCTATGACCAGGATTCCATGGGCAAAGCTGACTTCA |
| | | TGGGCCGGACCTTCGCCAAACCCCTGGTGAAGATGGC |
| | | AGACGAGGCGTACTGCCCACCCCGCTTCCCACCTCAG |
| | | CTCGAGTACTACCAGATCTACCGTGGCAACGCCACAG |
| | | CTGGAGACCTGCTGGCGGCCTTCGAGCTGCTGCAGAT |
| | | TGGACCAGCAGGGAAGGCTGACCTGCCCCCCATCAAT |
| | | GGCCCGGTGGACGTGGACCGAGGTCCCATCATGCCC |
| | | GTGCCCATGGGCATCCGGCCCGTGCTCAGCAAGTACC |
| | | GAGTGGAGGTGCTGTTCTGGGGCCTACGGGACCTAAA |
| | | GCGGGTGAACCTGGCCCAGGTGGACCGGCCACGGGT |
| | | GGACATCGAGTGTGCAGGGAAGGGGGTGCAGTCGTC |
| | | CCTGATCCACAATTATAAGAAGAACCCCAACTTCAACA |
| | | CCCTCGTCAAGTGGTTTGAAGTGGACCTCCCAGAGAA |
| | | CGAGCTGCTGCACCCGCCCTTGAACATCCGTGTGGTG |
| | | GACTGCCGGGCCTTCGGTCGCTACACACTGGTGGGCT |
| | | CCCATGCCGTCAGCTCCCTGCGACGCTTCATCTACCG |
| | | GCCCCCAGACCGCTCGGCCCCCAGCTGGAACACCAC |
| | | GGTCAGGCTTCTCCGGCGCTGCCGTGTGCTGTGCAAT |
| | | GGGGGCTCCTCCTCTCACTCCACAGGGGAGGTTGTG |
| | | GTGACTATGGAGCCAGAGGTACCCATCAAGAAACTGG |
| | | AGACCATGGTGAAGCTGGACGCGACTTCTGAAGCTGT |
| | | TGTCAAGGTGGATGTGGCTGAGGAGGAGAAGGAGAA |
| | | GAAGAAGAAGAAGAAGGGCACTGCGGAGGAGCCAGA |
| | | GGAGGAGGAGCCAGACGAGAGCATGCTGGACTGGTG |
| | | GTCCAAGTACTTTGCCTCCATTGACACCATGAAGGAGC |
| | | AACTTCGACAACAAGAGCCCTCTGGAATTGACTTGGA |
| | | GGAGAAGGAGGAAGTGGACAATACCGAGGGCCTGAA |
| | | GGGGTCAATGAAGGGCAAGGAGAAGGCAAGGGCTGC |
| | | CAAAGAGGAGAAGAAGAAGAAAACTCAGAGCTCTGGC |
| | | TCTGGCCAGGGGTCCGAGGCCCCCGAGAAGAAGAAA |
| | | CCCAAGATTGATGAGCTTAAGGTATACCCCAAAGAGCT |
| | | GGAGTCCGAGTTTGATAACTTTGAGGACTGGCTGCAC |
| | | ACTTTCAACTTGCTTCGGGGCAAGACCGGGGATGATG |
| | | AGGATGGCTCCACCGAGGAGGAGCGCATTGTGGGAC |
| | | GCTTCAAGGGCTCCCTCTGCGTGTACAAAGTGCCACT |
| | | CCCAGAGGACGTGTCCCGGGAAGCCGGCTACGACTC |
| | | CACCTACGGCATGTTCCAGGGCATCCCGAGCAATGAC |
| | | CCCATCAATGTGCTGGTCCGAGTCTATGTGGTCCGGG |
| | | CCACGGACCTGCACCCTGCTGACATCAACGGCAAAGC |
| | | TGACCCCTACATCGCCATCCGGCTAGGCAAGACTGAC |
| | | ATCCGCGACAAGGAGAACTACATCTCCAAGCAGCTCA |
| | | ACCCTGTCTTTGGGAAGTCCTTTGACATCGAGGCCTC |
| | | CTTCCCCATGGAATCCATGCTGACGGTGGCTGTGTAT |
| | | GACTGGGACCTGGTGGGCACTGATGACCTCATTGGGG |
| | | AAACCAAGATCGACCTGGAGAACCGCTTCTACAGCAA |
| | | GCACCGCGCCACCTGCGGCATCGCCCAGACCTACTC |
| | | CACACATGGCTACAATATCTGGCGGGACCCCATGAAG |
| | | CCCAGCCAGATCCTGACCCGCCTCTGCAAAGACGGCA |
| | | AAGTGGACGGCCCCCACTTTGGGCCCCCTGGGAGAG |
| | | TGAAGGTGGCCAACCGCGTCTTCACTGGGCCCTCTGA |
| | | GATTGAGGACGAGAACGGTCAGAGGAAGCCCACAGA |
| | | CGAGCATGTGGCGCTGTTGGCCCTGAGGCACTGGGA |
| | | GGACATCCCCCGCGCAGGCTGCCGCCTGGTGCCAGA |
| | | GCATGTGGAGACGAGGCCGCTGCTCAACCCCGACAA |
| | | GCCGGGCATCGAGCAGGGCCGCCTGGAGCTGTGGGT |
| | | GGACATGTTCCCCATGGACATGCCAGCCCCTGGGACG |
| | | CCTCTGGACATCTCACCTCGGAAGCCCAAGAAGTACG |
| | | AGCTGCGGGTCATCATCTGGAACACAGATGAGGTGGT |
| | | CTTGGAGGACGACGACTTCTTCACAGGGGAGAAGTCC |
| | | AGTGACATCTTCGTGAGGGGGTGGCTGAAGGGCCAG |
| | | CAGGAGGACAAGCAGGACACAGACGTCCACTACCACT |
| | | CCCTCACTGGCGAGGGCAACTTCAACTGGCGCTACCT |
| | | GTTCCCCTTCGACTACCTGGGGGGGGAGGAGAAGATC |
| | | GTCATCTCCAAGAAGGAGTCCATGTTCTCCTGGGACG |
| | | AGACCGAGTACAAGATCCCCGCGCGGCTCACCCTGCA |
| | | GATCTGGGATGCGGACCACTTCTCCGCTGACGACTTC |
| | | CTGGGGGCCATCGAGCTGGACCTGAACCGGTTCCCG |
| | | CGGGGCGCAAAGACAGCCAAGCAGTGCACCATGGAG |
| | | ATGGCCACCGGGGAGGTGGACGTGCCCCTCGTGTCC |
| | | ATCTTCAAGCAAAAGCGCGTCAAAGGCTGGTGGCCCC |
| | | TCCTGGCCCGCAATGAGAACGATGAGTTTGAGCTCAC |
| | | GGGCAAGGTGGAGGCTGAGCTGCATTTACTGACAGCA |
| | | GAGGAGGCAGAGAAGAACCCAGTGGGCCTGGCCCGC |
| | | AATGAACCTGACCCCCTAGAGAAACCCAACCGGCCCG |
| | | ACACGGCCTTCGTCTGGTTCCTCAACCCTCTCAAGTCC |
| | | ATCAAGTACCTCATCTGCACCCGGTACAAGTGGCTCAT |
| | | CATCAAGATCGTGCTGGCGCTGTTGGGGCTGCTCATG |
| | | TTGGGGCTCTTCCTCTACAGCCTCCCTGGCTACATGG |
| | | TCAAAAAGCTCCTTGGGGCATGAACGGCCGCTATGCT |
| | | AGCTTGGTACCAAGGGCGGATCCTGCATAGAGCTCGC |
| | | TGATCAGCCTCGACTGTGCCTTCTAGTTGCCAGCCATC |
| | | TGTTGTTTGCCCCTCCCCCGTGCCTTCCTTGACCCTG |
| | | GAAGGTGCCACTCCCACTGTCCTTTCCTAATAAAATGA |
| | | GGAAATTGCATCGCATTGTCTGAGTAGGTGTCATTCTA |
| | | TTCTGGGGGGTGGGGTGGGGCAGGACAGCAAGGGG |
| | | GAGGATTGGGAAGACAATAGCAGGCATCTCGAGTTAA |
| | | GGGCGAATTCCCGATAAGGATCTTCCTAGAGCATGGC |
| | | TACGTAGATAAGTAGCATGGCGGGTTAATCATTAACTA |
| | | CAAGGAACCCCTAGTGATGGAGTTGGCCACTCCCTCT |
| | | CTGCGCGCTCGCTCGCTCACTGAGGCCGGGCGACCA |
| | | AAGGTCGCCCGACGCCCGGGCTTTGCCCGGGCGGCC |
| | | TCAGTGAGCGAGCGAGCGCGCAGCCTTAATTAACCTA |
| | | ATTCACTGGCCGTCGTTTTACAACGTCGTGACTGGGAA |
| | | AACCCTGGCGTTACCCAACTTAATCGCCTTGCAGCACA |
| | | TCCCCCTTTCGCCAGCTGGCGTAATAGCGAAGAGGCC |
| | | CGCACCGATCGCCCTTCCCAACAGTTGCGCAGCCTGA |
| | | ATGGCGAATGGGACGCGCCCTGTAGCGGCGCATTAA |
| | | GCGCGGCGGGTGTGGTGGTTACGCGCAGCGTGACCG |
| | | CTACACTTGCCAGCGCCCTAGCGCCCGCTCCTTTCGC |
| | | TTTCTTCCCTTCCTTTCTCGCCACGTTCGCCGGCTTTC |
| | | CCCGTCAAGCTCTAAATCGGGGGCTCCCTTTAGGGTT |
| | | CCGATTTAGTGCTTTACGGCACCTCGACCCCAAAAAAC |
| | | TTGATTAGGGTGATGGTTCACGTAGTGGGCCATCGCC |
| | | CTGATAGACGGTTTTTCGCCCTTTGACGTTGGAGTCCA |
| | | CGTTCTTTAATAGTGGACTCTTGTTCCAAACTGGAACA |
| | | ACACTCAACCCTATCTCGGTCTATTCTTTTGATTTATAA |
| | | GGGATTTTGCCGATTTCGGCCTATTGGTTAAAAAATGA |
| | | GCTGATTTAACAAAAATTTAACGCGAATTTTAACAAAAT |
| | | ATTAACGCTTACAATTTAGGTGGCACTTTTCGGGGAAA |
| | | TGTGCGCGGAACCCCTATTTGTTTATTTTTCTAAATACA |
| | | TTCAAATATGTATCCGCTCATGAGACAATAACCCTGAT |
| | | AAATGCTTCAATAATATTGAAAAAGGAAGAGTATGAGC |
| | | CATATTCAACGGGAAACGTCGAGGCCGCGATTAAATT |
| | | CCAACATGGATGCTGATTTATATGGGTATAAATGGGCT |
| | | CGCGATAATGTCGGGCAATCAGGTGCGACAATCTATC |
| | | GCTTGTATGGGAAGCCCGATGCGCCAGAGTTGTTTCT |
| | | GAAACATGGCAAAGGTAGCGTTGCCAATGATGTTACA |
| | | GATGAGATGGTCAGACTAAACTGGCTGACGGAATTTAT |
| | | GCCTCTTCCGACCATCAAGCATTTTATCCGTACTCCTG |
| | | ATGATGCATGGTTACTCACCACTGCGATCCCCGGAAA |
| | | AACAGCATTCCAGGTATTAGAAGAATATCCTGATTCAG |
| | | GTGAAAATATTGTTGATGCGCTGGCAGTGTTCCTGCG |
| | | CCGGTTGCATTCGATTCCTGTTTGTAATTGTCCTTTTAA |
| | | CAGCGATCGCGTATTTCGTCTTGCTCAGGCGCAATCA |
| | | CGAATGAATAACGGTTTGGTTGATGCGAGTGATTTTGA |
| | | TGACGAGCGTAATGGCTGGCCTGTTGAACAAGTCTGG |
| | | AAAGAAATGCATAAACTTTTGCCATTCTCACCGGATTC |
| | | AGTCGTCACTCATGGTGATTTCTCACTTGATAACCTTAT |
| | | TTTTGACGAGGGGAAATTAATAGGTTGTATTGATGTTG |
| | | GACGAGTCGGAATCGCAGACCGATACCAGGATCTTGC |
| | | CATCCTATGGAACTGCCTCGGTGAGTTTTCTCCTTCAT |
| | | TACAGAAACGGCTTTTTCAAAAATATGGTATTGATAATC |
| | | CTGATATGAATAAATTGCAGTTTCATTTGATGCTCGATG |
| | | AGTTTTTCTAACTGTCAGACCAAGTTTACTCATATATAC |
| | | TTTAGATTGATTTAAAACTTCATTTTTAATTTAAAAGGAT |
| | | CTAGGTGAAGATCCTTTTTGATAATCTCATGACCAAAAT |
| | | CCCTTAACGTGAGTTTTCGTTCCACTGAGCGTCAGACC |
| | | CCGTAGAAAAGATCAAAGGATCTTCTTGAGATCCTTTT |
| | | TTTCTGCGCGTAATCTGCTGCTTGCAAACAAAAAAACC |
| | | ACCGCTACCAGCGGTGGTTTGTTTGCCGGATCAAGAG |
| | | CTACCAACTCTTTTTCCGAAGGTAACTGGCTTCAGCAG |
| | | AGCGCAGATACCAAATACTGTTCTTCTAGTGTAGCCGT |
| | | AGTTAGGCCACCACTTCAAGAACTCTGTAGCACCGCC |
| | | TACATACCTCGCTCTGCTAATCCTGTTACCAGTGGCTG |
| | | CTGCCAGTGGCGATAAGTCGTGTCTTACCGGGTTGGA |
| | | CTCAAGACGATAGTTACCGGATAAGGCGCAGCGGTCG |
| | | GGCTGAACGGGGGGTTCGTGCACACAGCCCAGCTTG |
| | | GAGCGAACGACCTACACCGAACTGAGATACCTACAGC |
| | | GTGAGCTATGAGAAAGCGCCACGCTTCCCGAAGGGAG |
| | | AAAGGCGGACAGGTATCCGGTAAGCGGCAGGGTCGG |
| | | AACAGGAGAGCGCACGAGGGAGCTTCCAGGGGGAAA |
| | | CGCCTGGTATCTTTATAGTCCTGTCGGGTTTCGCCACC |
| | | TCTGACTTGAGCGTCGATTTTTGTGATGCTCGTCAGGG |
| | | GGGCGGAGCCTATGGAAAAACGCCAGCAACGCGGCC |
| | | TTTTTACGGTTCCTGGCCTTTTGCTGGCCTTTTGCTCA |
| | | CATGTTCTTTCCTGCGTTATCCCCTGATTCTGTGGATA |
| | | ACCGTATTACCGCCTTTGAGTGAGCTGATACCGCTCG |
| | | CCGCAGCCGAACGACCGAGCGCAGCGAGTCAGTGAG |
| | | CGAGGAAGCGGAAGAGCGCCCAATACGCAAACCGCC |
| | | TCTCCCCGCGCGTTGGCCGATTCATTAATGCAGCTGG |
| | | CACGACAGGTTTCCCGACTGGAAAGCGGGCAGTGAG |
| | | CGCAACGCAATTAATGTGAGTTAGCTCACTCATTAGGC |
| | | ACCCCAGGCTTTACACTTTATGCTTCCGGCTCGTATGT |
| | | TGTGTGGAATTGTGAGCGGATAACAATTTCACACAGGA |
| | | AACAGCTATGACCATGATTACGCCAGATTTAATTAAGG |
| 83 | 5′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | CMV i.e enhancer at positions | GGGAGTGGCCAACTCCATCACTAGGGGTTCCTTGTAG |
| | 230-594 | TTAATGATTAACCCGCCATGCTACTTATCTACGTAGCC |
| | CMV enhancer at positions | ATGCTCTAGGAAGATCGGAATTCGCCCTTAAGCTAGC |
| | 296-599 | GGCGCGCCGGTACCTAGTTATTAATAGTAATCAATTAC |
| | Chicken β-actin promoter at | GGGGTCATTAGTTCATAGCCCATATATGGAGTTCCGC |
| | positions 596-878 | GTTACATAACTTACGGTAAATGGCCCGCCTGGCTGAC |
| | Exon 1 at positions 879-971 | CGCCCAACGACCCCCGCCCATTGACGTCAATAATGAC |
| | Chimeric intron at positions | GTATGTTCCCATAGTAACGCCAATAGGGACTTTCCATT |
| | 971-1172 | GACGTCAATGGGTGGAGTATTTACGGTAAACTGCCCA |
| | Kozak sequence at positions | CTTGGCAGTACATCAAGTGTATCATATGCCAAGTACGC |
| | 1189-1198 | CCCCTATTGACGTCAATGACGGTAAATGGCCCGCCTG |
| | N-terminal portion of human | GCATTATGCCCAGTACATGACCTTATGGGACTTTCCTA |
| | OTOF isoform 5 at positions | CTTGGCAGTACATCTACGTATTAGTCATCGCTATTACC |
| | 1195-3600 | ATGGTCGAGGTGAGCCCCACGTTCTGCTTCACTCTCC |
| | Splice donor sequence at | CCATCTCCCCCCCCTCCCCACCCCCAATTTTGTATTTA |
| | positions 3601-3684 | TTTATTTTTTAATTATTTTGTGCAGCGATGGGGGCGGG |
| | AP head sequence at positions | GGGGGGGGGGGGGCGCGCGCCAGGCGGGGCGGGG |
| | 3691-3977 | CGGGGCGAGGGGCGGGGGCGGGGGCGAGGCGGAGAG |
| | ITR at positions 4071-4200 | GTGCGGCGGCAGCCAATCAGAGCGGCGCGCTCCGAA |
| | M13 fwd at positions 4219-4235 | AGTTTCCTTTTATGGCGAGGCGGCGGCGGCGGCGGC |
| | f1 ori at positions 4377-4832 | CCTATAAAAAGCGAAGCGCGCGGCGGGCGGGAGTCG |
| | AmpR promoter at positions | CTGCGCGCTGCCTTCGCCCCGTGCCCCGCTCCGCCG |
| | 4858-4962 | CCGCCTCGCGCCGCCCGCCCCGGCTCTGACTGACCG |
| | KanR at positions 4963-5772 | CGTTACTCCCACAGGTGAGCGGGCGGGACGGCCCTT |
| | mutBsmBI at positions 4963- | CTCCTCCGGGCTGTAATTAGCGCTTGGTTTAATGACG |
| | 5772 | GCTTGTTTCTTTTCTGTGGCTGCGTGAAAGCCTTGAGG |
| | ori at positions 5943-6531 | GGCTCCGGGAGCTAGAGCCTCTGCTAACCATGTTCAT |
| | CAP binding site at positions | GCCTTCTTCTTTTTCCTACAGCTCCTGGGCAACGTGCT |
| | 6819-6840 | GGTTATTGTGCTGTCTCATCATTTTGGCAAAGAATTCTA |
| | lac promoter at positions 6855- | GCGGCCGCCACCATGGCCTTGCTCATCCACCTCAAGA |
| | 6885 | CAGTCTCGGAGCTGCGGGGCAGGGGCGACCGGATCG |
| | lac operator at positions 6835- | CCAAAGTGACTTTCCGAGGGCAATCCTTCTACTCTCGG |
| | 6909 | GTCCTGGAGAACTGTGAGGATGTGGCTGACTTTGATG |
| | M13 rev at positions 6917-6933 | AGACATTTCGGTGGCCGGTGGCCAGCAGCATCGACAG |
| | Transgene to be transferred | AAATGAGATGCTGGAGATTCAGGTTTTCAACTACAGCA |
| | into vector in dual vector | AAGTCTTCAGCAACAAGCTCATCGGGACCTTCCGCAT |
| | system at positions 12-4200 | GGTGCTGCAGAAGGTGGTAGAGGAGAGCCATGTGGA |
| | | GGTGACTGACACGCTGATTGATGACAACAATGCTATCA |
| | | TCAAGACCAGCCTGTGCGTGGAGGTCCGGTATCAGGC |
| | | CACTGACGGCACAGTGGGCTCCTGGGACGATGGGGA |
| | | CTTCCTGGGAGATGAGTCTCTTCAAGAGGAAGAGAAG |
| | | GACAGCCAAGAGACGGATGGACTGCTCCCAGGCTCC |
| | | CGGCCCAGCTCCCGGCCCCCAGGAGAGAAGAGCTTC |
| | | CGGAGAGCCGGGAGGAGCGTGTTCTCCGCCATGAAG |
| | | CTCGGCAAAAACCGGTCTCACAAGGAGGAGCCCCAAA |
| | | GACCAGATGAACCGGCGGTGCTGGAGATGGAAGACC |
| | | TTGACCATCTGGCCATTCGGCTAGGAGATGGACTGGA |
| | | TCCCGACTCGGTGTCTCTAGCCTCAGTCACAGCTCTC |
| | | ACCACTAATGTCTCCAACAAGCGATCTAAGCCAGACAT |
| | | TAAGATGGAGCCAAGTGCTGGGCGGCCCATGGATTAC |
| | | CAGGTCAGCATCACGGTGATCGAGGCCCGGCAGCTG |
| | | GTGGGCTTGAACATGGACCCTGTGGTGTGCGTGGAG |
| | | GTGGGTGACGACAAGAAGTACACATCCATGAAGGAGT |
| | | CCACTAACTGCCCCTATTACAACGAGTACTTCGTCTTC |
| | | GACTTCCATGTCTCTCCGGATGTCATGTTTGACAAGAT |
| | | CATCAAGATTTCGGTGATTCACTCCAAGAACCTGCTGC |
| | | GCAGTGGCACCCTGGTGGGCTCCTTCAAAATGGACGT |
| | | GGGAACCGTGTACTCGCAGCCAGAGCACCAGTTCCAT |
| | | CACAAGTGGGCCATCCTGTCTGACCCCGATGACATCT |
| | | CCTCGGGGCTGAAGGGCTACGTGAAGTGTGACGTTGC |
| | | CGTGGTGGGCAAAGGGGACAACATCAAGACGCCCCA |
| | | CAAGGCCAATGAGACCGACGAAGATGACATTGAGGGG |
| | | AACTTGCTGCTCCCCGAGGGGGTGCCCCCCGAACGC |
| | | CAGTGGGCCCGGTTCTATGTGAAAATTTACCGAGCAG |
| | | AGGGGCTGCCCCGTATGAACACAAGCCTCATGGCCAA |
| | | TGTAAAGAAGGCTTTCATCGGTGAAAACAAGGACCTC |
| | | GTGGACCCCTACGTGCAAGTCTTCTTTGCTGGCCAGA |
| | | AGGGCAAGACTTCAGTGCAGAAGAGCAGCTATGAGCC |
| | | CCTGTGGAATGAGCAGGTCGTCTTTACAGACCTCTTCC |
| | | CCCCACTCTGCAAACGCATGAAGGTGCAGATCCGAGA |
| | | CTCGGACAAGGTCAACGACGTGGCCATCGGCACCCAC |
| | | TTCATTGACCTGCGCAAGATTTCTAATGACGGAGACAA |
| | | AGGCTTCCTGCCCACACTGGGCCCAGCCTGGGTGAAC |
| | | ATGTACGGCTCCACACGTAACTACACGCTGCTGGATG |
| | | AGCATCAGGACCTGAACGAGGGCCTGGGGGAGGGTG |
| | | TGTCCTTCCGGGCCCGGCTCCTGCTGGGCCTGGCTGT |
| | | GGAGATCGTAGACACCTCCAACCCTGAGCTCACCAGC |
| | | TCCACAGAGGTGCAGGTGGAGCAGGCCACGCCCATC |
| | | TCGGAGAGCTGTGCAGGTAAAATGGAAGAATTCTTTCT |
| | | CTTTGGAGCCTTCCTGGAGGCCTCAATGATCGACCGG |
| | | AGAAACGGAGACAAGCCCATCACCTTTGAGGTCACCA |
| | | TAGGCAACTATGGGAACGAAGTTGATGGCCTGTCCCG |
| | | GCCCCAGCGGCCTCGGCCCCGGAAGGAGCCGGGGG |
| | | ATGAGGAAGAAGTAGACCTGATTCAGAACGCAAGTGA |
| | | TGACGAGGCCGGTGATGCCGGGGACCTGGCCTCAGT |
| | | CTCCTCCACTCCACCAATGCGGCCCCAGGTCACCGAC |
| | | AGGAACTACTTCCATCTGCCCTACCTGGAGCGAAAGC |
| | | CCTGCATCTACATCAAGAGCTGGTGGCCGGACCAGCG |
| | | CCGCCGCCTCTACAATGCCAACATCATGGACCACATT |
| | | GCCGACAAGCTGGAAGAAGGCCTGAACGACATACAGG |
| | | AGATGATCAAAACGGAGAAGTCCTACCCTGAGCGTCG |
| | | CCTGCGGGGCGTCCTGGAGGAGCTGAGCTGTGGCTG |
| | | CTGCCGCTTCCTCTCCCTCGCTGACAAGGACCAGGGC |
| | | CACTCATCCCGCACCAGGCTTGACCGGGAGCGCCTCA |
| | | AGTCCTGCATGAGGGAGCTGGTAAGTATCAAGGTTAC |
| | | AAGACAGGTTTAAGGAGACCAATAGAAACTGGGCTTG |
| | | TCGAGACAGAGAAGACTCTTGCGTTTCTGAGCTAGCC |
| | | CCCGGGTGCGCGGCGTCGGTGGTGCCGGCGGGGGG |
| | | CGCCAGGTCGCAGGCGGTGTAGGGCTCCAGGCAGGC |
| | | GGCGAAGGCCATGACGTGCGCTATGAAGGTCTGCTCC |
| | | TGCACGCCGTGAACCAGGTGCGCCTGCGGGCCGCGC |
| | | GCGAACACCGCCACGTCCTCGCCTGCGTGGGTCTCTT |
| | | CGTCCAGGGGCACTGCTGACTGCTGCCGATACTCGG |
| | | GGCTCCCGCTCTCGCTCTCGGTAACATCCGGCCGGG |
| | | CGCCGTCCTTGAGCACATAGCCTGGACCGTTTCGTCG |
| | | ACCTCGAGTTAAGGGCGAATTCCCGATAAGGATCTTC |
| | | CTAGAGCATGGCTACGTAGATAAGTAGCATGGCGGGT |
| | | TAATCATTAACTACAAGGAACCCCTAGTGATGGAGTTG |
| | | GCCACTCCCTCTCTGCGCGCTCGCTCGCTCACTGAGG |
| | | CCGGGCGACCAAAGGTCGCCCGACGCCCGGGCTTTG |
| | | CCCGGGCGGCCTCAGTGAGCGAGCGAGCGCGCAGCC |
| | | TTAATTAACCTAATTCACTGGCCGTCGTTTTACAACGTC |
| | | GTGACTGGGAAAACCCTGGCGTTACCCAACTTAATCG |
| | | CCTTGCAGCACATCCCCCTTTCGCCAGCTGGCGTAAT |
| | | AGCGAAGAGGCCCGCACCGATCGCCCTTCCCAACAGT |
| | | TGCGCAGCCTGAATGGCGAATGGGACGCGCCCTGTA |
| | | GCGGCGCATTAAGCGCGGCGGGTGTGGTGGTTACGC |
| | | GCAGCGTGACCGCTACACTTGCCAGCGCCCTAGCGC |
| | | CCGCTCCTTTCGCTTTCTTCCCTTCCTTTCTCGCCACG |
| | | TTCGCCGGCTTTCCCCGTCAAGCTCTAAATCGGGGGC |
| | | TCCCTTTAGGGTTCCGATTTAGTGCTTTACGGCACCTC |
| | | GACCCCAAAAAACTTGATTAGGGTGATGGTTCACGTAG |
| | | TGGGCCATCGCCCTGATAGACGGTTTTTCGCCCTTTG |
| | | ACGTTGGAGTCCACGTTCTTTAATAGTGGACTCTTGTT |
| | | CCAAACTGGAACAACACTCAACCCTATCTCGGTCTATT |
| | | CTTTTGATTTATAAGGGATTTTGCCGATTTCGGCCTATT |
| | | GGTTAAAAAATGAGCTGATTTAACAAAAATTTAACGCG |
| | | AATTTTAACAAAATATTAACGCTTACAATTTAGGTGGCA |
| | | CTTTTCGGGGAAATGTGCGCGGAACCCCTATTTGTTTA |
| | | TTTTTCTAAATACATTCAAATATGTATCCGCTCATGAGA |
| | | CAATAACCCTGATAAATGCTTCAATAATATTGAAAAAGG |
| | | AAGAGTATGAGCCATATTCAACGGGAAACGTCGAGGC |
| | | CGCGATTAAATTCCAACATGGATGCTGATTTATATGGG |
| | | TATAAATGGGCTCGCGATAATGTCGGGCAATCAGGTG |
| | | CGACAATCTATCGCTTGTATGGGAAGCCCGATGCGCC |
| | | AGAGTTGTTTCTGAAACATGGCAAAGGTAGCGTTGCCA |
| | | ATGATGTTACAGATGAGATGGTCAGACTAAACTGGCTG |
| | | ACGGAATTTATGCCTCTTCCGACCATCAAGCATTTTAT |
| | | CCGTACTCCTGATGATGCATGGTTACTCACCACTGCGA |
| | | TCCCCGGAAAAACAGCATTCCAGGTATTAGAAGAATAT |
| | | CCTGATTCAGGTGAAAATATTGTTGATGCGCTGGCAGT |
| | | GTTCCTGCGCCGGTTGCATTCGATTCCTGTTTGTAATT |
| | | GTCCTTTTAACAGCGATCGCGTATTTCGTCTTGCTCAG |
| | | GCGCAATCACGAATGAATAACGGTTTGGTTGATGCGA |
| | | GTGATTTTGATGACGAGCGTAATGGCTGGCCTGTTGA |
| | | ACAAGTCTGGAAAGAAATGCATAAACTTTTGCCATTCT |
| | | CACCGGATTCAGTCGTCACTCATGGTGATTTCTCACTT |
| | | GATAACCTTATTTTTGACGAGGGGAAATTAATAGGTTG |
| | | TATTGATGTTGGACGAGTCGGAATCGCAGACCGATAC |
| | | CAGGATCTTGCCATCCTATGGAACTGCCTCGGTGAGT |
| | | TTTCTCCTTCATTACAGAAACGGCTTTTTCAAAAATATG |
| | | GTATTGATAATCCTGATATGAATAAATTGCAGTTTCATT |
| | | TGATGCTCGATGAGTTTTTCTAACTGTCAGACCAAGTT |
| | | TACTCATATATACTTTAGATTGATTTAAAACTTCATTTTT |
| | | AATTTAAAAGGATCTAGGTGAAGATCCTTTTTGATAATC |
| | | TCATGACCAAAATCCCTTAACGTGAGTTTTCGTTCCAC |
| | | TGAGCGTCAGACCCCGTAGAAAAGATCAAAGGATCTT |
| | | CTTGAGATCCTTTTTTTCTGCGCGTAATCTGCTGCTTG |
| | | CAAACAAAAAAACCACCGCTACCAGCGGTGGTTTGTTT |
| | | GCCGGATCAAGAGCTACCAACTCTTTTTCCGAAGGTAA |
| | | CTGGCTTCAGCAGAGCGCAGATACCAAATACTGTTCTT |
| | | CTAGTGTAGCCGTAGTTAGGCCACCACTTCAAGAACTC |
| | | TGTAGCACCGCCTACATACCTCGCTCTGCTAATCCTGT |
| | | TACCAGTGGCTGCTGCCAGTGGCGATAAGTCGTGTCT |
| | | TACCGGGTTGGACTCAAGACGATAGTTACCGGATAAG |
| | | GCGCAGCGGTCGGGCTGAACGGGGGGTTCGTGCACA |
| | | CAGCCCAGCTTGGAGCGAACGACCTACACCGAACTGA |
| | | GATACCTACAGCGTGAGCTATGAGAAAGCGCCACGCT |
| | | TCCCGAAGGGAGAAAGGCGGACAGGTATCCGGTAAG |
| | | CGGCAGGGTCGGAACAGGAGAGCGCACGAGGGAGCT |
| | | TCCAGGGGGAAACGCCTGGTATCTTTATAGTCCTGTC |
| | | GGGTTTCGCCACCTCTGACTTGAGCGTCGATTTTTGTG |
| | | ATGCTCGTCAGGGGGGCGGAGCCTATGGAAAAACGC |
| | | CAGCAACGCGGCCTTTTTACGGTTCCTGGCCTTTTGCT |
| | | GGCCTTTTGCTCACATGTTCTTTCCTGCGTTATCCCCT |
| | | GATTCTGTGGATAACCGTATTACCGCCTTTGAGTGAGC |
| | | TGATACCGCTCGCCGCAGCCGAACGACCGAGCGCAG |
| | | CGAGTCAGTGAGCGAGGAAGCGGAAGAGCGCCCAAT |
| | | ACGCAAACCGCCTCTCCCCGCGCGTTGGCCGATTCAT |
| | | TAATGCAGCTGGCACGACAGGTTTCCCGACTGGAAAG |
| | | CGGGCAGTGAGCGCAACGCAATTAATGTGAGTTAGCT |
| | | CACTCATTAGGCACCCCAGGCTTTACACTTTATGCTTC |
| | | CGGCTCGTATGTTGTGTGGAATTGTGAGCGGATAACA |
| | | ATTTCACACAGGAAACAGCTATGACCATGATTACGCCA |
| | | GATTTAATTAAGG |

### Vectors for the Expression of OTOF

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes a portion of OTOF, as well as, e.g., additional sequence elements used for the expression of these agents and/or the integration of these polynucleotide sequences into the genome of a mammalian cell. Certain vectors that can be used for the expression of OTOF include plasmids that contain regulatory sequences, such as promoter and enhancer regions, which direct gene transcription. Other useful vectors for expression of OTOF contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the compositions and methods described herein are recombinant nucleic acid constructs that include (1) a heterologous sequence to be expressed (e.g., a polynucleotide encoding an N-terminal or C-terminal portion of an OTOF protein) and (2) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S. For targeting cochlear hair cells, AAV1, AAV2, AAV6, AAV9, Anc80, Anc80L65, DJ/9, 7m8, and PHP.B may be particularly useful. Serotypes evolved for transduction of the retina may also be used in the methods and compositions described herein. The first and second nucleic acid vectors in the compositions and methods described herein may have the same serotype or different serotypes. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the use of AAV vectors for delivering a functional OTOF protein requires the use of a dual vector system, in in which the first member of the dual vector system encodes an N-terminal portion of an OTOF protein and the second member encodes a C-terminal portion of an OTOF protein such that, upon administration of the dual vector system to a cell, the polynucleotide sequences contained within the two vectors can join to form a single sequence that results in the production of a full-length OTOF protein. In some embodiments, the protein is an OTOF isoform 5 protein. In some embodiments, the protein is an OTOF isoform 1 protein.

In some embodiments, the first member of the dual vector system will also include, in 5′ to 3′ order, a first inverted terminal repeat (“ITR”); a promoter (e.g., a Myo15 promoter); a Kozak sequence; an N-terminal portion of an OTOF coding sequence; a splice donor sequence; an AP gene fragment (e.g., an AP head sequence); and a second ITR; and the second member of the dual vector system will include, in 5′ to 3′ order, a first ITR; an AP gene fragment (e.g., an AP head sequence); a splice acceptor sequence; a C-terminal portion of an OTOF coding sequence; a polyA sequence; and a second ITR. In some embodiments, the N-terminal portion of the OTOF coding sequence and the C-terminal portion of the OTOF coding sequence do not overlap and are joined in a cell (e.g., by recombination at the overlapping region (the AP gene fragment), or by concatemerization of the ITRs) to produce the full-length OTOF amino sequence (e.g., for OTOF isoform 1, the sequence set forth in SEQ ID NO: 1, or for OTOF isoform 5, the sequence set forth in SEQ ID NO: 5). In particular embodiments, the N-terminal portion of the OTOF coding sequence encodes amino acids 1-802 of OTOF (e.g., amino acids 1-802 of SEQ ID NO: 1 or SEQ ID NO: 5, corresponding to SEQ ID NO: 73) and the C-terminal portion of the OTOF coding sequence encodes amino acids 803-1997 of OTOF (e.g., amino acids 803-1997 of SEQ ID NO: 1, or amino acids 803-1997 of SEQ ID NO: 5, corresponding to SEQ ID NO: 74).

In some embodiments, the first member of the dual vector system includes the Myo15 promoter of SEQ ID NO: 38 (also represented by nucleotides 235-1199 of SEQ ID NO: 81) operably linked to nucleotides that encode the N-terminal 802 amino acids of the OTOF isoform 5 protein (amino acids 1-802 of SEQ ID NO: 5), which are encoded by exons 1-20 of the native polynucleotide sequence encoding that protein. In certain embodiments, the nucleotide sequence that encodes the N-terminal amino acids of the OTOF isoform 5 protein is nucleotides 1222-3627 of SEQ ID NO: 81. In some embodiments, the nucleotide sequence that encodes the N-terminal amino acids of the OTOF isoform 5 protein is any nucleotide sequence that, by redundancy of the genetic code, encodes amino acids 1-802 of SEQ ID NO: 5. The nucleotide sequences that encodes the OTOF isoform 5 protein can be partially or fully codon-optimized for expression. In some embodiments, the first member of the dual vector system includes the Kozak sequence corresponding to nucleotides 1216-1225 of SEQ ID NO: 81. In some embodiments, the first member of the dual vector system includes the splice donor sequence corresponding to nucleotides 3628-3711 of SEQ ID NO: 81. In some embodiments, the first member of the dual vector system includes the AP head sequence corresponding to nucleotides 3718-4004 of SEQ ID NO: 81. In particular embodiments, the first member of the dual vector system includes nucleotides 235-4004 of SEQ ID NO: 81 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 12-141 of SEQ ID NO: 81 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4098-4227 of SEQ ID NO: 81 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., any of SEQ ID NOs: 75, 77, 79, 80, 81, or 83), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 12-4227 of SEQ ID NO: 81.

In some embodiments, the second member of the dual vector system includes nucleotides that encode the C-terminal 1195 amino acids of the OTOF isoform 5 protein (amino acids 803-1997 of SEQ ID NO: 5) immediately followed by a stop codon. In certain embodiments, the nucleotide sequence that encodes the C-terminal amino acids of the OTOF isoform 5 protein is nucleotides 587-4174 of SEQ ID NO: 82. In some embodiments, the nucleotide sequence that encodes the C-terminal amino acids of the OTOF isoform 5 protein is any nucleotide sequence that, by redundancy of the genetic code, encodes amino acids 803-1997 of SEQ ID NO: 5. The nucleotide sequences that encode the OTOF isoform 5 protein can be partially or fully codon-optimized for expression. In some embodiments, the second member of the dual vector system includes the splice acceptor sequence corresponding to nucleotides 538-586 of SEQ ID NO: 82. In some embodiments, the second member of the dual vector system includes the AP head sequence corresponding to nucleotides 229-515 of SEQ ID NO: 82. In some embodiments, the second member of the dual vector system includes the poly(A) sequence corresponding to nucleotides 4217-4438 of SEQ ID NO: 82. In particular embodiments, the second member of the dual vector system includes nucleotides 229-4438 of SEQ ID NO: 82 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 12-141 of SEQ ID NO: 82 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4526-4655 of SEQ ID NO: 82 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., any of SEQ ID NOs: 76, 78, or 82), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 12-4655 of SEQ ID NO: 82.

In some embodiments, the dual vector system is an AAV1 dual vector system.

In some embodiments, the dual vector system is an AAV9 dual vector system.

### Pharmaceutical Compositions

The nucleic acid vectors (e.g., AAV vectors) described herein may be incorporated into a vehicle for administration into a patient, such as a human patient suffering from biallelic OTOF mutations, as described herein. Pharmaceutical compositions containing vectors, such as viral vectors, that contain a polynucleotide encoding a portion of an OTOF protein can be prepared using methods known in the art. For example, such compositions can be prepared using, e.g., physiologically acceptable carriers, excipients, or stabilizers (Remington's Pharmaceutical Sciences 16th edition, Osol, A. Ed. (1980); incorporated herein by reference), and in a desired form, e.g., in the form of lyophilized formulations or aqueous solutions.

Mixtures of the nucleic acid vectors (e.g., AAV vectors) described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject with biallelic OTOF mutations by a variety of routes, such as local administration to the inner ear (e.g., administration into the perilymph or endolymph, e.g., to or through the oval window, round window, or horizontal canal, e.g., administration to a cochlear hair cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patients age, body weight, sex, severity of the disease being treated, the patient's diet, and the patient's excretion rate. Compositions may be administered once, or more than once (e.g., once annually, twice annually, three times annually, bi-monthly, monthly, or bi-weekly). In some embodiments, the first and second nucleic acid vectors are administered simultaneously (e.g., in one composition). In some embodiments, the first and second nucleic acid vectors are administered sequentially (e.g., the second nucleic acid vector is administered immediately after the first nucleic acid vector, or 5 minutes, 10 minutes, 15 minutes, 20 minutes, 25 minutes, 30 minutes, 45 minutes, 1 hour, 2 hours, 8 hours, 12 hours, 1 day, 2 days, 7 days, two weeks, 1 month or more after the first nucleic acid vector). The first and second nucleic acid vector can have the same serotype or different serotypes (e.g., AAV serotypes).

Subjects that may be treated as described herein are subjects having or at risk of developing sensorineural hearing loss or auditory neuropathy due to biallelic OTOF mutations that are 25 years of age or older (e.g., 25-50, 25-45, 25-40, 25-35, 25-30, 30-50, 30-45, 30-40, 30-35, 35-50, 35-45, 35-40, 40-50, 40-45, or 45-50 years old, e.g., 25, 26, 27, 28, 29, 30, 31, 32, 33, 34, 35, 36, 37, 38, 39, 40, 41, 42, 43, 44, 45, 46, 47, 48, 49, or 50 years old). Subjects may also be treated as described herein if they have biallelic OTOF mutations and are identified as having detectable indicators of outer hair cell integrity (the presence of otoacoustic emissions and/or cochlear microphonics) and/or inner hair cell integrity (the presence of a summating potential) (e.g., identified as having detectable otoacoustic emissions, cochlear microphonics, and/or summating potential prior to treatment). Accordingly, the methods described herein may include a step of assessing outer hair cell integrity and inner hair cell integrity prior to treatment of a subject. The compositions and methods described herein can be used to treat subjects having a mutation in OTOF (e.g., a mutation that reduces OTOF function or expression, or an OTOF mutation associated with sensorineural hearing loss or auditory neuropathy), subjects having a family history of autosomal recessive sensorineural hearing loss or auditory neuropathy (e.g., a family history of OTOF-related hearing loss) or subjects whose OTOF mutational status and/or OTOF activity level is unknown. The methods described herein may include a step of screening a subject for a mutation in OTOF prior to treatment with or administration of the compositions described herein. A subject can be screened for an OTOF mutation using standard methods known to those of skill in the art (e.g., genetic testing). The methods described herein may also include a step of assessing hearing in a subject prior to treatment with or administration of the compositions described herein. Hearing can be assessed using standard tests, such as audiometry, ABR, electrocochleography (ECOG), and otoacoustic emissions. The compositions and methods described herein may also be administered as a preventative treatment to patients at risk of developing hearing loss or auditory neuropathy, e.g., patients who have a family history of inherited hearing loss or patients carrying an OTOF mutation who do not yet exhibit hearing loss or impairment.

Treatment may include administration of a composition containing the nucleic acid vectors (e.g., AAV viral vectors) described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the cochlea. In cases in which the nucleic acid vectors are AAV vectors (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S vectors), the AAV vectors may have a titer of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear). In some embodiments, the nucleic acid vectors (e.g., AAV vectors) are administered in an amount sufficient to transduce at least 20% of the subject's inner hair cells with both the first vector and the second vector (e.g., at least 20%, 30%, 40%, 50%, 60%, 70%, 80%, 90%, 95% or more of the subject's inner hair cells are transduced with both vectors of the dual vector system).

The compositions described herein are administered in an amount sufficient to improve hearing, improve speech discrimination, increase WT OTOF expression (e.g., expression in a cochlear hair cell, e.g., an inner hair cell), or increase OTOF function. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100% or more) compared to hearing measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss or auditory neuropathy (e.g., in subjects who carry a mutation in OTOF or have a family history of autosomal recessive hearing loss but do not exhibit hearing impairment, or in subjects exhibiting mild to moderate hearing loss). OTOF expression may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100% or more) compared to OTOF expression prior to administration of the compositions described herein. OTOF function may be evaluated directly (e.g., using electrophysiological methods or imaging methods to assess exocytosis) or indirectly based on hearing tests, and may be increased by 5% or more (e.g., 5%, 10%, 15%, 20%, 30%, 40%, 50%, 60%, 70%, 80%, 90%, 100% or more) compared to OTOF function prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in treating a subject 25 years of age or older with biallelic OTOF mutations (e.g., to treat sensorineural hearing loss or auditory neuropathy in such a subject), or for use in treating a subject having biallelic OTOF mutations that is identified as having detectable otoacoustic emissions, detectable cochlear microphonics, and/or detectable summating potential (e.g., to treat sensorineural hearing loss or auditory neuropathy in such a subject). Compositions may include nucleic acid vectors (e.g., AAV vectors) described herein (e.g., a first nucleic acid vector containing a polynucleotide that encodes an N-terminal portion of an OTOF protein and a second nucleic acid vector containing a polynucleotide that encodes a C-terminal portion of an OTOF protein), optionally packaged in an AAV virus capsid (e.g., AAV1, AAV9, AAV2, AAV8, Anc80, Anc80L65, DJ/9, or 7m8). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1—ABR Recovery in 32- and 52-Week-Old OTOF Deficient Mice Treated with OTOF Dual Vectors

Animals progressively lose hearing due to age, which is partly due to losing outer hair cell function. Otoferlin deficient animals have absent ABRs (inner hair cell function) but present distortion product otoacoustic emissions (DPOAEs) (outer hair cell function). Like other aging animals, otoferlin null animals lose outer hair cell function and DPOAEs with age.

Older OTOF homozygous mutant (OTOF-Q828X) animals up to 52 weeks of age were dosed with dual hybrid AAV1-Myo15-hOTOF vectors at a dose of 3.9×10¹⁰ vg/ear to test the treatment window of efficacy considering possible outer hair cell loss and DPOAE elevation at later ages. The first vector contained the Myo15 promoter of SEQ ID NO: 38 operably linked to a polynucleotide containing exons 1-20 of a polynucleotide encoding an OTOF isoform 5 protein (SEQ ID NO: 71), a splice donor sequence 3′ of the polynucleotide sequence, and an AP recombinogenic region (SEQ ID NO: 65) 3′ of the splice donor sequence; and the second vector contained an AP recombinogenic region (SEQ ID NO: 65), a splice acceptor sequence 3′ of the recombinogenic region, a polynucleotide 3′ of the splice acceptor sequence containing exons 21-45 and 47 of a polynucleotide encoding an OTOF isoform 5 protein (SEQ ID NO: 72), and a poly(A) sequence.

Baseline DPOAEs were recorded in 32-week-old (n=15) and 52-week-old (n=15) animals. In 32-week-old animals, 4/15 had elevated baseline DPOAE, whereas 7/15 52-week-old animals had elevated baseline DPOAE.

Animals were dosed with vehicle (n=5/age group) or dual hybrid AAV1-Myo15-hOTOF (n=10/age group) through the round window under isoflurane anesthesia. Animals were allowed to recover after surgery according to the protocol. DPOAE and ABR were tested four and eight weeks post-delivery.

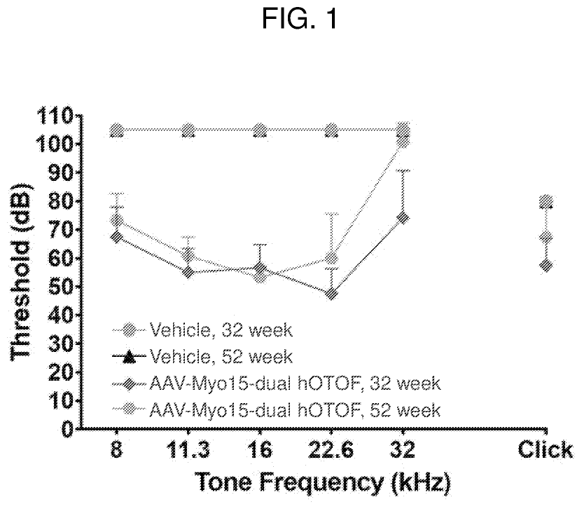

ABR recovery was seen in 10/10 of the 32-week-old virus-treated animals and 9/10 of the 52-week-old virus-treated animals, including the animals that had baseline DPOAE elevation at both four and eight weeks post-op. ABR recovery at four weeks post-treatment is shown in FIG. 1. The best recovery was seen at 22.6 kHz tone frequency and was similar to what is seen in younger animals.

### Example 2—Characterization of Hair Cell Loss in the OTOF-Q828X Mouse Model

The OTOF-Q828X mouse model was developed to mimic human congenital deafness resulting from otoferlin loss. The human otoferlin Q829X mutation (reference SNP rs80356593) is a well-studied stop-gain mutation in exon 22, resulting in truncation of the otoferlin protein after 828 amino acids of the 1997 amino acid coding sequence. CRISPR-mediated knock-in was used to generate the Otof-Q828X mouse line on an FVB strain background with a targeted mutation in mouse OTOF (mOtof) that mimics this human allele.

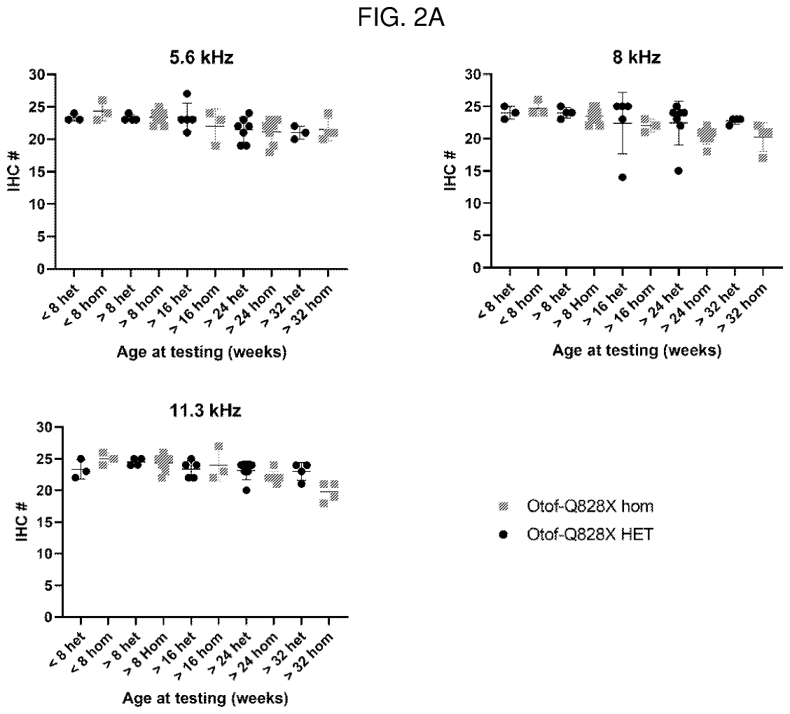

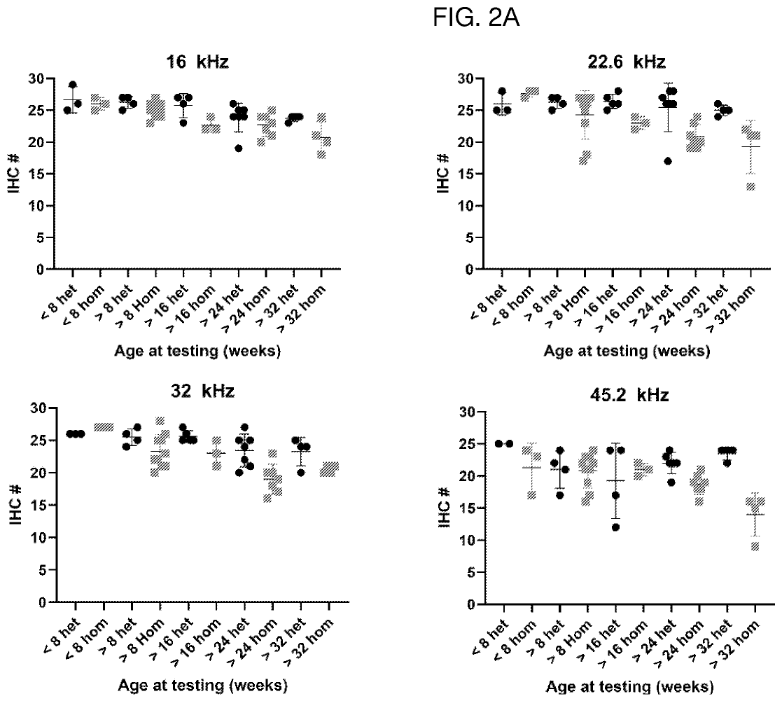

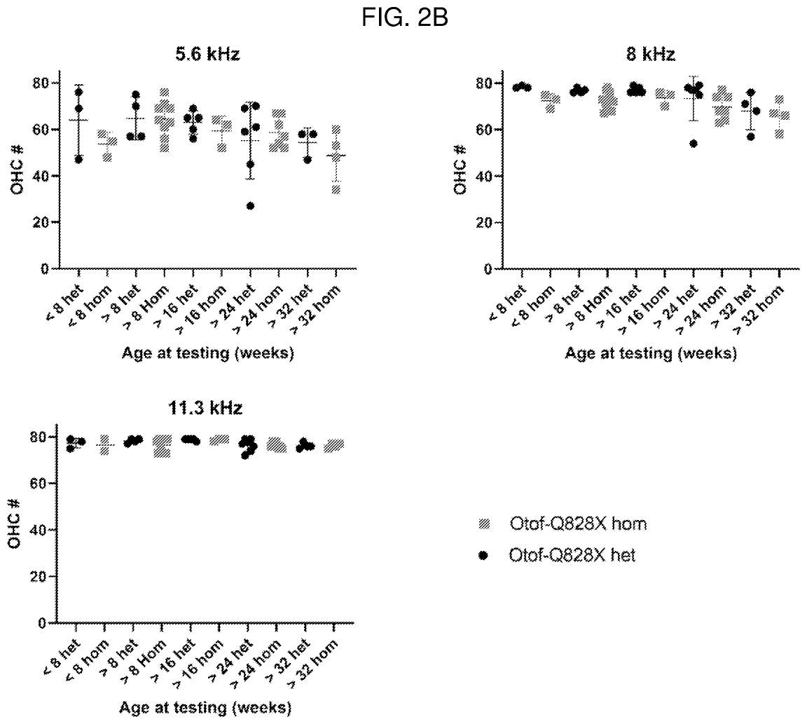

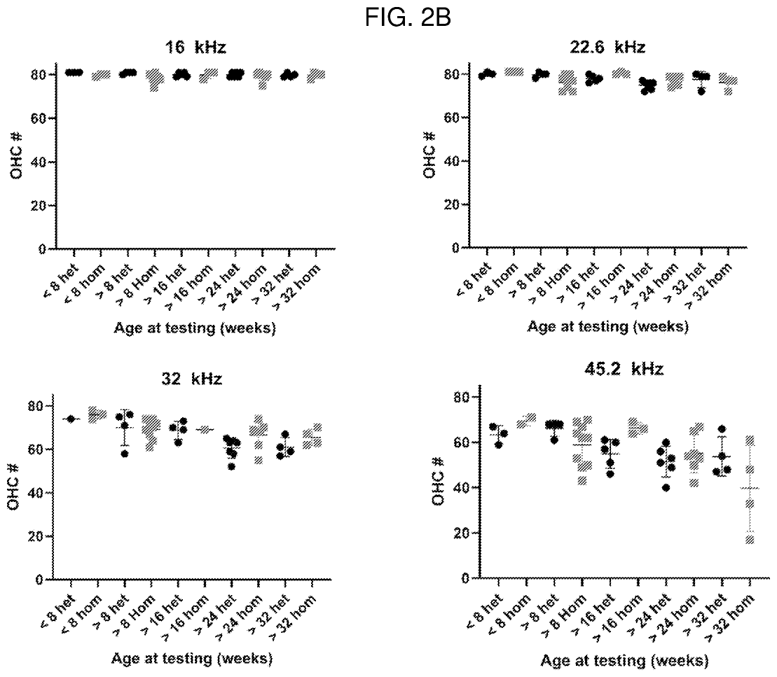

An experiment was performed to evaluate hair cell loss in homozygous Otof-Q828X (Otof-Q828X hom) and heterozygous (Otof-Q828X het) mice. Numbers of IHCs (FIG. 2A) and OHCs (FIG. 2B) were counted from 5 to 42 weeks of age in the cochlear regions corresponding to 5.6 kHz, 8 kHz, 11.3 kHz, 16 kHz, 22.6 kHz, 32 kHz, and 45.2 kHz for Otof-Q828X het or hom mice. Discrete IHCs and OHCs were counted by staining for a hair cell-specific marker. Fifty ears were evaluated for this analysis.

There was a statistically significant loss in IHC count with increasing age for all tested frequencies in Otof-Q828X hom mice. A similar trend in IHC count was observed in het mice for fewer frequencies (Kendall's rank correlation). IHC counts in Otof-828X hom and het animals were stable up to 16 weeks (FIG. 2A). Beginning after 16 weeks, Otof-Q828X hom animals showed a decrease in IHC counts starting at 22.6-45.2 kHz and after 24 weeks at lower frequencies (8-16 kHz). Loss of IHC counts in Otof-Q828X het mice started after 24 weeks for 16 and 32 kHz. After 32 weeks, there were over 75% of IHCs retained for most tested frequencies (<45.2 kHz) (FIG. 2A).

The outer hair cell numbers in the Otof-Q828X hom and het mice remained constant over the studied time course of 6 months over all frequencies except for 8 kHz for which het mice showed an age-related decrease in counts (Kendall's rank correlation). OHC counts in 5.6 kHz and 45.2 kHz were associated with greater variability, showing differences in counts between het and hom that were interspersed over the ages tested. The majority of OHCs remained after 32 weeks (FIG. 2B).

### Example 3—Relationship Between ABR Threshold Recovery and Otoferlin-Expressing Cell Number in Homozygous OTOF-Q828X Mutant Mice

The relationship between ABR threshold recovery and otoferlin-expressing cell number was examined across several studies in n=76 homozygous OTOF-Q828X mutant mice aged >4 weeks (between 4-weeks-old and 34-weeks-old) and receiving doses between 1.0×10⁹ and 6.4×10¹⁰ vg/ear, using otoferlin dual hybrid vector systems with the AAV1 or AAV2quadYF capsid and either the smCBA or Myo15 promoter. ABR thresholds were measured 4 to 34 weeks later when mice were between 10-weeks-old and 44-weeks-old. Dual hybrid vector systems administered during these studies included AAV2quadYF-smCBA (SEQ ID NO: 70)-mOTOF (administered to 34- and 29-week-old mice), AAV2quadYF-Myo15 (SEQ ID NO: 38)-mOTOF (administered to 29-week-old mice), AAV2quadYF-Myo15 (SEQ ID NO: 48)-mOTOF (administered to 29-week-old mice), AAV1-smCBA (SEQ ID NO: 70)-hOTOF (administered to 4- and 8-week-old mice), AAV1-Myo15 (SEQ ID NO: 38)-hOTOF (administered to 4- and 5-week-old mice), and AAV1-Myo15 (SEQ ID NO: 38)-mOTOF (administered to 9-week-old mice).

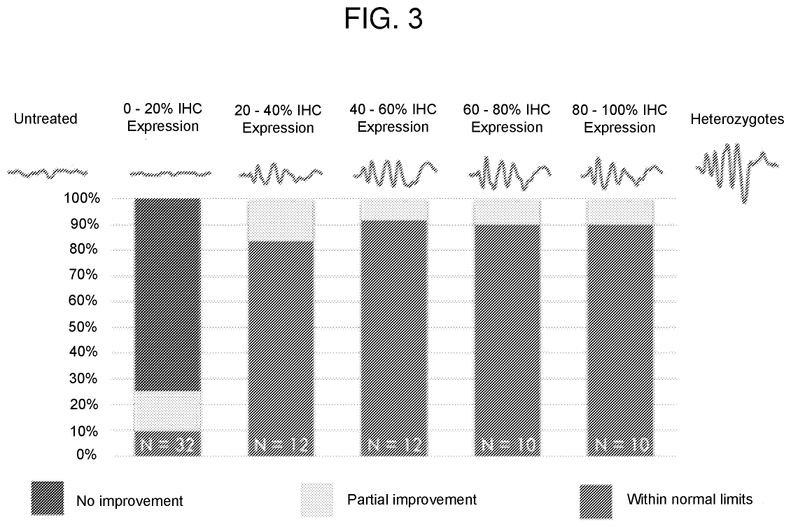

At 22 kHz, ABR thresholds entered the normal range (mean±2 SDs) when about 20% of IHCs expressed otoferlin (FIG. 3). Thresholds did not improve further with higher proportions of IHCs expressing otoferlin.

### Example 4—Administration of an OTOF Dual Vector System to a Subject Over 25 Years Old with Biallelic OTOF Mutations

According to the methods disclosed herein, a physician of skill in the art can treat a patient with biallelic OTOF mutations who is over 25 years old (e.g., 25-50, 25-45, 25-40, 25-35, 25-30, 30-50, 30-45, 30-40, 30-35, 35-50, 35-45, 35-40, 40-50, 40-45, or 45-50 years old, e.g., 25, 26, 27, 28, 29, 30, 31, 32, 33, 34, 35, 36, 37, 38, 39, 40, 41, 42, 43, 44, 45, 46, 47, 48, 49, or 50 years old) so as to prevent, reduce, or treat hearing loss or auditory neuropathy. To this end, a physician of skill in the art can administer to the human patient a composition containing a first nucleic acid vector (e.g., an AAV1 or AAV9 vector) containing a promoter operably linked to a polynucleotide encoding an N-terminal portion of an OTOF protein (e.g., human OTOF, e.g., an N-terminal portion of SEQ ID NO: 1 or SEQ ID NO: 5), and a second nucleic acid vector (e.g., an AAV1 or AAV9 vector) containing a polynucleotide encoding a C-terminal portion of an OTOF protein (e.g., human OTOF, e.g., a C-terminal portion of SEQ ID NO: 1 or SEQ ID NO: 5) and a poly(A) sequence. The dual vectors may be overlapping dual vectors, trans-splicing dual vectors, or dual hybrid vectors as described herein. For example, the vectors may be dual hybrid vectors in which the first vector contains a Myo15 promoter (e.g., SEQ ID NO: 36, 38, 39, 48, or 49) operably linked to exons 1-20 of a polynucleotide encoding an OTOF isoform 5 protein (e.g., human OTOF isoform 5, e.g., SEQ ID NO: 5, e.g., a polynucleotide having the sequence of SEQ ID NO: 71), a splice donor sequence 3′ of the polynucleotide sequence, and an AP recombinogenic region (e.g., an AP gene fragment, such as any one of SEQ ID NOs: 62-67, e.g., SEQ ID NO: 65) 3′ of the splice donor sequence, and in which the second vector contains an AP recombinogenic region (an AP gene fragment, such as any one of SEQ ID NOs: 62-67, e.g., SEQ ID NO: 65), a splice acceptor sequence 3′ of the recombinogenic region, a polynucleotide 3′ of the splice acceptor sequence that contains exons 21-45 and 47 of a polynucleotide encoding an OTOF isoform 5 protein (e.g., human OTOF isoform 5, e.g., SEQ ID NO: 5, e.g., a polynucleotide having the sequence of SEQ ID NO: 72), and a bGH poly(A) sequence. The composition containing the overlapping dual AAV vectors may be administered to the patient, for example, by local administration to the inner ear (e.g., injection through the round window membrane), to treat or prevent the development of sensorineural hearing loss or auditory neuropathy related to biallelic OTOF mutations.

Following administration of the composition to a patient, a practitioner of skill in the art can monitor the patient's improvement in response to the therapy, by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

Exemplary embodiments of the invention are described in the enumerated paragraphs below.
E1. A method of treating a human subject 25 years of age or older having biallelic otoferlin (OTOF) mutations, comprising administering to the subject a therapeutically effective amount of a dual vector system comprising:
   a first nucleic acid vector comprising a promoter operably linked to a first coding polynucleotide that encodes an N-terminal portion of an OTOF protein; and
   a second nucleic acid vector comprising a second coding polynucleotide that encodes a C-terminal portion of an OTOF protein and a poly(A) sequence positioned 3′ of the second coding polynucleotide;
   wherein neither the first nor the second nucleic acid vector encodes a full-length OTOF protein.
E2. The method of E1, wherein the first coding polynucleotide and the second coding polynucleotide do not overlap.
E3. The method of E1 or E2, wherein the first nucleic acid vector comprises a splice donor signal sequence positioned 3′ of the first coding polynucleotide and the second nucleic acid vector comprises a splice acceptor signal sequence positioned 5′ of the second coding polynucleotide.
E4. The method of E3, wherein the first nucleic acid vector comprises a first recombinogenic region positioned 3′ of the splice donor signal sequence and the second nucleic acid vector comprises a second recombinogenic region positioned 5′ of the splice acceptor signal sequence.
E5. The method of E4, wherein the first and second recombinogenic regions are the same.
E6. The method of E4 or E5, wherein the first or second recombinogenic region is an AP gene fragment or an F1 phage AK gene.
E7. The method of E6, wherein the F1 phage AK gene comprises or consists of the sequence of SEQ ID NO: 19.
E8. The method of E6, wherein the AP gene fragment comprises or consists of the sequence of any one of SEQ ID NOs: 62-67.
E9. The method of E8, wherein the AP gene fragment comprises or consists of the sequence of SEQ ID NO: 65.
E10. The method of any one of E3-E9, wherein the splice donor sequence comprises or consists of the sequence of SEQ ID NO: 20 or SEQ ID NO: 68.
E11. The method of any one of E3-E10, wherein the splice acceptor sequence comprises or consists of the sequence of SEQ ID NO: 21 or SEQ ID NO: 69.
E12. The method of any one of E4-E11, wherein the first nucleic acid vector further comprises a degradation signal sequence positioned 3′ of the recombinogenic region; and wherein the second nucleic acid vector further comprises a degradation signal sequence positioned between the recombinogenic region and the splice acceptor signal sequence.
E13. The method of E12, wherein the degradation signal sequence comprises or consists of the sequence of SEQ ID NO: 22.
E14. The method of any one of E1-E13, wherein the first and second coding polynucleotides are divided at an OTOF exon boundary.
E15. The method of E14, wherein the OTOF exon boundary is not within a portion of the first coding polynucleotide or second coding polynucleotide that encodes a C2 domain.
E16. The method of E1, wherein the first coding polynucleotide partially overlaps with the second coding polynucleotide.
E17. The method of E16, wherein the first coding polynucleotide overlaps with the second coding polynucleotide by at least 1 kilobase (kb).
E18. The method of E16 or E17, wherein the region of overlap between the first and second coding polynucleotides is centered at an OTOF exon boundary.
E19. The method of E18, wherein the first coding polynucleotide encodes an N-terminal portion of the OTOF protein and comprises an OTOF N-terminus to 500 bp 3′ of the exon boundary at the center of the overlap region; and the second coding polynucleotide encodes a C-terminal portion of the OTOF protein and comprises 500 bp 5′ of the exon boundary at the center of the overlap region to the OTOF C-terminus.
E20. The method of E18 or E19, wherein the OTOF exon boundary at the center of the overlap region is not within a portion of the first coding polynucleotide or second coding polynucleotide that encodes a C2 domain.
E21. The method of any one of E14, E15, and E18-E20, wherein the OTOF exon boundary is selected such that the first coding polynucleotide encodes an entire C2C domain and the second coding polynucleotide encodes an entire C2D domain.
E22. The method of any one of E14, E15, and E18-E21, wherein the OTOF exon boundary is an exon 19/20 boundary, an exon 20/21 boundary, or an exon 21/22 boundary.
E23. The method of any one of E14, E15, and E18-E20, wherein the OTOF exon boundary is selected such that the first coding polynucleotide encodes an entire C2D domain and the second coding polynucleotide encodes an entire C2E domain.
E24. The method of any one of E14, E15, E18-E20, and E23, wherein the OTOF exon boundary is an exon 26/27 boundary or an exon 28/29 boundary.
E25. The method of any one of E14, E18, and E19, wherein the OTOF exon boundary is within a portion of the first coding polynucleotide and the second coding polynucleotide that encodes a C2D domain.
E26. The method of any one of E14, E18, E19, and E25, wherein the OTOF exon boundary is an exon 24/25 boundary or an exon 25/26 boundary.
E27. The method of any one of E1-E26, wherein each of the first and second coding polynucleotides encode about half of the OTOF protein sequence.
E28. The method of any one of E1-E27, wherein the first nucleic acid vector and the second nucleic acid vector do not comprise OTOF untranslated regions (UTRs).
E29. The method of any one of E1-E27, wherein the first nucleic acid vector comprises an OTOF 5′ UTR.
E30. The method of any one of E1-E27 and E29, wherein the second nucleic acid vector comprises an OTOF 3′ UTR.
E31. The method of any one of E1-E30, wherein the first and second coding polynucleotides that encode the OTOF protein do not comprise introns.
E32. The method of any one of E1-E31, wherein the OTOF protein is a mammalian OTOF protein.
E33. The method of E32, wherein the OTOF protein is a human OTOF protein.
E34. The method of any one of E1-E33, wherein the OTOF protein has at least 85% sequence identity (e.g., 85%, 86%, 87%, 88%, 89%, 90%, 91%, 92%, 93%, 94%, 95%, 96%, 97%, 98%, 99%, or 100% sequence identity) to the sequence of SEQ ID NO: 1, SEQ ID NO: 2, SEQ ID NO:3, SEQ ID NO: 4, or SEQ ID NO: 5.
E35. The method of E34, wherein the OTOF protein has the sequence of SEQ ID NO: 1.
E36. The method of E34, wherein the OTOF protein has the sequence of SEQ ID NO: 5.
E37. The method of any one of E1-E33, wherein the OTOF protein comprises the sequence of SEQ ID NO: 1, SEQ ID NO: 2, SEQ ID NO:3, SEQ ID NO: 4, or SEQ ID NO: 5 or a variant thereof having one or more conservative amino acid substitutions.
E38. The method of E37, wherein no more than 10% (e.g., 10%, 9%, 8%, 7%, 6%, 5%, 4%, 3%, 2%, 1%, or less) of the amino acids in the OTOF protein variant are conservative amino acid substitutions.
E39. The method of any one of E1-E33, wherein the OTOF protein is encoded by any one of SEQ ID NOs: 10-14.
E40. The method of any one of E1-E33, wherein the first coding polynucleotide encodes amino acids 1-802 of SEQ ID NO: 1 or SEQ ID NO: 5 and the second coding polynucleotide encodes amino acids 803-1997 of SEQ ID NO: 1 or SEQ ID NO: 5.
E41. The method of any one of E1-E33, wherein the N-terminal portion of the OTOF protein consists of the sequence of SEQ ID NO: 73 or a variant thereof having one or more conservative amino acid substitutions.
E42. The method of E41, wherein no more than 10% (e.g., 10%, 9%, 8%, 7%, 6%, 5%, 4%, 3%, 2%, 1%, or less) of the amino acids in the N-terminal portion of the OTOF protein variant are conservative amino acid substitutions.
E43. The method of E41, wherein the N-terminal portion of the OTOF protein consists of the sequence of SEQ ID NO: 73.
E44. The method of any one of E1-E33 and E43, the N-terminal portion of the OTOF protein is encoded by the sequence of SEQ ID NO: 71.
E45. The method of any one of E1-E33 and E41-E44, wherein the C-terminal portion of the OTOF protein consists of the sequence of SEQ ID NO: 74 or a variant thereof having one or more conservative amino acid substitutions.
E46. The method of E45, wherein no more than 10% (e.g., 10%, 9%, 8%, 7%, 6%, 5%, 4%, 3%, 2%, 1%, or less) of the amino acids in the C-terminal portion of the OTOF protein variant are conservative amino acid substitutions.
E47. The method of E45, wherein the C-terminal portion of the OTOF protein consists of the sequence of SEQ ID NO: 74.
E48. The method of any one of E1-E33, E41-E44, and E47, wherein the C-terminal portion of the OTOF protein is encoded by the sequence of SEQ ID NO: 72.
E49. The method of any one of E1-E48, wherein the first nucleic acid vector comprises a Kozak sequence 3′ of the promoter and 5′ of the first coding polynucleotide that encodes the N-terminal portion of the OTOF protein.
E50. The method of any one of E1-E49, wherein the promoter is a ubiquitous promoter.
E51. The method of E50, wherein the ubiquitous promoter is a CAG promoter, a cytomegalovirus (CMV) promoter, a chicken β-actin promoter, a truncated CMV-chicken β-actin promoter (smCBA), a CB7 promoter, a hybrid CMV enhancer/human β-actin promoter, a human β-actin promoter, an elongation factor-1a (EF1α) promoter, or a phosphoglycerate kinase (PGK) promoter.
E52. The method of any one of E1-E49, wherein the promoter is a cochlear hair cell-specific promoter.
E53. The method of E52, wherein the cochlear hair cell-specific promoter is a myosin 15 (Myo15) promoter, a myosin 7A (Myo7A) promoter, a myosin 6 (Myo6) promoter, a POU class 4 homeobox 3 (POU4F3) promoter, an atonal BHLH transcription factor 1 (ATOH1) promoter, a LIM homeobox 3 (LHX3) promoter, an α9 acetylcholine receptor (α9AChR) promoter, or an α10 acetylcholine receptor (α10AChR) promoter.
E54. The method of any one of E1-E49, wherein the promoter is an inner hair cell-specific promoter.
E55. The method of E54, wherein the inner hair cell-specific promoter is a fibroblast growth factor 8 (FGF8) promoter, a vesicular glutamate transporter 3 (VGLUT3) promoter, an OTOF promoter, or a calcium binding protein 2 (CABP2) promoter.
E56. The method of E1, wherein the first nucleic acid vector comprises a polynucleotide sequence comprising the sequence of nucleotides 2272 to 6041 of SEQ ID NO: 75.
E57. The method of E1 or E56, wherein the first nucleic acid vector comprises a polynucleotide sequence comprising or consisting of the sequence of nucleotides 2049 to 6264 of SEQ ID NO: 75.
E58. The method of E1, wherein the first nucleic acid vector comprises a polynucleotide sequence comprising the sequence of nucleotides 182 to 3949 of SEQ ID NO: 77.
E59. The method of E1 or E58, wherein the first nucleic acid vector comprises a polynucleotide sequence comprising or consisting of the sequence of nucleotides 19 to 4115 of SEQ ID NO: 77.
E60. The method of E1, wherein the first nucleic acid vector comprises a polynucleotide sequence comprising the sequence of nucleotides 2267 to 6014 of SEQ ID NO: 79.
E61. The method of E1 or E60, wherein the first nucleic acid vector comprises a polynucleotide sequence comprising or consisting of the sequence of nucleotides 2049 to 6237 of SEQ ID NO: 79.
E62. The method of E1, wherein the first nucleic acid vector comprises a polynucleotide sequence comprising the sequence of nucleotides 177 to 3924 of SEQ ID NO: 80.
E63. The method of E1 or E62, wherein the first nucleic acid vector comprises a polynucleotide sequence comprising or consisting of the sequence of nucleotides 19 to 4090 of SEQ ID NO: 80.
E64. The method of any one of E1, E56, E57, E60, and E61, wherein the second nucleic acid vector comprises a polynucleotide sequence comprising the sequence of nucleotides 2267 to 6476 of SEQ ID NO: 76.
E65. The method of any one of E1, E56, E57, E60, E61, and E64, wherein the second nucleic acid vector comprises a polynucleotide sequence comprising or consisting of the sequence of nucleotides 2049 to 6693 of SEQ ID NO: 76.
E66. The method of any one of E1, E58, E59, E62, and E63, wherein the second nucleic acid vector comprises a polynucleotide sequence comprising the sequence of nucleotides 187 to 4396 of SEQ ID NO: 78.
E67. The method of any one of E1, E58, E59, E62, E63, and E66, wherein the second nucleic acid vector comprises a polynucleotide sequence comprising or consisting of the sequence of nucleotides 19 to 4589 of SEQ ID NO: 78.
E68. The method of E1, wherein the first nucleic acid vector comprises a polynucleotide sequence comprising the sequence of nucleotides 235 to 4004 of SEQ ID NO: 81.
E69. The method of E1 or E62, wherein the first nucleic acid vector comprises a polynucleotide sequence comprising or consisting of the sequence of nucleotides 12 to 4227 of SEQ ID NO: 81.
E70. The method of E1, wherein the first nucleic acid vector comprises a polynucleotide sequence comprising the sequence of nucleotides 230 to 3977 of SEQ ID NO: 83.
E71. The method of E1 or E70, wherein the first nucleic acid vector comprises a polynucleotide sequence comprising or consisting of the sequence of nucleotides 12 to 4200 of SEQ ID NO: 83.
E72. The method of any one of E1 and E68-E71, wherein the second nucleic acid vector comprises a polynucleotide sequence comprising the sequence of nucleotides 229 to 4438 of SEQ ID NO: 82.
E73. The method of any one of E1 and E68-E72, wherein the second nucleic acid vector comprises a polynucleotide sequence comprising or consisting of the sequence of nucleotides 12 to 4655 of SEQ ID NO: 82.
E74. The method of any one of E1-E73, wherein the first and second nucleic acid vectors comprise an inverted terminal repeat (ITR) at each end of the nucleic acid sequence.
E75. The method of E74, wherein the ITR is an AAV2 ITR or has at least 80% sequence identity (e.g., 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) to an AAV2 ITR.
E76. The method of any one of E1-E75, wherein the poly(A) sequence is a bovine growth hormone (bGH) poly(A) signal sequence.
E77. The method of any one of E1-E76, wherein the second nucleic acid vector comprises a Woodchuck Hepatitis Virus Posttranscriptional Regulatory Element (WPRE).
E78. The method of E77, wherein the WPRE comprises or consists of the sequence of SEQ ID NO: 23 or SEQ ID NO: 61.
E79. The method of any one of E1-E78, wherein the first and second nucleic acid vectors are adeno-associated virus (AAV) vectors.
E80. The method of E79, wherein the AAV vectors have AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S capsids.
E81. The method of any one of E1-E80, wherein the subject is 30 years of age or older.
E82. The method of any one of E1-E81, wherein the subject is 35 years of age or older.
E83. The method of any one of E1-E82, wherein the subject is 40 years of age or older.
E84. The method of any one of E1-E83, wherein the subject is 45 years of age or older.
E85. The method of any one of E1-E84, wherein the subject is no older than 50 years old.
E86. The method of any one of E1-E85, wherein the subject has been identified as having biallelic OTOF mutations.
E87. The method of any one of E1-E85, wherein the method further comprises identifying the subject as having biallelic OTOF mutations prior to administering the dual vector system.
E88. The method of any one of E1-E87, wherein the subject is identified as having detectable otoacoustic emissions.
E89. The method of any one of E1-E88, wherein the subject is identified as having detectable cochlear microphonics.
E90. The method of any one of E1-E89, wherein the subject is identified as having a detectable summating potential.
E91. The method of any one of E1-E90, wherein the subject has or is identified as having Deafness, Autosomal Recessive 9 (DFNB9).
E92. The method of any one of E1-E91, wherein the method further comprises evaluating the hearing of the subject prior to administering the dual vector system.
E93. The method of any one of E1-E92, wherein the dual vector system is administered to the inner ear.
E94. The method of E93, wherein the dual vector system is administered by injection through the round window membrane, injection into a semicircular canal, canalostomy, insertion of a catheter through the round window membrane, transtympanic injection, or intratympanic injection.
E95. The method of any one of E1-E94, wherein the method further comprises evaluating the hearing of the subject after administering the dual vector system.
E96. The method of any one of E1-E95, wherein the dual vector system is administered in an amount sufficient to prevent or reduce hearing loss, delay the development of hearing loss, slow the progression of hearing loss, improve hearing, improve speech discrimination, or improve hair cell function.
E97. The method of any one of E1-E96, wherein the first vector and the second vector are administered concurrently.
E98. The method of any one of E1-E96, wherein the first vector and the second vector are administered sequentially.
E99. The method of any one of E1-E98, wherein the first vector and the second vector are administered at a concentration of about 1×10⁷ vector genomes (VG)/ear to about 2×10¹⁵ VG/ear.
E100. The method of any one of E1-E99, wherein the first vector and the second vector are administered in amounts that together are sufficient to transduce at least 20% of the subject's inner hair cells with both the first vector and the second vector.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A method of treating a human subject 25 years of age or older having biallelic otoferlin (OTOF) mutations, comprising administering to the subject a therapeutically effective amount of a dual vector system comprising:
a first nucleic acid vector comprising a promoter operably linked to a first coding polynucleotide that encodes an N-terminal portion of an OTOF protein; and
a second nucleic acid vector comprising a second coding polynucleotide that encodes a C-terminal portion of an OTOF protein and a poly(A) sequence positioned 3′ of the second coding polynucleotide;
wherein neither the first nor the second nucleic acid vector encodes a full-length OTOF protein.

2. The method of claim 1, wherein the first coding polynucleotide and the second coding polynucleotide do not overlap.

3. The method of claim 1 or 2, wherein the first nucleic acid vector comprises a splice donor signal sequence positioned 3′ of the first coding polynucleotide and the second nucleic acid vector comprises a splice acceptor signal sequence positioned 5′ of the second coding polynucleotide.

4. The method of claim 3, wherein the first nucleic acid vector comprises a first recombinogenic region positioned 3′ of the splice donor signal sequence and the second nucleic acid vector comprises a second recombinogenic region positioned 5′ of the splice acceptor signal sequence.

5. The method of claim 4, wherein the first and second recombinogenic regions are the same.

6. The method of claim 4 or 5, wherein the first or second recombinogenic region is an AP gene fragment or an F1 phage AK gene.

7. The method of any one of claims 4-6, wherein the first nucleic acid vector further comprises a degradation signal sequence positioned 3′ of the recombinogenic region; and wherein the second nucleic acid vector further comprises a degradation signal sequence positioned between the recombinogenic region and the splice acceptor signal sequence.

8. The method of any one of claims 1-7, wherein the first and second coding polynucleotides are divided at an OTOF exon boundary.

9. The method of claim 1, wherein the first coding polynucleotide partially overlaps with the second coding polynucleotide.

10. The method of claim 9, wherein the first coding polynucleotide overlaps with the second coding polynucleotide by at least 1 kilobase (kb).

11. The method of claim 9 or 10, wherein the region of overlap between the first and second coding polynucleotides is centered at an OTOF exon boundary.

12. The method of claim 11, wherein the first coding polynucleotide encodes an N-terminal portion of the OTOF protein and comprises an OTOF N-terminus to 500 bp 3′ of the exon boundary at the center of the overlap region; and the second coding polynucleotide encodes a C-terminal portion of the OTOF protein and comprises 500 bp 5′ of the exon boundary at the center of the overlap region to the OTOF C-terminus.

13. The method of any one of claims 8, 11, and 12, wherein the OTOF exon boundary is selected such that the first coding polynucleotide encodes an entire C2C domain and the second coding polynucleotide encodes an entire C2D domain.

14. The method of any one of claims 8 and 11-13, wherein the OTOF exon boundary is an exon 19/20 boundary, an exon 20/21 boundary, or an exon 21/22 boundary.

15. The method of any one of claims 8, 11, and 12, wherein the OTOF exon boundary is selected such that the first coding polynucleotide encodes an entire C2D domain and the second coding polynucleotide encodes an entire C2E domain.

16. The method of any one of claims 8, 11, 12, and 15, wherein the OTOF exon boundary is an exon 26/27 boundary or an exon 28/29 boundary.

17. The method of any one of claims 8, 11, and 12, wherein the OTOF exon boundary is within a portion of the first coding polynucleotide and the second coding polynucleotide that encodes a C2D domain.

18. The method of any one of claims 8, 11, 12, and 17, wherein the OTOF exon boundary is an exon 24/25 boundary or an exon 25/26 boundary.

19. The method of any one of claims 1-18, wherein each of the first and second coding polynucleotides encode about half of the OTOF protein sequence.

20. The method of any one of claims 1-19, wherein the first nucleic acid vector and the second nucleic acid vector do not comprise OTOF untranslated regions (UTRs).

21. The method of any one of claims 1-19, wherein the first nucleic acid vector comprises an OTOF 5′ UTR.

22. The method of any one of claims 1-19 and 21, wherein the second nucleic acid vector comprises an OTOF 3′ UTR.

23. The method of any one of claims 1-22, wherein the first and second coding polynucleotides that encode the OTOF protein do not comprise introns.

24. The method of any one of claims 1-23, wherein the OTOF protein is a mammalian OTOF protein.

25. The method of claim 24, wherein the OTOF protein is a human OTOF protein.

26. The method of claim any one of claims 1-25, wherein the OTOF protein has at least 85% identity to the sequence of SEQ ID NO: 1, SEQ ID NO: 2, SEQ ID NO:3, SEQ ID NO: 4, or SEQ ID NO: 5.

27. The method of claim 26, wherein the OTOF protein has the sequence of SEQ ID NO: 1.

28. The method of claim 26, wherein the OTOF protein has the sequence of SEQ ID NO: 5.

29. The method of any one of claims 1-25, wherein the OTOF protein comprises the sequence of SEQ ID NO: 1, SEQ ID NO: 2, SEQ ID NO:3, SEQ ID NO: 4, or SEQ ID NO: 5 or a variant thereof having one or more conservative amino acid substitutions.

30. The method of claim 29, wherein no more than 10% of the amino acids in the OTOF protein variant are conservative amino acid substitutions.

31. The method of any one of claims 1-30, wherein the first nucleic acid vector comprises a Kozak sequence 3′ of the promoter and 5′ of the first coding polynucleotide that encodes the N-terminal portion of the OTOF protein.

32. The method of any one of claims 1-31, wherein the promoter is a ubiquitous promoter.

33. The method of claim 32, wherein the ubiquitous promoter is a CAG promoter, a cytomegalovirus (CMV) promoter, a chicken β-actin promoter, a truncated CMV-chicken β-actin promoter (smCBA), a CB7 promoter, a hybrid CMV enhancer/human β-actin promoter, a human β-actin promoter, an elongation factor-1α (EF1α) promoter, or a phosphoglycerate kinase (PGK) promoter.

34. The method of any one of claims 1-31, wherein the promoter is a cochlear hair cell-specific promoter.

35. The method of claim 34, wherein the cochlear hair cell-specific promoter is a myosin 15 (Myo15) promoter, a myosin 7A (Myo7A) promoter, a myosin 6 (Myo6) promoter, a POU class 4 homeobox 3 (POU4F3) promoter, an atonal BHLH transcription factor 1 (ATOH1) promoter, a LIM homeobox 3 (LHX3) promoter, an α9 acetylcholine receptor (α9AChR) promoter, or an α10 acetylcholine receptor (α10AChR) promoter.

36. The method of any one of claims 1-31, wherein the promoter is an inner hair cell-specific promoter.

37. The method of claim 36, wherein the inner hair cell-specific promoter is a fibroblast growth factor 8 (FGF8) promoter, a vesicular glutamate transporter 3 (VGLUT3) promoter, an OTOF promoter, or a calcium binding protein 2 (CABP2) promoter.

38. The method of any one of claims 1-37, wherein the first and second nucleic acid vectors comprise an inverted terminal repeat (ITR) at each end of the nucleic acid sequence.

39. The method of claim 38, wherein the ITR is an AAV2 ITR or has at least 80% sequence identity to an AAV2 ITR.

40. The method of any one of claims 1-39, wherein the second nucleic acid vector comprises a Woodchuck Hepatitis Virus Posttranscriptional Regulatory Element (WPRE).

41. The method of any one of claims 1-40, wherein the first and second nucleic acid vectors are adeno-associated virus (AAV) vectors.

42. The method of any one of claims 1-41, wherein the subject is 30 years of age or older.

43. The method of any one of claims 1-42, wherein the subject is 35 years of age or older.

44. The method of any one of claims 1-43, wherein the subject is 40 years of age or older.

45. The method of any one of claims 1-44, wherein the subject is 45 years of age or older.

46. The method of any one of claims 1-45, wherein the subject is no older than 50 years old.

47. The method of any one of claims 1-46, wherein the subject has been identified as having biallelic OTOF mutations.

48. The method of any one of claims 1-47, wherein the method further comprises identifying the subject as having biallelic OTOF mutations prior to administering the dual vector system.

49. The method of any one of claims 1-48, wherein the subject is identified as having detectable otoacoustic emissions.

50. The method of any one of claims 1-49, wherein the subject is identified as having detectable cochlear microphonics.

51. The method of any one of claims 1-50, wherein the subject is identified as having a detectable summating potential.

52. The method of any one of claims 1-51, wherein the subject has or is identified as having Deafness, Autosomal Recessive 9 (DFNB9).

53. The method of any one of claims 1-52, wherein the dual vector system is administered to the inner ear.

54. The method of any one of claims 1-53, wherein the first vector and the second vector are administered concurrently.

55. The method of any one of claims 1-54, wherein the first vector and the second vector are administered at a concentration of about 1×10⁷ vector genomes (VG)/ear to about 2×10¹⁵ VG/ear.

56. The method of any one of claims 1-55, wherein the first vector and the second vector are administered in amounts that together are sufficient to transduce at least 20% of the subject's inner hair cells with both the first vector and the second vector.


SEQUENCE LISTING (84 sequences)

SEQ ID NO: 1 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 2 - protein, 1230 aa, Homo sapiens
MIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 3 - protein, 1230 aa, Homo sapiens
MIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 4 - protein, 1307 aa, Homo sapiens
MMTDTQDGPSESSQIMRSLTPLINREEAFGEAGEAGLWPSITHTPDSQEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 5 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 6 - protein, 1997 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRRAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTVRLLRGCHRLRNGGPSSRPTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKFLLLFLLLLLFALFLYSLPGYLAKKILGA

SEQ ID NO: 7 - protein, 1977 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRRAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKFLLLFLLLLLFALFLYSLPGYLAKKILGA

SEQ ID NO: 8 - protein, 1992 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRSKGREKTKGGRDGEHKAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLALFLYSLPGYMVKKLLGA

SEQ ID NO: 9 - protein, 1977 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRRAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLALFLYSLPGYMVKKLLGA

SEQ ID NO: 10 - DNA, 5979 bp, Homo sapiens
atggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagcaaaggcagagagaagaccaagggaggcagagatggcgagcacaaagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctga

SEQ ID NO: 11 - DNA, 4969 bp, Homo sapiens
ccgtgagttctgcccaggccctgtgagctcaccagagccacagactcacagcccagaggtggcttcttccttcaggaactgaagaacccccatgaacaccaacatctccaggttctgagaacagaacctgggaaattgatgacttcctcatgatgaccgatactcaggatggccctagcgagagctcccagatcatgaggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacgggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacgagcttcatctggttcctgaaccctctcaagtcggctcgctacttcttgtggcacacgtatcgctggctgctcctcaaactgttgctgctcctgctgctgctcctcctcctcgccctgttcctctactctgtgcctggctacctggtcaagaaaatcctcggggcctgagcccagtggcctcctggccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 12 - DNA, 4771 bp, Homo sapiens
ccgtgagttctgcccaggccctgtgagctcaccagagccacagactcacagcccagaggtggcttcttccttcaggaactgaagaacccccatgaacaccaacatctccaggttctgagaacagaacctgggaaattgatgacttcctcatgatgaccgatactcaggatggccctagcgagagctcccagatcatgaggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacgggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 13 - DNA, 5123 bp, Homo sapiens
ccgtgagttctgcccaggccctgtgagctcaccagagccacagactcacagcccagaggtggcttcttccttcaggaactgaagaacccccatgaacaccaacatctccaggttctgagaacagaacctgggaaattgatgacttcctcatgatgaccgatactcaggatggccctagcgagagctcccagatcatgaggtccctcactcccctgatcaacagggaggaggcatttggggaggctggggaggcggggctgtggcccagcatcacccacactcctgattcacaggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacgagcttcatctggttcctgaaccctctcaagtcggctcgctacttcttgtggcacacgtatcgctggctgctcctcaaactgttgctgctcctgctgctgctcctcctcctcgccctgttcctctactctgtgcctggctacctggtcaagaaaatcctcggggcctgagcccagtggcctcctggccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 14 - DNA, 5994 bp, Homo sapiens
atggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatga

SEQ ID NO: 15 - DNA, 7125 bp, Mus musculus
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacagtcaggctgctccggggctgccacaggctgcgcaatgggggcccctcttctcgccccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaatcggccggacacaagcttcatctggttcttgaaccctctcaagtctgcccgctacttcctgtggcatacctaccgctggctactcctcaaattcctgctgctcttcctcctgctgctgctcttcgccctgtttctctactctctgcctggctacctggccaagaagatccttggggcctgagccctgcagtcgcctaggcctgccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 16 - DNA, 7065 bp, Mus musculus
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaatcggccggacacaagcttcatctggttcttgaaccctctcaagtctgcccgctacttcctgtggcatacctaccgctggctactcctcaaattcctgctgctcttcctcctgctgctgctcttcgccctgtttctctactctctgcctggctacctggccaagaagatccttggggcctgagccctgcagtcgcctaggcctgccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 17 - DNA, 6907 bp, Mus musculus
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagcaaaggcagagagaagaccaagggaggcagagatggcgagcacaaagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 18 - DNA, 6862 bp, Mus musculus
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 19 - DNA, 77 bp, Artificial Sequence
  Synthetic Construct
gggattttgccgatttcggcctattggttaaaaaatgagctgatttaacaaaaatttaacgcgaattttaacaaaat

SEQ ID NO: 20 - DNA, 82 bp, Artificial Sequence
  Synthetic Construct
gtaagtatcaaggttacaagacaggtttaaggagaccaatagaaactgggcttgtcgagacagagaagactcttgcgtttct

SEQ ID NO: 21 - DNA, 51 bp, Artificial Sequence
  Synthetic Construct
gataggcacctattggtcttactgacatccactttgcctttctctccacag

SEQ ID NO: 22 - DNA, 48 bp, Artificial Sequence
  Synthetic Construct
gcctgcaagaactggttcagcagcctgagccacttcgtgatccacctg

SEQ ID NO: 23 - DNA, 548 bp, Artificial Sequence
  Synthetic Construct
gatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcga

SEQ ID NO: 24 - DNA, 454 bp, Mus musculus
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 25 - DNA, 1157 bp, Mus musculus
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 26 - DNA, 76 bp, Mus musculus
cccatgtcagctgcttgtgctttccagagacaaaacaggaataatagatgtcattaaatatacattgggccccagg

SEQ ID NO: 27 - DNA, 95 bp, Mus musculus
agcctgagcctcctttccatctctgtggaggcagacataggacccccaacaaacagcatgcaggttgggagccagccacaggacccaggtaaggg

SEQ ID NO: 28 - DNA, 171 bp, Artificial Sequence
  Synthetic Construct
cccatgtcagctgcttgtgctttccagagacaaaacaggaataatagatgtcattaaatatacattgggccccaggagcctgagcctcctttccatctctgtggaggcagacataggacccccaacaaacagcatgcaggttgggagccagccacaggacccaggtaaggg

SEQ ID NO: 29 - DNA, 171 bp, Artificial Sequence
  Synthetic Construct
agcctgagcctcctttccatctctgtggaggcagacataggacccccaacaaacagcatgcaggttgggagccagccacaggacccaggtaagggcccatgtcagctgcttgtgctttccagagacaaaacaggaataatagatgtcattaaatatacattgggccccagg

SEQ ID NO: 30 - DNA, 184 bp, Mus musculus
cccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggg

SEQ ID NO: 31 - DNA, 82 bp, Mus musculus
tgaggtgggagctgggctctccctgatgtattattcagctccctggagttggccagctcctgttacactggccacagccctg

SEQ ID NO: 32 - DNA, 106 bp, Mus musculus
cacaggcctcccagtctaccctcactagaaaacaacacccaggcactttccaccacctctcaaaggtgaaacccaaggctggtctagagaatgaattatggatcct

SEQ ID NO: 33 - DNA, 188 bp, Artificial Sequence
  Synthetic Construct
tgaggtgggagctgggctctccctgatgtattattcagctccctggagttggccagctcctgttacactggccacagccctgcacaggcctcccagtctaccctcactagaaaacaacacccaggcactttccaccacctctcaaaggtgaaacccaaggctggtctagagaatgaattatggatcct

SEQ ID NO: 34 - DNA, 188 bp, Artificial Sequence
  Synthetic Construct
cacaggcctcccagtctaccctcactagaaaacaacacccaggcactttccaccacctctcaaaggtgaaacccaaggctggtctagagaatgaattatggatccttgaggtgggagctgggctctccctgatgtattattcagctccctggagttggccagctcctgttacactggccacagccctg

SEQ ID NO: 35 - DNA, 430 bp, Mus musculus
tgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcct

SEQ ID NO: 36 - DNA, 1611 bp, Artificial Sequence
  Synthetic Construct
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactatggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 37 - DNA, 1611 bp, Artificial Sequence
  Synthetic Construct
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 38 - DNA, 965 bp, Artificial Sequence
  Synthetic Construct
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 39 - DNA, 520 bp, Artificial Sequence
  Synthetic Construct
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 40 - DNA, 235 bp, Homo sapiens
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttcc

SEQ ID NO: 41 - DNA, 688 bp, Homo sapiens
tgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 42 - DNA, 77 bp, Homo sapiens
cccctgtcagctgctcactcctttccagagacaaaacaggaataatagacatcattaaatatacatagggccccagg

SEQ ID NO: 43 - DNA, 82 bp, Homo sapiens
tgagccggcagcctggctccccaccccatgtattattcagctcctgagagccagccagctcctgttacactgaccgcagccc

SEQ ID NO: 44 - DNA, 111 bp, Homo sapiens
cacaggcctcataggcaggcttccccatcctggtaaacaacacccacacactttctactactgctctagggtgaaacccaaggcgctctagaggagatgaattatggatcc

SEQ ID NO: 45 - DNA, 193 bp, Artificial Sequence
  Synthetic Construct
tgagccggcagcctggctccccaccccatgtattattcagctcctgagagccagccagctcctgttacactgaccgcagccccacaggcctcataggcaggcttccccatcctggtaaacaacacccacacactttctactactgctctagggtgaaacccaaggcgctctagaggagatgaattatggatcc

SEQ ID NO: 46 - DNA, 193 bp, Artificial Sequence
  Synthetic Construct
cacaggcctcataggcaggcttccccatcctggtaaacaacacccacacactttctactactgctctagggtgaaacccaaggcgctctagaggagatgaattatggatcctgagccggcagcctggctccccaccccatgtattattcagctcctgagagccagccagctcctgttacactgaccgcagccc

SEQ ID NO: 47 - DNA, 499 bp, Homo sapiens
tgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatcc

SEQ ID NO: 48 - DNA, 1262 bp, Artificial Sequence
  Synthetic Construct
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttagcctagagtccttggggtctgtcactggccaccctcgcatcccaggctgcaggaaactgaggcccagagaggacaaggactttcctggacccacacagccagtcagtgacagagcctagggtctgagccaggcctgacccaacctccatttctgcctctctacccctgcccccgccccaacacacacacacacacaagtggagttccactgaaacgcccctccttgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 49 - DNA, 649 bp, Artificial Sequence
  Synthetic Construct
gcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcgggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgc

SEQ ID NO: 50 - DNA, 240 bp, Mus musculus
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 51 - DNA, 95 bp, Mus musculus
tttatgaggtgggagctgggctctccctgatgtattattcagctccctggagttggccagctcctgttacactggccacagccctgggcatccgc

SEQ ID NO: 52 - DNA, 185 bp, Mus musculus
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 53 - DNA, 335 bp, Artificial Sequence
  Synthetic Construct
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 54 - DNA, 425 bp, Artificial Sequence
  Synthetic Construct
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 55 - DNA, 280 bp, Mus musculus
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 56 - DNA, 520 bp, Artificial Sequence
  Synthetic Construct
tttatgaggtgggagctgggctctccctgatgtattattcagctccctggagttggccagctcctgttacactggccacagccctgggcatccgcctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 57 - DNA, 520 bp, Artificial Sequence
  Synthetic Construct
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 58 - DNA, 520 bp, Artificial Sequence
  Synthetic Construct
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 59 - DNA, 964 bp, Artificial Sequence
  Synthetic Construct
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 60 - DNA, 519 bp, Artificial Sequence
  Synthetic Construct
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 61 - DNA, 425 bp, Artificial Sequence
  Synthetic Construct
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 62 - DNA, 865 bp, Homo sapiens
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 63 - DNA, 437 bp, Homo sapiens
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtga

SEQ ID NO: 64 - DNA, 428 bp, Homo sapiens
gctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 65 - DNA, 287 bp, Homo sapiens
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 66 - DNA, 290 bp, Homo sapiens
cgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcagg

SEQ ID NO: 67 - DNA, 288 bp, Homo sapiens
cgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 68 - DNA, 84 bp, Artificial Sequence
  Synthetic Construct
gtaagtatcaaggttacaagacaggtttaaggagaccaatagaaactgggcttgtcgagacagagaagactcttgcgtttctga

SEQ ID NO: 69 - DNA, 49 bp, Artificial Sequence
  Synthetic Construct
taggcacctattggtcttactgacatccactttgcctttctctccacag

SEQ ID NO: 70 - DNA, 943 bp, Artificial Sequence
  Synthetic Construct
ggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggca

SEQ ID NO: 71 - DNA, 2406 bp, Homo sapiens
atggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctg

SEQ ID NO: 72 - DNA, 3588 bp, Homo sapiens
gaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatga

SEQ ID NO: 73 - protein, 802 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMREL

SEQ ID NO: 74 - protein, 1195 aa, Homo sapiens
ENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 75 - DNA, 10032 bp, Artificial Sequence
  Synthetic Construct
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccaattctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 76 - DNA, 10461 bp, Artificial Sequence
  Synthetic Construct
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 77 - DNA, 6678 bp, Artificial Sequence
  Synthetic Construct
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccgaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 78 - DNA, 7152 bp, Artificial Sequence
  Synthetic Construct
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 79 - DNA, 10005 bp, Artificial Sequence
  Synthetic Construct
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 80 - DNA, 6653 bp, Artificial Sequence
  Synthetic Construct
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 81 - DNA, 6986 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccaattctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 82 - DNA, 7414 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 83 - DNA, 6959 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 84 - DNA, 953 bp, Artificial Sequence
  Synthetic Construct
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag